(19) United States
(12) Patent Application Publication
(10) Pub. No.: US 2016/0218296 A1
(43) Pub. Date: Jul. 28, 2016

(54) AMINE DERIVATIVES, MATERIAL FOR ORGANIC ELECTROLUMINESCENT DEVICE COMPRISING THE SAME AND ORGANIC ELECTRLUMINESCENT DEVICE USING THE SAME

(71) Applicant: Samsung Display Co., Ltd., Yongin-si (KR)

(72) Inventors: Koushin MATSUOKA, Yokohama (JP); Hiroaki ITOI, Yokohama (JP)

(21) Appl. No.: 14/990,746

(22) Filed: Jan. 7, 2016

(30) Foreign Application Priority Data
Jan. 23, 2015 (JP) 2015-011303

(51) Int. Cl. H01L 51/00; C07D 279/26; C07D 219/14; C07D 265/38

(52) U.S. Cl. CPC H01L 51/0061; C07D 265/38; C07D 279/26; C07D 219/14; H01L 51/5056

(57) ABSTRACT

A novel amine derivative is represented by the following Formula 1:

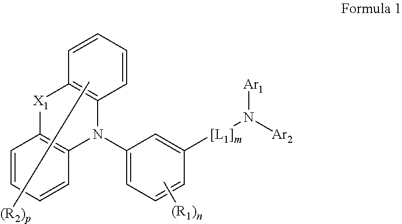

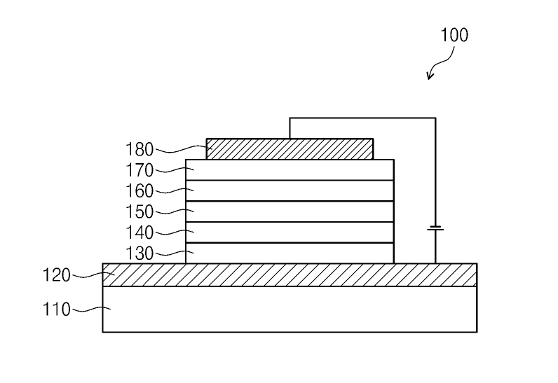

An organic electroluminescent (EL) device includes: an anode, a cathode, an emission layer between the anode and the cathode, and a plurality of layers between the anode and the emission layer, wherein a material for an organic EL device including the amine derivative is in at least one layer selected from the plurality of layers between the anode and the emission layer. In a blue to bluish green region of an organic EL device, the driving voltage of the organic EL device may be decreased, and the emission efficiency thereof may be significantly improved.

### CROSS-REFERENCE TO RELATED APPLICATION

This application claims priority to and the benefit of Japanese Patent Application No. 2015-011303, filed on Jan. 23, 2015, the entire content of which is incorporated herein by reference.

### BACKGROUND

1. Field

Recently, the developments of organic electroluminescent (EL) displays have been actively conducted. Also, the developments of organic EL devices, which are self-luminescent light emitting devices used in the organic EL displays, have been actively conducted.

2. Description of the Related Art

A structure of the organic EL device may include, for example, an anode, a hole transport layer positioned on the anode, an emission layer positioned on the hole transport layer, an electron transport layer positioned on the emission layer and a cathode positioned on the electron transport layer.

In the organic EL device as described above, holes and electrons respectively injected from the anode and the cathode recombine in an emission layer to generate excitons. The emission of light may be then realized via the transition of these generated excitons to a ground state. As a hole transport material used in the hole transport layer, an amine derivative including nitrogen combined (e.g., coupled) with a nitrogen-containing 6-membered ring via a p-biphenylene group or a 2,7-fluorenylene group has been disclosed. An amine derivative including a nitrogen atom combined (e.g., coupled) with a carbon atom of an acridane ring has also been disclosed.

However, organic EL devices using the disclosed amine derivatives as hole transport materials have high driving voltage and low emission efficiency. Thus, a material for decreasing the driving voltage of the organic EL device and improving its emission efficiency is desired.

### SUMMARY

One or more aspects of embodiments of the present disclosure herein relate to amine derivatives, a material for an organic electroluminescent device including the amine derivatives, and an organic electroluminescent device using the material.

According to embodiments of the present disclosure, an amine derivative and a novel and improved material for an organic EL device including the amine derivative may decrease the driving voltage and improve the emission efficiency of an organic EL device using such material.

An embodiment of the present disclosure provides an amine derivative represented by the following Formula 1:

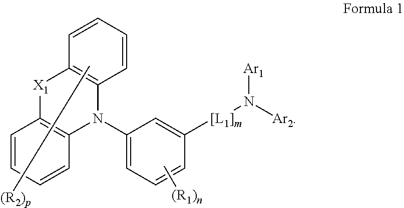

In Formula 1, Ar₁ and Ar₂ may be each independently selected from a substituted or unsubstituted aryl group having 6 to 30 carbon atoms for forming a ring and a substituted or unsubstituted heteroaryl group having 2 to 30 carbon atoms for forming a ring,

L₁ may be a divalent linker,

X₁ may be selected from C(R₃)₂, C═O, Si(R₃)₂, N—R₃, O, S, SO and SO₂,

R₁, R₂ and R₃ may be each independently selected from a substituted or unsubstituted an linear, branched or cyclic alkyl group having 1 to 16 carbon atoms, a substituted or unsubstituted aryl group having 6 to 36 carbon atoms for forming a ring, a substituted or unsubstituted heteroaryl group having 2 to 32 carbon atoms for forming a ring, and an aryl group or a heteroaryl group formed via condensation of a plurality of adjacent substituents,

n may be an integer selected from 0 to 4,

m may be an integer selected from 1 to 4, and

p may be an integer selected from 0 to 8.

According to embodiments of the present disclosure, the driving voltage of the organic EL device may be decreased, and the emission efficiency thereof may be improved by using the amine derivative.

In an embodiment, L₁ may be selected from a substituted or unsubstituted arylene group having 6 to 30 carbon atoms for forming a ring and a substituted or unsubstituted heteroarylene group having 2 to 30 carbon atoms for forming a ring.

According to embodiments of the present disclosure, the driving voltage of the organic EL device may be decreased, and the emission efficiency thereof may be improved by using the amine derivative.

In an embodiment, Ar₁ and Ar₂ may be each independently selected from a group substituted with a substituent not including a nitrogen atom and an unsubstituted aryl group or heteroaryl group, the aryl group or the heteroaryl group not including a nitrogen atom.

According to embodiments of the present disclosure, the driving voltage of the organic EL device may be decreased, and the emission efficiency thereof may be improved by using the amine derivative.

In an embodiment, Ar₁ may be a group substituted with a substituent not including a nitrogen atom or an unsubstituted aryl group or heteroaryl group not including a nitrogen atom, and Ar₂ may be represented by the following Formula 2:

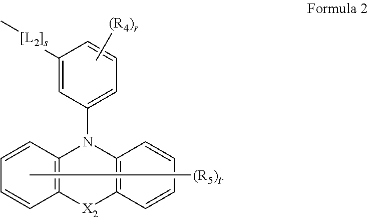

In Formula 2, L₂ may be a divalent linker,

X₂ may be selected from C(R₆)₂, C═O, Si(R₆)₂, N—R₆, O, S, SO and SO₂,

R₄, R₅ and R₆ may be each independently selected from a substituted or unsubstituted linear, branched or cyclic alkyl group having 1 to 16 carbon atoms, a substituted or unsubstituted aryl group having 6 to 36 carbon atoms for forming a ring, a substituted or unsubstituted heteroaryl group having 2 to 32 carbon atoms for forming a ring, and an aryl group or a heteroaryl group formed via condensation of a plurality of adjacent substituents,

r may be an integer selected from 0 to 4,

s may be an integer selected from 1 to 4, and

t may be an integer selected from 0 to 8.

According to embodiments of the present disclosure, the driving voltage of the organic EL device may be decreased, and the emission efficiency thereof may be improved by using the amine derivative.

In an embodiment of the present disclosure, a material for an organic EL device may include an amine derivative represented by the following Formula 1:

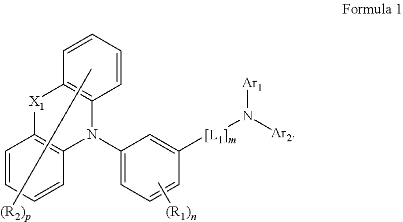

In Formula 1, Ar₁ and Ar₂ may be each independently selected from a substituted or unsubstituted aryl group having 6 to 30 carbon atoms for forming a ring and a substituted or unsubstituted heteroaryl group having 2 to 30 carbon atoms for forming a ring,

L₁ may be a divalent linker,

X₁ may be selected from C(R₃)₂, C═O, Si(R₃)₂, N—R₃, O, S, SO and SO₂,

R₁, R₂ and R₃ may be each independently selected from a substituted or unsubstituted linear, branched or cyclic alkyl group having 1 to 16 carbon atoms, a substituted or unsubstituted aryl group having 6 to 36 carbon atoms for forming a ring, a substituted or unsubstituted heteroaryl group having 2 to 32 carbon atoms for forming a ring, and an aryl group or a heteroaryl group formed via condensation of a plurality of adjacent substituents,

n may be an integer selected from 0 to 4,

m may be an integer selected from 1 to 4, and

p may be an integer selected from 0 to 8.

According to embodiments of the present disclosure, the driving voltage of the organic EL device may be decreased, and the emission efficiency thereof may be improved.

In an embodiment, L₁ may be selected from a substituted or unsubstituted arylene group having 6 to 30 carbon atoms for forming a ring and a substituted or unsubstituted heteroarylene group having 2 to 30 carbon atoms for forming a ring.

According to embodiments of the present disclosure, the driving voltage of the organic EL device may be decreased, and the emission efficiency thereof may be improved.

In an embodiment, Ar₁ and Ar₂ may be each independently selected from a group substituted with a substituent not including a nitrogen atom and an unsubstituted aryl group or heteroaryl group, the aryl group or the heteroaryl group not including a nitrogen atom.

According to embodiments of the present disclosure, the driving voltage of the organic EL device may be decreased, and the emission efficiency thereof may be improved.

In an embodiment, Ar₁ may be a group substituted with a substituent not including a nitrogen atom or an unsubstituted aryl group or heteroaryl group not including a nitrogen atom, and Ar₂ may be represented by the following Formula 2:

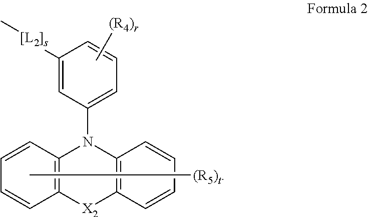

In Formula 2, L₂ may be a divalent linker,

X₂ may be selected from C(R₆)₂, C═O, Si(R₆)₂, N—R₆, O, S, SO and SO₂,

R₄, R₅ and R₆ may be each independently selected from a substituted or unsubstituted linear, branched or cyclic alkyl group having 1 to 16 carbon atoms, a substituted or unsubstituted aryl group having 6 to 36 carbon atoms for forming a ring, a substituted or unsubstituted heteroaryl group having 2 to 32 carbon atoms for forming a ring, and an aryl group or a heteroaryl group formed via condensation of a plurality of adjacent substituents,

r may be an integer selected from 0 to 4,

s may be an integer selected from 1 to 4, and

t may be an integer selected from 0 to 8.

According to embodiments of the present disclosure, the driving voltage of the organic EL device may be decreased, and the emission efficiency thereof may be improved.

In an embodiment of the present disclosure, an organic EL device includes an anode, a cathode, an emission layer between the anode and the cathode, and a plurality of layers between the anode and the emission layer, and the material for an organic EL device is in at least one layer selected from the plurality of layers between the anode and the emission layer.

According to embodiments of the present disclosure, the driving voltage of the organic EL device may be decreased, and the emission efficiency thereof may be improved.

As described above, the driving voltage of the organic EL device may be decreased, and the emission efficiency thereof may be improved according to the present disclosure.

### BRIEF DESCRIPTION OF THE DRAWINGS

The accompanying drawing is included to provide a further understanding of the present disclosure, and is incorporated in and constitutes a part of this specification. The drawing illustrates example embodiments of the present disclosure and, together with the description, serves to explain principles of the present disclosure. The drawing is a cross-sectional view illustrating the schematic configuration of an organic EL device according to an embodiment of the present disclosure.

### DETAILED DESCRIPTION

Example embodiments of the present disclosure will be described below in more detail with reference to the accompanying drawing. In the specification and drawing, elements having substantially the same function will be designated by the same reference numerals, and repeated explanations thereof will not be provided.

### 1. Configuration of Amine Derivative and Material for Organic EL Device Including the Same

An amine derivative and a material for an organic EL device according to embodiments of the present disclosure may decrease the driving voltage and improve the emission efficiency of an organic EL device. In embodiments where the material for an organic EL device including the amine derivative is used as a hole transport material, the driving voltage of the organic EL device may be decreased and the emission efficiency thereof may be improved. First, the configurations of an amine derivative and a material for an organic EL device including the same according to an embodiment will be explained. The amine derivative according to an embodiment of the present disclosure is represented by the following Formula 1, and the material for an organic EL device according to an embodiment of the present disclosure includes the amine derivative represented by the following Formula 1:

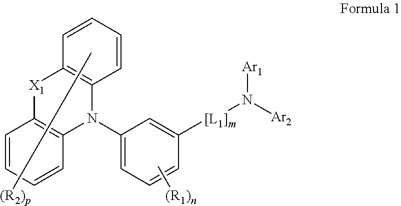

In the amine derivative represented by Formula 1, the nitrogen atom of an amine moiety is combined (e.g., coupled) with a nitrogen-containing 6-membered condensed structure (such as phenoxazine, phenothiazine, acridane, and/or acridone) via an m-phenylene group.

In the above Formula 1, Ar₁ and Ar₂ may be each independently selected from a substituted or unsubstituted aryl group having 6 to 30 carbon atoms for forming a ring and a substituted or unsubstituted heteroaryl group having 2 to 30 carbon atoms for forming a ring. In some embodiments, Ar₁ and Ar₂ may be the same as or different from each other, and may combine (e.g., be coupled) to each other to form a ring. As used herein, the expression “atoms for forming ring” may refer to “ring-forming atoms.”

For example, Ar₁ and Ar₂ may each independently be an aryl group or a heteroaryl group including a substituent that does not include a nitrogen atom. In some embodiments, Ar₁ and Ar₂ may be a group substituted with a substituent that does not include a nitrogen atom or Ar₁ and Ar₂ may each independently be an unsubstituted aryl group or a heteroaryl group, and the aryl group or the heteroaryl group may not include a nitrogen atom.

In Formula 1, non-limiting examples of Ar₁ and Ar₂ may include a substituted or unsubstituted phenyl group, biphenyl group, terphenyl group, napthyl group, anthryl group, phenanthrenyl group, fluorenyl group, indenyl group, pyrenyl group, fluoranthenyl group, triphenylenyl group, perylenyl group, naphthylphenyl group, biphenylenyl group, and/or the like.

In some embodiments, in Formula 1, non-limiting examples of Ar₁ and Ar₂ may include a substituted or unsubstituted pyridyl group, quinolyl group, isoquinolyl group, indolyl group, bezoxazolyl group, benzothiazolyl group, quinoxalyl group, benzoimidazolyl group, indazolyl group, benzofuranyl group, isobenzofuranyl group, dibenzofuranyl group, phenoxazinyl group, phenothiazinyl group, acridinyl group, phenazinyl group, benzothiophenyl group, dibenzothiophenyl group, phenazasilinyl group, and/or the like.

In Formula 1, the substituent of the aryl group and the heteroaryl group forming Ar₁ and Ar₂ may include an aryl group and/or a heteroaryl group having 1 to 20 carbon atoms for forming a ring and may be selected from the example groups provided above in connection with the aryl group and the heteroaryl group for forming Ar₁ and Ar₂. In some embodiments, the substituent of Ar₁ and Ar₂ may be selected from an alkyl group (e.g., a methyl group, an ethyl group, a tert-butyl group, and/or the like), an alkoxy group, an aryloxy group, an alkylthio group, an arylthio group, a dialkylamino group, a diarylamino group, a silyl group (e.g., a trialkylsilyl group, an alkyldiarylsilyl group, a dialkylarylsilyl group, a triarylsilyl group, and/or the like), and/or the like. The substituents of Ar₁ and Ar₂ may each independently be substituted with the same functional group as the one forming the substituent or may combine (e.g., may be coupled) to each other to form a ring.

In Formula 1, L₁ may be a divalent linker. As the divalent linker, for example, an alkylene group, an alkenylene group, an alkynylene group, an arylene group, a heteroarylene group, —O—, —S—, —SO—, —SO₂, —CO—, —NR₉—, —SiR₉R₉—, and/or the like may be used, without limitation. Here, R₉ may be selected from a substituted or unsubstituted linear, branched or cyclic alkyl group having 1 to 16 carbon atoms, a substituted or unsubstituted aryl group having 6 to 36 carbon atoms for forming a ring, a substituted or unsubstituted heteroaryl group having 2 to 32 carbon atoms for forming a ring, and an aryl group or a heteroaryl group formed by condensation of a plurality of adjacent substituents. For example, adjacent R₉(s) may be coupled to each other to form an aryl or a heteroaryl group. Descriptions of the functional groups included in R₉ are the same as those provided in connection with R₁.

As the alkylene group, for example, a linear, branched or cyclic alkylene group having 1 to 16 carbon atoms, for example, a methylene group, an ethylene group, a dimethylmethylene group, a 1,4-cyclohexylene group, and/or the like may be used.

The alkenylene group may include, for example, a linear, branched or cyclic alkenylene group having 2 to 16 carbon atoms, for example, a vinylene group, a butadienylene group, a 1,2-cyclohexenylene group, and/or the like.

The alkynylene group may include a linear, branched or cyclic alkynylene group having 2 to 4 carbon atoms, for example, an acetylenylene group, a diacetylenylene group, and/or the like.

As the arylene group, a substituted or unsubstituted arylene group having 6 to 30 carbon atoms for forming a ring may be used. As the heteroarylene group, a substituted or unsubstituted heteroarylene group having 2 to 30 carbon atoms for forming a ring may be used. The substituted or unsubstituted arylene group and heteroarylene group may each independently include a divalent group formed by removing one more hydrogen atom from any one of the aryl groups and/or the heteroaryl groups described in connection with Ar₁ and Ar₂. For example, a phenylene group, a biphenylene group, a naphthylene group, a pyridylene group, a quinolylene group, and/or the like may be used. The substituents of the arylene group and the heteroarylene group may be each independently described the same as the substituents of the aryl group and the heteroaryl group for forming Ar₁ and Ar₂.

For example, L₁ may be selected from an alkylene group, an alkenylene group, an alkynylene group, an arylene group, a heteroarylene group, —O—, —S—, —CO—, and —NR₇—, and in some embodiments, may be selected from the alkenylene group, the alkynylene group, the arylene group, the heteroarylene group, —O—, —S—, and —NR₇—. For example, a substituted or unsubstituted arylene group having 6 to 30 carbon atoms for forming a ring, or a substituted or unsubstituted heteroarylene group having 2 to 30 carbon atoms for forming a ring, may be used.

In Formula 1, X₁ may be selected from C(R₃)₂, C═O, Si(R₃)₂, N—R₃, O, S, SO, and SO₂. In C(R₃)₂ and Si(R₃)₂, a plurality of R₃(s) may be the same as or different from each other, and adjacent R₃(s) may combine (e.g., may be coupled) to each other to form a ring.

In Formula 1, R₁, R₂ and R₃ may be each independently selected from a substituted or unsubstituted linear, branched or cyclic alkyl group having 1 to 16 carbon atoms, a substituted or unsubstituted aryl group having 6 to 36 carbon atoms for forming a ring, a substituted or unsubstituted heteroaryl group having 2 to 32 carbon atoms for forming a ring, and an aryl group or heteroaryl group formed via condensation of a plurality of adjacent substituents. For example, adjacent R₁(s) may be coupled to each other to form an aryl or a heteroaryl group, adjacent R₂(s) may be coupled to each other to form an aryl or a heteroaryl group, and adjacent R₃(s) may be coupled to each other to form an aryl or a heteroaryl group.

The alkyl group having 1 to 16 carbon atoms may be a linear alkyl group (e.g., a methyl group, an ethyl group, a propyl group, a butyl group, an octyl group, a decyl group, a pentadecyl group, and/or the like), a branched alkyl group (e.g., a tert-butyl group and/or the like), or a cyclic alkyl group (e.g., a cyclopropyl group, a cyclobutyl group, a cyclopentyl group, a cyclohexyl group, a cyclooctyl group, and/or the like).

As the aryl group having 6 to 36 carbon atoms for forming a ring and the heteroaryl group having 2 to 32 carbon atoms for forming a ring, example functional groups described in connection with the aryl group and the heteroaryl group for forming Ar₁ and Ar₂ may be used, for example, a benzoheptaphenyl group may be used.

In addition, n may be an integer from 0 to 4, m may be an integer from 1 to 4, and p may be an integer from 0 to 8. When n, m and p are each independently greater than or equal to 2, a plurality of R₁(s), a plurality of L₁(s), and a plurality of R₂(s) may be respectively the same as or different from each other. In some embodiments, m may be 1 or 2, and in some embodiments, m may be 1. In some embodiments, n is an integer selected from 0 to 2, and in some embodiments, n is 0. In some embodiments, p is an integer selected from 0 to 2, and in some embodiments, p is 0.

In Formula 1, Ar₁ may be a group substituted with a substituent not including a nitrogen atom or Ar₁ may be an unsubstituted aryl group or a heteroaryl group not including a nitrogen atom, and Ar₂ may be represented by the following Formula 2:

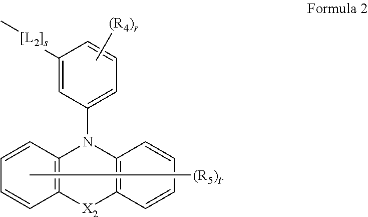

Ar₁ may be an aryl group or a heteroaryl group not including a nitrogen atom selected from among the example aryl groups and the heteroaryl groups for forming Ar₁ and Ar₂ in Formula 1. The substituent of Ar₁ may be a substituent not including a nitrogen atom selected from among the example substituents of the aryl group and the heteroaryl group for forming Ar₁ and Ar₂ in Formula 1.

In Formula 2, L₂ is a divalent linker, and may include, for example, an alkylene group, an alkenylene group, an alkynylene group, an arylene group, a heteroarylene group, —O—, —S—, —SO—, —SO₂—, —CO—, —NR₇—, —SiR₇R₇—, and/or the like, without limitation, similar to L₁ in Formula 1. Descriptions of the functional groups included in L₂ may be the same as those provided in connection with L₁, and thus detailed descriptions thereof will not be provided again. In some embodiments, L₂ may be the alkylene group, the alkenylene group, the alkynylene group, the arylene group, the heteroarylene group, —O—, —S—, —CO—, or —CR₇—, and in some embodiments, may be the alkenylene group, the alkynylene group, the arylene group, the heteroarylene group, —O—, —S—, or —CR₇—. For example, L₂ may be a substituted or unsubstituted arylene group having 6 to 30 carbon atoms for forming a ring, or a substituted or unsubstituted heteroarylene group having 2 to 30 carbon atoms for forming a ring.

In Formula 2, X₂ may be selected from C(R₆)₂, C═O, Si(R₆)₂, N—R₆, O, S, SO and SO₂. In C(R₆)₂ and Si(R₆)₂, a plurality of R₆(s) may be the same as or different from each other, and adjacent R₆(s) may combine (e.g., may be coupled) to each other to form a ring.

In Formula 2, R₄, R₅ and R₆ may be each independently selected from a substituted or unsubstituted linear, branched or cyclic alkyl group having 1 to 16 carbon atoms, a substituted or unsubstituted aryl group having 6 to 36 carbon atoms for forming a ring, a substituted or unsubstituted heteroaryl group having 2 to 32 carbon atoms for forming a ring, and an aryl group or a heteroaryl group formed via condensation of a plurality of adjacent substituents. For example, adjacent R₄(s) may be coupled to each other to form an aryl or a heteroaryl group, adjacent R₅(s) may be coupled to each other to form an aryl or a heteroaryl group, and adjacent R₆(s) may be coupled to each other to form an aryl or a heteroaryl group.

Examples of the alkyl group having 1 to 16 carbon atoms, the aryl group having 6 to 36 carbon atoms for forming a ring and the heteroaryl group having 2 to 32 carbon atoms for forming a ring, may be the same groups as those provided in connection with R₁, R₂ and R₃ in Formula 1.

In addition, r may be an integer selected from 0 to 4, s may be an integer selected from 1 to 4, and t may be an integer selected from 0 to 8. When r, s and t are each independently greater than or equal to 2, a plurality of R₄(s), a plurality of L₂(s), and a plurality of R₅(s), may be respectively the same as or different from each other. In some embodiments, r in Formula 2 may be the same as n in Formula 1, s in Formula 2 may be the same as m in Formula 1, and t in Formula 2 may be same as p in Formula 1 (s=m, r=n and t=p).

In embodiments where an emission layer includes a blue luminescent material or a green luminescent material, the amine derivative represented by Formula 1 according to an embodiment of the present disclosure may further improve the emission efficiency of the organic EL device.

The material for an organic EL device including the amine derivative represented by Formula 1 according to an embodiment of the present disclosure may be included in at least one layer disposed (e.g., positioned) between an emission layer and an anode in an organic EL device. For example, the material for an organic EL device including the amine derivative represented by Formula 1 may be included in the hole transport layer and the hole injection layer of the organic EL device. When the amine derivative according to an embodiment of the present disclosure is used in the hole transport layer, the hole transport layer may be near (e.g., adjacent to) the emission layer or the amine derivative may be used in a stacking layer adjacent to the emission layer in a multilayered structure of the hole transport layer. However, in the organic EL device, a layer including the amine derivative represented by Formula 1 is not limited to the above-described layers. For example, the amine derivative represented by Formula 1 may be included in one of organic layers disposed between an anode and a cathode in an organic EL device, and in some embodiments, may be included in an emission layer.

In this disclosure, “branched” alkyl may refer to a branched alkyl group having 3 and more carbon atoms.

The organic EL device using the material for an organic EL device having the above-mentioned configuration may have decreased driving voltage and significantly improved emission efficiency in the organic EL device. Non-limiting examples of the amine derivative included in the material for an organic EL device will be illustrated hereinafter. However, the amine derivative according to an embodiment of the present disclosure is not limited to the following compounds. In some embodiments, the amine derivative represented by Formula 1 may be represented by at least one selected from the following Compounds 1 to 208, without limitation:

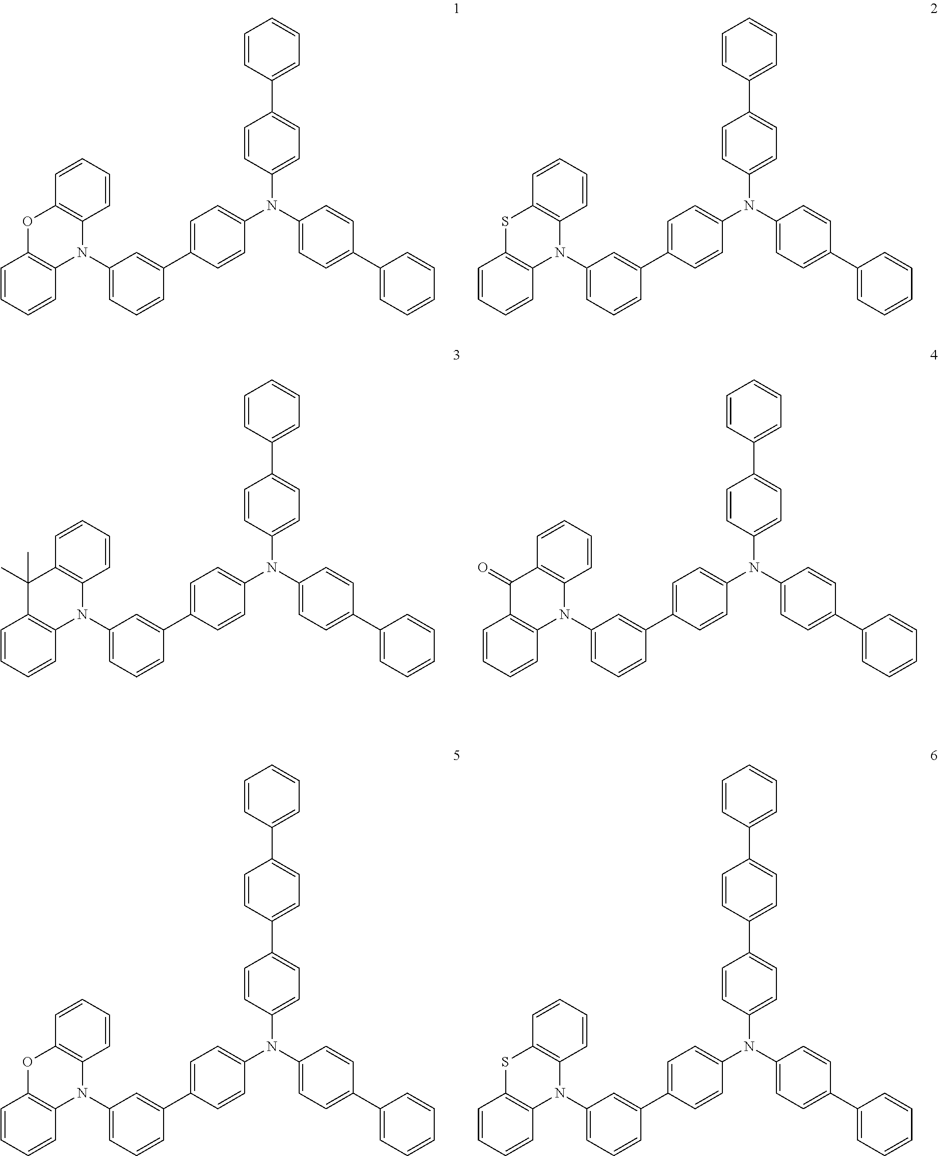

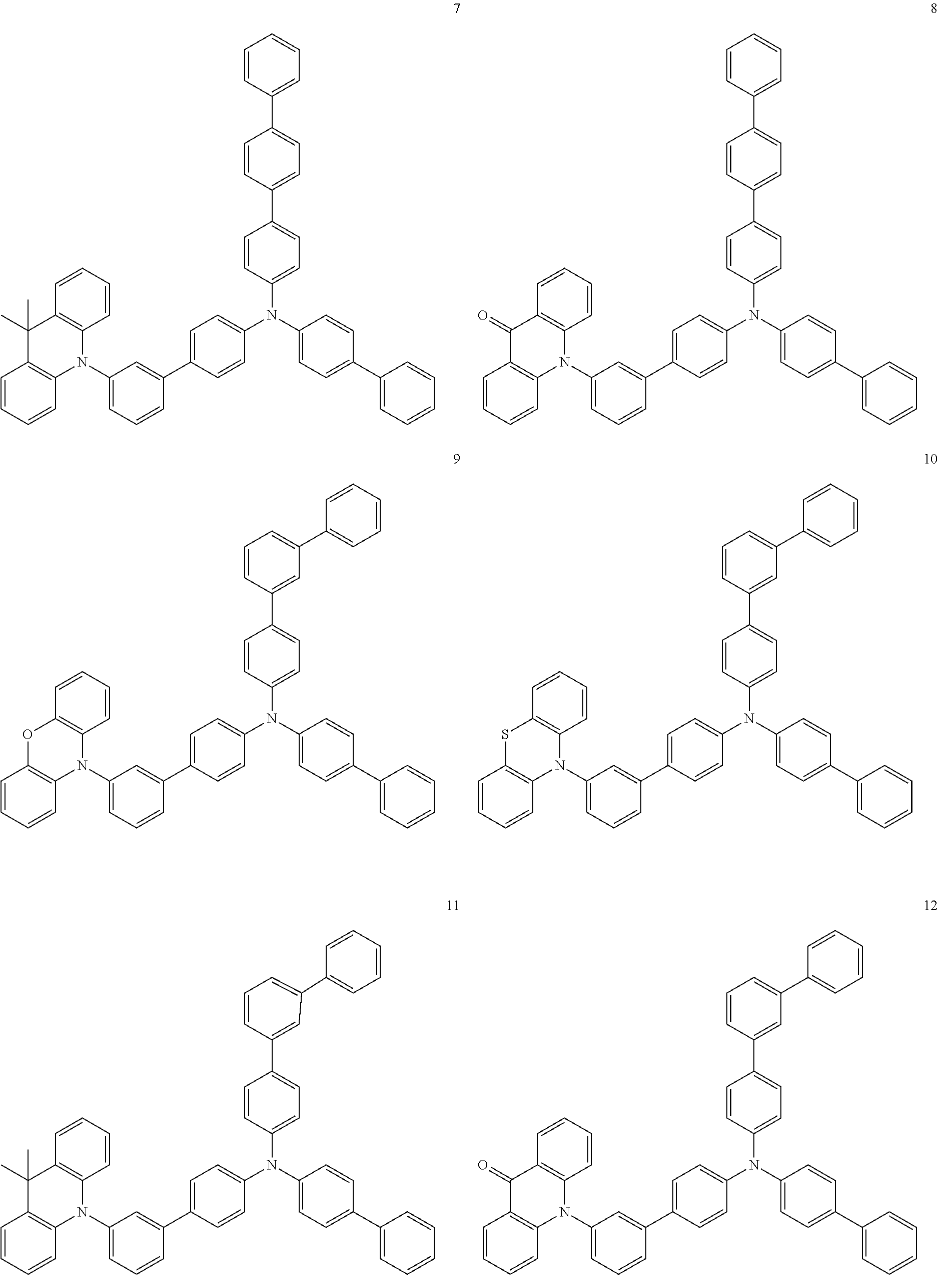

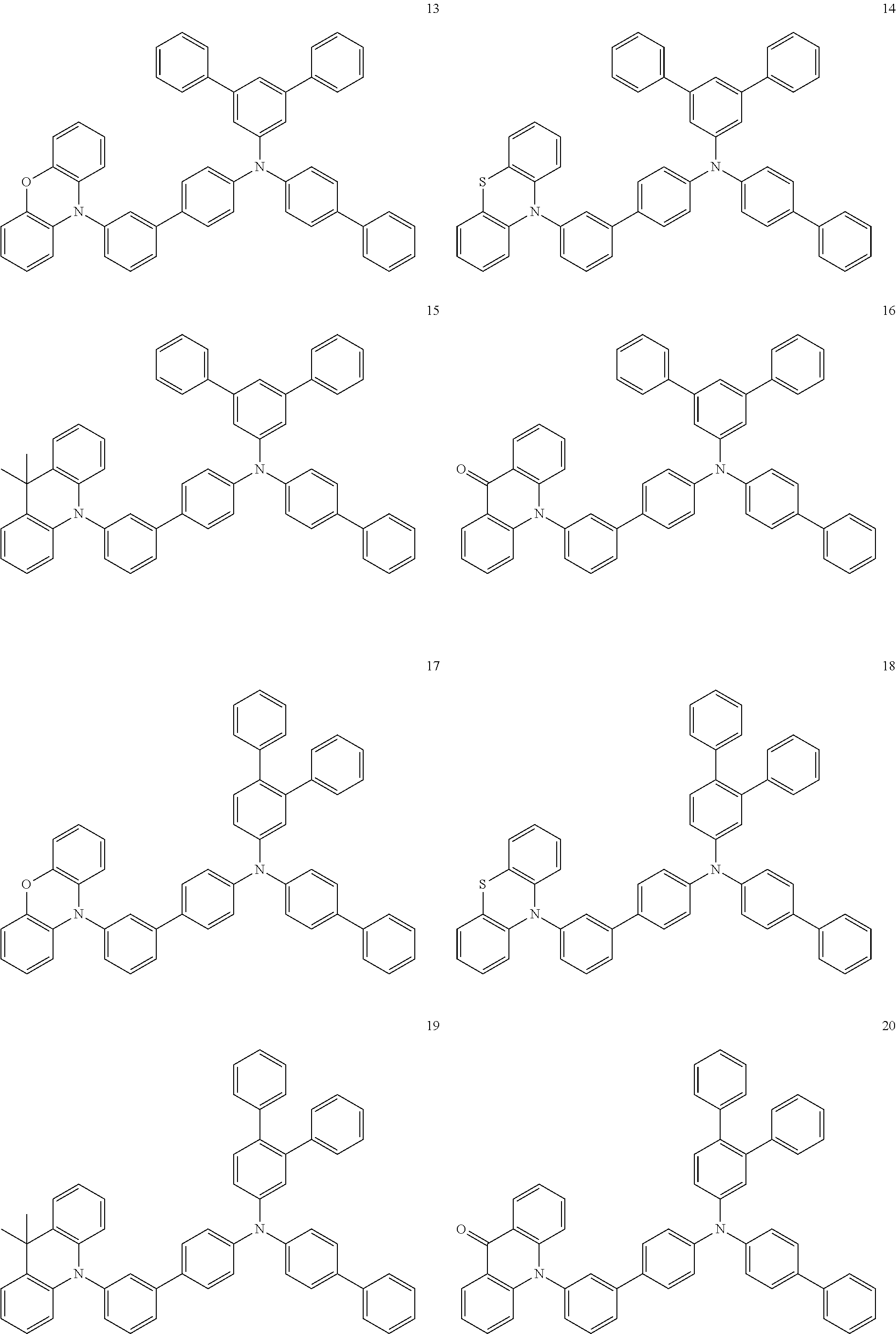

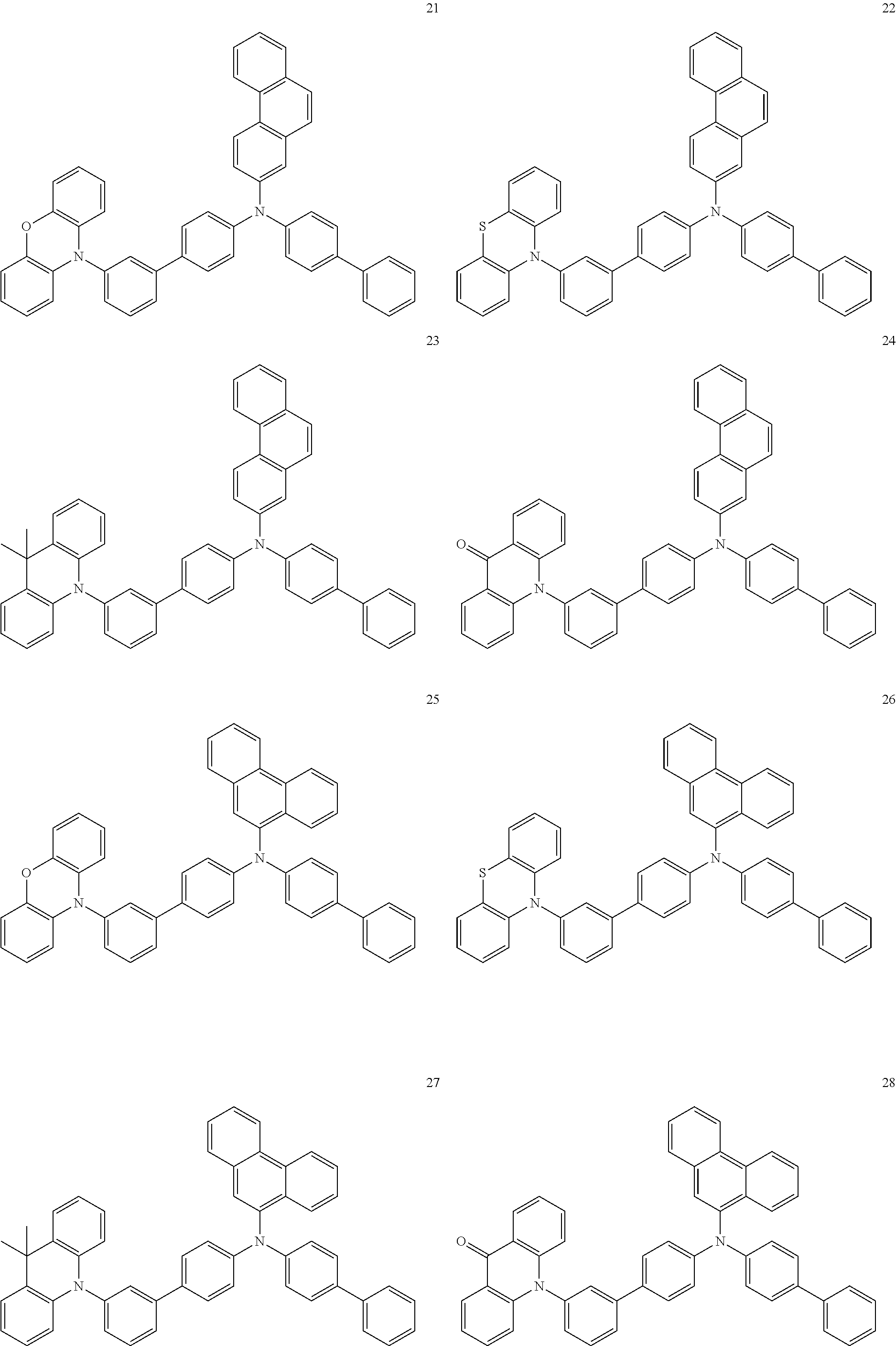

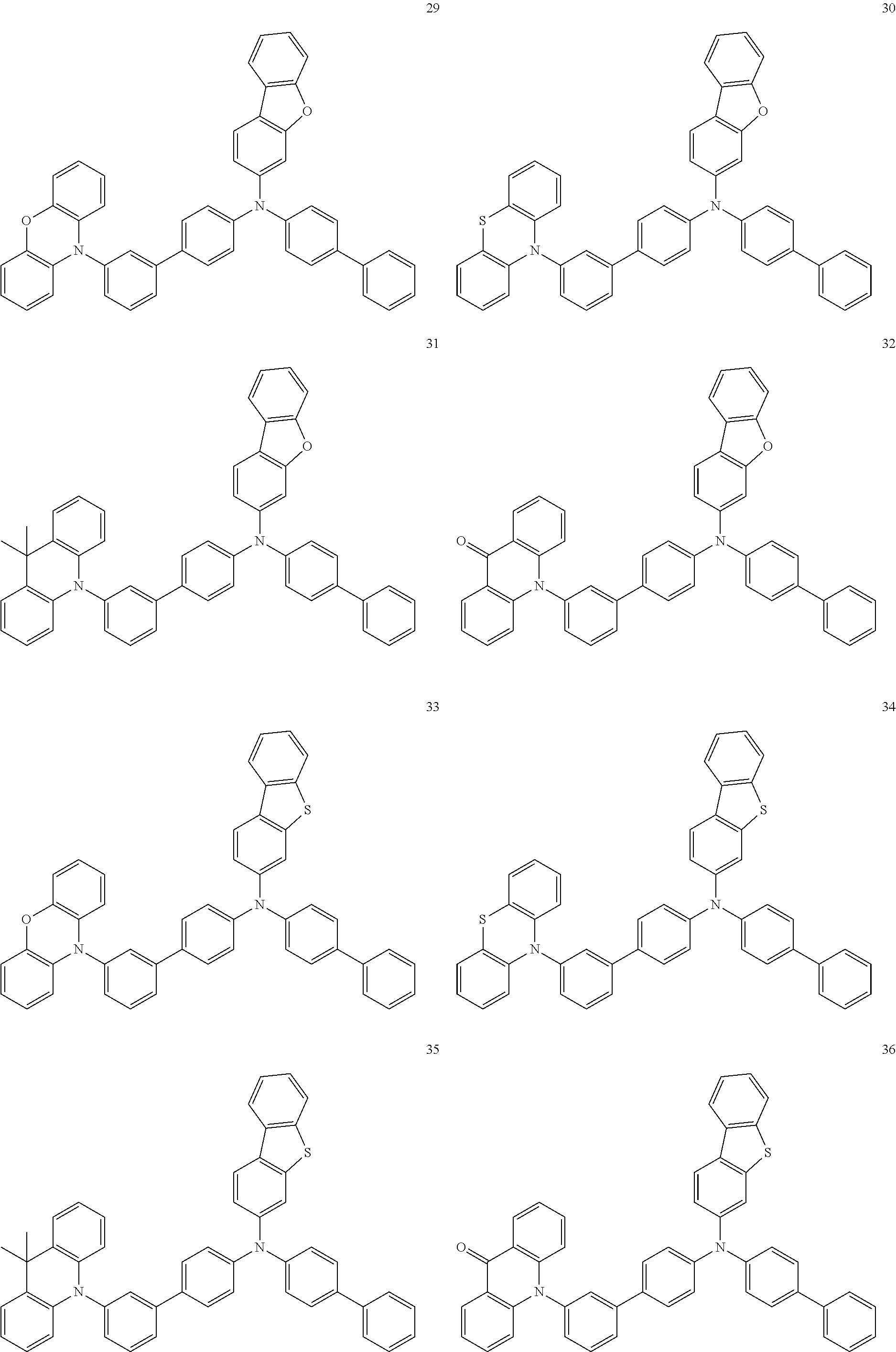

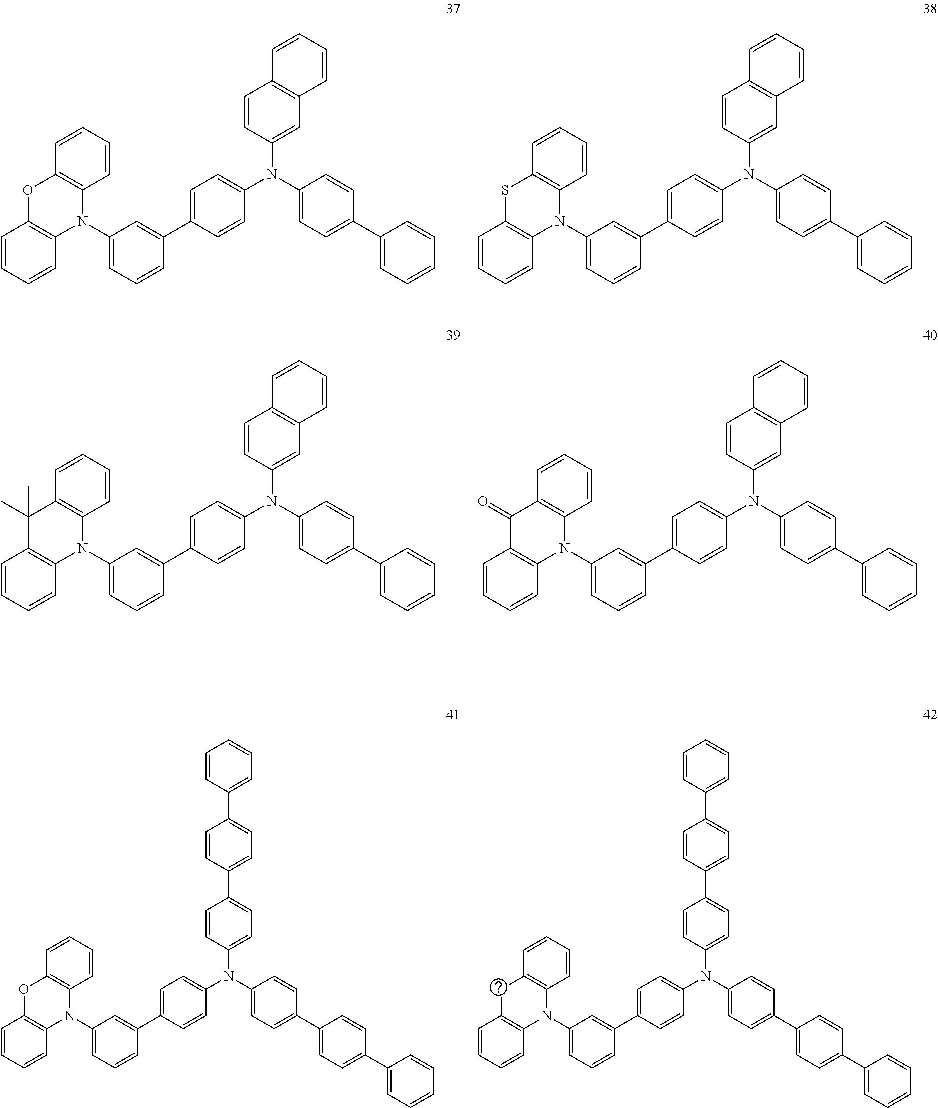

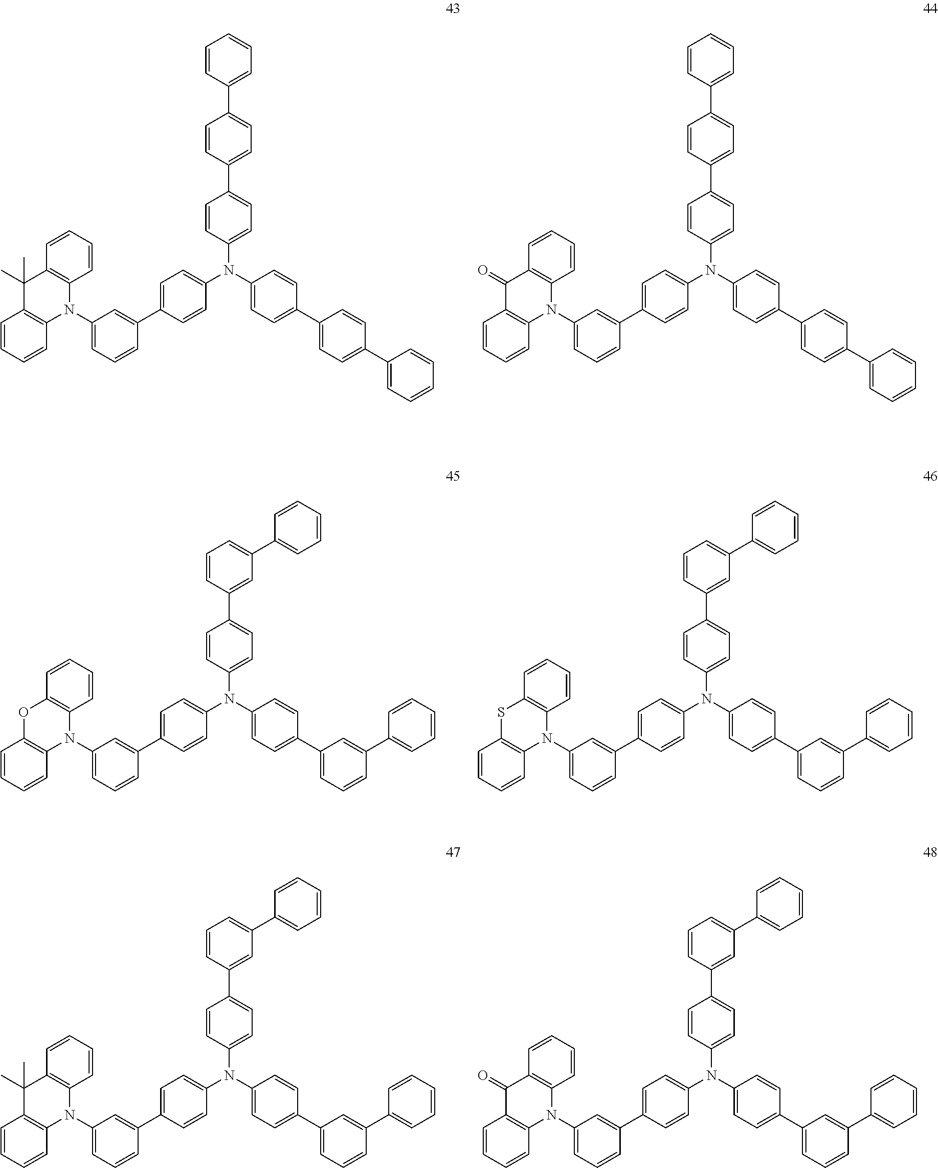

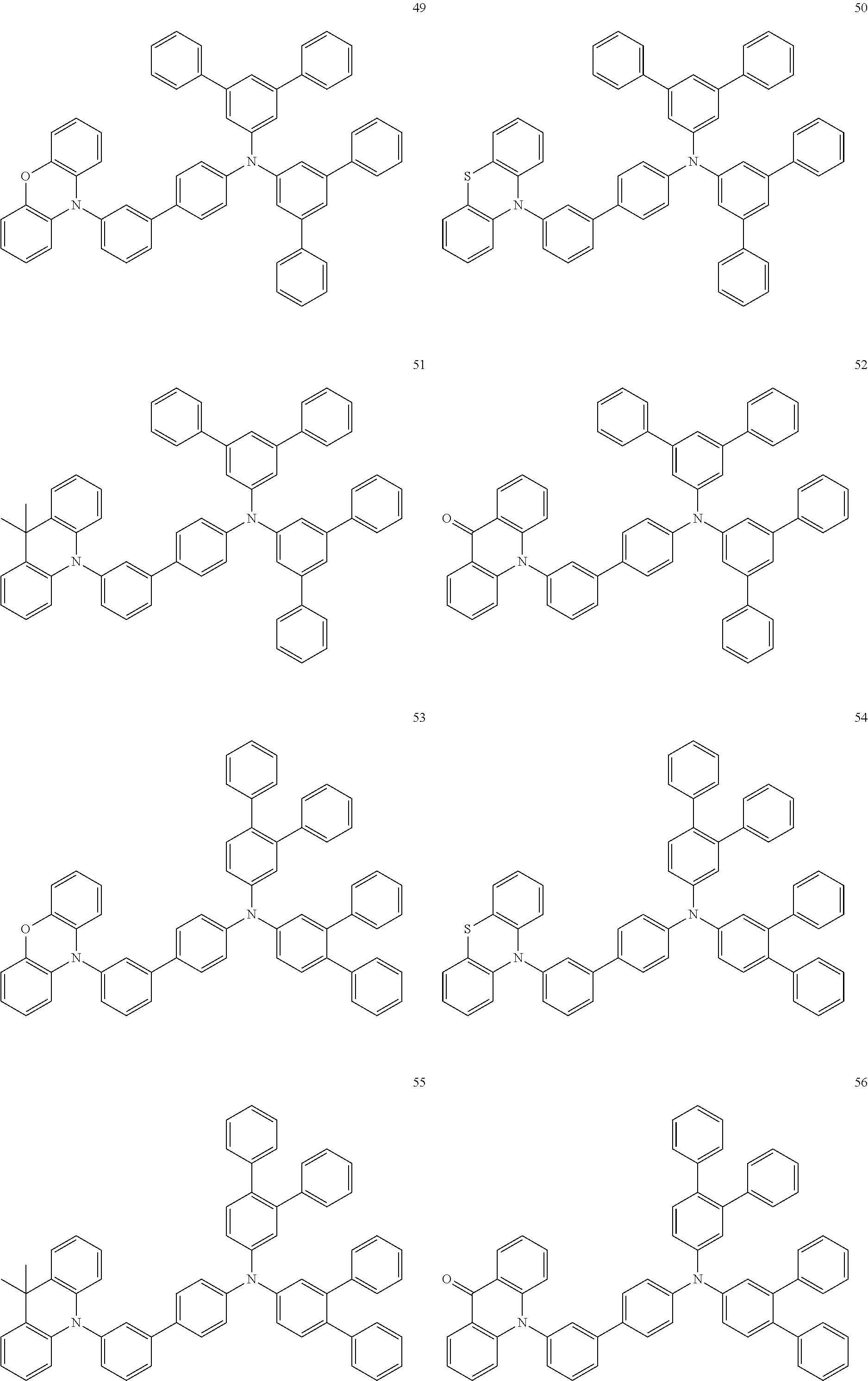

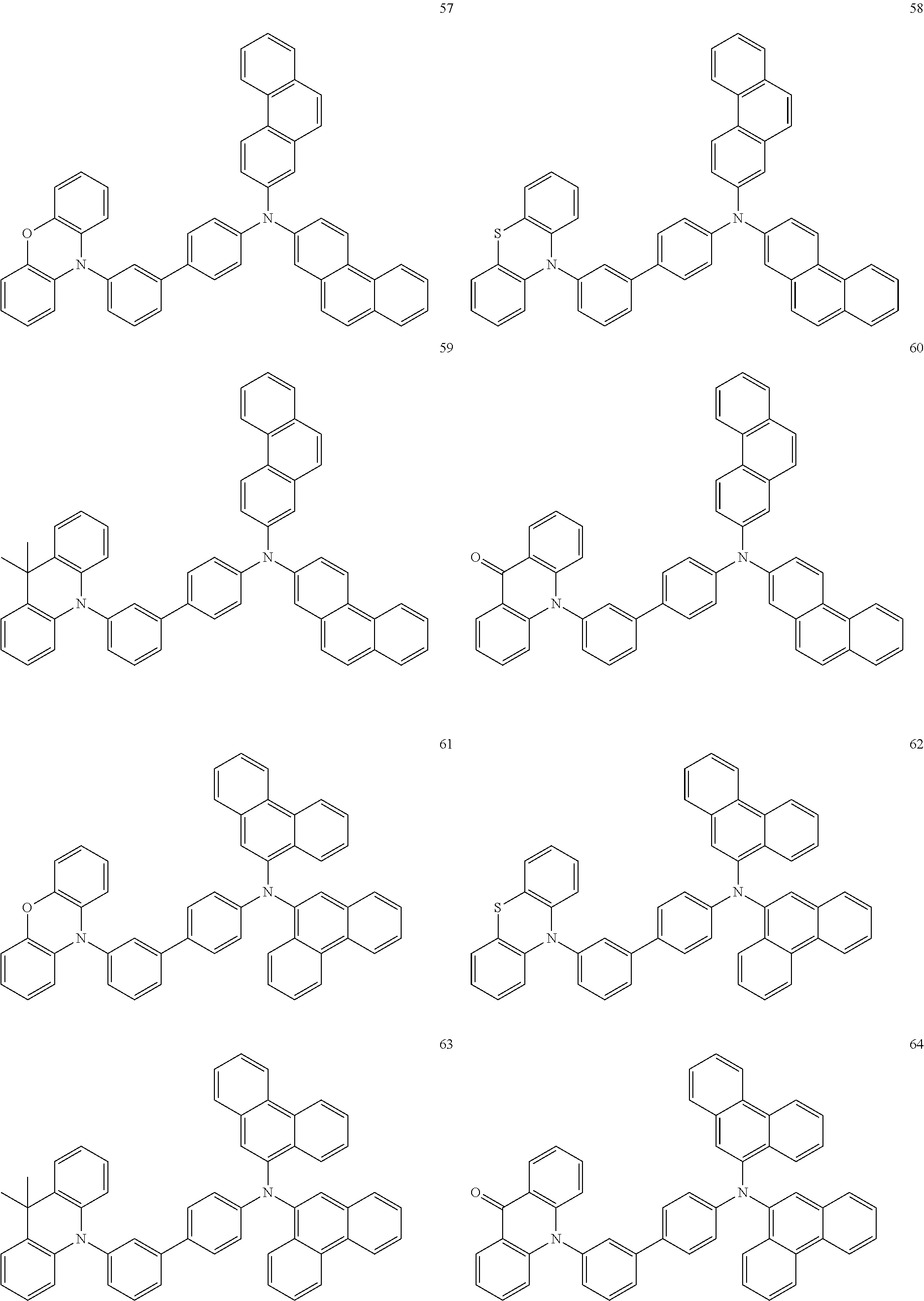

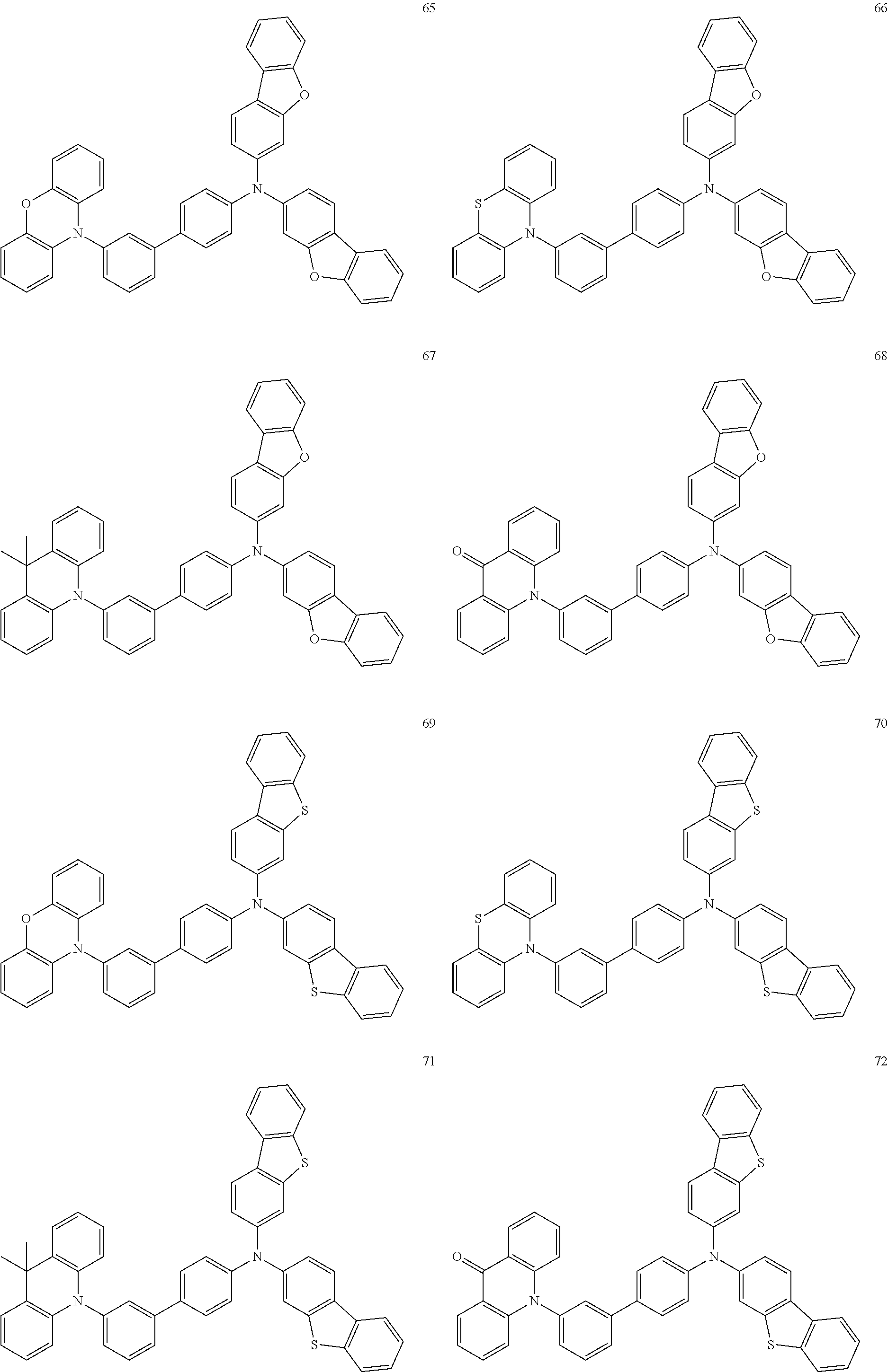

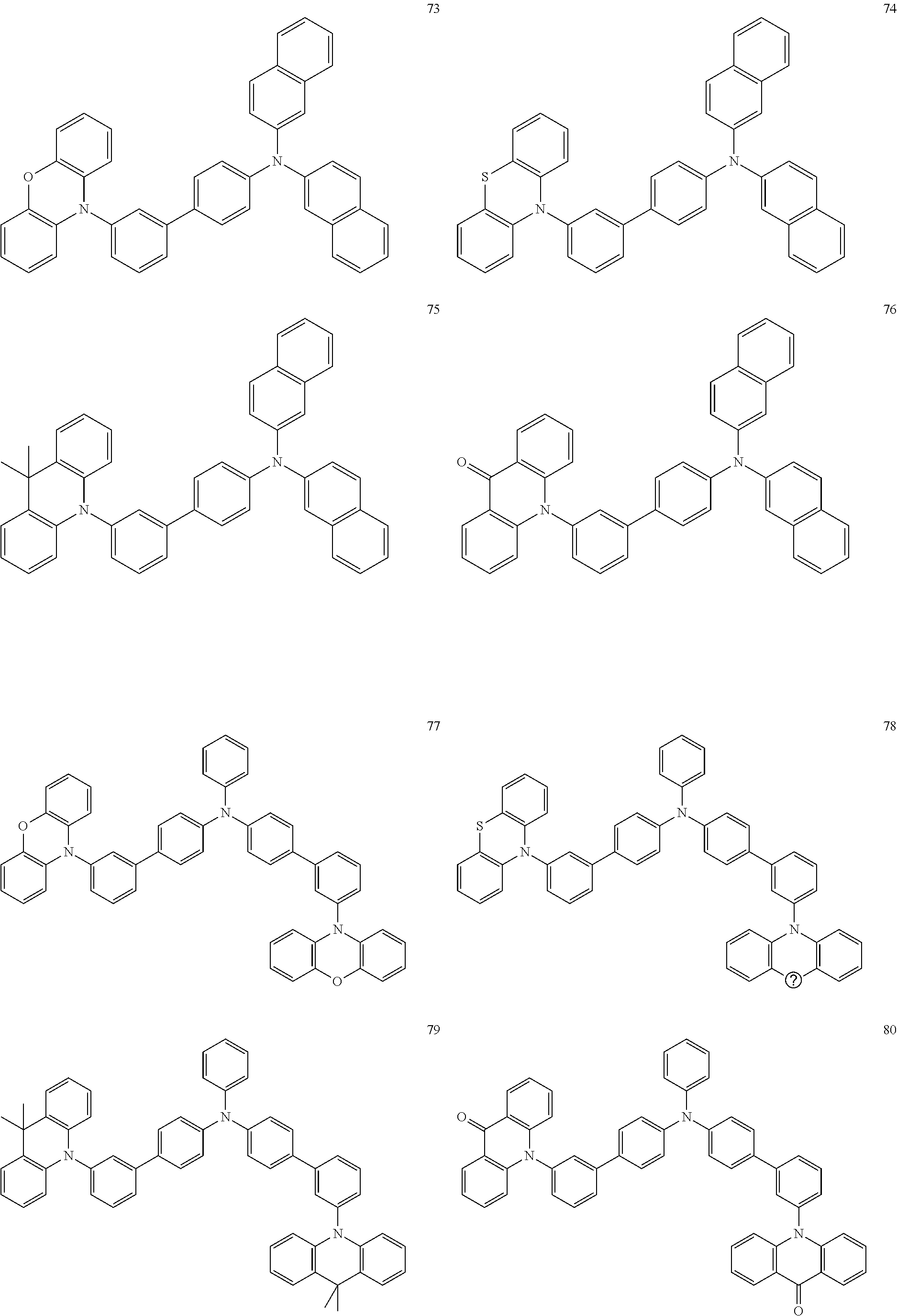

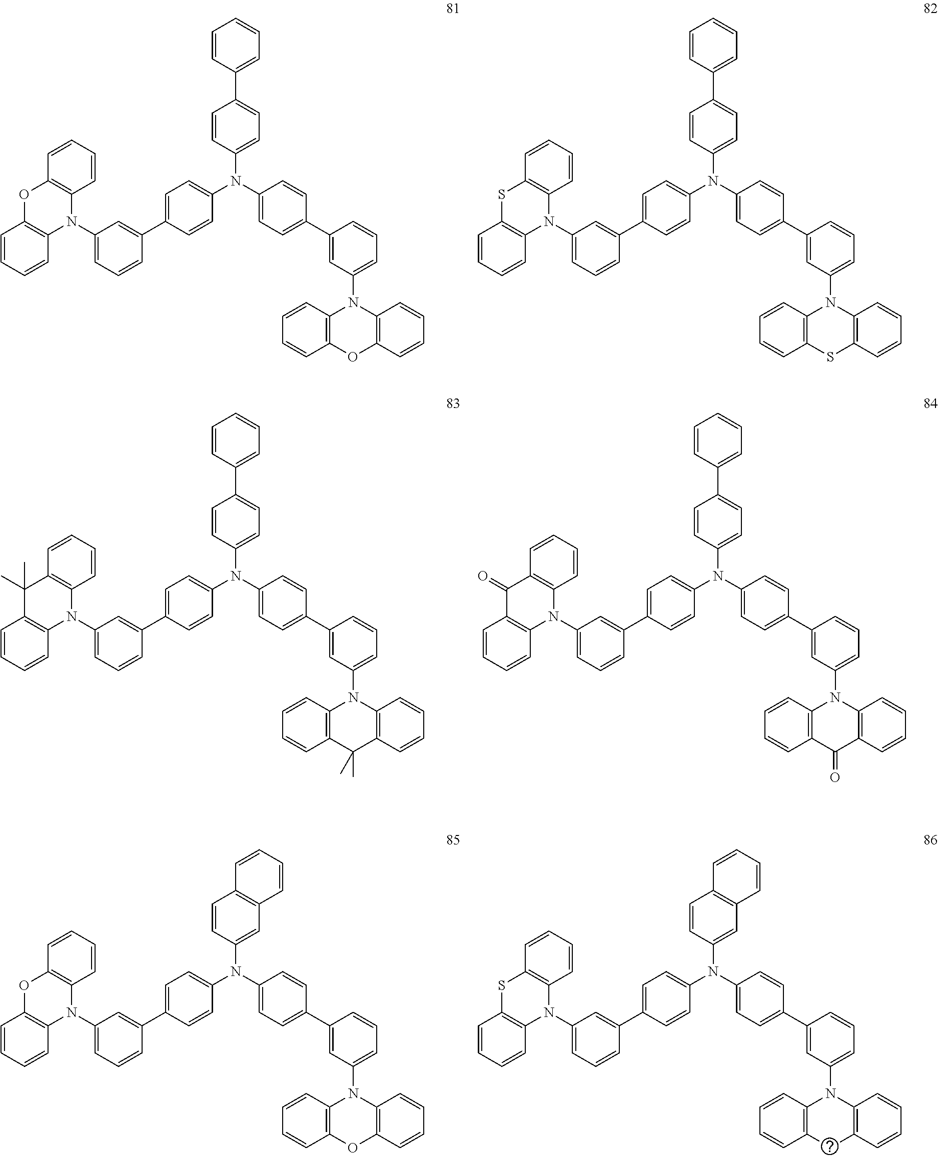

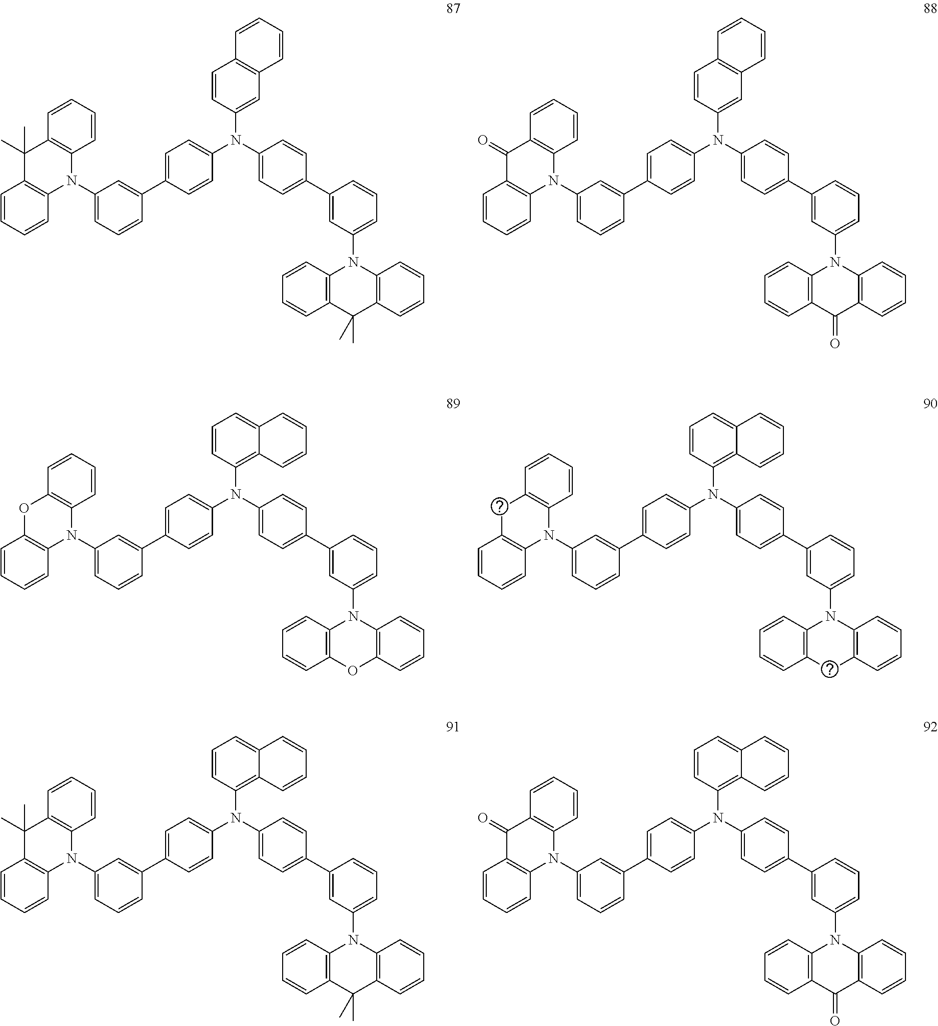

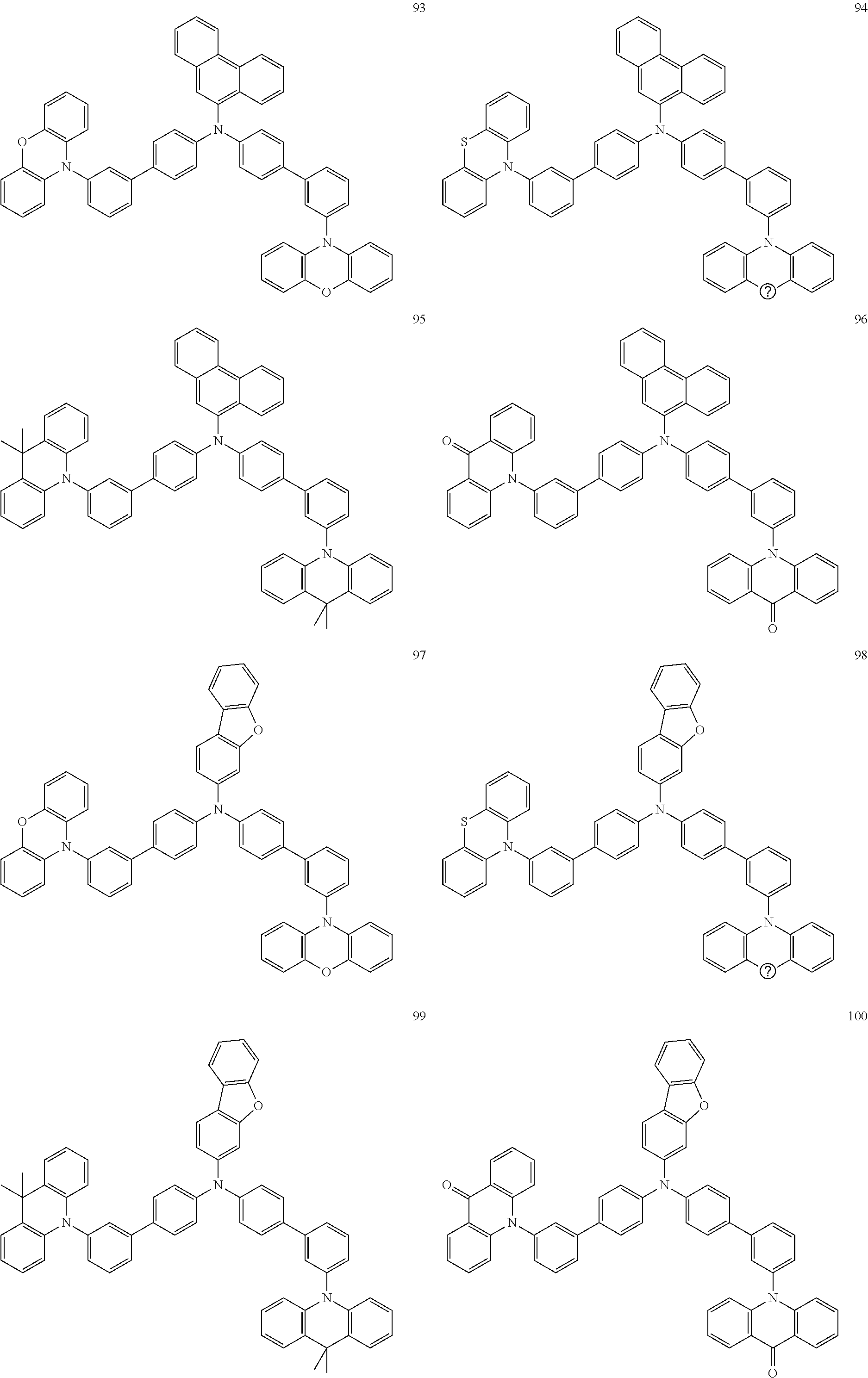

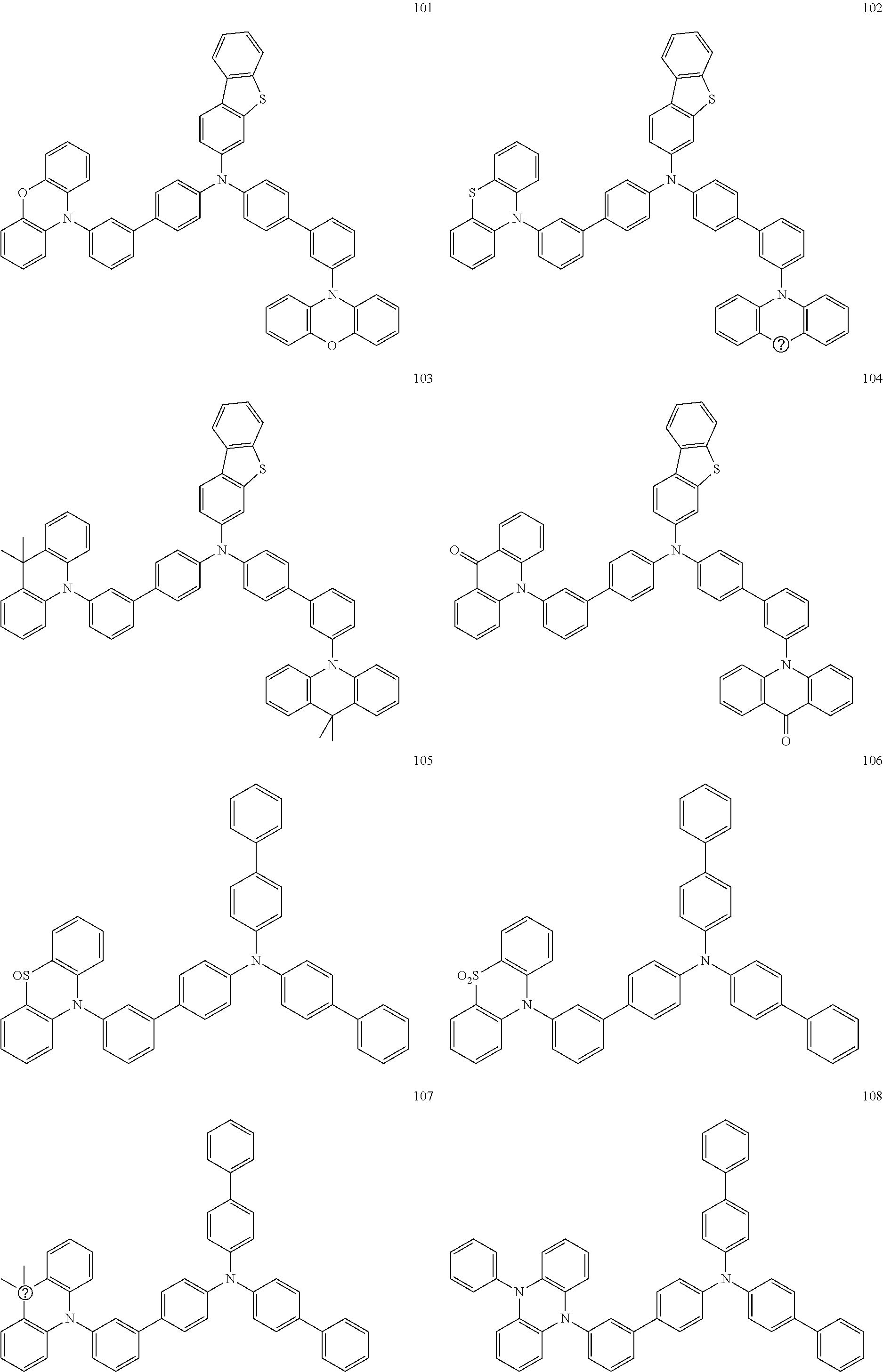

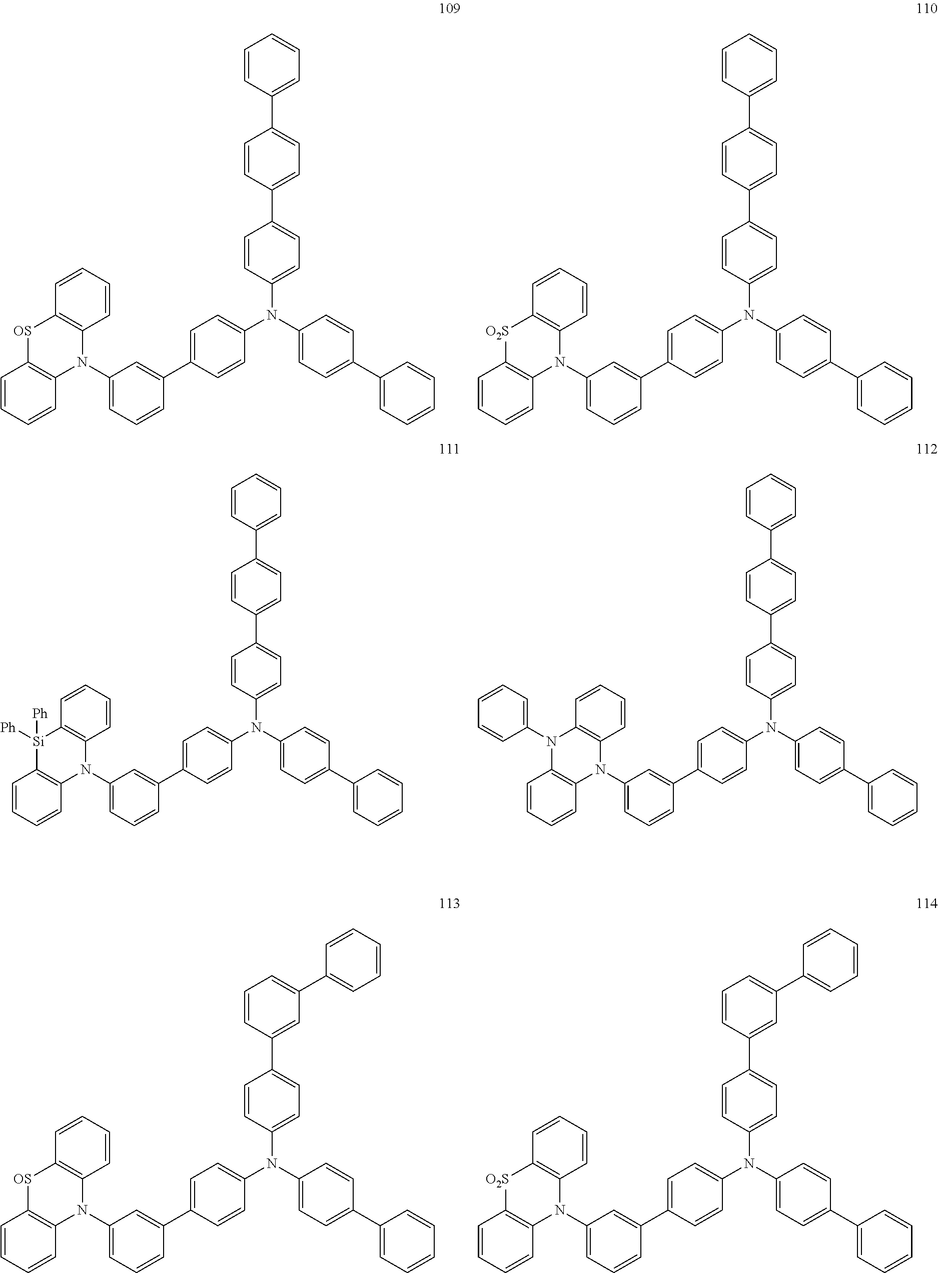

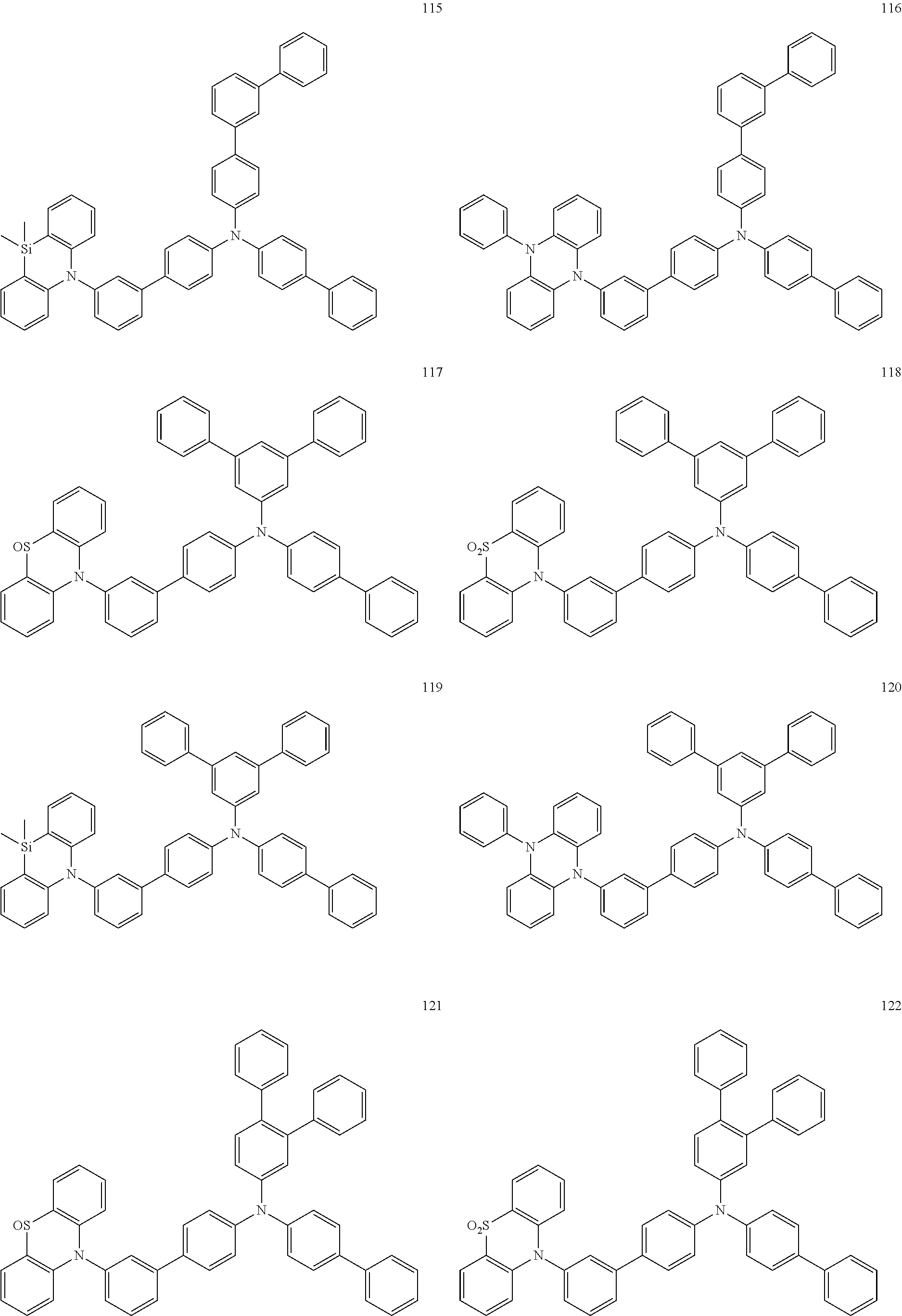

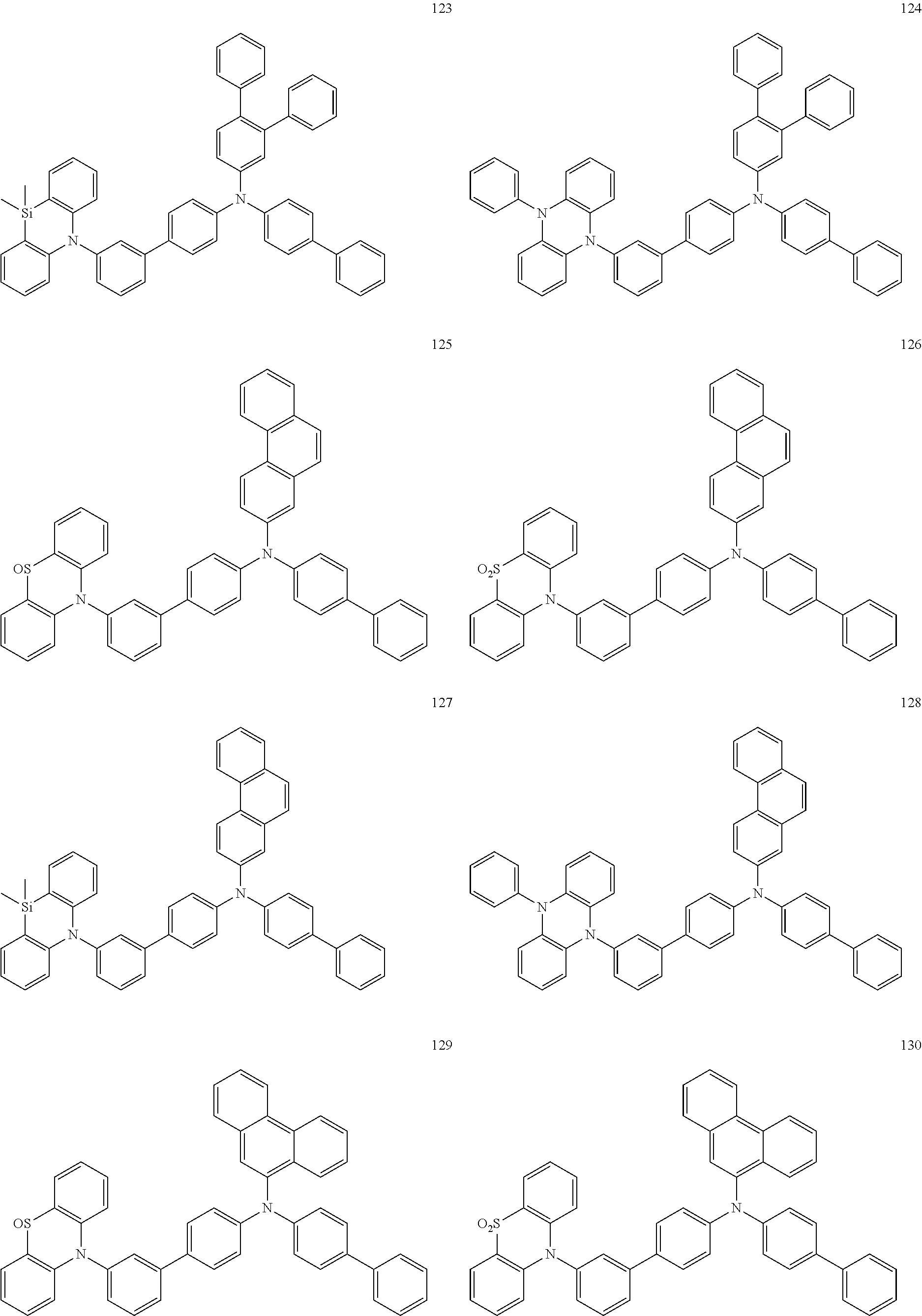

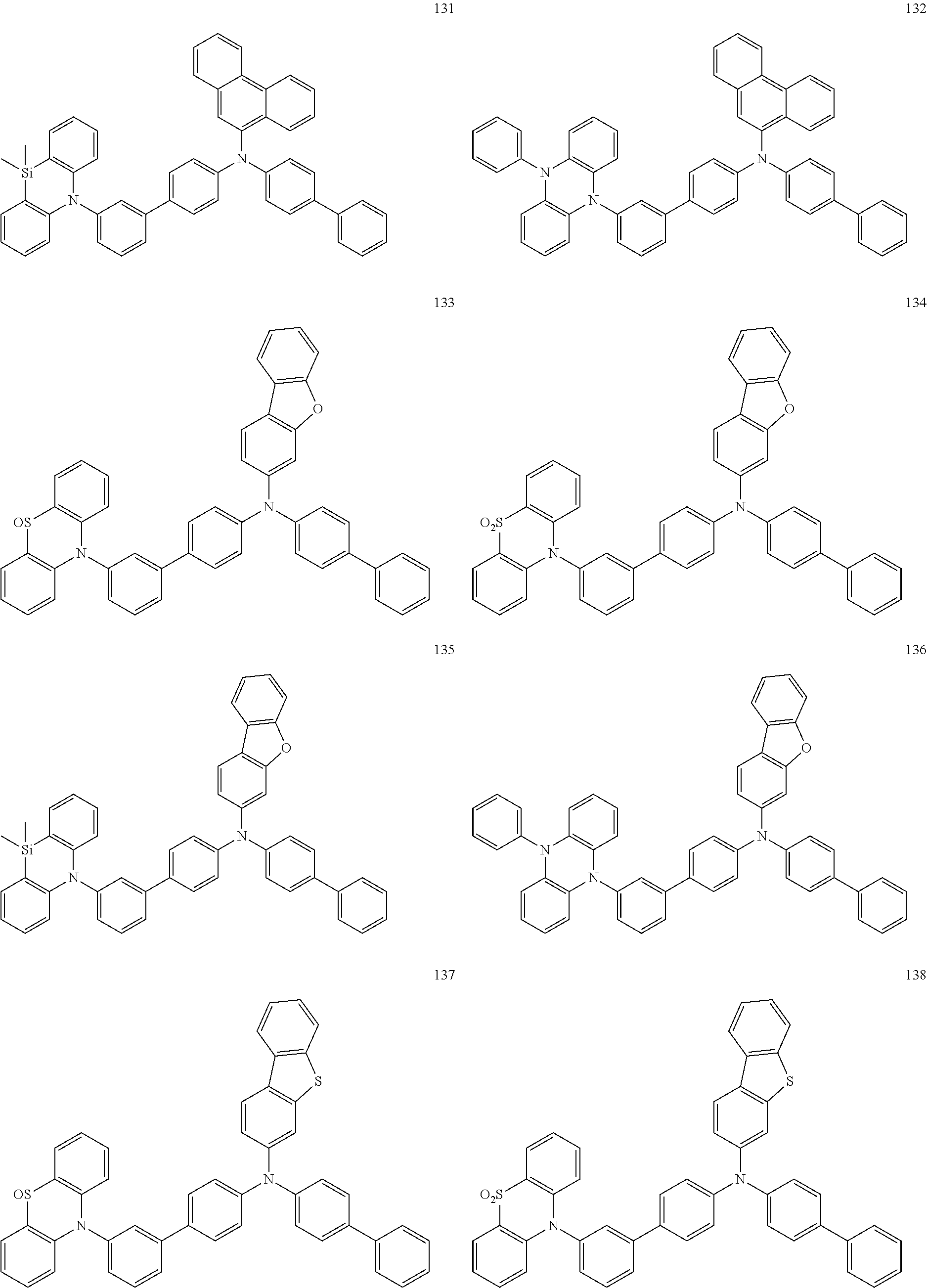

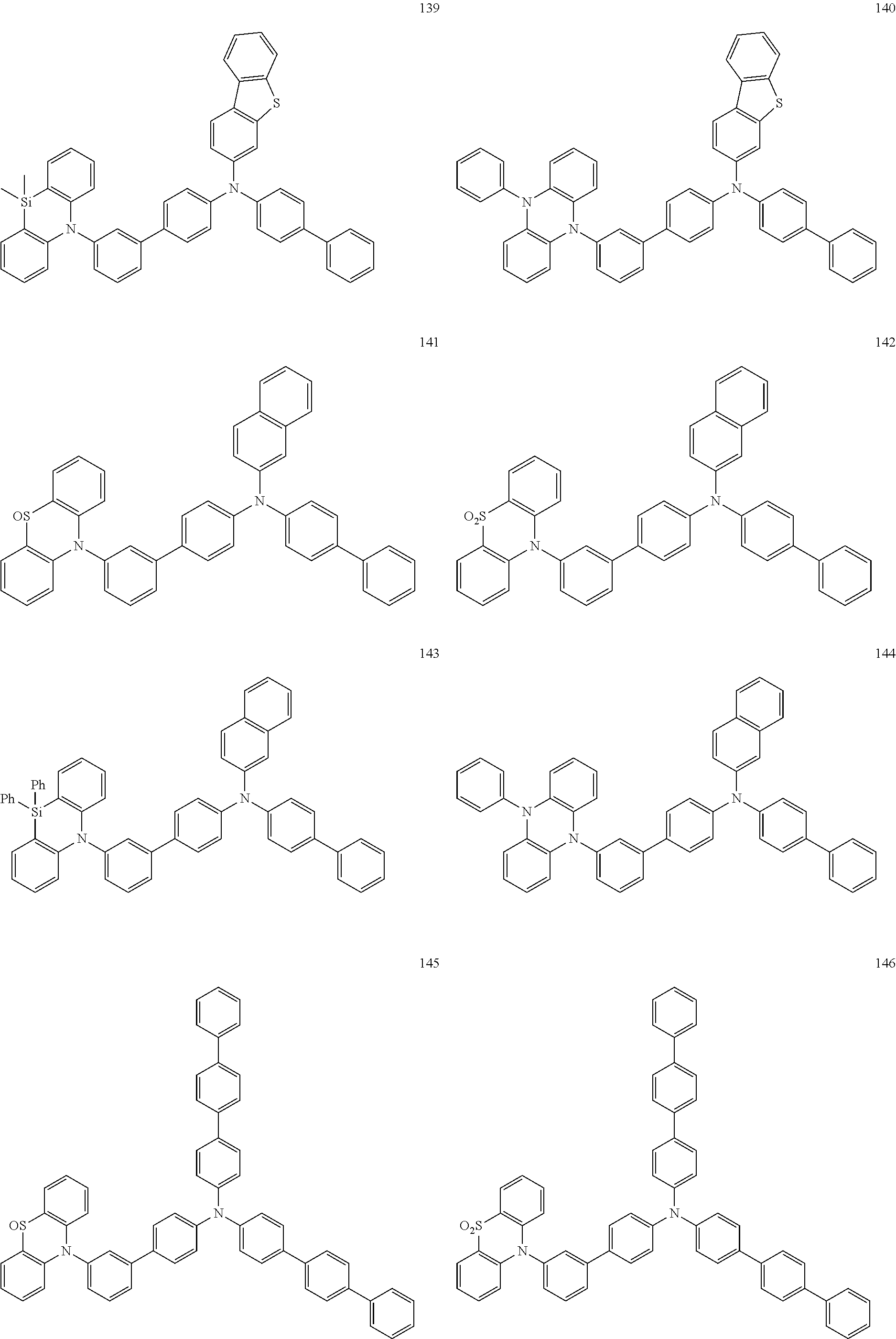

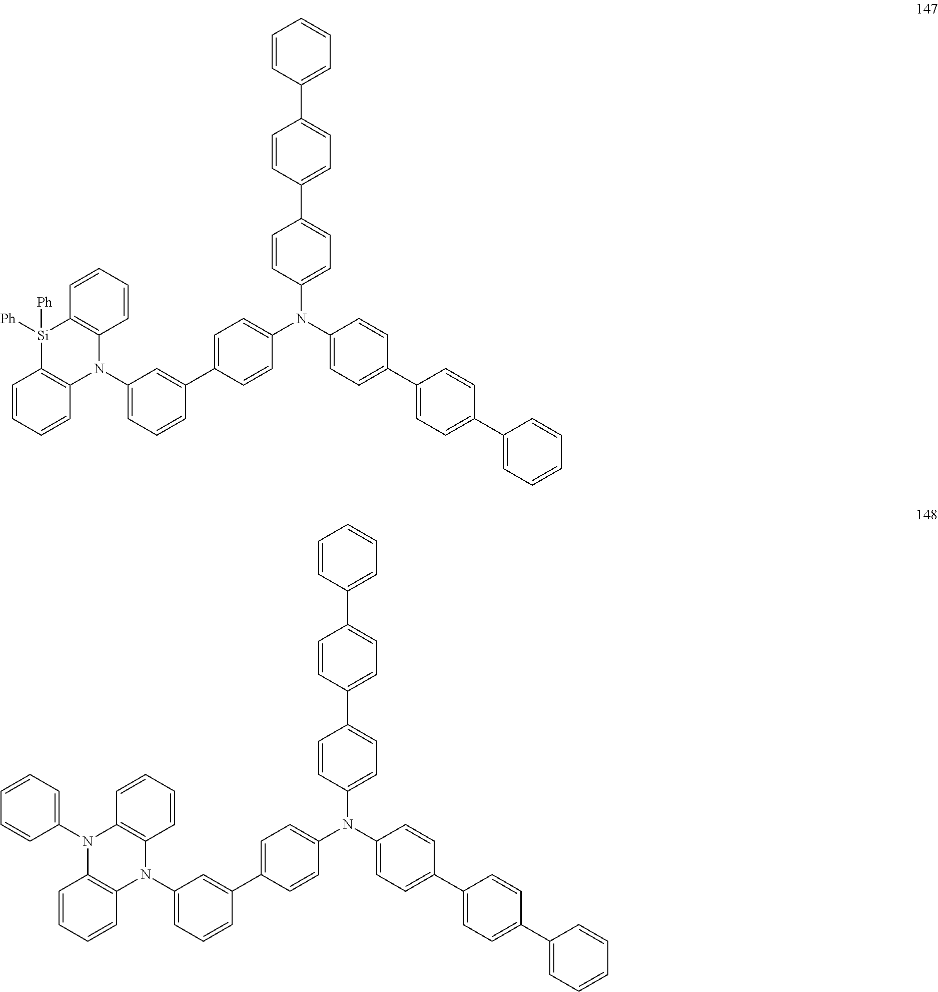 [SMILES: c1ccc(-c2ccc(-c3ccc(N(c4ccc(-c5ccc(-c6ccccc6)cc5)cc4)c4ccc(-c5cccc(N6c7ccccc7[Si](c7ccccc7)(c7ccccc7)c7ccccc76)c5)cc4)cc3)cc2)cc1 ; c1ccc(-c2ccc(-c3ccc(N(c4ccc(-c5ccc(-c6ccccc6)cc5)cc4)c4ccc(-c5cccc(N6c7ccccc7N(c7ccccc7)c7ccccc76)c5)cc4)cc3)cc2)cc1]

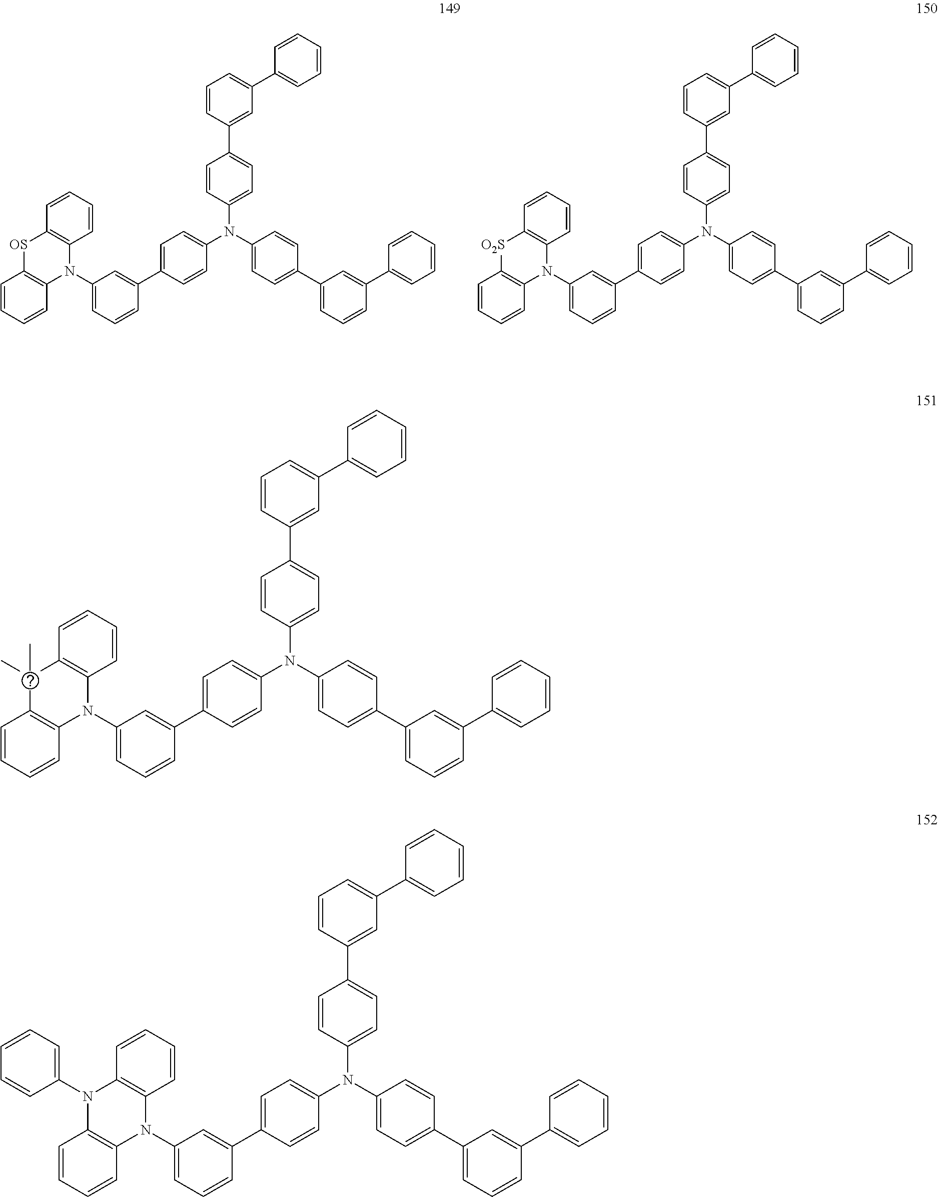

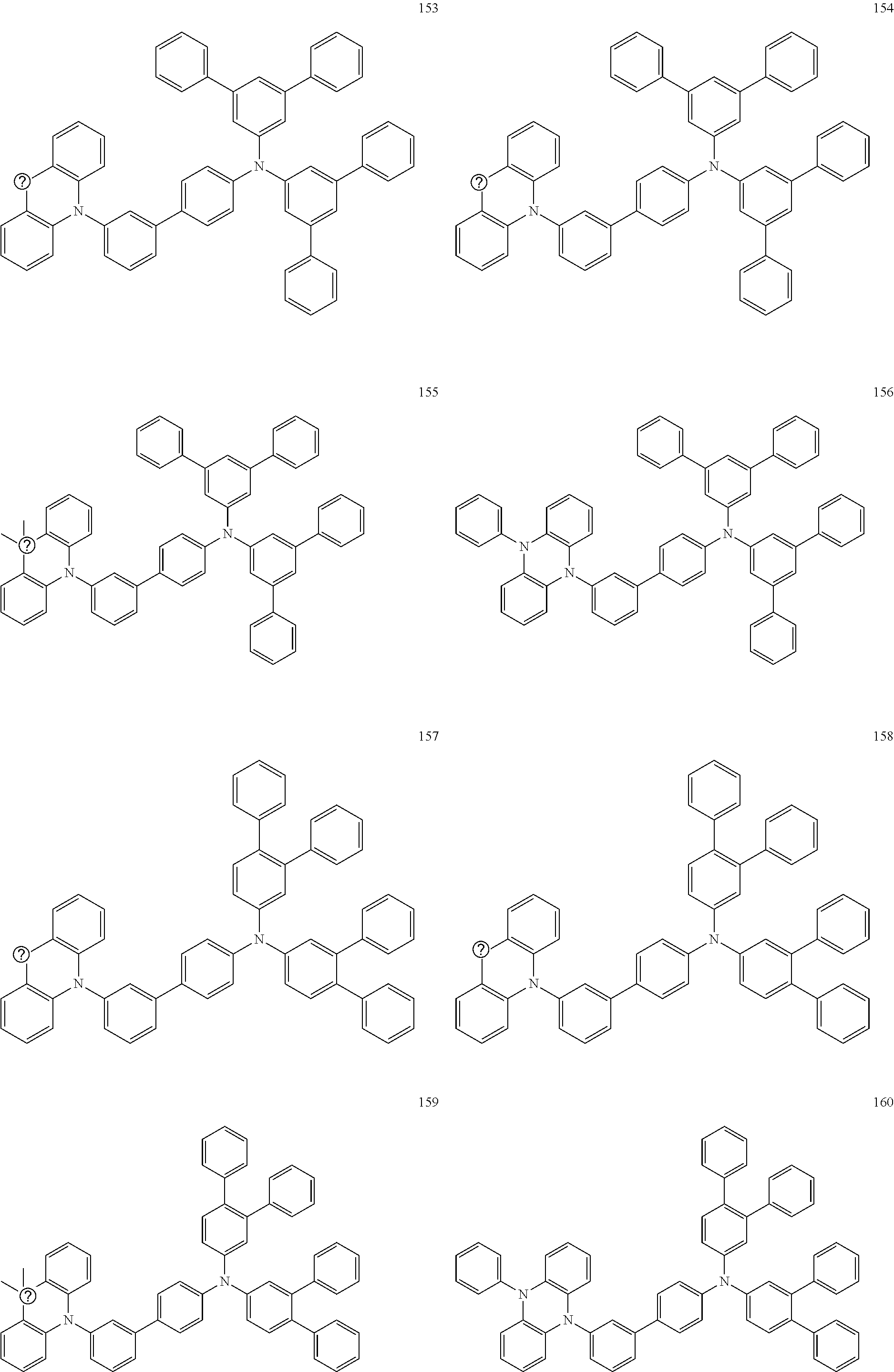

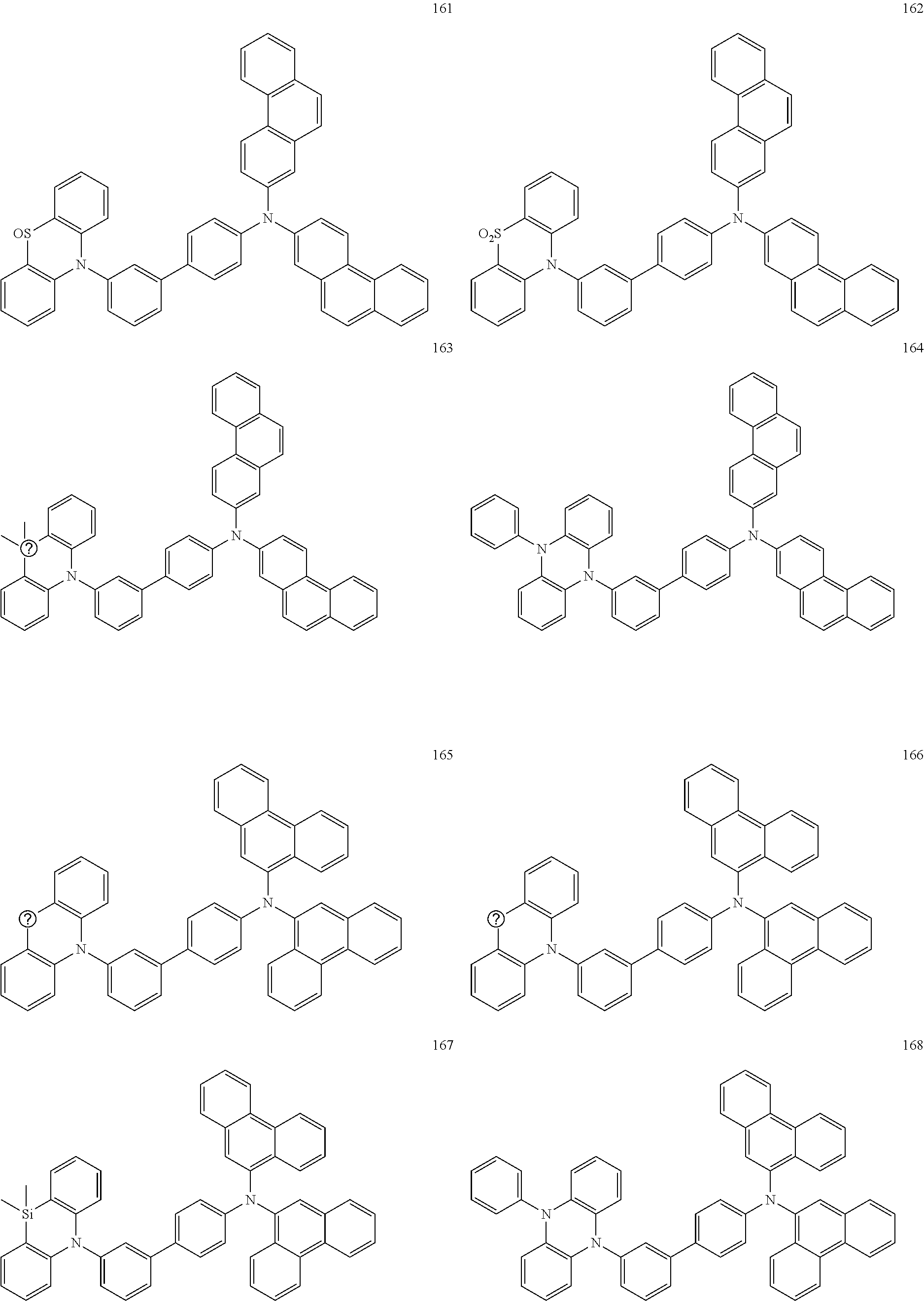

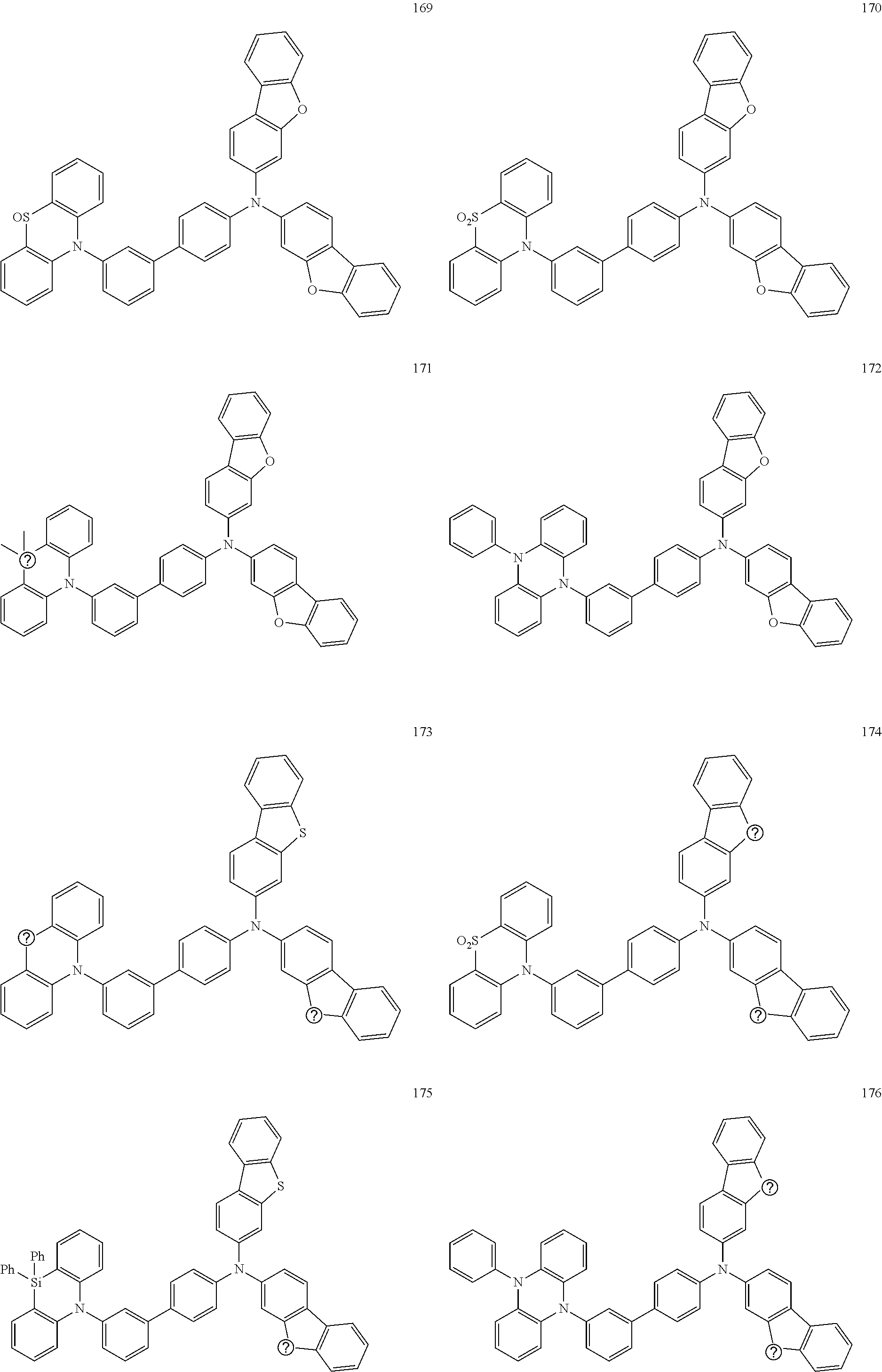

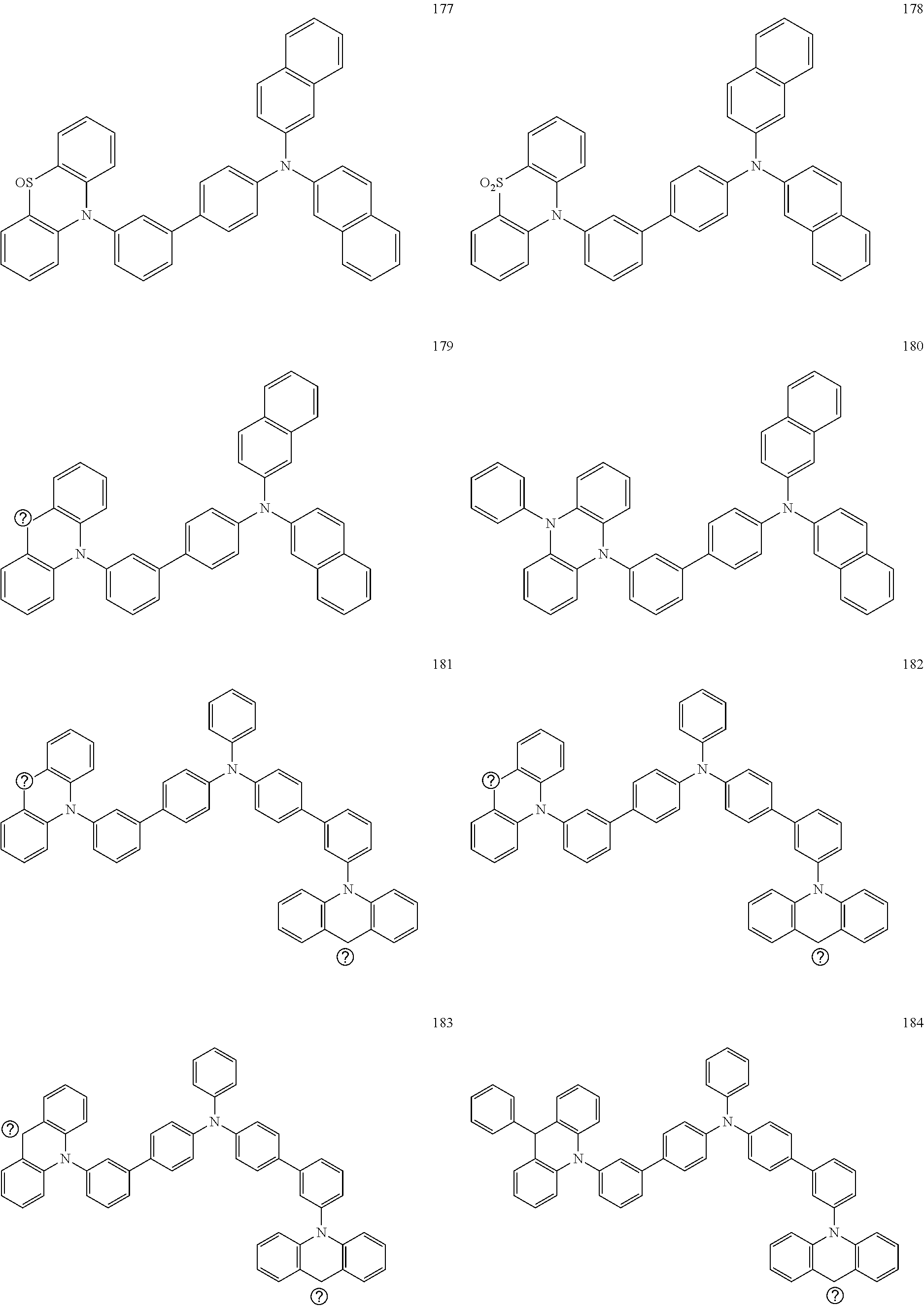

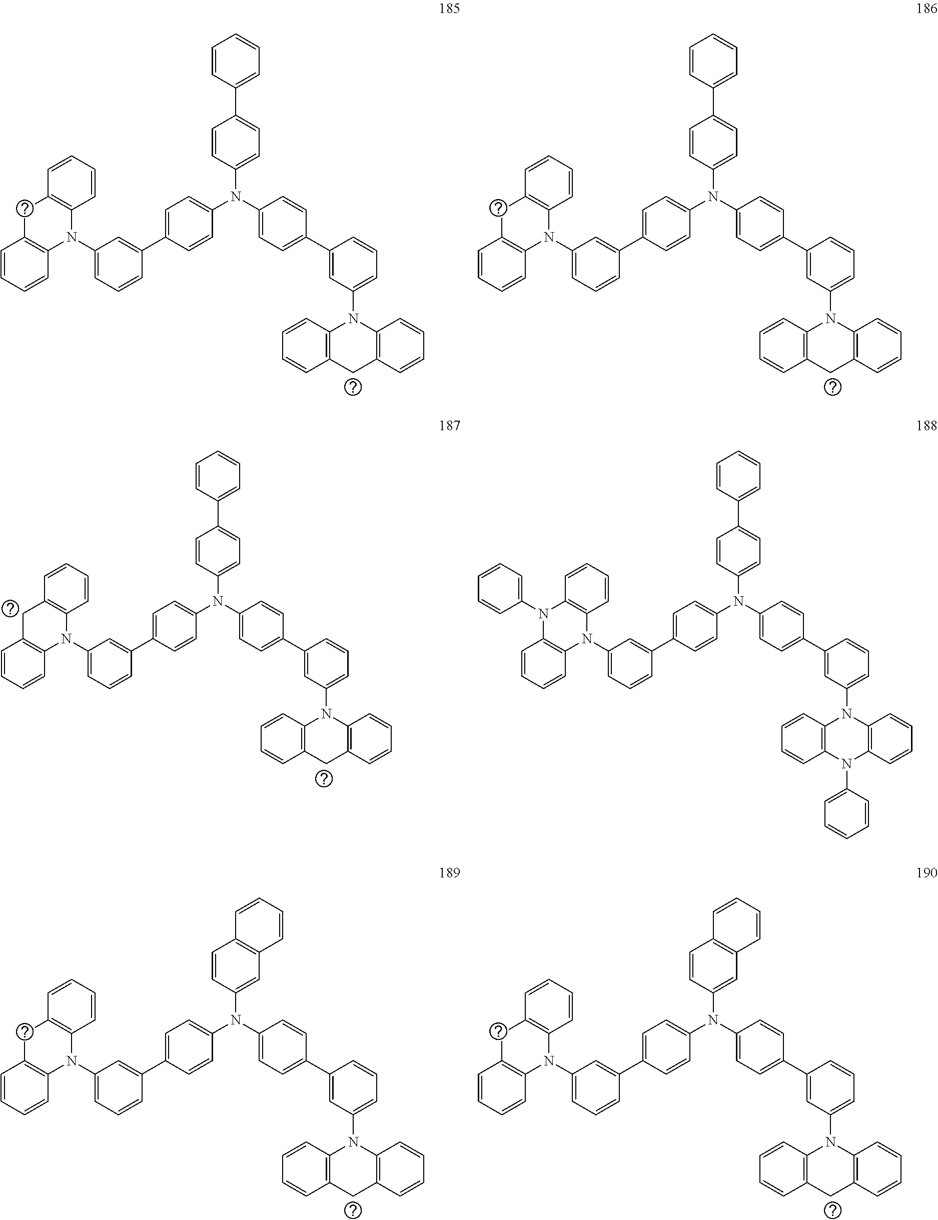

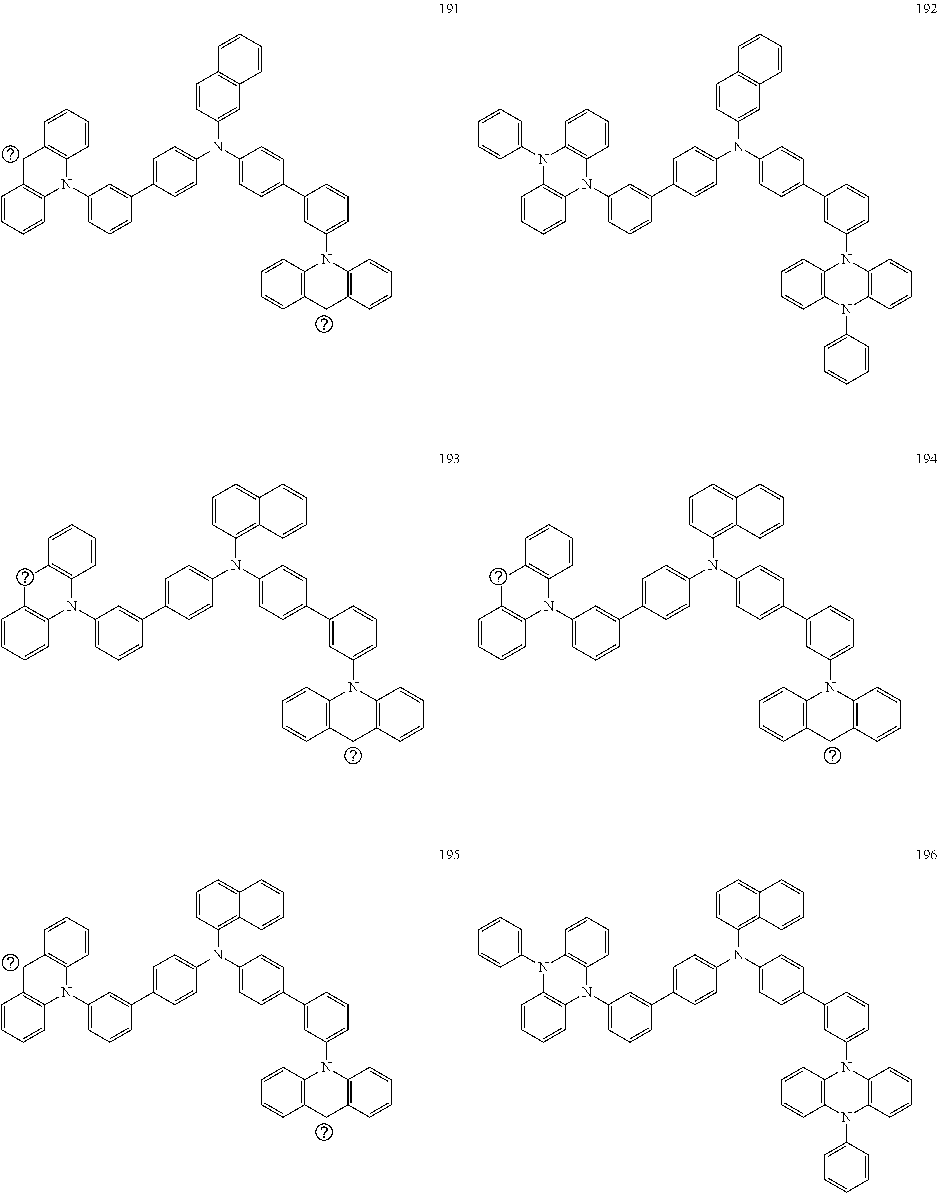

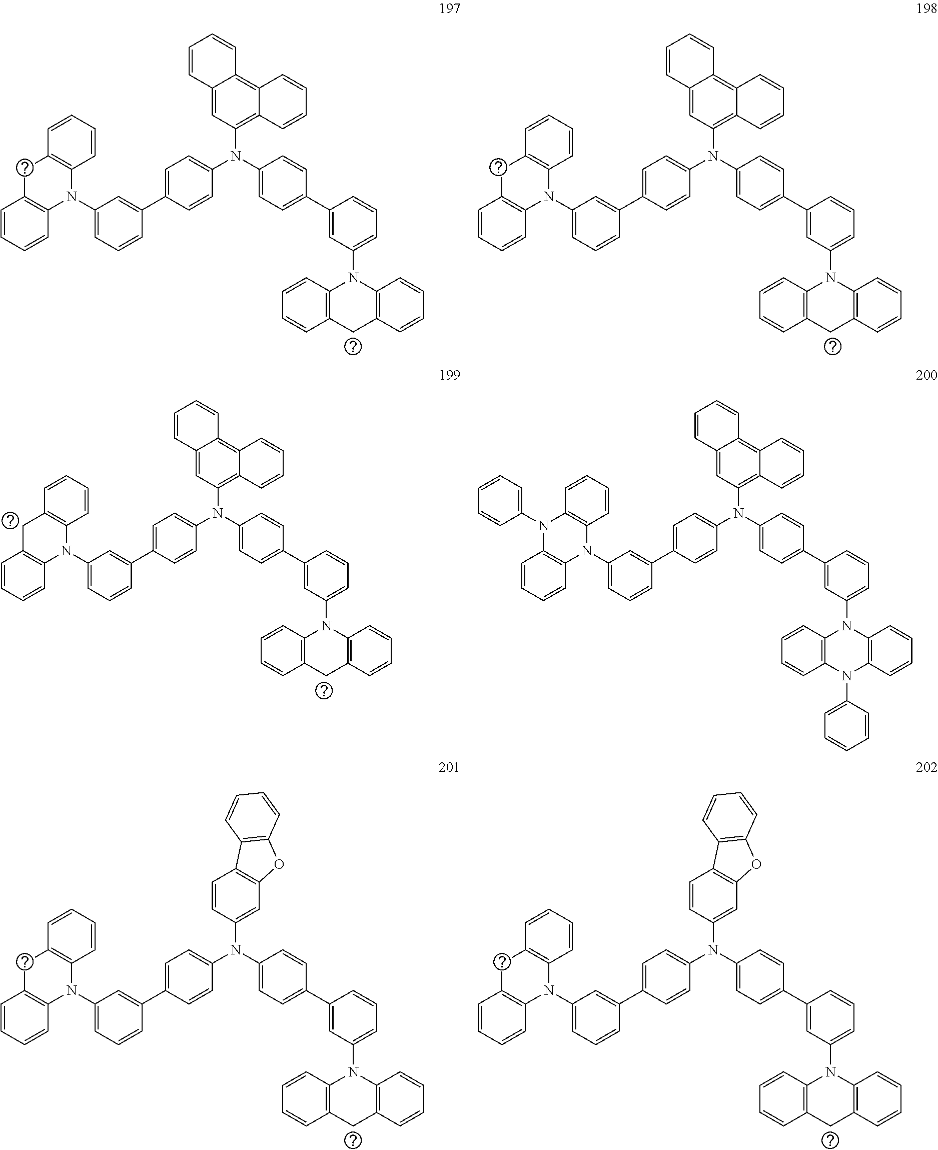

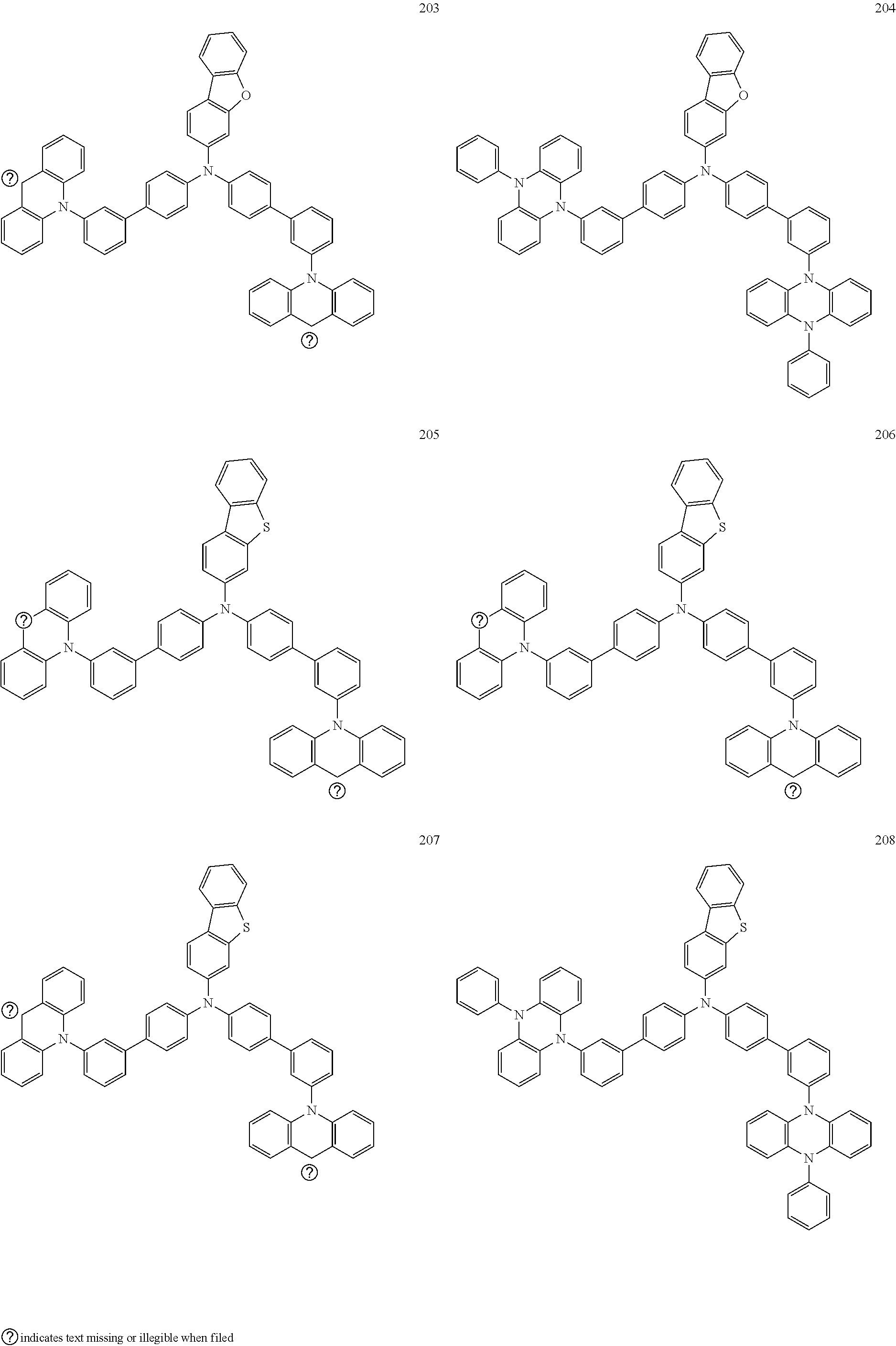

The amine derivative according to an embodiment of the present disclosure may be synthesized by the following Reactions (1) to (3):

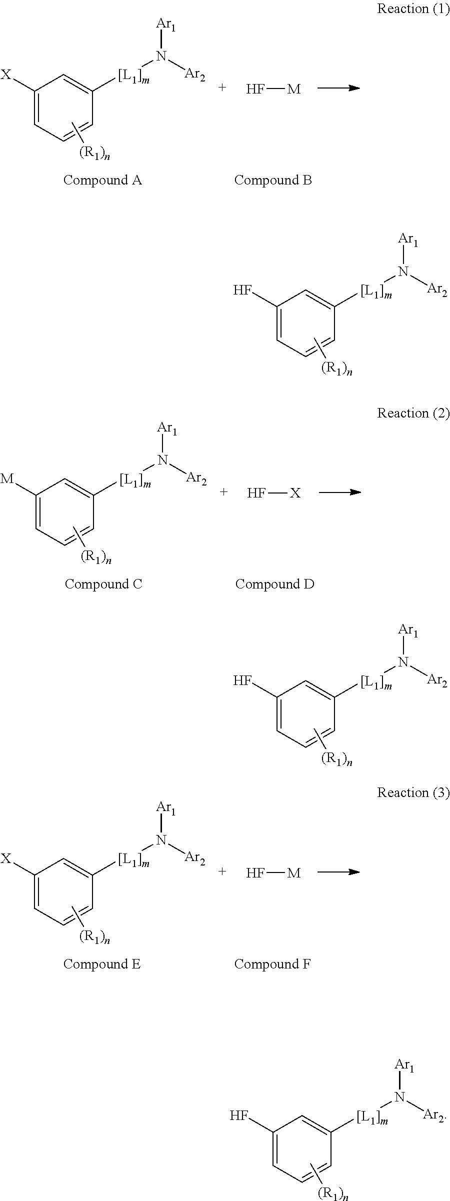

In Reactions (1) to (3), Hf is represented by the following Formula 3:

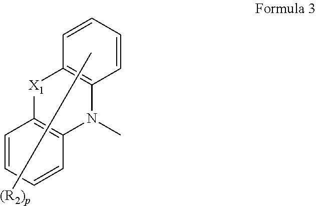

In Reaction (1), when Compound A is a compound including a leaving group such as halogen (X) at a meta position, and Compound B is a compound including a metal (M) such as boron (B), the amine derivative according to an embodiment of the present disclosure may be synthesized via a coupling reaction of Compound A and Compound B.

In Reaction (2), when Compound C is a compound including a leaving group such as a metal (M) (including, for example, boron) at a meta position, and Compound D is a compound including halogen (X), the amine derivative according to an embodiment of the present disclosure may be synthesized via a coupling reaction of Compound C and Compound D.

In Reaction (3), when Compound E is a compound including a leaving group such as halogen (X) at a meta position, and Compound F is a compound including hydrogen (H), the amine derivative according to an embodiment of the present disclosure may be synthesized via a coupling reaction of Compound E and Compound F.

However, the synthesis of the amine derivative according to an embodiment is not limited to the synthetic examples of Reactions (1) to (3). In some embodiments, the synthesis of the amine derivative may follow a scheme of performing the coupling reaction of any of Reactions (1) to (3), and then may include the act of introducing Ar₁ and Ar₂.

### 2. Regarding Organic EL Device Using Material for Organic EL Device Including Amine Derivative

An organic EL device using a material for an organic EL device according to embodiments of the present disclosure will be described with reference to the drawing. The drawing is a schematic cross-sectional view of an organic EL device according to an embodiment of the present disclosure.

As shown in the drawing, an organic EL device 100 according to an embodiment of the present disclosure may be provided with a substrate 110, a first electrode 120 positioned on the substrate 110, a hole injection layer 130 positioned on the first electrode 120, a hole transport layer 140 positioned on the hole injection layer 130, an emission layer 150 positioned on the hole transport layer 140, an electron transport layer 160 positioned on the emission layer 150, an electron injection layer 170 positioned on the electron transport layer 160 and a second electrode 180 positioned on the electron injection layer 170.

The material for an organic EL device according to an embodiment of the present disclosure may be included in at least one selected from the hole transport layer and the emission layer. For example, the material for an organic EL device may be included in both layers. In some embodiments, the material for an organic EL device may be included in the hole transport layer 140.

Each organic thin layer positioned between the first electrode 120 and the second electrode 180 of the organic EL device may be formed by various suitable synthesis methods such as an evaporation method.

The substrate 110 may be any suitable substrate capable of being used in an organic EL device. For example, the substrate 110 may be a glass substrate, a semiconductor substrate, or a transparent plastic substrate.

The first electrode 120 may be, for example, an anode and may be formed by an evaporation method, a sputtering method, and/or the like on the substrate 110. For example, the first electrode 120 may be formed as a transmission type electrode (e.g., transmission electrode) using a metal, an alloy, a conductive compound, and/or the like having high work function. The first electrode 120 may be formed using, for example, transparent and highly conductive indium tin oxide (ITO), indium zinc oxide (IZO), tin oxide (SnO₂), zinc oxide (ZnO), and/or the like. In some embodiments, the anode 120 may be formed as a reflection type electrode (e.g., reflection electrode) using magnesium (Mg), aluminum (Al), and/or the like.

On the first electrode 120, the hole injection layer 130 may be formed. The hole injection layer 130 is a layer that may facilitate the injection of holes from the first electrode 120 and may be formed, for example, on the first electrode 120 to a thickness from about 10 nm to about 150 nm. The hole injection layer 130 may be formed using any suitable material. Non-limiting examples of suitable materials for forming the hole injection layer may include, for example, triphenylamine-containing polyether ketone (TPAPEK), 4-isopropyl-4′-methyldiphenyliodoniumtetrakis(pentaflorophenyl)borate (PPBI), N,N′-diphenyl-N,N′-bis-[4-(phenyl-m-tolyl-amino)-phenyl]-biphenyl-4,4′-diamine (DNTPD), a phthalocyanine compound such as copper phthalocyanine, 4,4′,4″-tris(3-methyl phenylamino)triphenylamine (m-MTDATA), N,N′-di(1-naphthyl)-N,N′-diphenylbenzidine (NPB), 4,4′,4″-tris{N,N-diamino}triphenylamine (TDATA), 4,4′,4″-tris(N,N-2-naphthylphenylamino)triphenylamine (2-TNATA), polyaniline/dodecylbenzenesulfonic acid (Pani/DBSA), poly(3,4-ethylenedioxythiophene)/poly(4-styrenesulfonate) (PEDOT/PSS), polyaniline/camphorsulfonic acid (Pani/CSA), polyaniline/poly(4-styrenesulfonate (PANI/PSS), and/or the like.

On the hole injection layer 130, the hole transport layer 140 may be formed. The hole transport layer 140 may be formed by stacking a plurality of layers. The hole transport layer 140 is a layer including a hole transport material having hole transporting function and may be formed, for example, on the hole injection layer 130 to a thickness from about 10 nm to about 150 nm. The hole transport layer 140 may be formed using the material for an organic EL device according to an embodiment of the present disclosure. In embodiments where the material for an organic EL device according to an embodiment of the present disclosure is used as a host material of the emission layer 150, the hole transport layer 140 may be formed using any suitable hole transport material. Non-limiting examples of the suitable hole transport material may include, for example, 1,1-bis[(di-4-tolylamino)phenyl]cyclohexane (TAPC), a carbazole derivative such as N-phenyl carbazole and/or polyvinyl carbazole, N,N′-bis(3-methylphenyl)-N,N′-diphenyl-[1,1-biphenyl]-4,4′-diamine (TPD), 4,4′,4″-tris(N-carbazolyl)triphenylamine (TCTA), N,N′-di(1-naphthyl)-N,N′-diphenylbenzidine (NPB), and/or the like.

On the hole transport layer 140, the emission layer 150 may be formed. The emission layer 150 may be a layer emitting light via fluorescence, phosphorescence, and/or the like and may be formed to a thickness from about 10 nm to about 60 nm. The material of the emission layer 150 may be any suitable luminescent material and may be selected from a fluoranthene derivative, a pyrene derivative, an arylacetylene derivative, a fluorene derivative, a perylene derivative, a chrysene derivative, and/or the like, without specific limitation. In some embodiments, the pyrene derivative, the perylene derivative, and/or the anthracene derivative may be used. For example, as the material of the emission layer 150, an anthracene derivative represented by the following Formula 4 may be used:

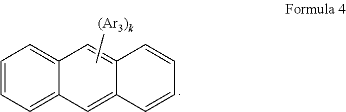

In the above Formula 4, Arₐ may be selected from hydrogen, deuterium, a substituted or unsubstituted alkyl group having 1 to 50 carbon atoms, a substituted or unsubstituted cycloalkyl group having 3 to 50 carbon atoms for forming a ring, a substituted or unsubstituted alkoxy group having 1 to 50 carbon atoms, a substituted or unsubstituted aralkyl group having 7 to 50 carbon atoms, a substituted or unsubstituted aryloxy group having 6 to 50 carbon atoms for forming a ring, a substituted or unsubstituted arylthio group having 6 to 50 carbon atoms for forming a ring, a substituted or unsubstituted alkoxycarbonyl group having 2 to 50 carbon atoms, a substituted or unsubstituted aryl group having 6 to 50 carbon atoms for forming a ring, a heteroaryl group having 5 to 50 carbon atoms for forming a ring, a substituted or unsubstituted silyl group, a carboxyl group, a halogen atom, a cyano group, a nitro group and a hydroxyl group, and k may be an integer from 1 to 10.

In Formula 4, Arₐ may include a phenyl group, a biphenyl group, a terphenyl group, a naphthyl group, a phenylnaphthyl group, a naphthylphenyl group, an anthryl group, a phenanthryl group, a fluorenyl group, an indenyl group, a pyrenyl group, an acetonaphthenyl group, a fluoranthenyl group, a triphenylenyl group, a pyridyl group, a furanyl group, a pyranyl group, a thienyl group, a quinolyl group, an isoquinolyl group, a benzofuranyl group, a benzothienyl group, an indolyl group, a carbazolyl group, a benzoxazolyl group, a benzothiazolyl group, a quinoxalyl group, a benzoimidazolyl group, a pyrazolyl group, a dibenzofuranyl group, a dibenzothienyl group, and/or the like. For example, the phenyl group, the biphenyl group, the terphenyl group, the fluorenyl group, the carbazolyl group, the dibenzofuranyl group, and/or the like may be used.

A compound represented by Formula 4 may include compounds represented by the following structures a-1 to a-12. However, the compound represented by Formula 4 is not limited thereto.

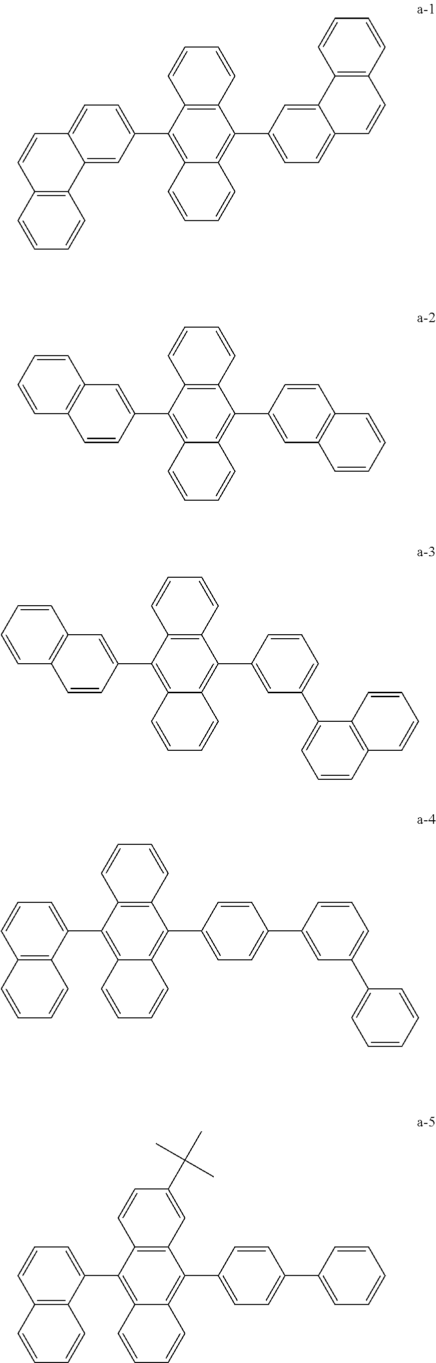

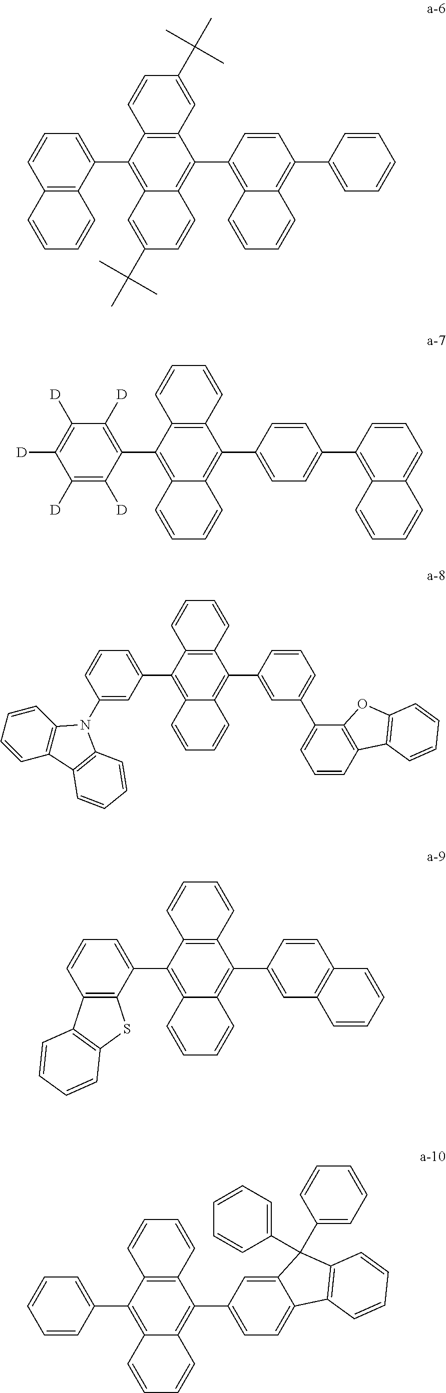

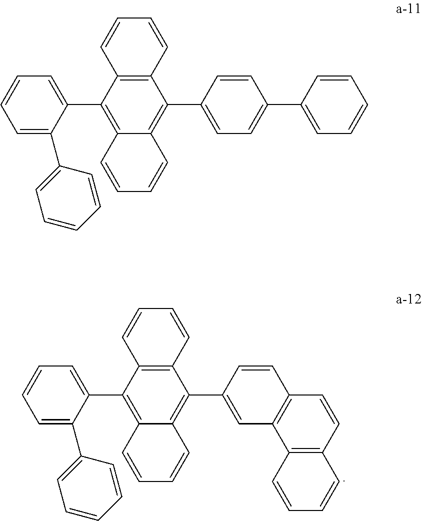

The emission layer 150 may include a dopant such as a styryl derivative (e.g., 1,4-bis[2-(3-N-ethylcarbazolyl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalene-2-yl)vinyl)phenyl)-N-phenylbenzeneamine (N-BDAVBi)), perylene and/or the derivative thereof (e.g., 2,5,8,11-tetra-t-butylperylene (TBPe)), pyrene and/or the derivative thereof (e.g., 1,1-dipyrene, 1,4-dipyrenylbenzene and/or 1,4-bis(N,N-diphenylamino)pyrene), but embodiments of the present disclosure are not limited thereto.

On the emission layer 150, an electron transport layer 160 including, for example, tris(8-hydroxyquinolinato)aluminum (Alq3) and/or a material having a nitrogen-containing aromatic ring (e.g., a material including a pyridine ring such as 1,3,5-tri[(3-pyridyl)-phen-3-yl]benzene, a material including a triazine ring such as 2,4,6-tris(3′-(pyridin-3-yl)biphenyl-3-yl)-1,3,5-triazine, and/or a material including an imidazole derivative such as 2-(4-(N-phenylbenzoimidazolyl-1-ylphenyl)-9,10-dinaphthylanthracene) may be formed. The electron transport layer 160 is a layer including an electron transport material with electron transporting function and may be formed on the emission layer 150 to a thickness from about 15 nm to about 50 nm. On the electron transport layer 160, the electron injection layer 170 may be formed using a material including, for example, lithium fluoride (LiF), lithium-8-quinolinato (Liq), and/or the like. The electron injection layer 170 may facilitate the injection of electrons from the second electrode 180 and may be formed to a thickness from about 0.3 nm to about 9 nm.

In addition, on the electron injection layer 170, the second electrode 180 may be formed. The second electrode 180 may be, for example, a cathode. For example, the second electrode 180 may be formed as a reflection type electrode (e.g., reflection electrode) using a metal, an alloy, a conductive compound, and/or the like having low work function. The second electrode 180 may be formed using, for example, lithium (Li), magnesium (Mg), aluminum (Al), aluminum-lithium (Al—Li), calcium (Ca), magnesium-indium (Mg—In), magnesium-silver (Mg—Ag), and/or the like. In some embodiments, the second electrode 180 may be formed as a transmission type electrode (e.g., transmission electrode) using ITO, IZO, and/or the like. Each of the above-mentioned layers may be formed by selecting one or more appropriate layer forming methods such as a vacuum evaporation method, a sputtering method and/or various coating methods, in consideration of the respective materials used.

As described above, an embodiment of the structure of the organic EL device 100 according to an embodiment of the present disclosure was explained. The organic EL device 100 including the material for an organic EL device according to an embodiment of the present disclosure has a decreased driving voltage and improved emission efficiency.

However, the structure of the organic EL device 100 according to an embodiment of the present disclosure is not limited to the above-described embodiments. For example, the organic EL device 100 according to an embodiment of the present disclosure may be formed using various other suitable structures of organic EL devices. For example, the organic EL device 100 may not be provided with (e.g., may exclude) one or more layers selected from the hole injection layer 130, the electron transport layer 160 and the electron injection layer 170. In addition, each layer of the organic EL device 100 may be formed as a single layer or a plurality of layers (e.g., to have a multi-layer structure).

In some embodiments, the organic EL device 100 may be provided with a hole blocking layer between the electron transport layer 160 and the emission layer 150 to prevent or reduce the diffusion of triplet excitons or holes into the electron transport layer 160. The hole blocking layer may be formed using, for example, an oxadiazole derivative, a triazole derivative and/or a phenanthroline derivative.

### Examples

Hereinafter, the organic EL device according to an embodiment of the present disclosure will be explained in more detail by referring to examples and comparative examples. However, the following examples are provided only for illustration of the organic EL device according to the present disclosure, and the organic EL device according to the present disclosure is not limited thereto.

### Synthetic Example 1

### Synthesis of Example Compound 1

Example Compound 1 was synthesized according to the following synthetic scheme:

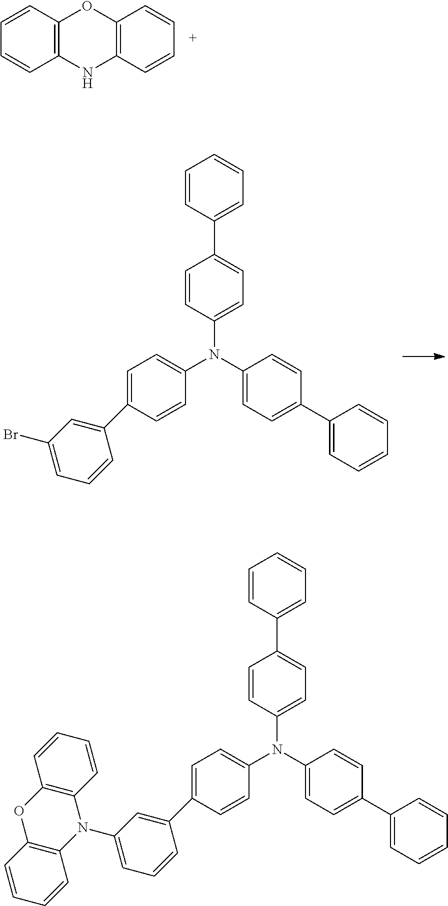 [SMILES: c1ccc2c(c1)Nc1ccccc1O2 ; Brc1cccc(-c2ccc(N(c3ccc(-c4ccccc4)cc3)c3ccc(-c4ccccc4)cc3)cc2)c1 ; c1ccc(-c2ccc(N(c3ccc(-c4ccccc4)cc3)c3ccc(-c4cccc(N5c6ccccc6Oc6ccccc65)c4)cc3)cc2)cc1]

### Example Compound 1

Under an Ar atmosphere, 2.50 g of N-[1,1′-biphenyl]-4-yl-N-(3′-bromo[1,1′-biphenyl]-4-yl)-[1,1′-biphenyl]-4-amine, 0.91 g of phenoxazine, 0.326 g of Pd₂(dba)₃.CHCl₃, 1.34 g of sodium tert-butoxide (t-BuONa), 65 mL of dehydrated toluene and 0.49 mL of a 2 M tert-butylphosphine ((t-Bu)₃P)/dehydrated toluene solution were added to a 200 mL three necked flask, followed by heating and stirring the resultant at about 90° C. for about 12 hours. After air cooling the obtained mixture, water was added, an organic layer was separated, and solvents were distilled. The crude product thus obtained was separated by silica gel column chromatography (using a mixture solvent of dichloromethane and hexane) and recrystallized using a mixture solvent of toluene/hexane to produce 1.70 g of Example Compound 1 as a white solid (Yield 78%). The identification of the product was conducted by detecting molecular ion peaks using fast atom bombardment mass spectrometry (FAB-MS), and a value of 654.27 (C₄₈H₃₄N₂O) was obtained.

### Synthetic Example 2

### Synthesis of Example Compound 2

Example Compound 2 was synthesized according to the following synthetic scheme:

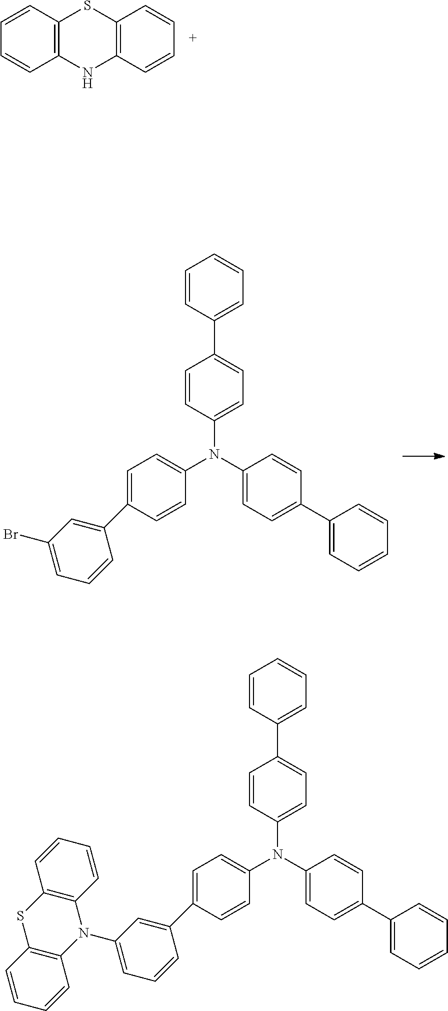 [SMILES: c1ccc2c(c1)Nc1ccccc1S2 ; Brc1cccc(-c2ccc(N(c3ccc(-c4ccccc4)cc3)c3ccc(-c4ccccc4)cc3)cc2)c1 ; c1ccc(-c2ccc(N(c3ccc(-c4ccccc4)cc3)c3ccc(-c4cccc(N5c6ccccc6Sc6ccccc65)c4)cc3)cc2)cc1]

### Example Compound 2

Under an Ar atmosphere, 1.00 g of phenothiazine, 2.50 g of N-[1,1′-biphenyl]-4-yl-N-(3′-bromo[1,1′-biphenyl]-4-yl)-[1,1′-biphenyl]-4-amine, 0.327 g of Pd₂(dba)₃.CHCl₃, 1.32 g of t-BuONa, 65 mL of dehydrated toluene and 0.49 mL of a 2 M (t-Bu)₃P/dehydrated toluene solution were added to a 200 mL three necked flask, followed by heating and stirring the resultant at about 90° C. for about 12 hours. After air cooling the obtained mixture, water was added, an organic layer was separated, and solvents were distilled. The crude product thus obtained was separated by silica gel column chromatography (using a mixture solvent of dichloromethane and hexane) and recrystallized using a mixture solvent of toluene/hexane to produce 1.61 g of Example Compound 2 as a white solid (Yield 72%). The identification of the product was conducted by detecting molecular ion peaks using FAB-MS, and a value of 670.24 (C₄₈H₃₄N₂S) was obtained.

### Synthetic Example 3

### Synthesis of Example Compound 3

Example Compound 3 was synthesized according to the following synthetic scheme:

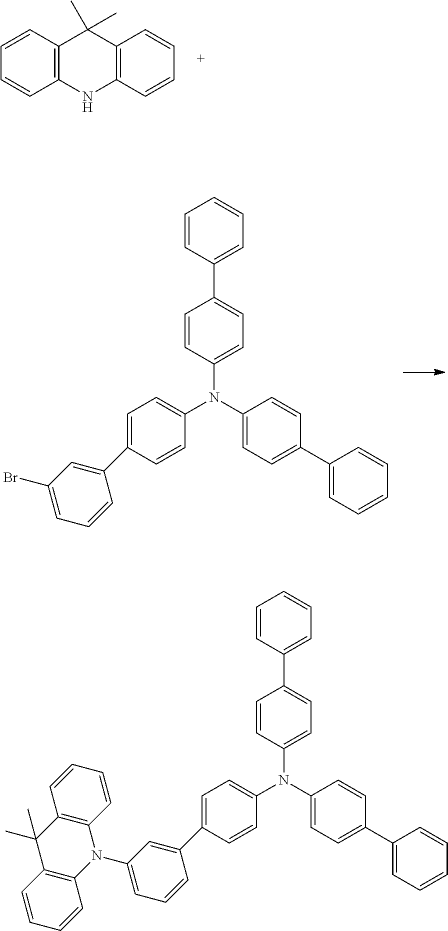 [SMILES: CC1(C)c2ccccc2Nc2ccccc21 ; Brc1cccc(-c2ccc(N(c3ccc(-c4ccccc4)cc3)c3ccc(-c4ccccc4)cc3)cc2)c1 ; CC1(C)c2ccccc2N(c2cccc(-c3ccc(N(c4ccc(-c5ccccc5)cc4)c4ccc(-c5ccccc5)cc4)cc3)c2)c2ccccc21]

### Example Compound 3

Under an Ar atmosphere, 2.50 g of N-[1,1′-biphenyl]-4-yl-N-(3′-bromo[1,1′-biphenyl]-4-yl)-[1,1′-biphenyl]-4-amine, 1.04 g of 9,10-dihydro-9,9-dimethylacridine, 0.328 g of Pd₂(dba)₃.CHCl₃, 1.31 g of t-BuONa, 65 mL of dehydrated toluene and 0.49 mL of a 2 M (t-Bu)₃P/dehydrated toluene solution were added to a 200 mL three necked flask, followed by heating and stirring the resultant at about 90° C. for about 8 hours. After air cooling the obtained mixture, water was added, an organic layer was separated, and solvents were distilled. The crude product thus obtained was separated by silica gel column chromatography (using a mixture solvent of dichloromethane and hexane) and recrystallized using a mixture solvent of toluene/hexane to produce 2.71 g of Example Compound 3 as a white solid (Yield 88%). The identification of the product was conducted by detecting molecular ion peaks using FAB-MS, and a value of 680.32 (C₅₁H₄₀N₂) was obtained.

### Synthetic Example 4

### Synthesis of Example Compound 4

Example Compound 4 was synthesized according to the following synthetic scheme:

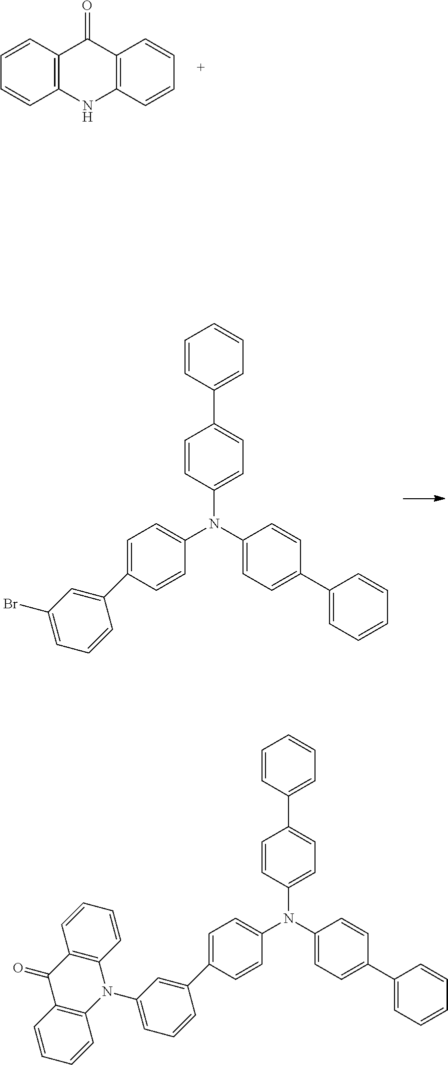 [SMILES: O=c1c2ccccc2[nH]c2ccccc12 ; Brc1cccc(-c2ccc(N(c3ccc(-c4ccccc4)cc3)c3ccc(-c4ccccc4)cc3)cc2)c1 ; O=c1c2ccccc2n(-c2cccc(-c3ccc(N(c4ccc(-c5ccccc5)cc4)c4ccc(-c5ccccc5)cc4)cc3)c2)c2ccccc12]

### Example Compound 4

4.720 g of N-[1,1′-biphenyl]-4-yl-N-(3′-bromo[1,1′-biphenyl]-4-yl)-[1,1′-biphenyl]-4-amine was dissolved in 39 mL of deaerated and dehydrated dimethylformamide (DMF), and 1.998 g of 9(10H)-acridone, 0.171 g of copper(I) iodide, 1.772 g of potassium carbonate (K₂CO₃), and 0.347 g of dipivaloylmethane were added thereto, followed by heating and stirring the resultant under refluxing conditions in an argon atmosphere for about 48 hours. After cooling the obtained mixture to room temperature, precipitated crystal was filtered and washed with water and hexane. The crude crystal thus obtained was separated by silica gel column chromatography (using a mixture solvent of dichloromethane and hexane) and recrystallized using a mixture solvent of chloroform-acetonitrile to produce 4.266 g of Example Compound 4 (Yield 62%). The identification of the product was conducted by detecting molecular ion peaks using FAB-MS, and a value of 666.27 (C₄₉H₃₄N₂O) was obtained.

### Synthetic Example 5

### Synthesis of Example Compound 83

Example Compound 83 was synthesized according to the following synthetic scheme:

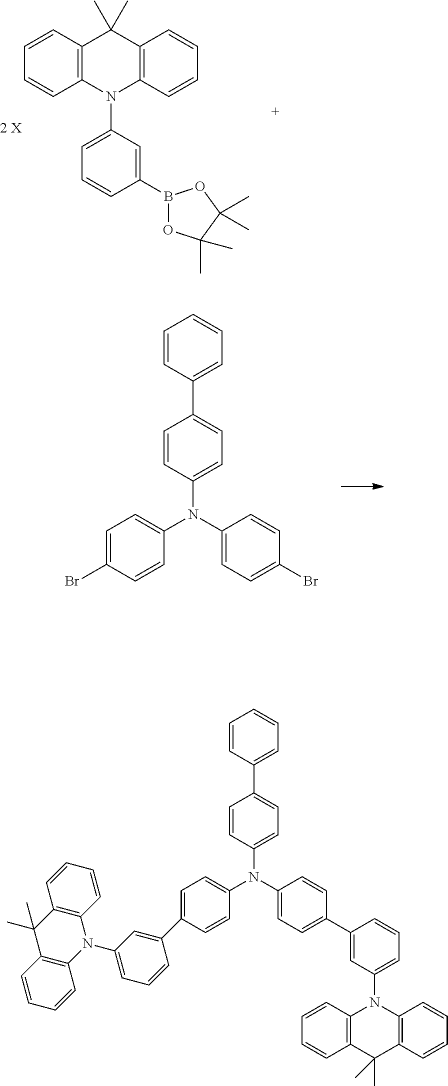 [SMILES: CC1(C)c2ccccc2N(c2cccc(B3OC(C)(C)C(C)(C)O3)c2)c2ccccc21 ; Brc1ccc(N(c2ccc(Br)cc2)c2ccc(-c3ccccc3)cc2)cc1 ; CC1(C)c2ccccc2N(c2cccc(-c3ccc(N(c4ccc(-c5ccccc5)cc4)c4ccc(-c5cccc(N6c7ccccc7C(C)(C)c7ccccc76)c5)cc4)cc3)c2)c2ccccc21]

### Example Compound 83

To a 500 mL three necked flask, 8.23 g of 9,10-dihydro-9,9-dimethyl-10-[3-(4,4,5,5-tetramethyl-1,3-dioxaborolan-2-yl)phenyl]acridine, 4.79 g of N,N-bis(4-bromophenyl)-[1,1′-biphenyl]-4-amine, 1.73 g of Pd(PPh₃)₄, and 5.53 g of potassium carbonate were added, followed by heating and stirring the resultant in a mixture solvent of 160 mL of toluene, 54 mL of water and 26 mL of ethanol at about 90° C. for about 12 hours. After air cooling the obtained mixture, water was added, an organic layer was separated, and solvents were distilled. The crude product thus obtained was separated by silica gel column chromatography (using a mixture solvent of dichloromethane and hexane) and recrystallized using a mixture solvent of methylene/ethanol to produce 4.53 g of Example Compound 83 as a white solid (Yield 51%). The identification of the product was conducted by detecting molecular ion peaks using FAB-MS, and a value of 887.42 (C₆₆H₅₃N₃) was obtained.

### Manufacturing of Organic EL Device

Then, an organic EL device was manufactured by the following method. First, on an ITO-glass substrate patterned and washed in advance, surface treatment using UV-ozone (O₃) was conducted. The layer thickness of the resulting ITO layer (the first electrode) was about 150 nm. After ozone treatment, the substrate was washed. After finishing washing, the substrate was set in a glass bell jar type evaporator (e.g., glass bell jar evaporator) for forming organic layers, and a hole injection layer, a HTL (a hole transport layer), an emission layer and an electron transport layer were evaporated one by one in a vacuum degree of about 10⁻⁴ to about 10⁻⁵ Pa. The material of the hole injection layer was 2-TNATA, and the thickness thereof was about 60 nm. The materials of the HTL were as shown in Table 1, and the thickness thereof was about 30 nm.

In addition, the thickness of the emission layer was about 25 nm. The host of a luminescent material was 9,10-di(2-naphthyl)anthracene (ADN). The dopant was 2,5,8,11-tetra-t-butylperylene (TBP). The doped amount of the dopant was about 3 wt % on the basis of the amount of the host. The material of the electron transport layer was Alq3, and the thickness thereof was about 25 nm. Subsequently, the substrate was transferred to a glass bell jar type evaporator (e.g., glass bell jar evaporator) for forming metal layers, and the electron injection layer and a cathode material were evaporated in a vacuum degree of about 10⁻⁴ to about 10⁻⁵ Pa. The material of the electron injection layer was LiF, and the thickness thereof was about 1.0 nm. The material of the second electrode was Al, and the thickness thereof was about 100 nm.

TABLE 1

| | | Current | | Emission |
|---|---|---|---|---|
| | | density | Voltage | efficiency |
| | HTL | (mA/cm²) | (V) | (cd/A) |
| Example 1 | Example | 10 | 6.5 | 7.2 |
| | Compound 1 | | | |
| Comparative | Comparative | 10 | 7.9 | 5.5 |
| Example 1 | Compound C1 | | | |
| Example 2 | Example | 10 | 6.5 | 7.3 |
| | Compound 2 | | | |
| Comparative | Comparative | 10 | 7.9 | 5.6 |
| Example 2 | Compound C2 | | | |
| Example 3 | Example | 10 | 6.5 | 7.4 |
| | Compound 3 | | | |
| Comparative | Comparative | 10 | 8.1 | 5.3 |
| Example 3 | Compound C3 | | | |
| Comparative | Comparative | 10 | 7.8 | 5.8 |
| Example 4 | Compound C4 | | | |
| Example 4 | Example | 10 | 6.6 | 7.1 |
| | Compound 4 | | | |
| Example 5 | Example | 10 | 6.8 | 7.2 |
| | Compound 83 | | | |

In Table 1, Comparative Compounds C1 to C4 are illustrated below. Comparative Compounds C1 to C3 are examples of the amine derivative in which the nitrogen atom of amine moiety is combined (e.g., coupled) with the nitrogen atom of a nitrogen-containing 6-membered condensed structure (e.g., phenoxazine in Comparative Compound C1) via a p-phenylene group. Comparative Compound C4 is an example of the amine derivative in which the nitrogen atom of amine is combined (e.g., coupled) with a carbon atom of a nitrogen-containing 6-membered condensed structure via an m-phenylene group.

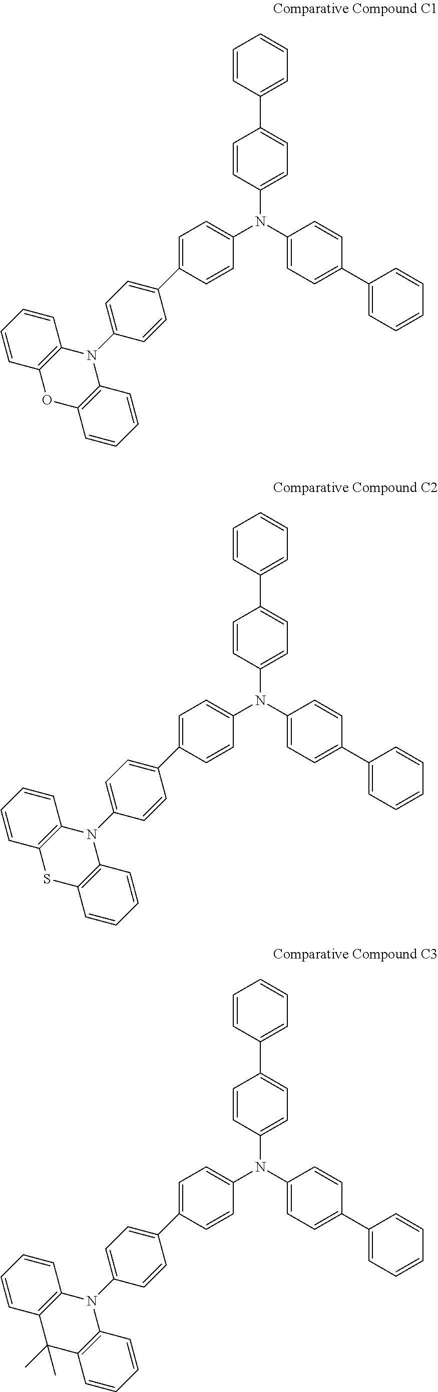 [SMILES: c1ccc(-c2ccc(N(c3ccc(-c4ccccc4)cc3)c3ccc(-c4ccc(N5c6ccccc6Oc6ccccc65)cc4)cc3)cc2)cc1 ; c1ccc(-c2ccc(N(c3ccc(-c4ccccc4)cc3)c3ccc(-c4ccc(N5c6ccccc6Sc6ccccc65)cc4)cc3)cc2)cc1 ; CC1(C)c2ccccc2N(c2ccc(-c3ccc(N(c4ccc(-c5ccccc5)cc4)c4ccc(-c5ccccc5)cc4)cc3)cc2)c2ccccc21]

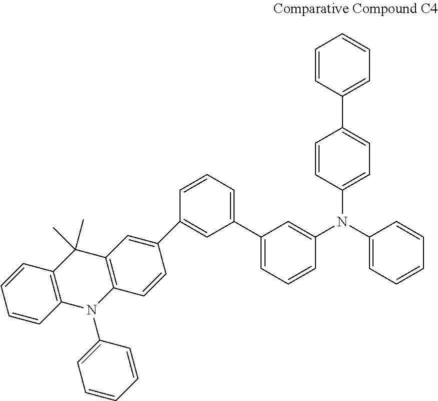 [SMILES: CC1(C)c2ccccc2N(c2ccccc2)c2ccc(-c3cccc(-c4cccc(N(c5ccccc5)c5ccc(-c6ccccc6)cc5)c4)c3)cc21]

### Evaluation of Properties

The driving voltage and the emission life of the organic EL devices thus manufactured were measured. Here, the luminescent properties of the organic EL devices 100 thus manufactured were evaluated using a C9920-11 brightness light distribution characteristics measurement system of HAMAMATSU Photonics Co. In addition, current density was measured at about 10 mA/cm², and half life was measured at 1,000 cd/m². The evaluation results are shown in Table 1.

Referring to Table 1, the organic EL devices according to Examples 1 to 5 in which hole transport layers HTL were formed using the amine derivatives according to embodiments of the present disclosure had decreased driving voltages and improved emission efficiencies when compared to those of the organic EL devices of Comparative Examples 1 to 4.

For example, the organic EL devices according to Examples 1 to 5 in which the hole transport layers HTL were formed using the amine derivatives according to embodiments of the present disclosure had decreased driving voltage and improved emission efficiency when compared to those of the organic EL devices of Comparative Examples 1 to 3 using amine derivatives, in which the nitrogen atom of the amine moiety is combined with the nitrogen atom of a nitrogen-containing 6-membered condensed structure via a p-phenylene group. In addition, the organic EL devices according to Examples 1 to 5 in which the hole transport layers HTL were formed using the amine derivatives according to embodiments of the present disclosure had decreased driving voltage and improved emission efficiency when compared to the organic EL device of Comparative Example 4 using an amine derivative, in which the nitrogen atom of the amine moiety is combined with a carbon atom of a nitrogen-containing 6-membered condensed structure via an m-phenylene group.

In Example Compounds 1, 2, 3, 4 and 83, the nitrogen atom of the amine moiety is combined with the nitrogen atom of a nitrogen-containing 6-membered condensed structure via an m-phenylene group. In contrast, in Comparative Compounds C1 to C3, the nitrogen atom of the amine moiety is combined with the nitrogen atom of a nitrogen-containing 6-membered condensation structure via an p-phenylene group. Accordingly, Example Compounds 1, 2, 3, 4 and 83 have more narrow conjugation diffusion of π electrons between nitrogen atoms when compared to that of Comparative Compounds C1 to C3, and thus electron transfer from an emission layer to a hole transport layer may be inhibited or reduced. Accordingly, a driving voltage of the organic EL device including any of Example Compounds 1, 2, 3, 4 and 83 is decreased and its emission efficiency is improved.

In Example Compounds 1, 2, 3, 4 and 83, amine moiety is combined with the nitrogen atom of a nitrogen-containing 6-membered condensed structure. In contrast, in Comparative Compound C4, amine moiety is combined with the carbon atom of a nitrogen-containing 6-membered condensed structure. Accordingly, Example Compounds 1, 2, 3, 4 and 83 have more narrow conjugation diffusion of π electrons between nitrogen atoms when compared to that of Comparative Compound C4, and thus electron transfer may also be inhibited or reduced. Accordingly, a driving voltage of the organic EL device including any of Example Compounds 1, 2, 3, 4 and 83 is decreased and its emission efficiency is improved.

As described above, in a blue to bluish green region of an organic EL device, the driving voltage of the organic EL device may be decreased, and the emission efficiency thereof may be significantly improved, according to embodiments of the present disclosure.

In example embodiments, when a material for an organic EL device includes the amine derivative represented by Formula 1, a driving voltage may be decreased, and emission efficiency may be significantly improved in an organic EL device using the material. Thus, the material for an organic EL device according to an embodiment of the present disclosure may be utilized in a variety of practical applications.

As used herein, expressions such as “at least one of,” “one of,” “at least one selected from,” and “one selected from,” when preceding a list of elements, modify the entire list of elements and do not modify the individual elements of the list. Further, the use of “may” when describing embodiments of the present invention refers to “one or more embodiments of the present invention.”

In addition, as used herein, the terms “use,” “using,” and “used” may be considered synonymous with the terms “utilize,” “utilizing,” and “utilized,” respectively.

As used herein, the terms “substantially,” “about,” and similar terms are used as terms of approximation and not as terms of degree, and are intended to account for the inherent deviations in measured or calculated values that would be recognized by those of ordinary skill in the art.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein. All such ranges are intended to be inherently described in this specification such that amending to expressly recite any such subranges would comply with the requirements of 35 U.S.C. §112(a) and 35 U.S.C. §132(a).

It will be understood that the above-disclosed embodiments are to be considered illustrative and not restrictive, and the appended claims and equivalents thereof are intended to cover all such modifications, enhancements, and other embodiments, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing description.

What is claimed is:

1. An amine derivative represented by the following Formula 1: 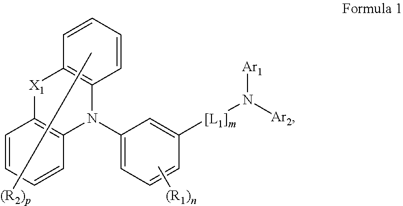
wherein Ar₁ and Ar₂ are each independently selected from a substituted or unsubstituted aryl group having 6 to 30 carbon atoms for forming a ring and a substituted or unsubstituted heteroaryl group having 2 to 30 carbon atoms for forming a ring,
L₁ is a divalent linker,
X₁ is selected from C(R₃)₂, C═O, Si(R₃)₂, N—R₃, O, S, SO and SO₂,
R₁, R₂ and R₃ are each independently selected from a substituted or unsubstituted linear, branched or cyclic alkyl group having 1 to 16 carbon atoms, a substituted or unsubstituted aryl group having 6 to 36 carbon atoms for forming a ring, a substituted or unsubstituted heteroaryl group having 2 to 32 carbon atoms for forming a ring, and an aryl group or a heteroaryl group formed via condensation of a plurality of adjacent substituents,
n is an integer selected from 0 to 4,
m is an integer selected from 1 to 4, and
p is an integer selected from 0 to 8.

2. The amine derivative of claim 1, wherein L₁ is selected from a substituted or unsubstituted arylene group having 6 to 30 carbon atoms for forming a ring and a substituted or unsubstituted heteroarylene group having 2 to 30 carbon atoms for forming a ring.

3. The amine derivative of claim 1, wherein Ar₁ and Ar₂ are each independently selected from a group substituted with a substituent not including a nitrogen atom, and an unsubstituted aryl group or heteroaryl group, the aryl group or the heteroaryl group not including a nitrogen atom.

4. The amine derivative of claim 1, wherein Ar₁ is a group substituted with a substituent not including a nitrogen atom or an unsubstituted aryl group or heteroaryl group not including a nitrogen atom, and
Ar₂ is represented by the following Formula 2: 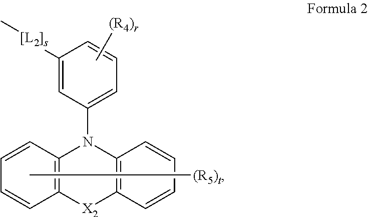
wherein L₂ is a divalent linker,
X₂ is selected from C(R₆)₂, C═O, Si(R₆)₂, N—R₆, O, S, SO and SO₂,
R₄, R₅ and R₆ are each independently selected from a substituted or unsubstituted linear, branched or cyclic alkyl group having 1 to 16 carbon atoms, a substituted or unsubstituted aryl group having 6 to 36 carbon atoms for forming a ring, a substituted or unsubstituted heteroaryl group having 2 to 32 carbon atoms for forming a ring, and an aryl group or a heteroaryl group formed via condensation of a plurality of adjacent substituents,
r is an integer selected from 0 to 4,
s is an integer selected from 1 to 4, and
t is an integer selected from 0 to 8.

5. The amine derivative of claim 4, wherein
s in Formula 2 is the same as m in Formula 1,
r in Formula 2 is the same as n in Formula 1, and
t in Formula 2 is the same as p in Formula 1.

6. The amine derivative of claim 1, wherein the amine derivative represented by Formula 1 is at least one selected from the following Compounds 1 to 208:

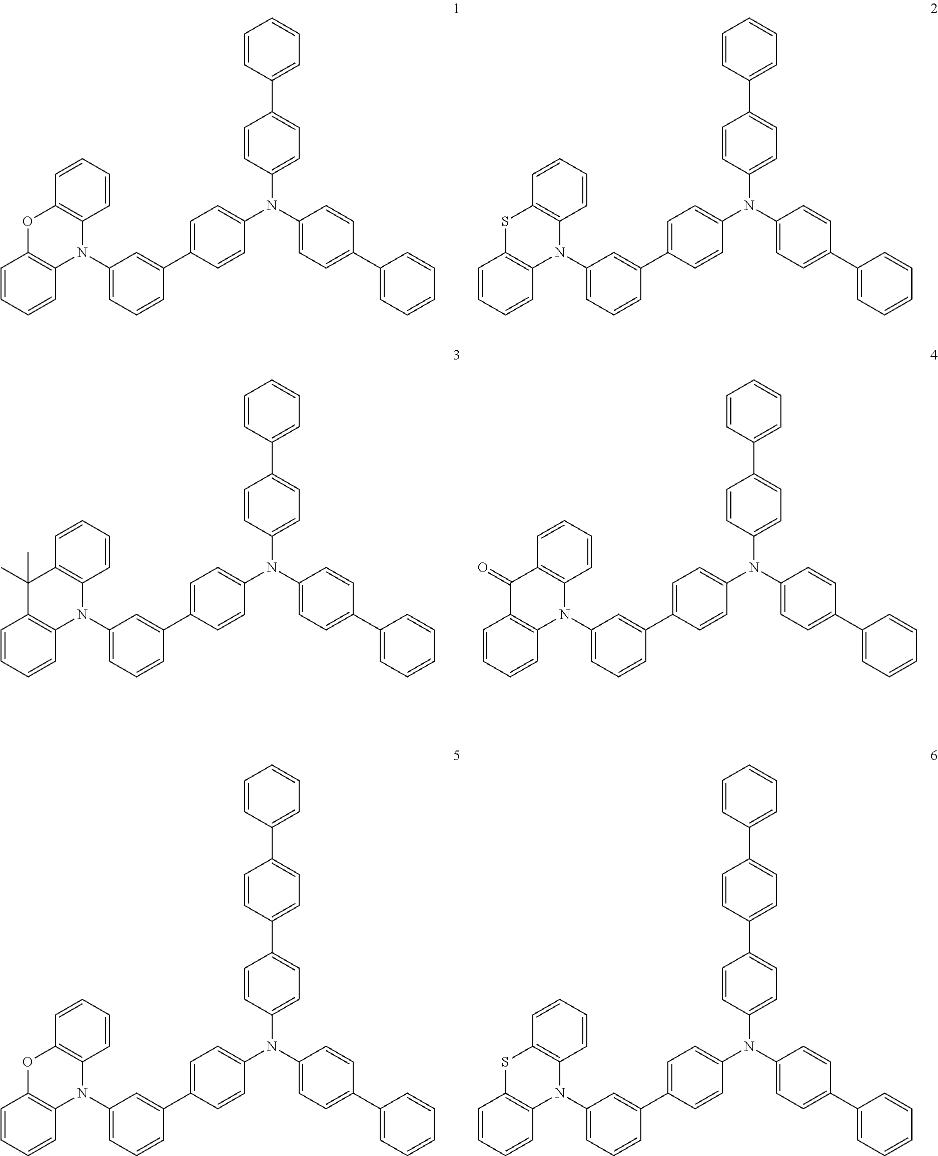

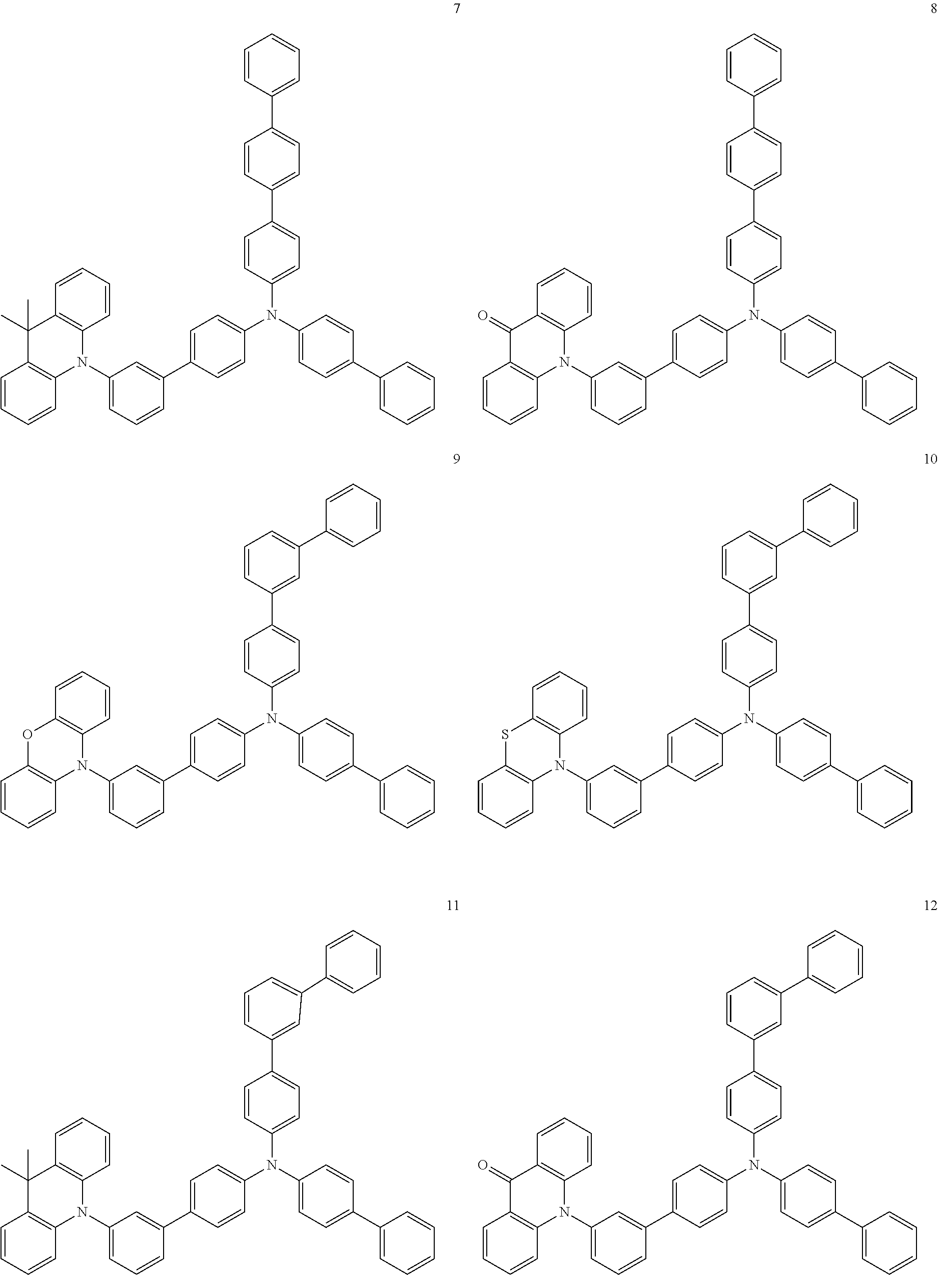

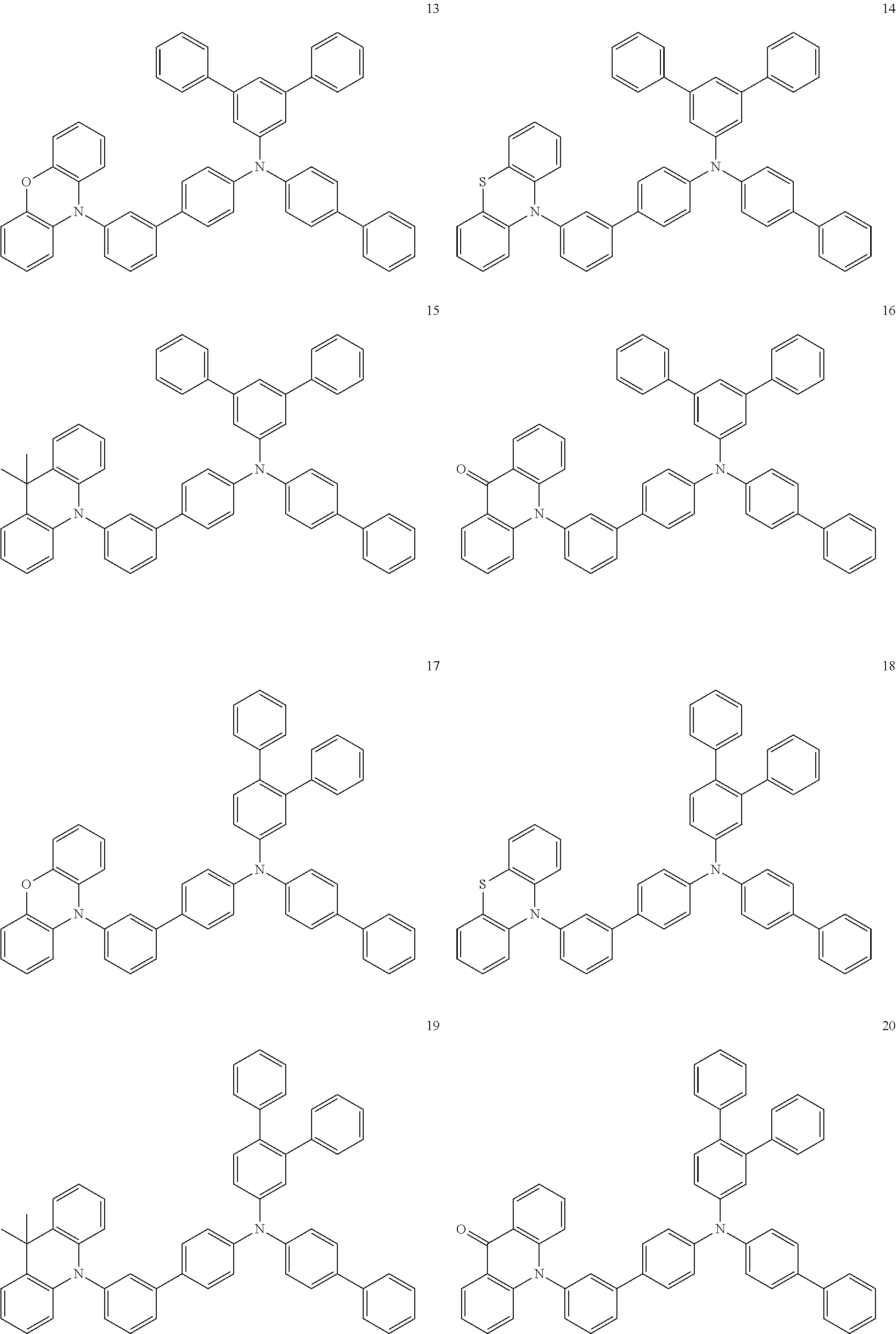

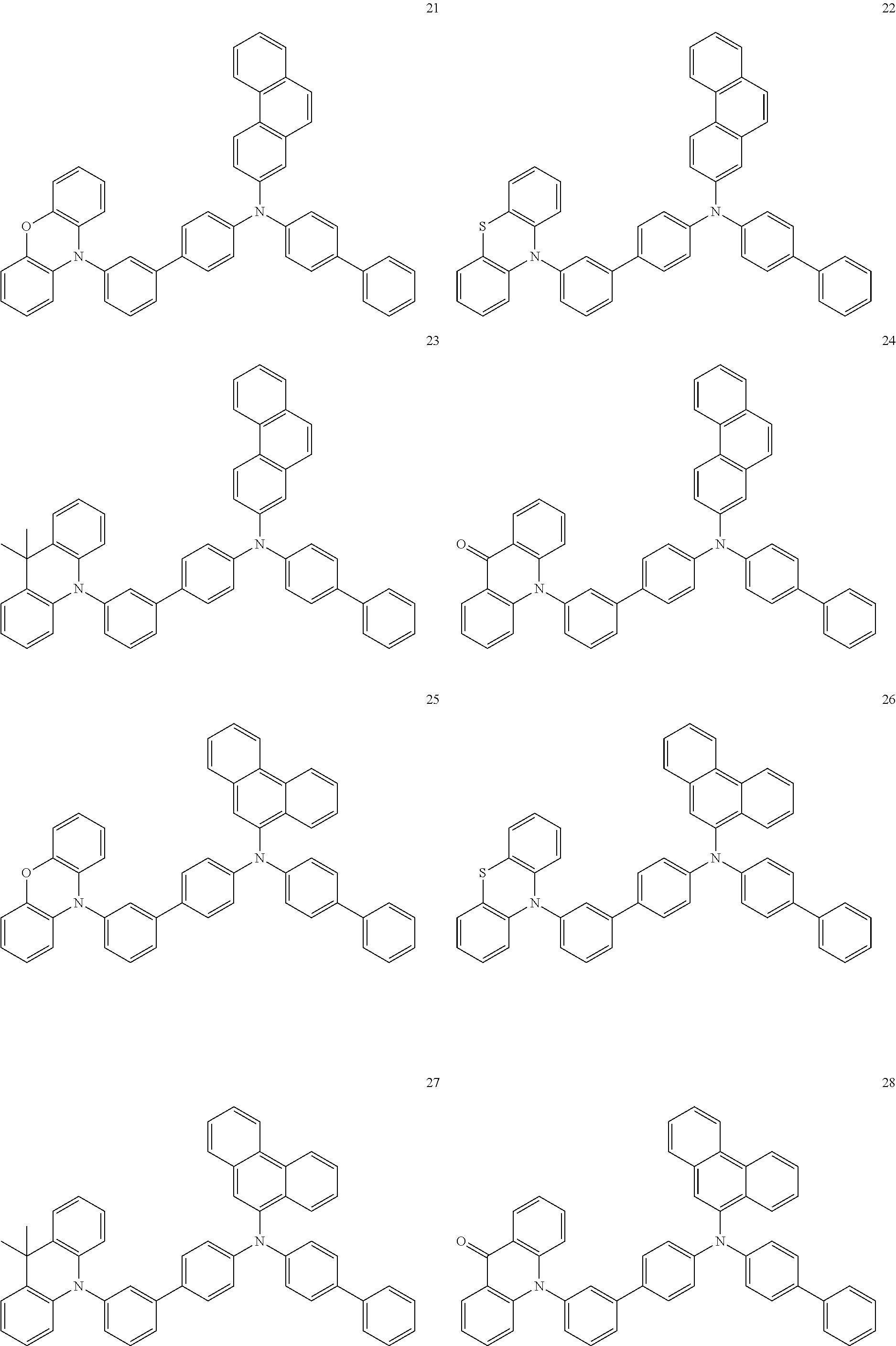

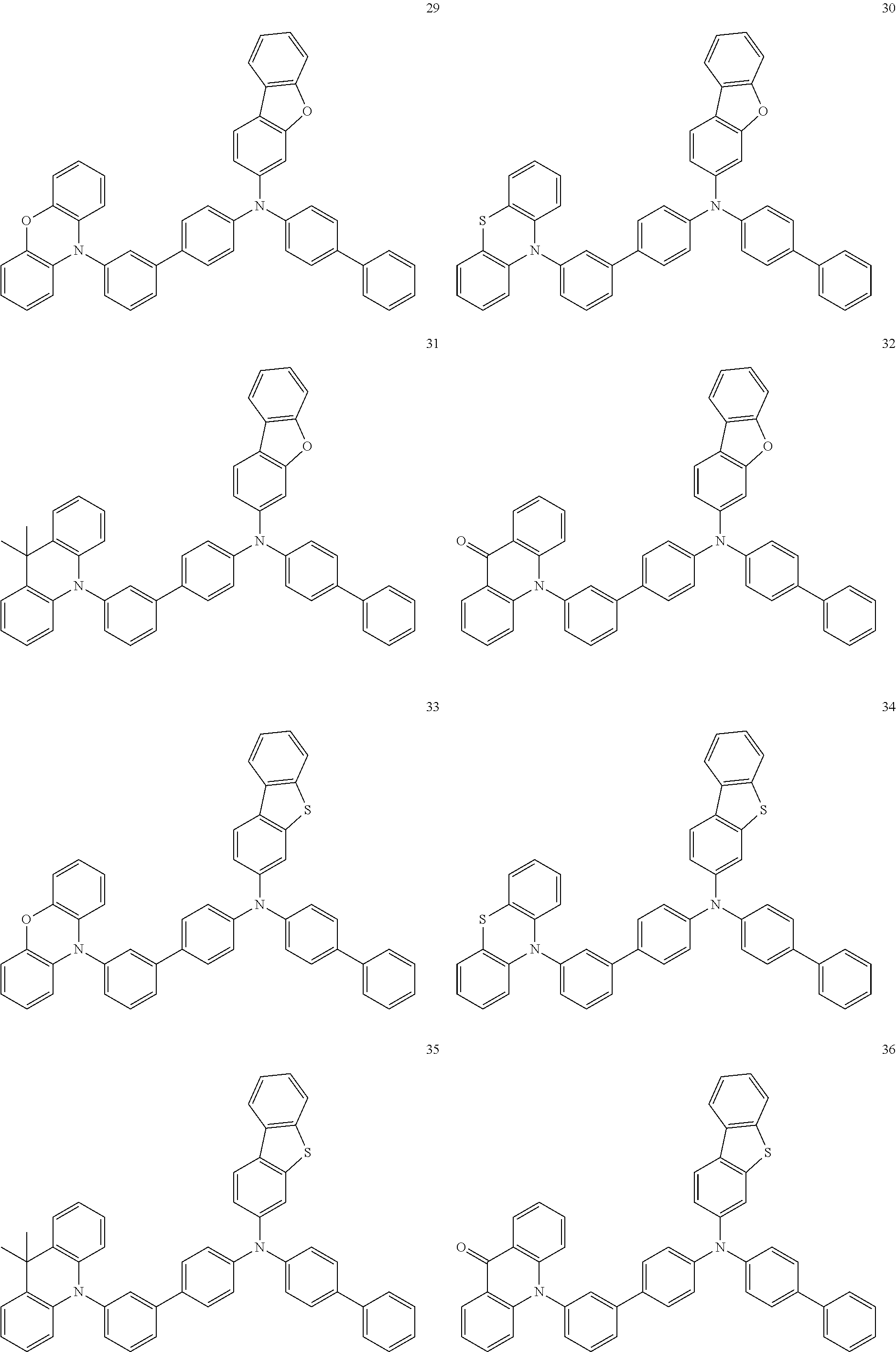

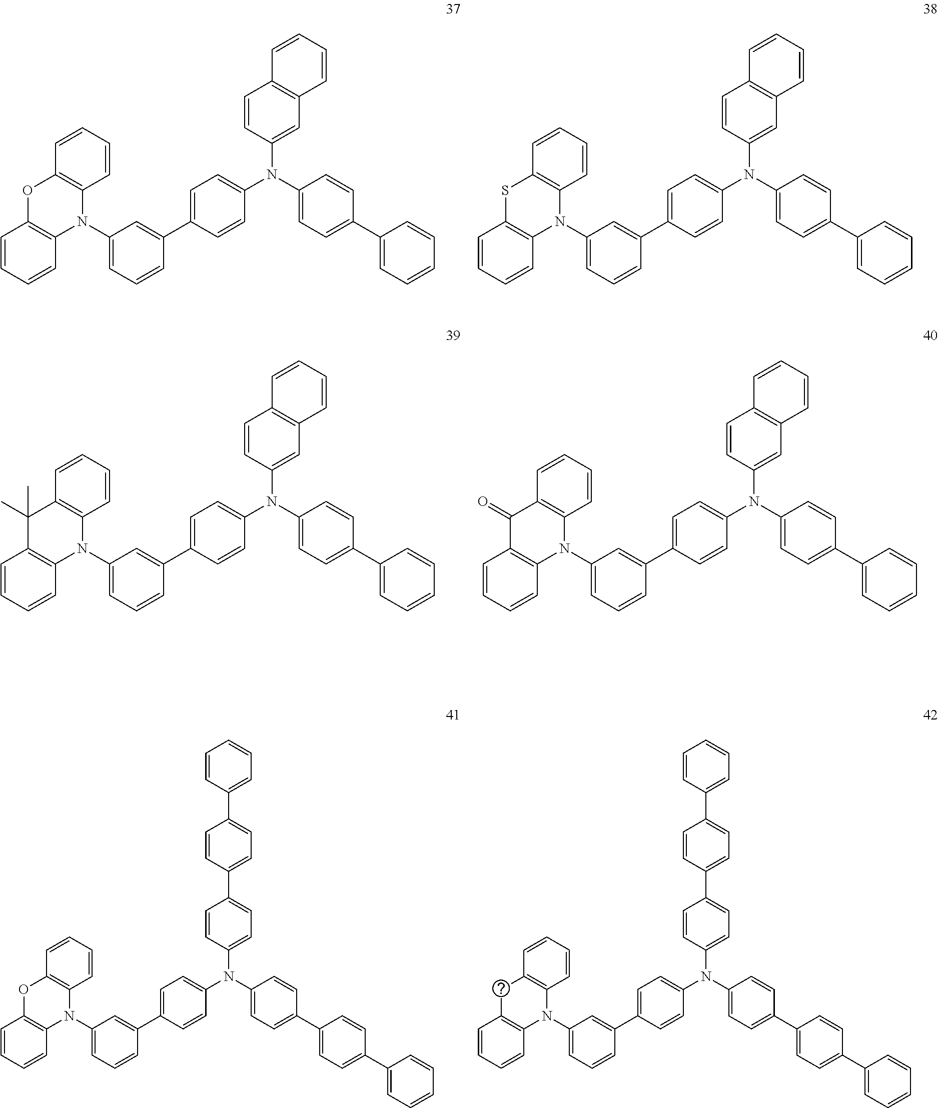

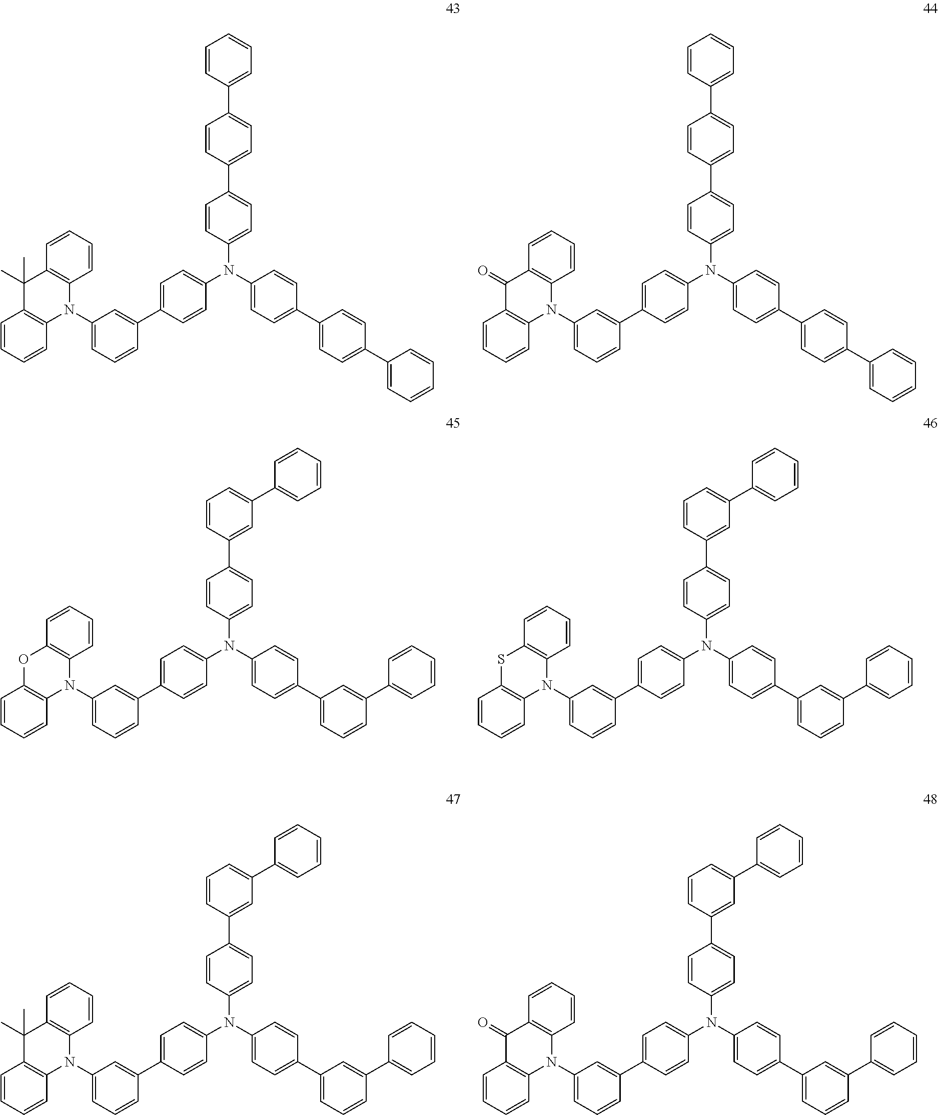

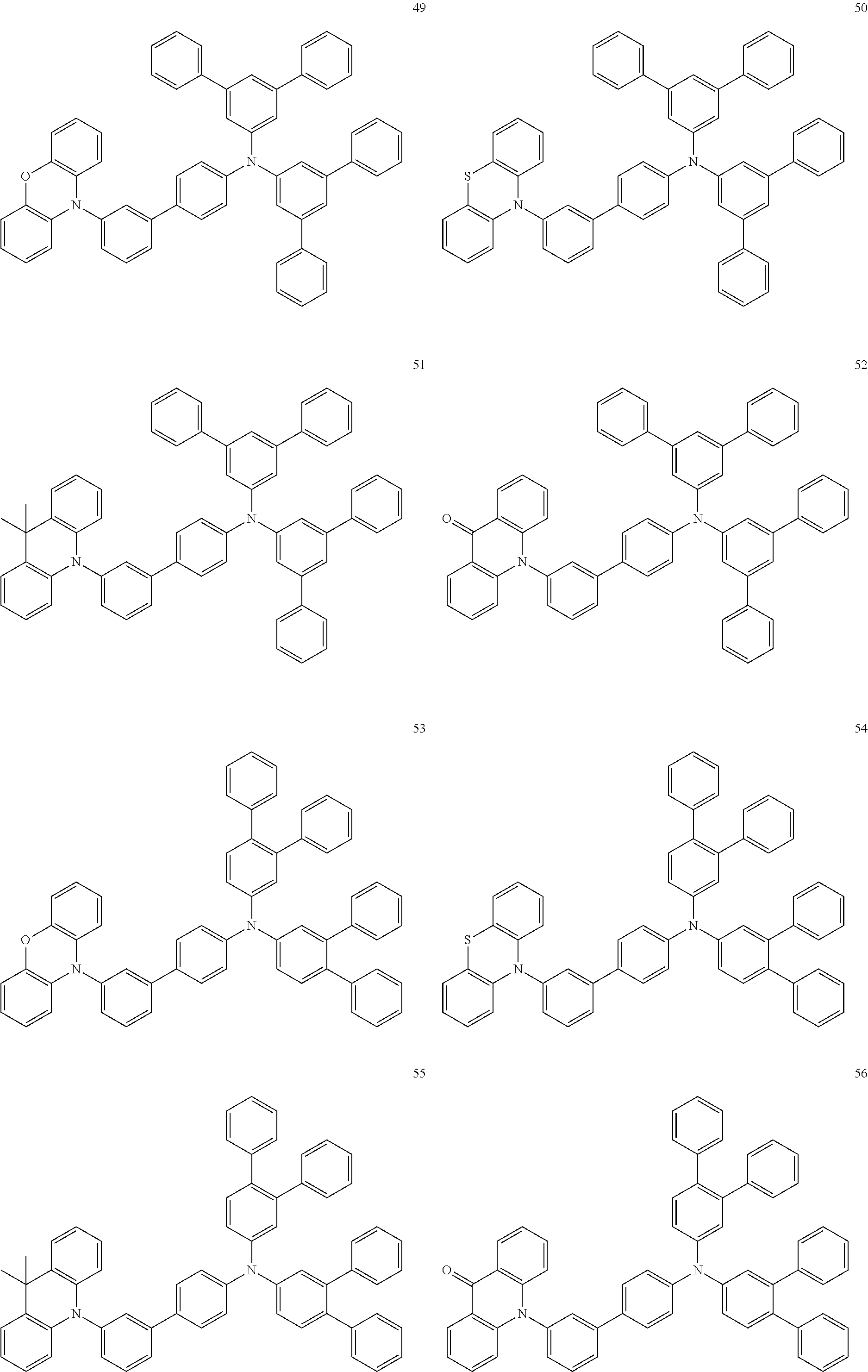

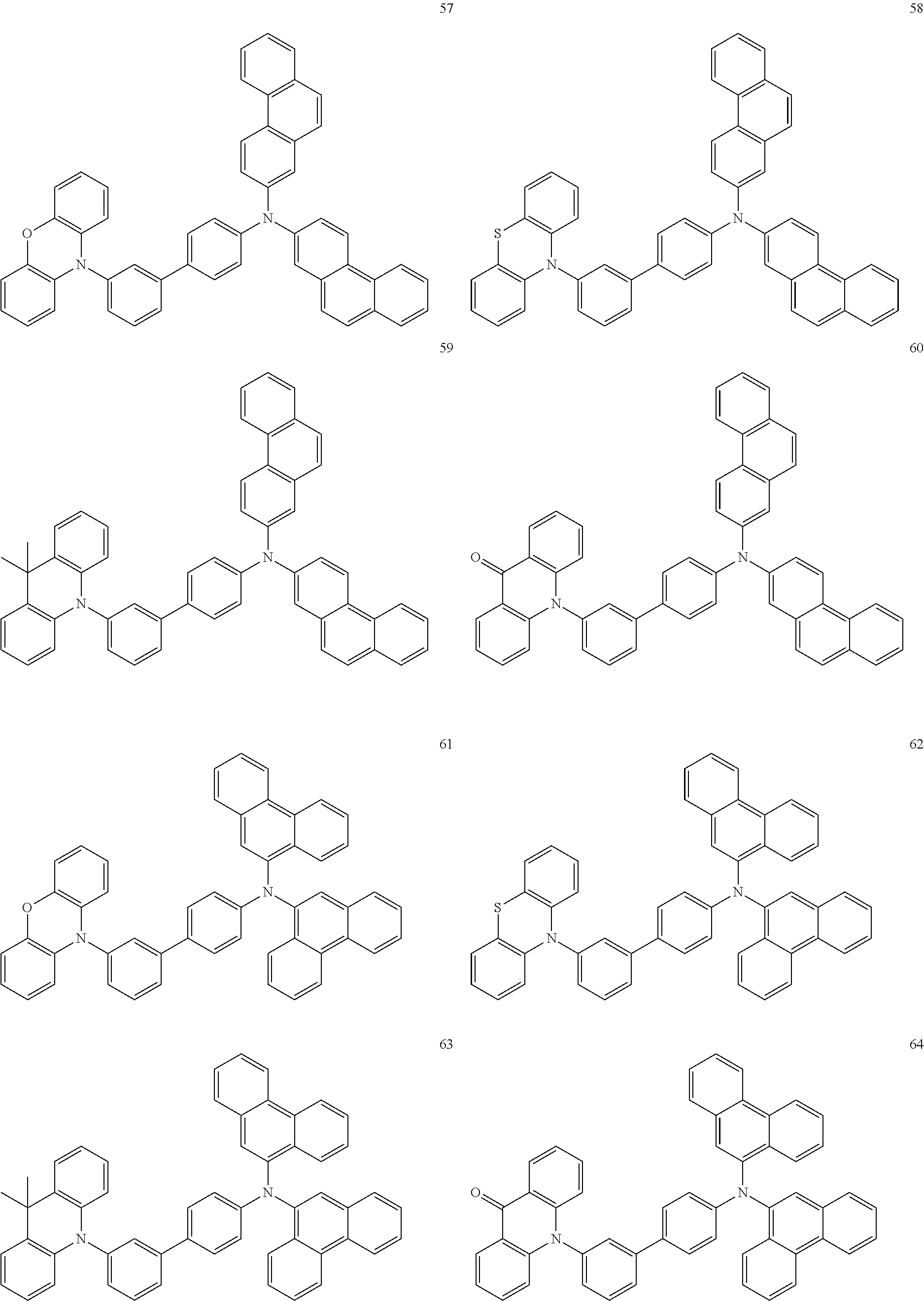

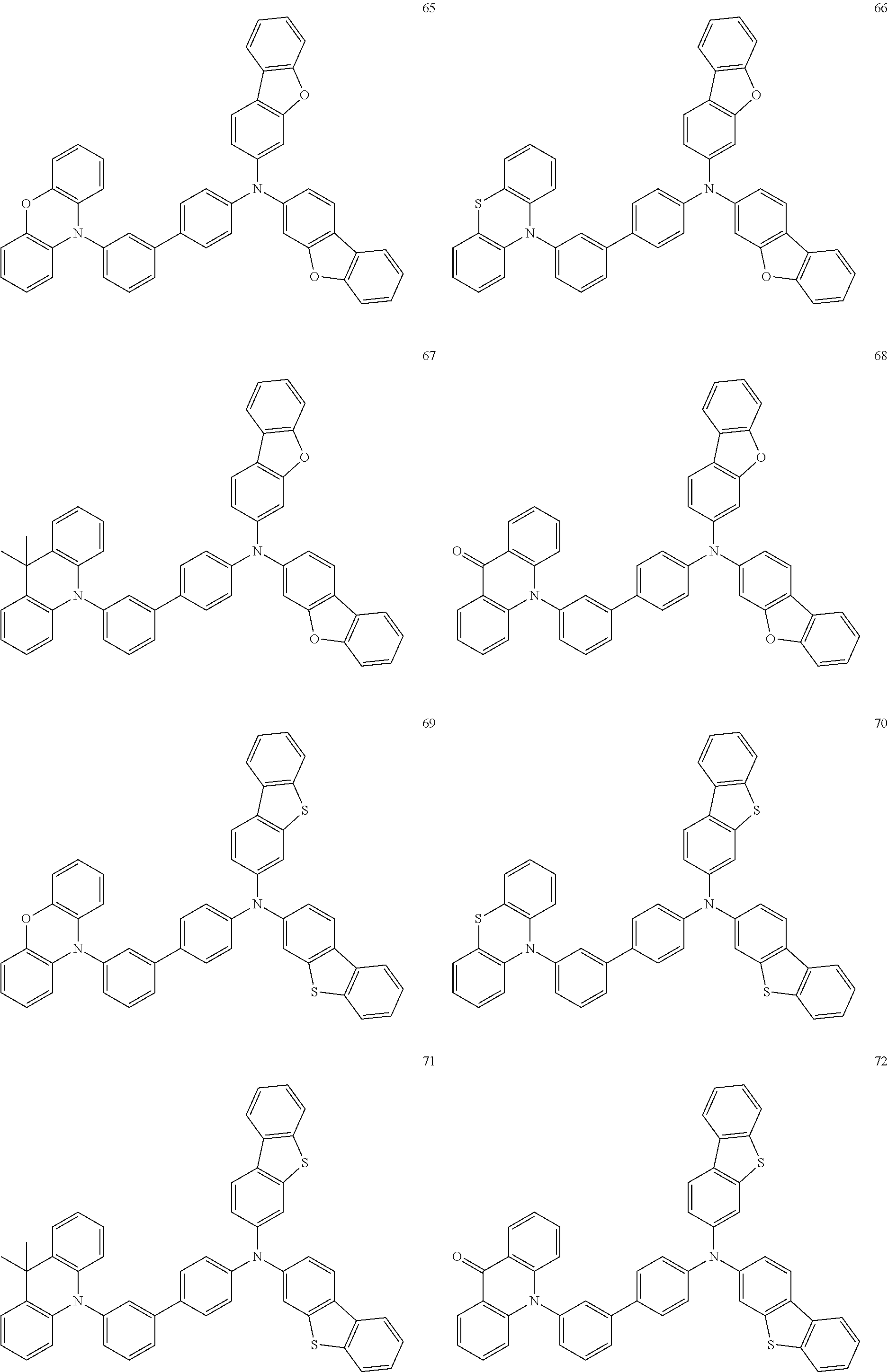

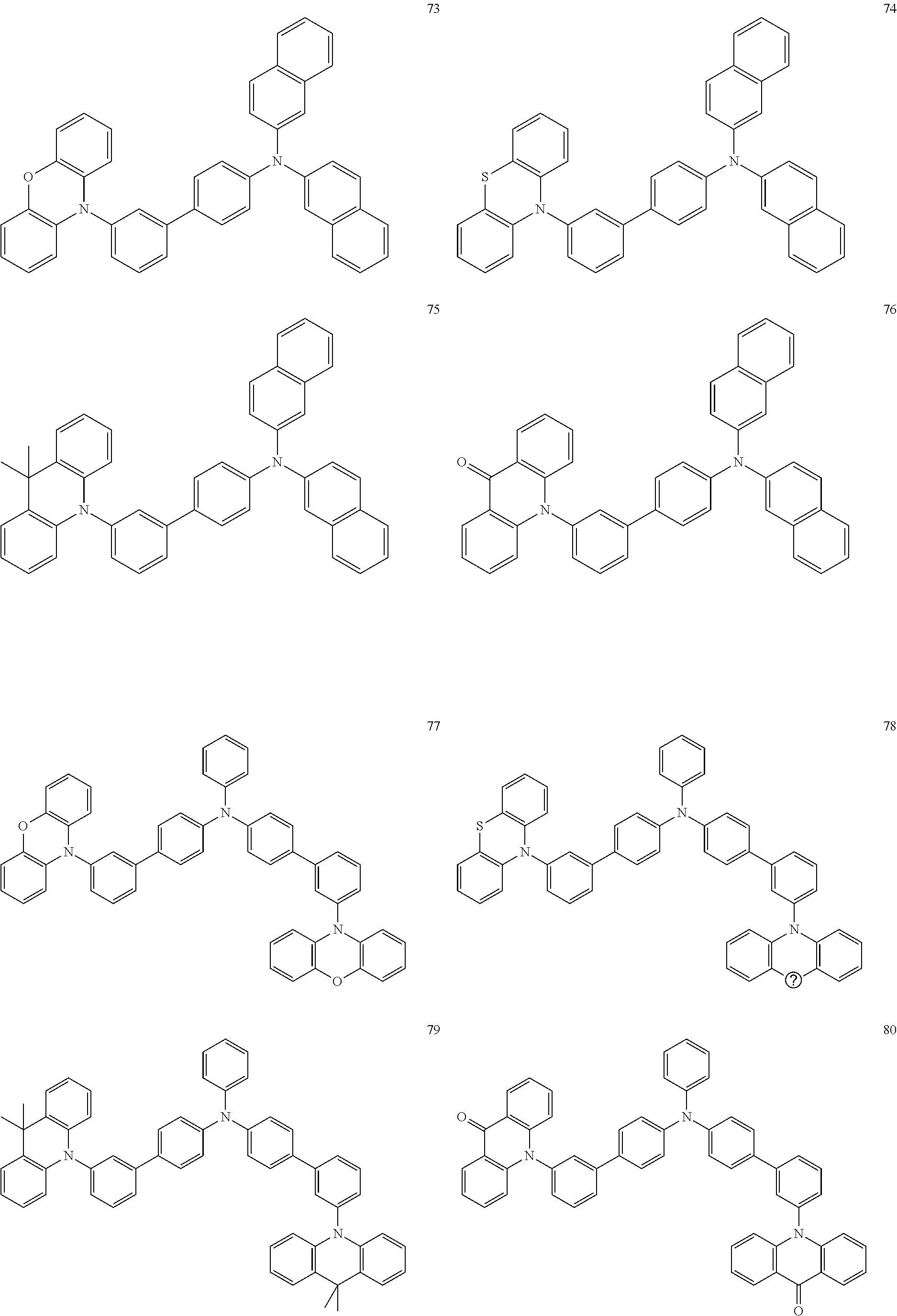

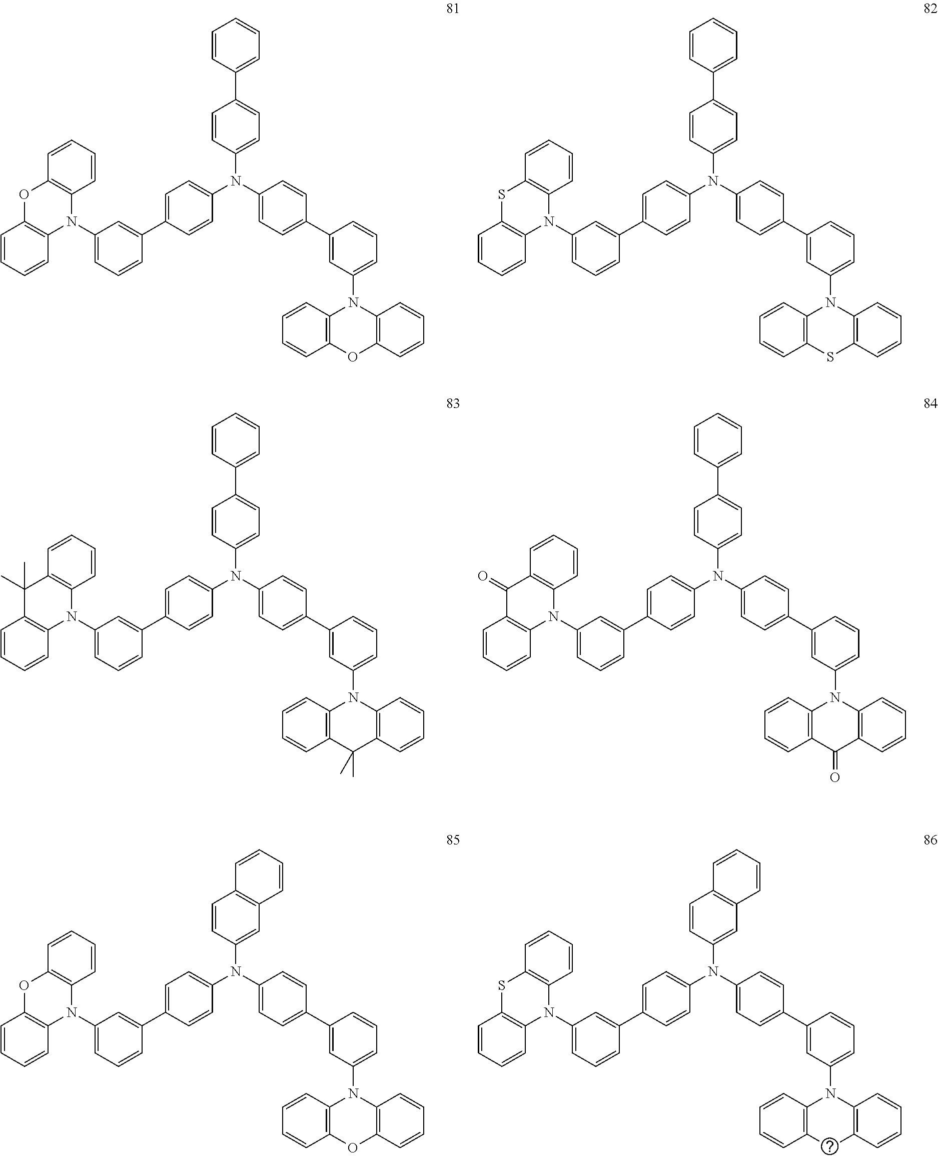

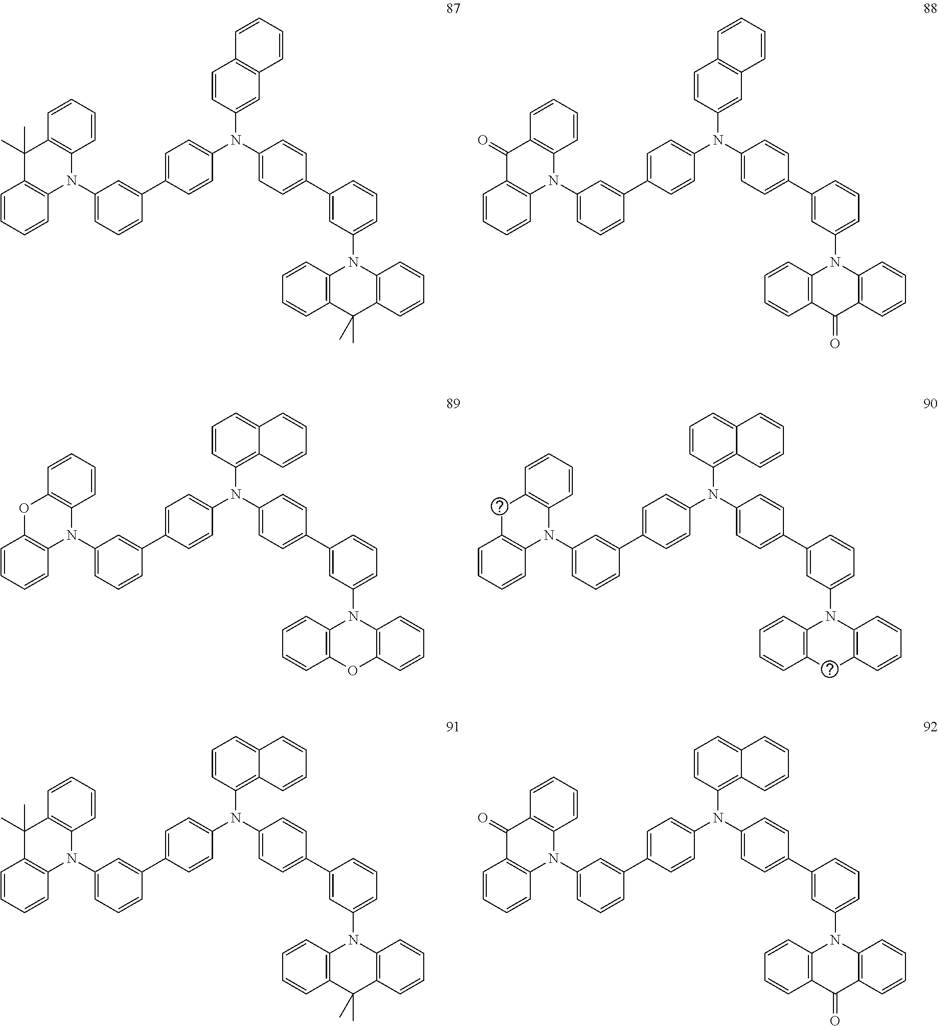

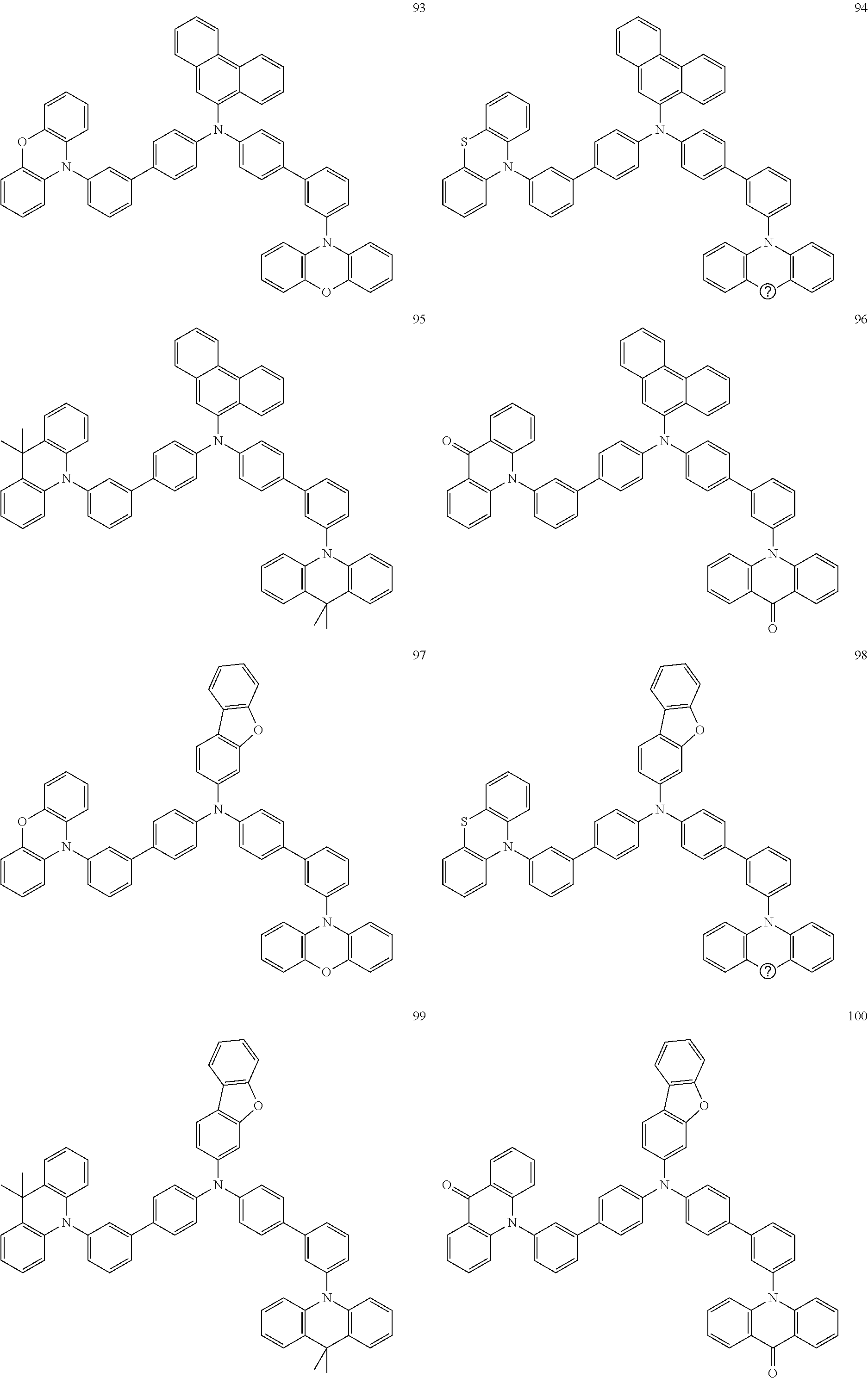

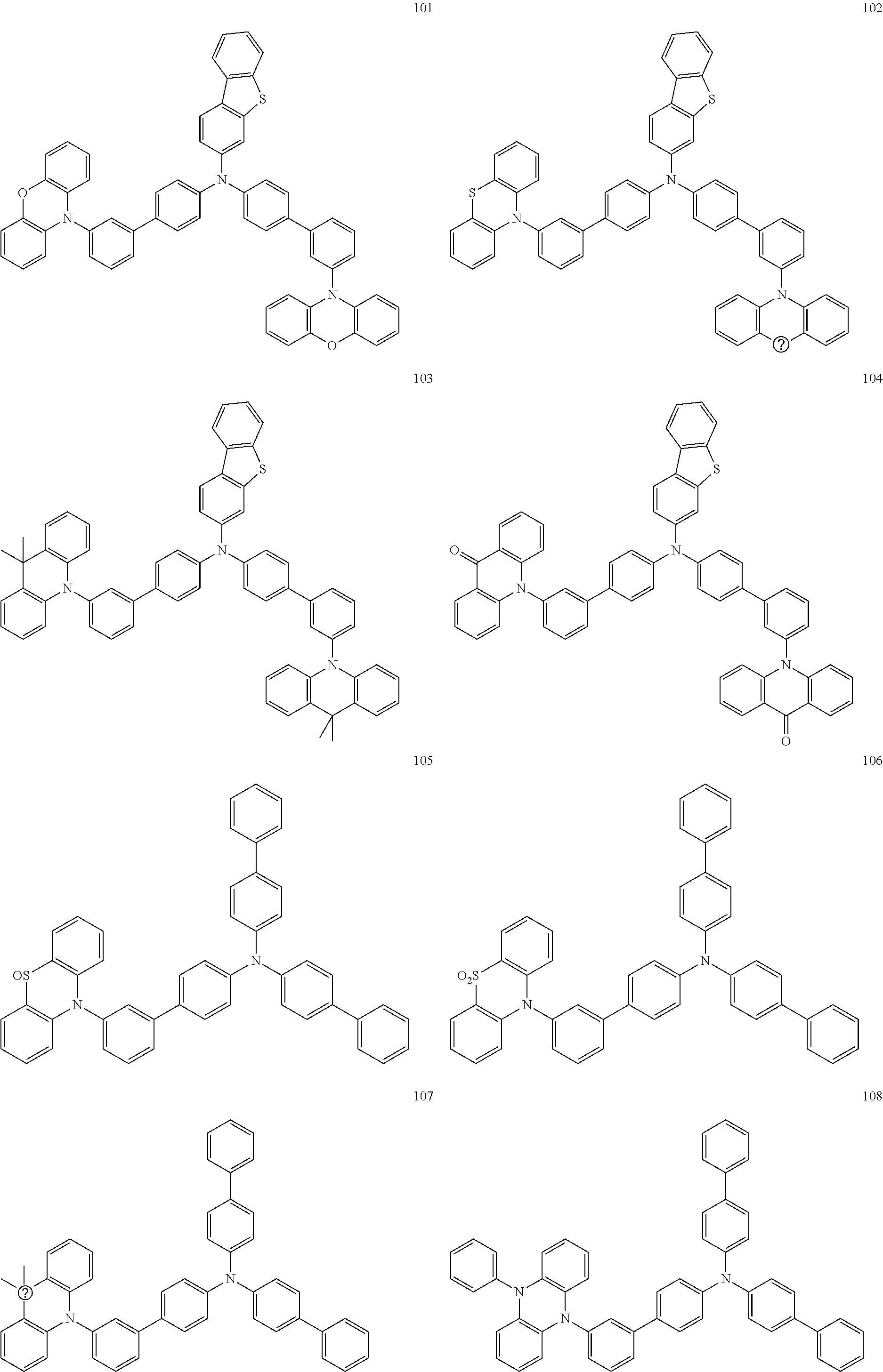

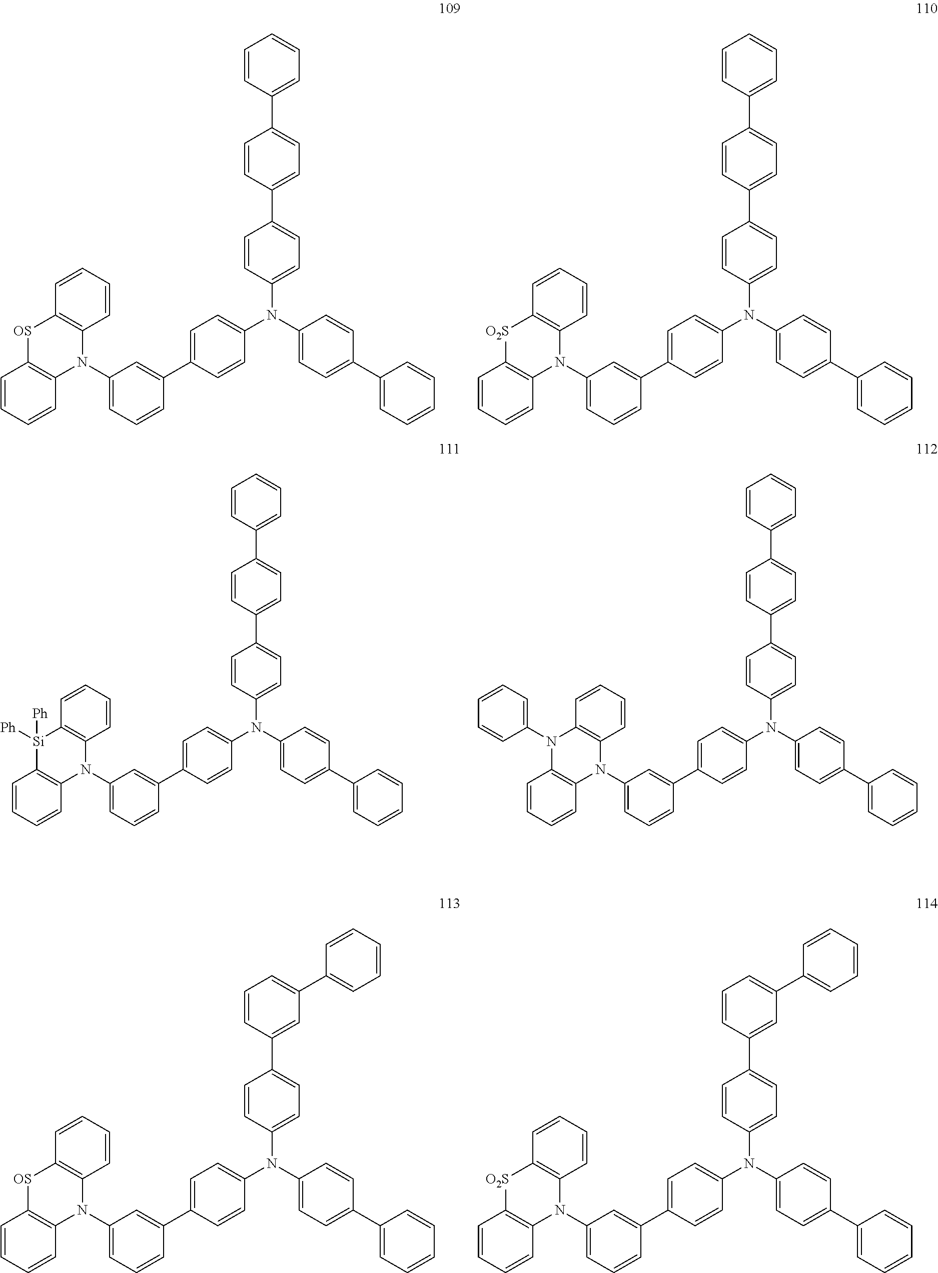

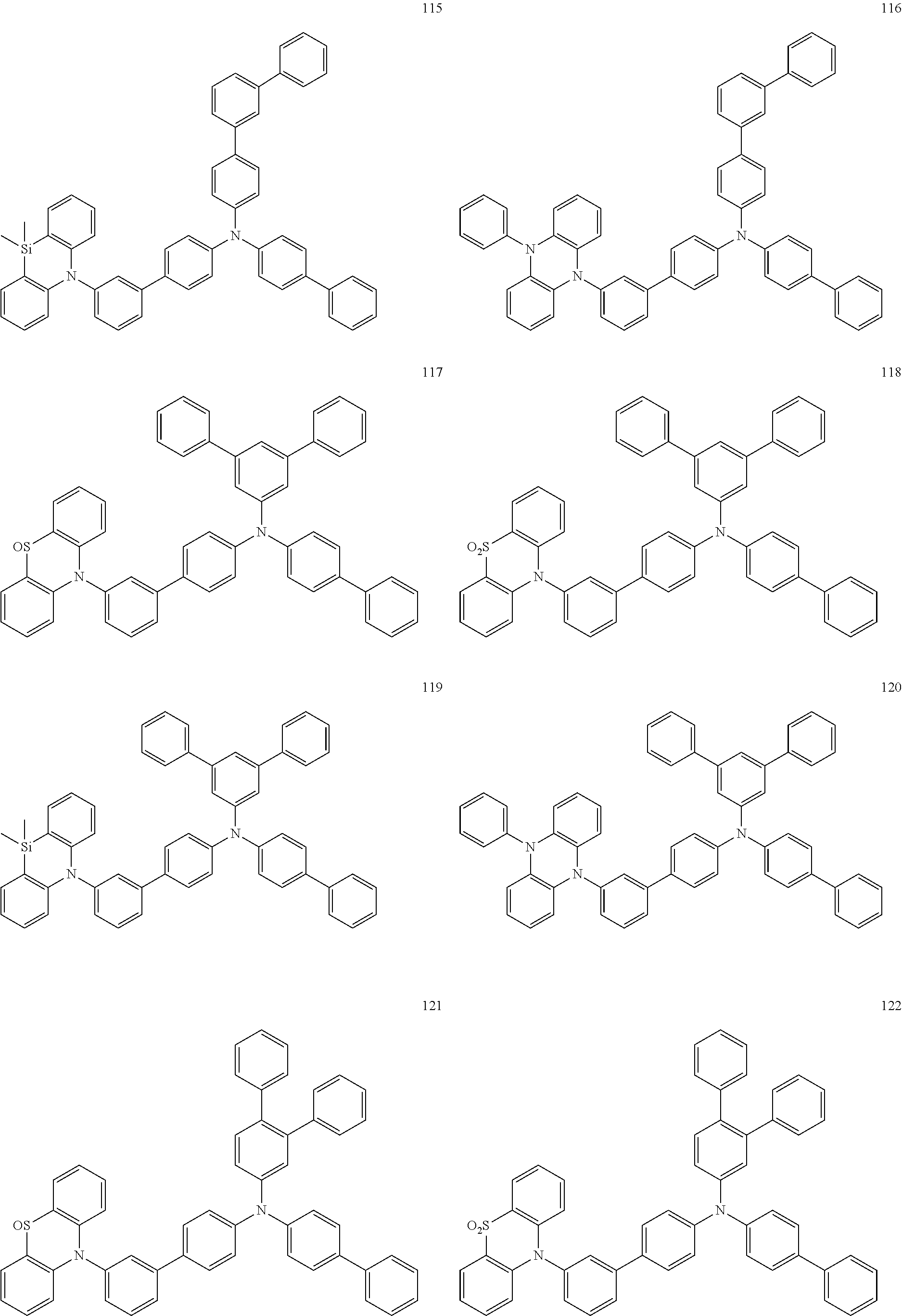

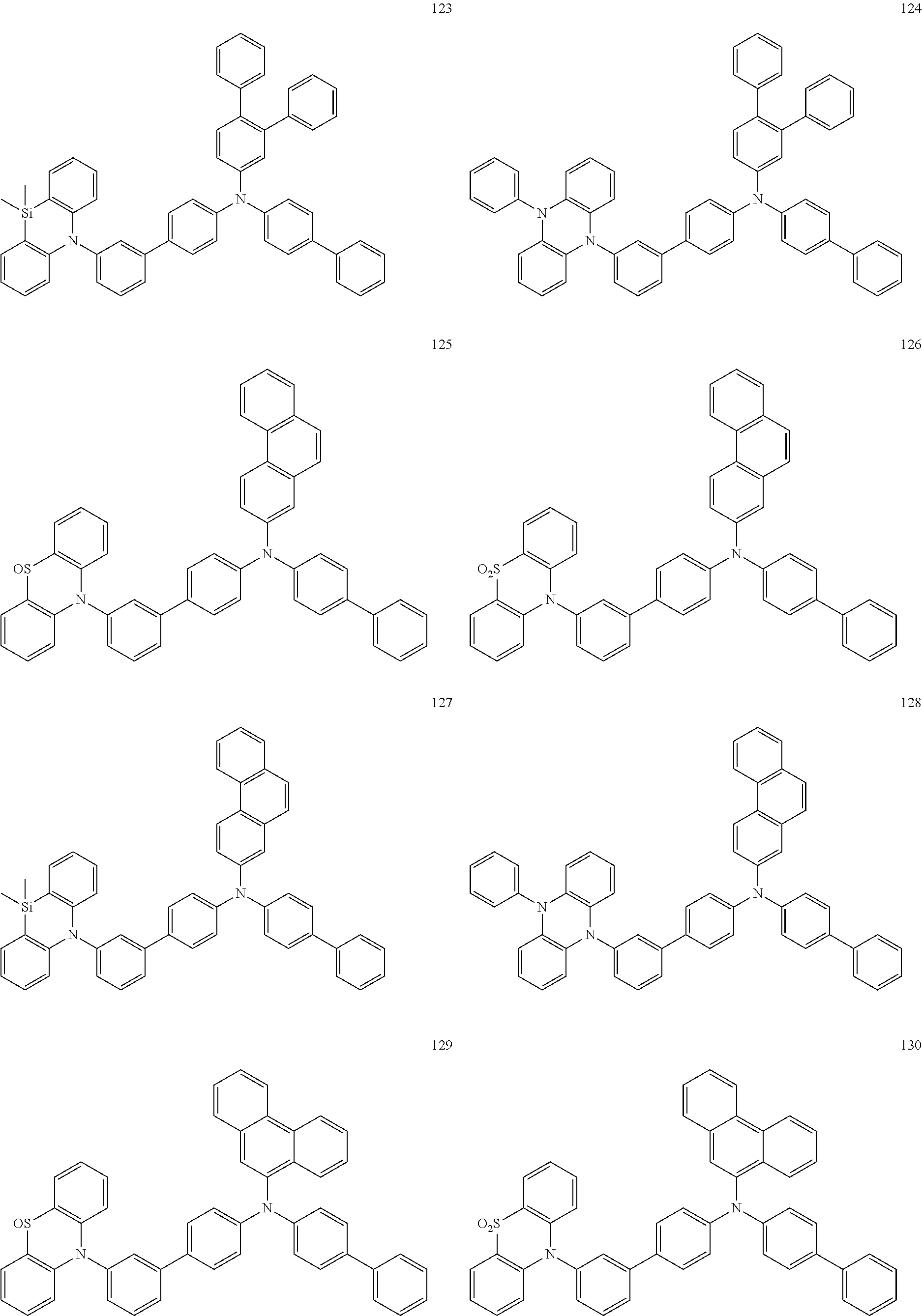

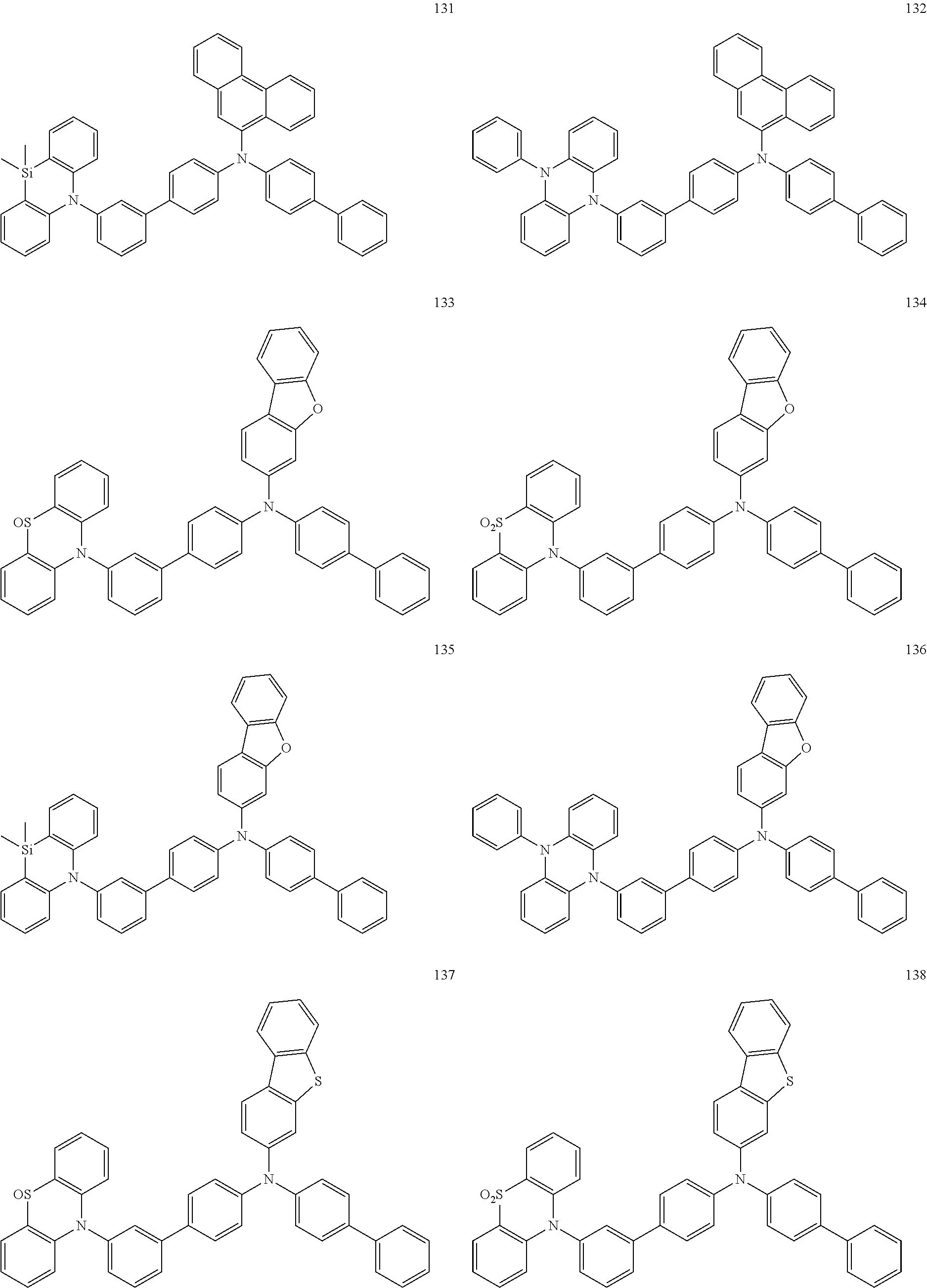

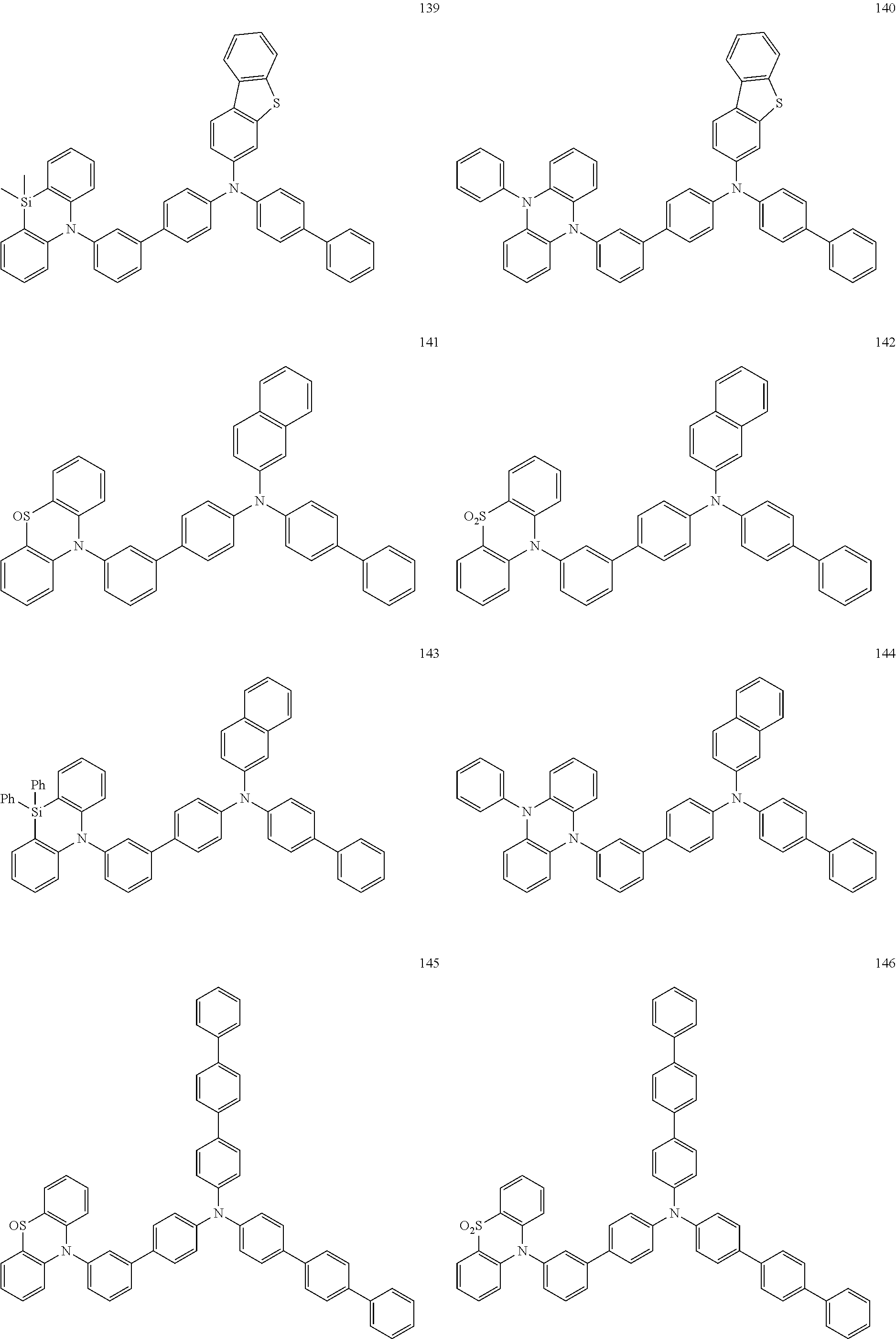

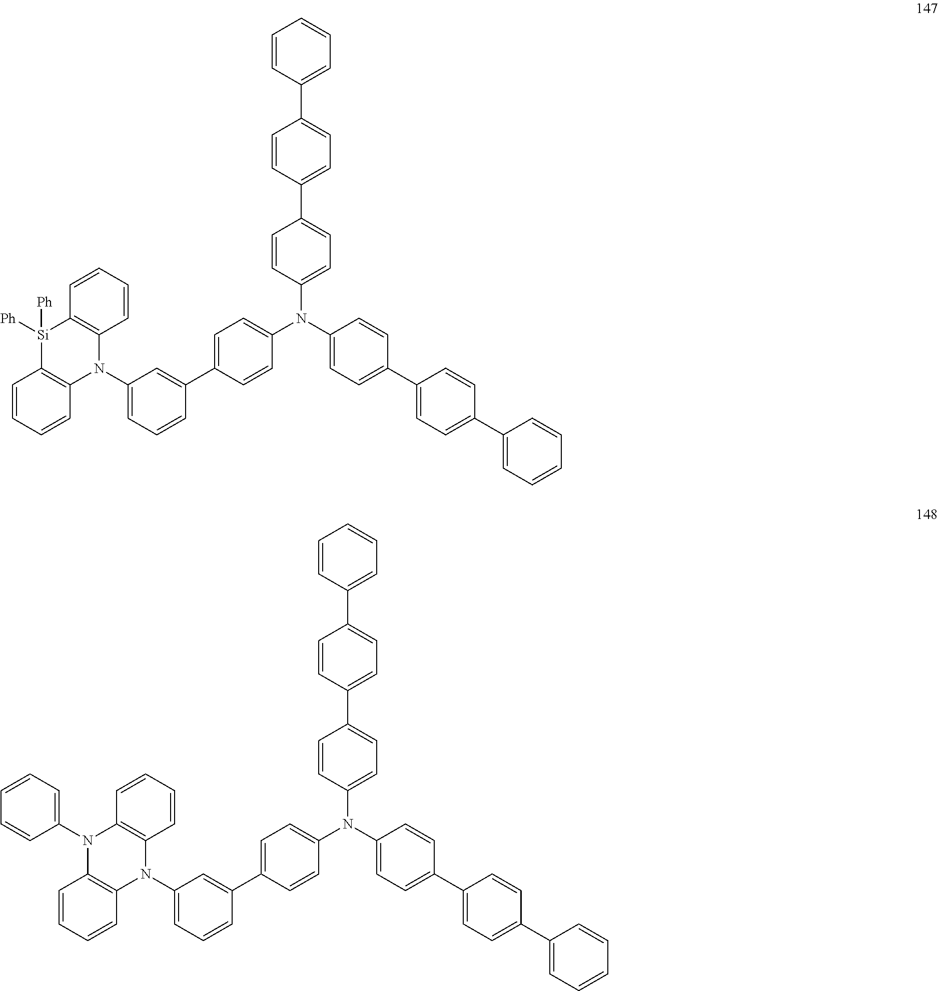 [SMILES: c1ccc(-c2ccc(-c3ccc(N(c4ccc(-c5ccc(-c6ccccc6)cc5)cc4)c4ccc(-c5cccc(N6c7ccccc7[Si](c7ccccc7)(c7ccccc7)c7ccccc76)c5)cc4)cc3)cc2)cc1 ; c1ccc(-c2ccc(-c3ccc(N(c4ccc(-c5ccc(-c6ccccc6)cc5)cc4)c4ccc(-c5cccc(N6c7ccccc7N(c7ccccc7)c7ccccc76)c5)cc4)cc3)cc2)cc1]

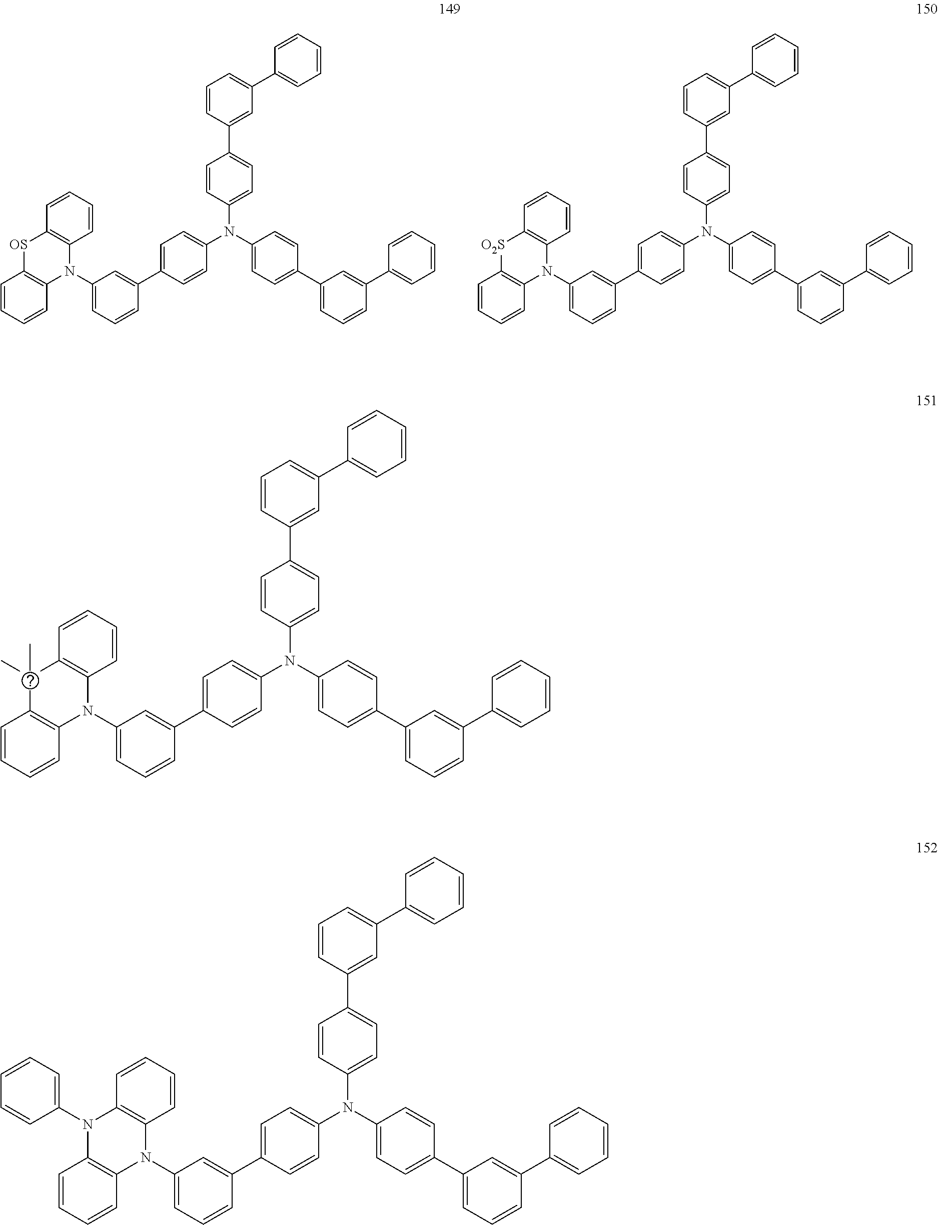

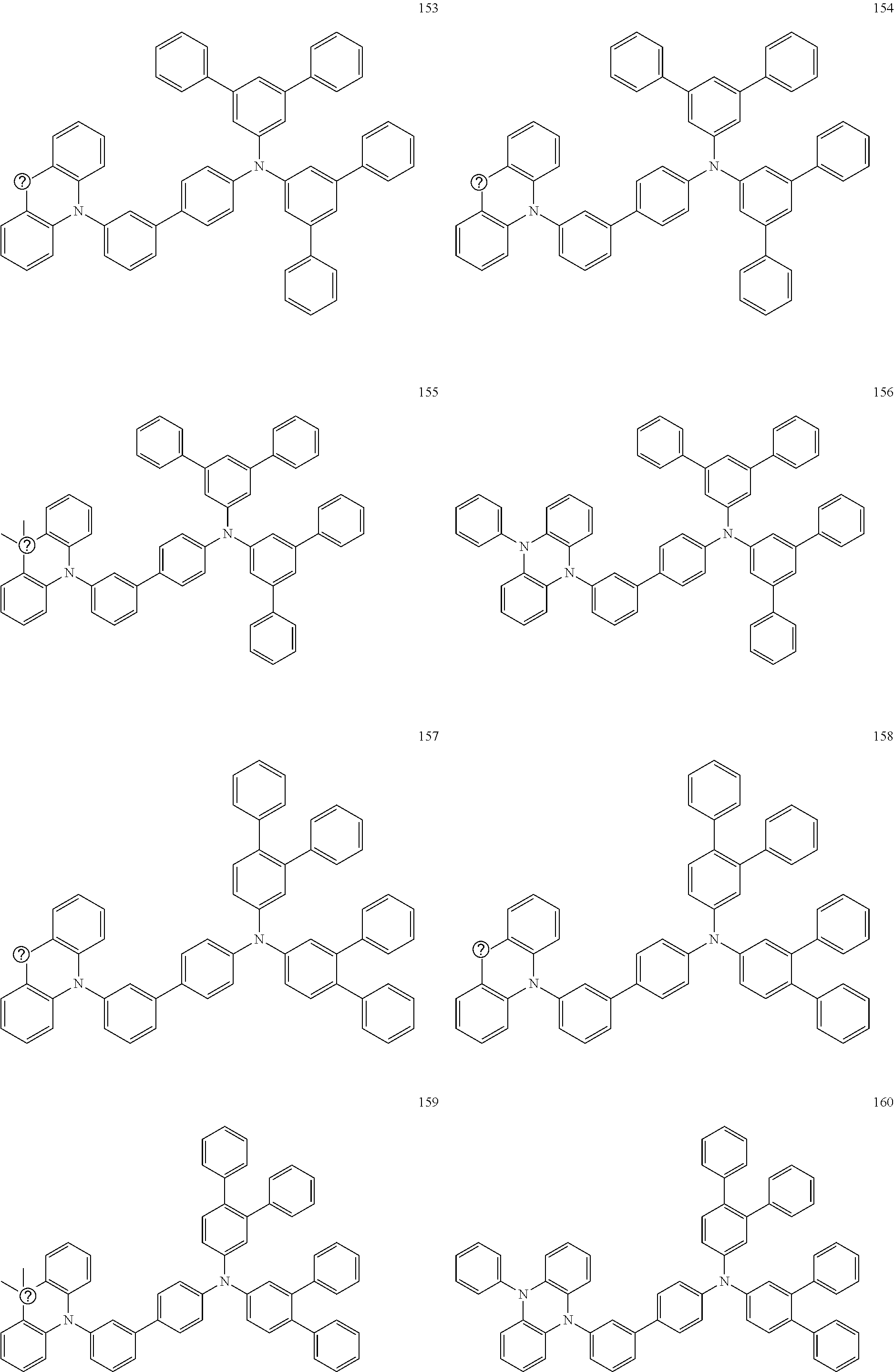

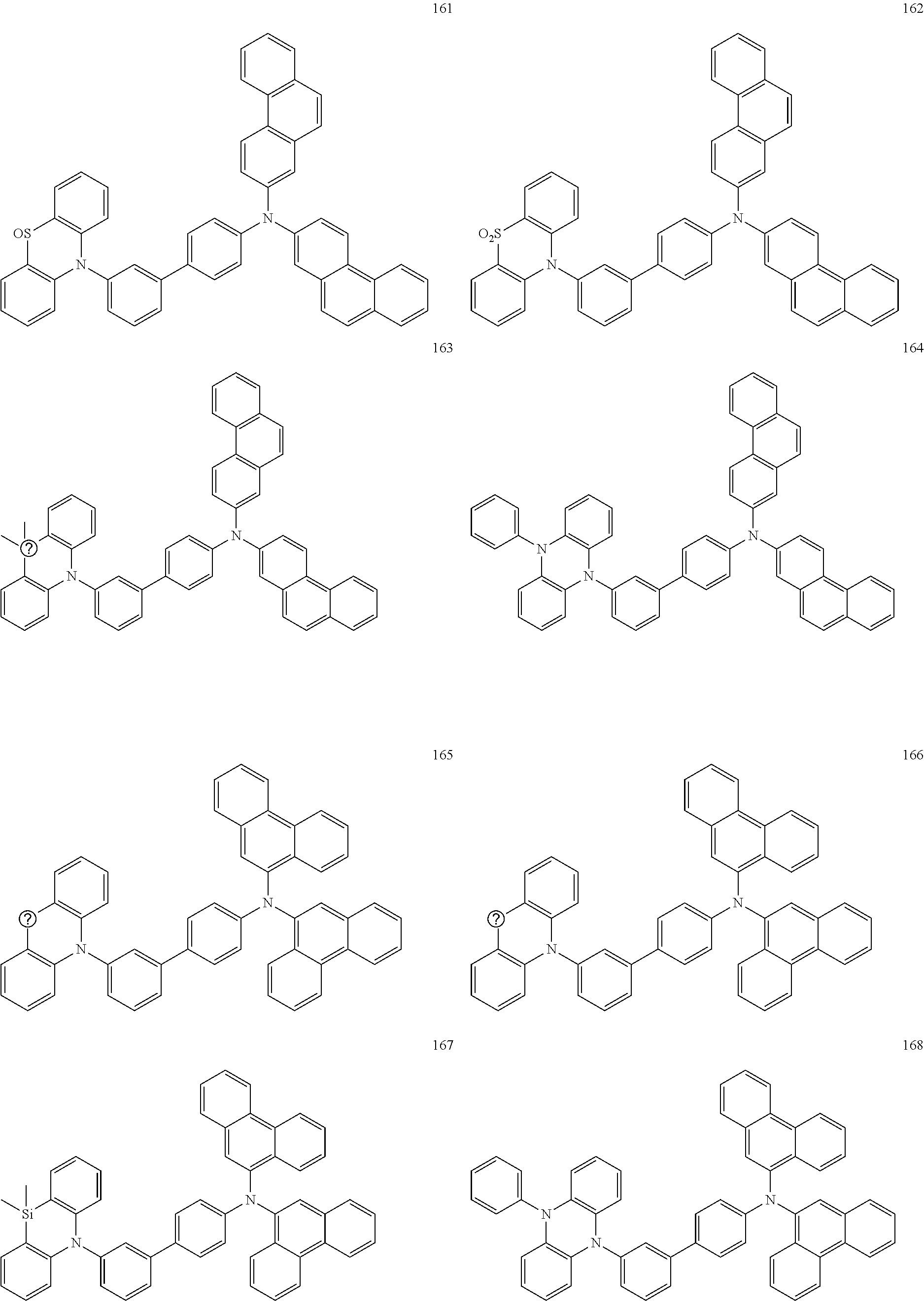

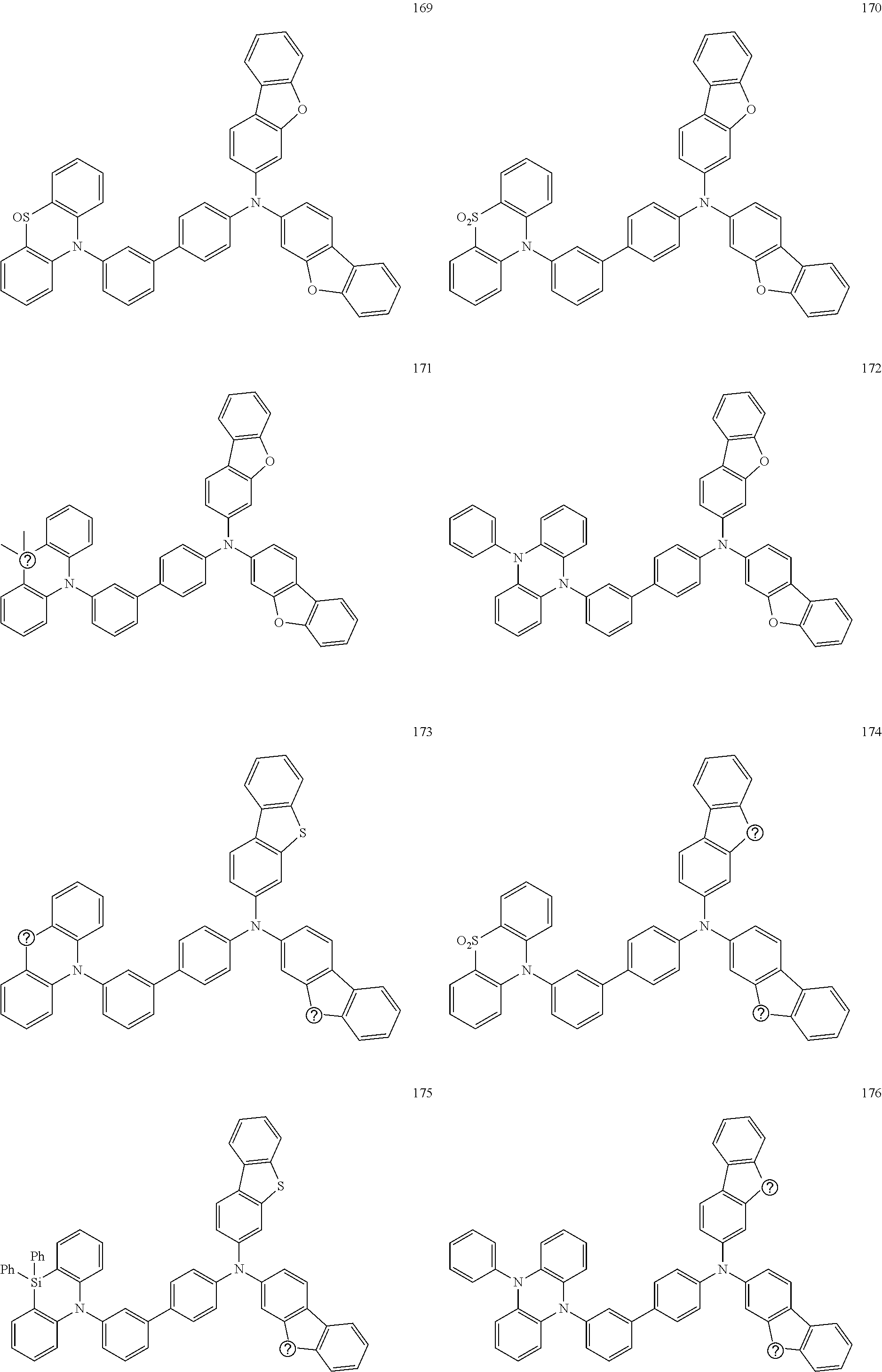

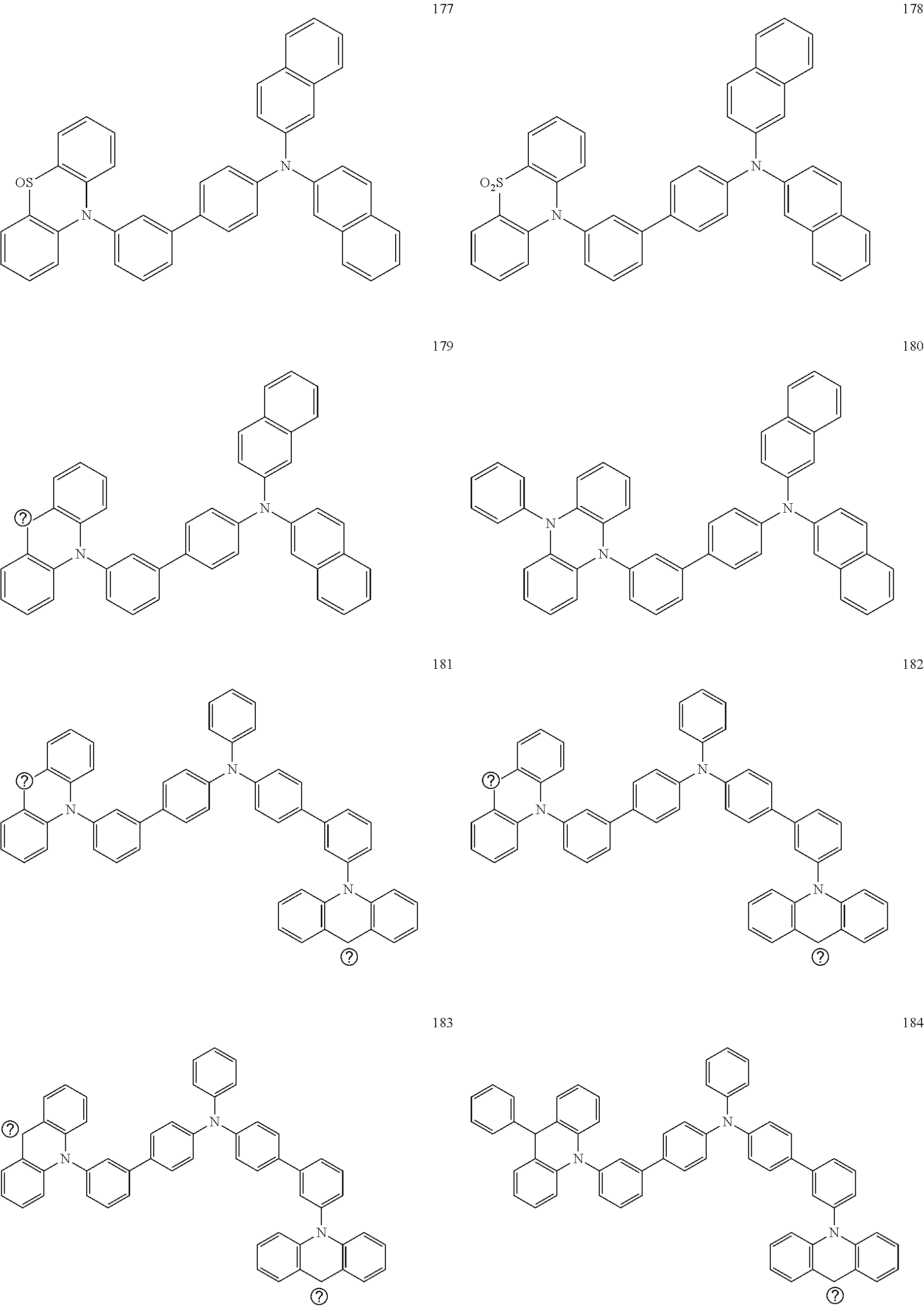

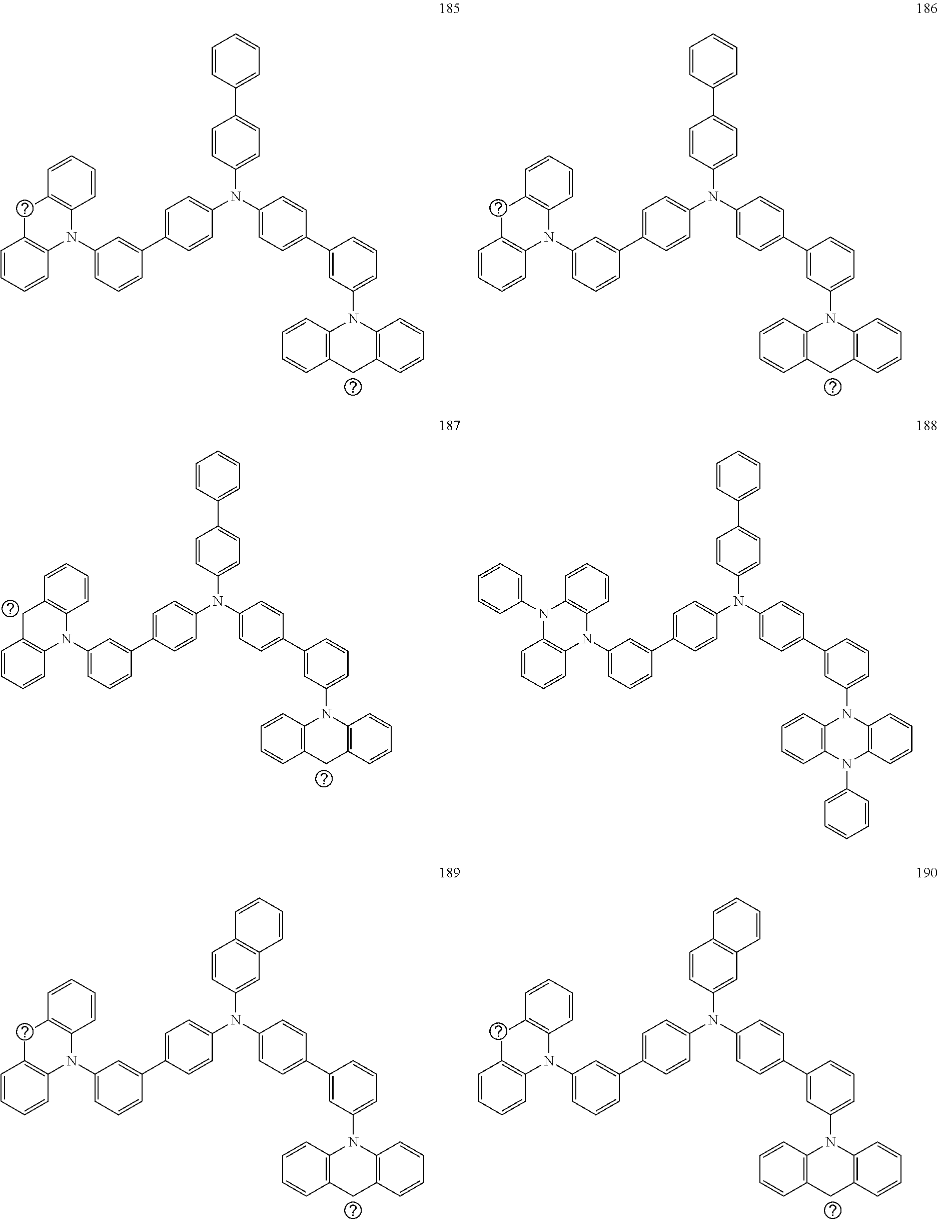

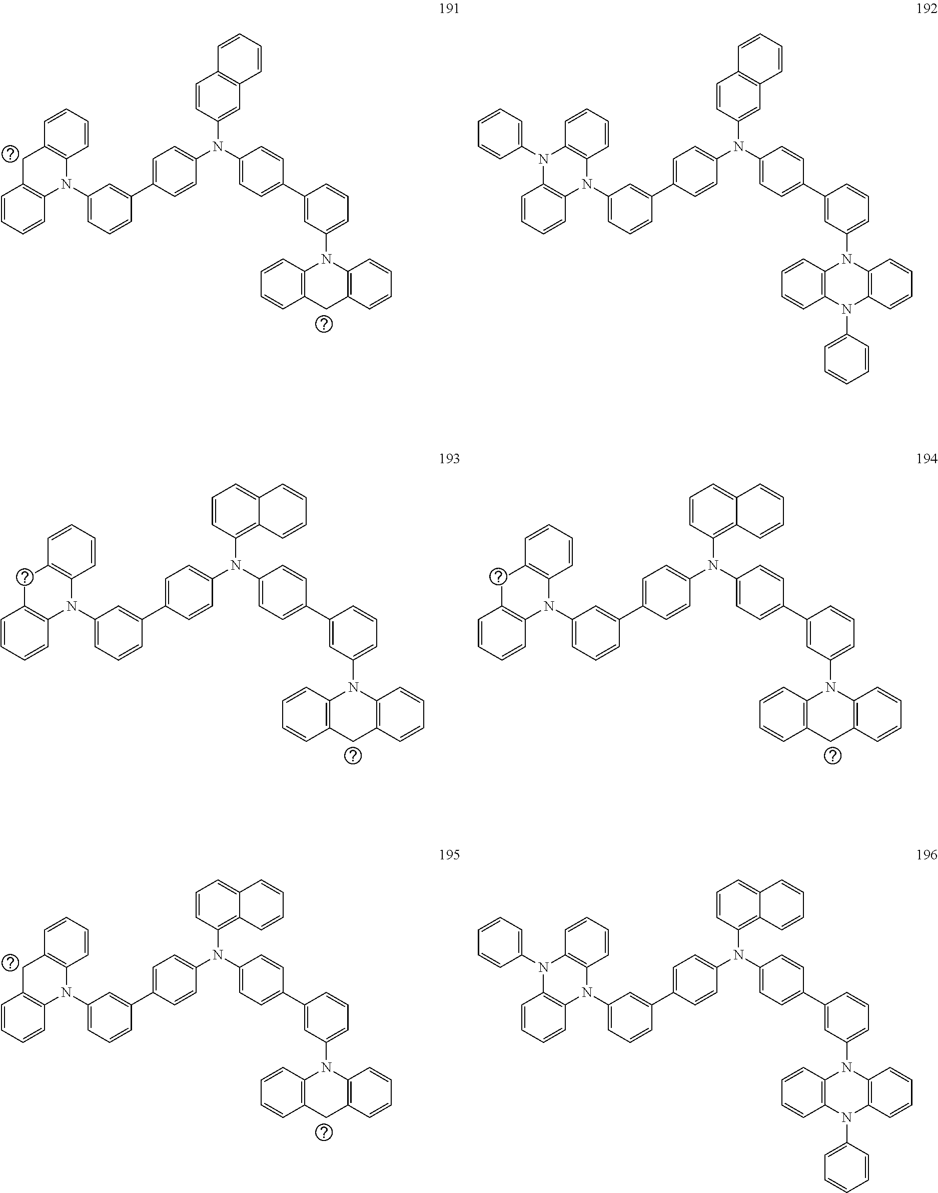

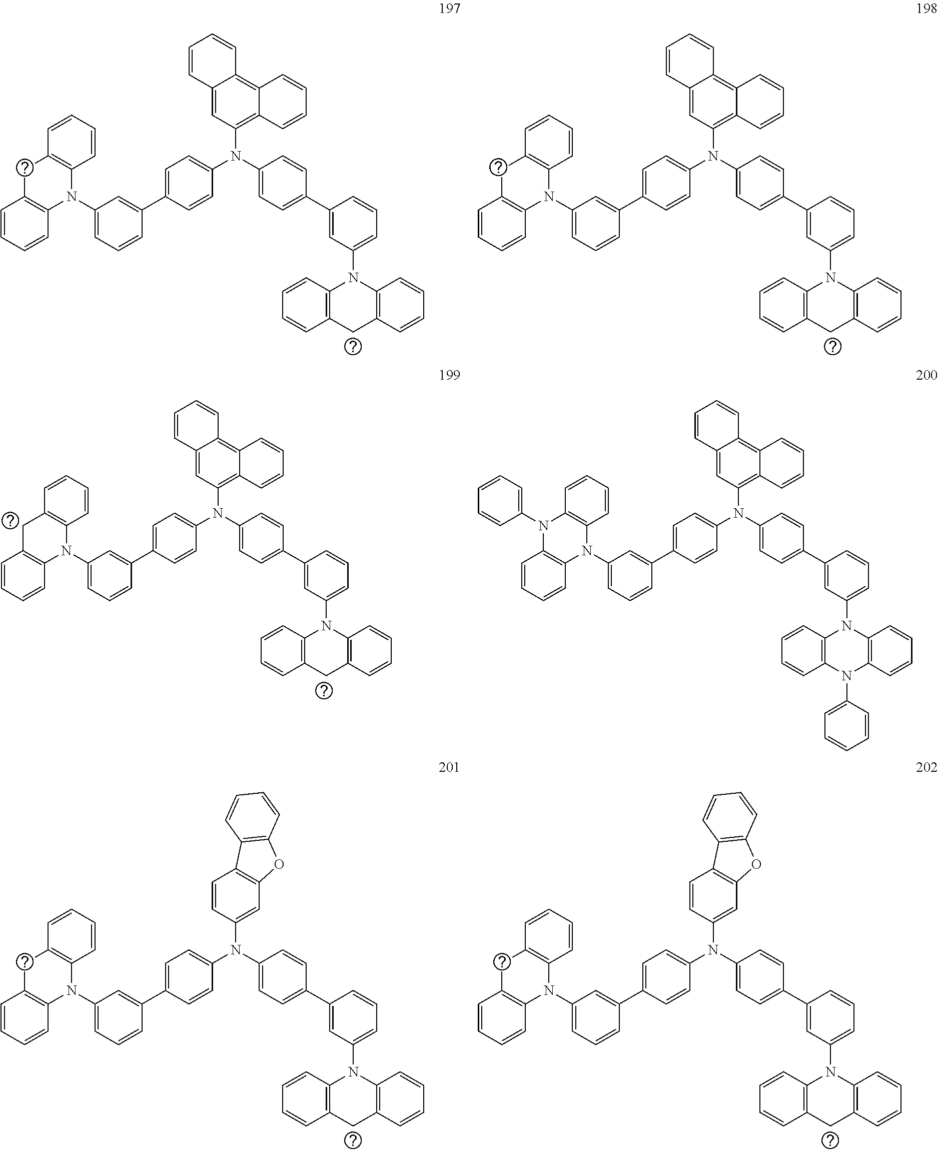

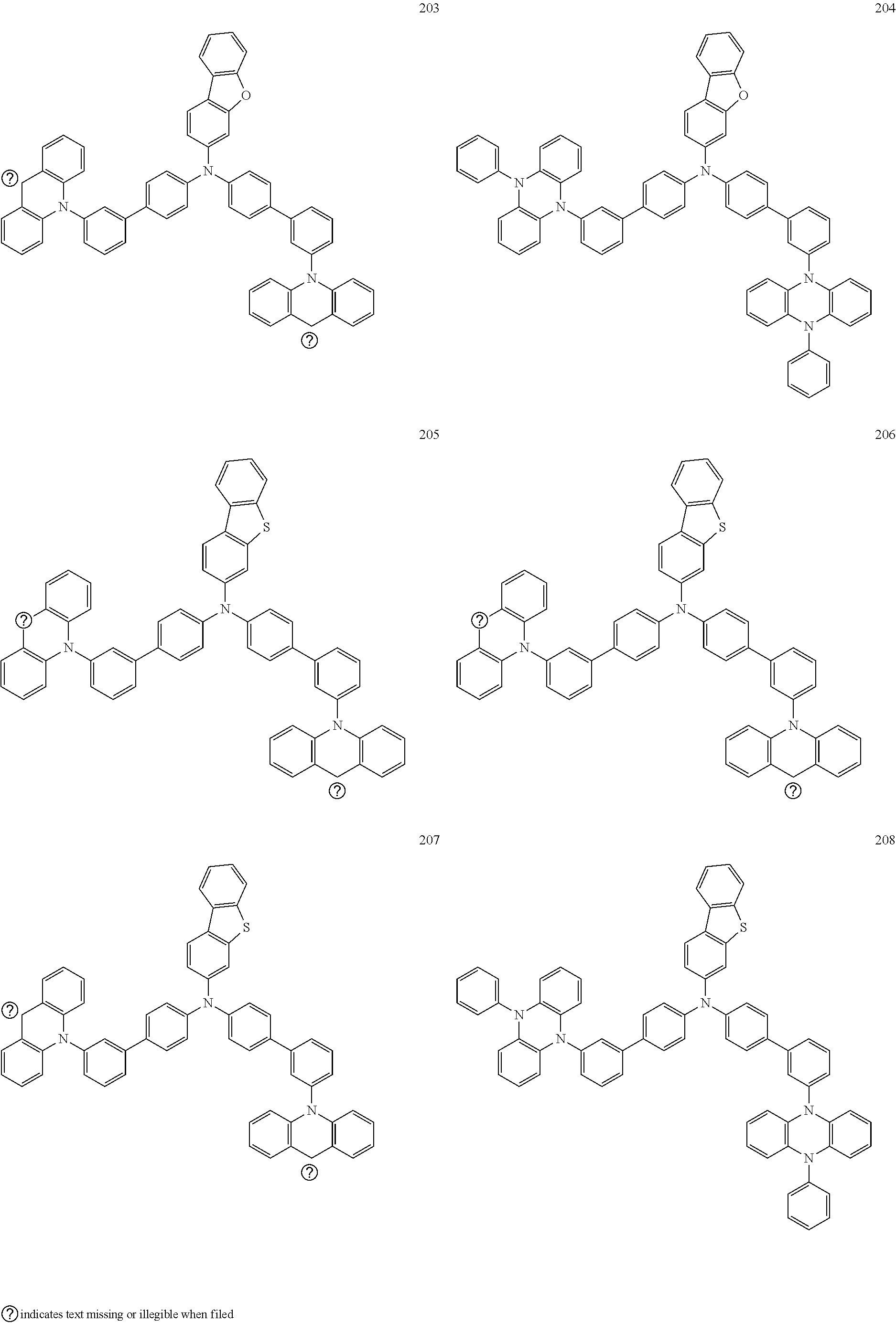

7. A material for an organic electroluminescent (EL) device comprising an amine derivative represented by the following Formula 1: 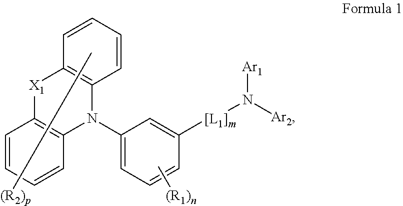
wherein Ar₁ and Ar₂ are each independently selected from a substituted or unsubstituted aryl group having 6 to 30 carbon atoms for forming a ring and a substituted or unsubstituted heteroaryl group having 2 to 30 carbon atoms for forming a ring,
L₁ is a divalent linker,
X₁ is selected from C(R₃)₂, C═O, Si(R₃)₂, N—R₃, O, S, SO and SO₂,
R₁, R₂ and R₃ are each independently selected from a substituted or unsubstituted linear, branched or cyclic alkyl group having 1 to 16 carbon atoms, a substituted or unsubstituted aryl group having 6 to 36 carbon atoms for forming a ring, a substituted or unsubstituted heteroaryl group having 2 to 32 carbon atoms for forming a ring, and an aryl group or a heteroaryl group formed via condensation of a plurality of adjacent substituents,
n is an integer selected from 0 to 4,
m is an integer selected from 1 to 4, and
p is an integer selected from 0 to 8.

8. The material for an organic EL device of claim 7, wherein L₁ is selected from a substituted or unsubstituted arylene group having 6 to 30 carbon atoms for forming a ring and a substituted or unsubstituted heteroarylene group having 2 to 30 carbon atoms for forming a ring.

9. The material for an organic EL device of claim 7, wherein Ar₁ and Ar₂ are each independently selected from a group substituted with a substituent not including a nitrogen atom and an unsubstituted aryl group or heteroaryl group, the aryl group or the heteroaryl group not including a nitrogen atom.

10. The material for an organic EL device of claim 7, wherein Ar₁ is a group substituted with a substituent not including a nitrogen atom or an unsubstituted aryl group or heteroaryl group not including a nitrogen atom, and
Ar₂ is represented by the following Formula 2: 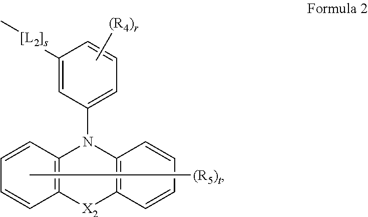
wherein L₂ is a divalent linker,
X₂ is selected from C(R₆)₂, C═O, Si(R₆)₂, N—R₆, O, S, SO and SO₂,
R₄, R₅ and R₆ are each independently selected from a substituted or unsubstituted linear, branched or cyclic alkyl group having 1 to 16 carbon atoms, a substituted or unsubstituted aryl group having 6 to 36 carbon atoms for forming a ring, a substituted or unsubstituted heteroaryl group having 2 to 32 carbon atoms for forming a ring, and an aryl group or a heteroaryl group formed via condensation of a plurality of adjacent substituents,
r is an integer selected from 0 to 4,
s is an integer selected from 1 to 4, and
t is an integer selected from 0 to 8.

11. The material for an organic EL device of claim 10, wherein
s in Formula 2 is the same as m in Formula 1,
r in Formula 2 is the same as n in Formula 1, and
t in Formula 2 is the same as p in Formula 1.

12. The material for an organic EL device of claim 7, wherein the amine derivative represented by Formula 1 is at least one selected from the following Compounds 1 to 208:

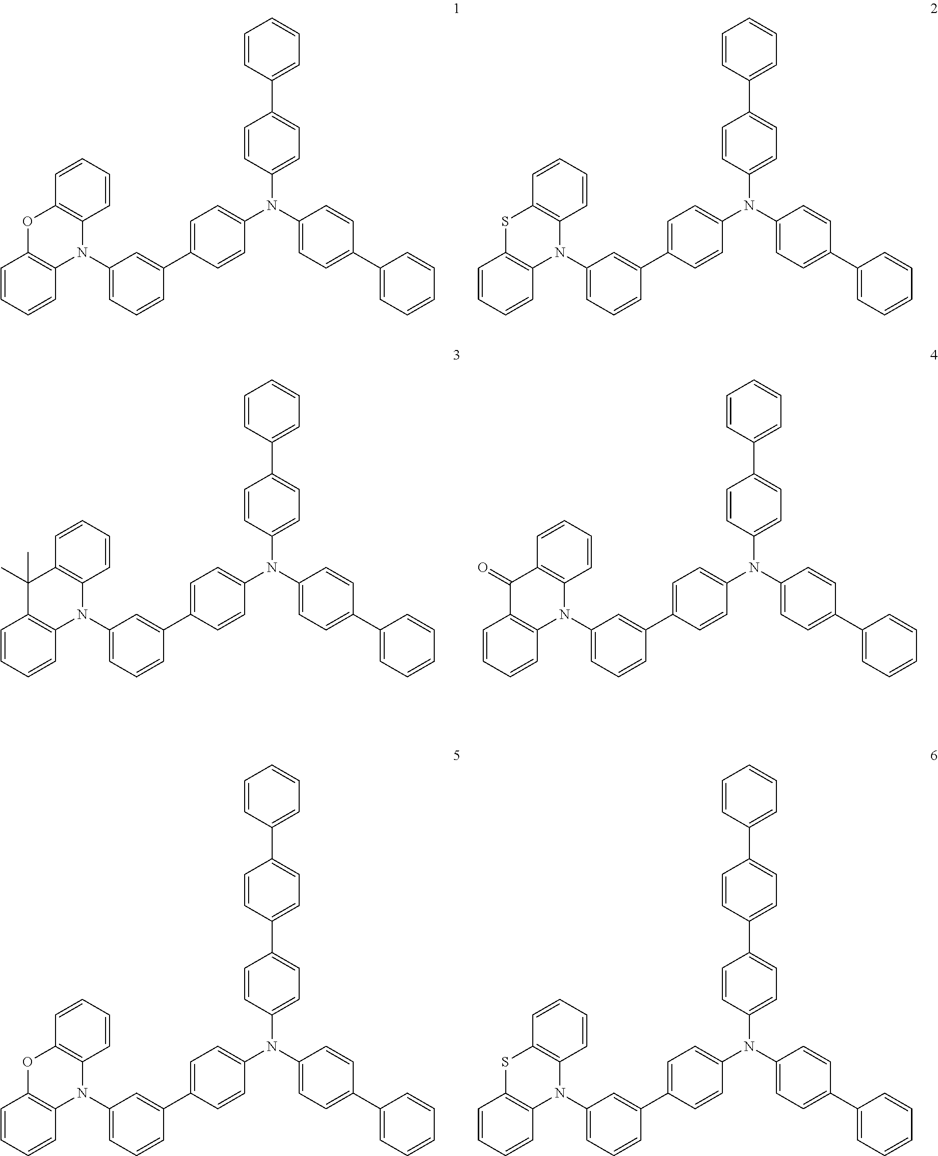

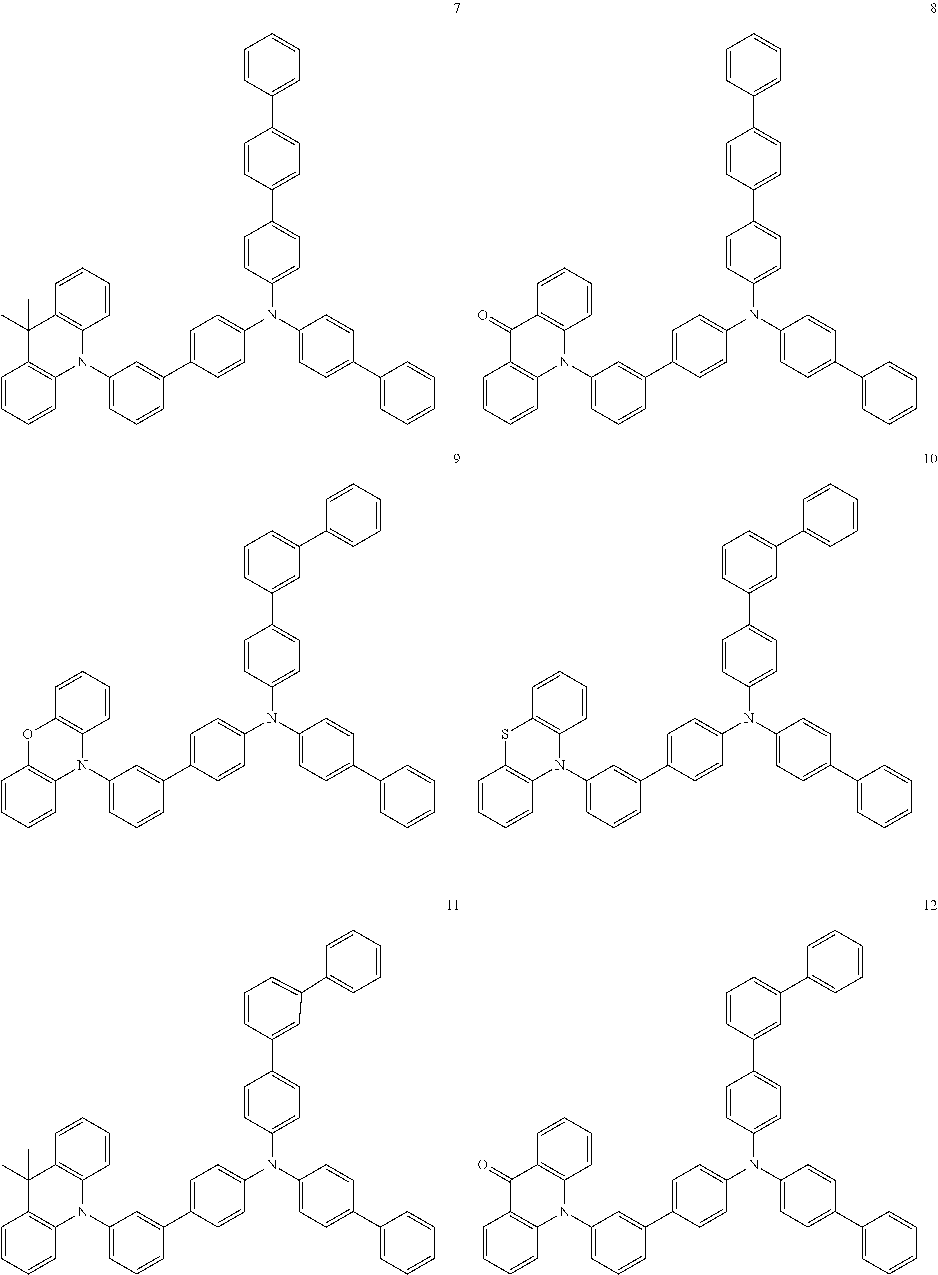

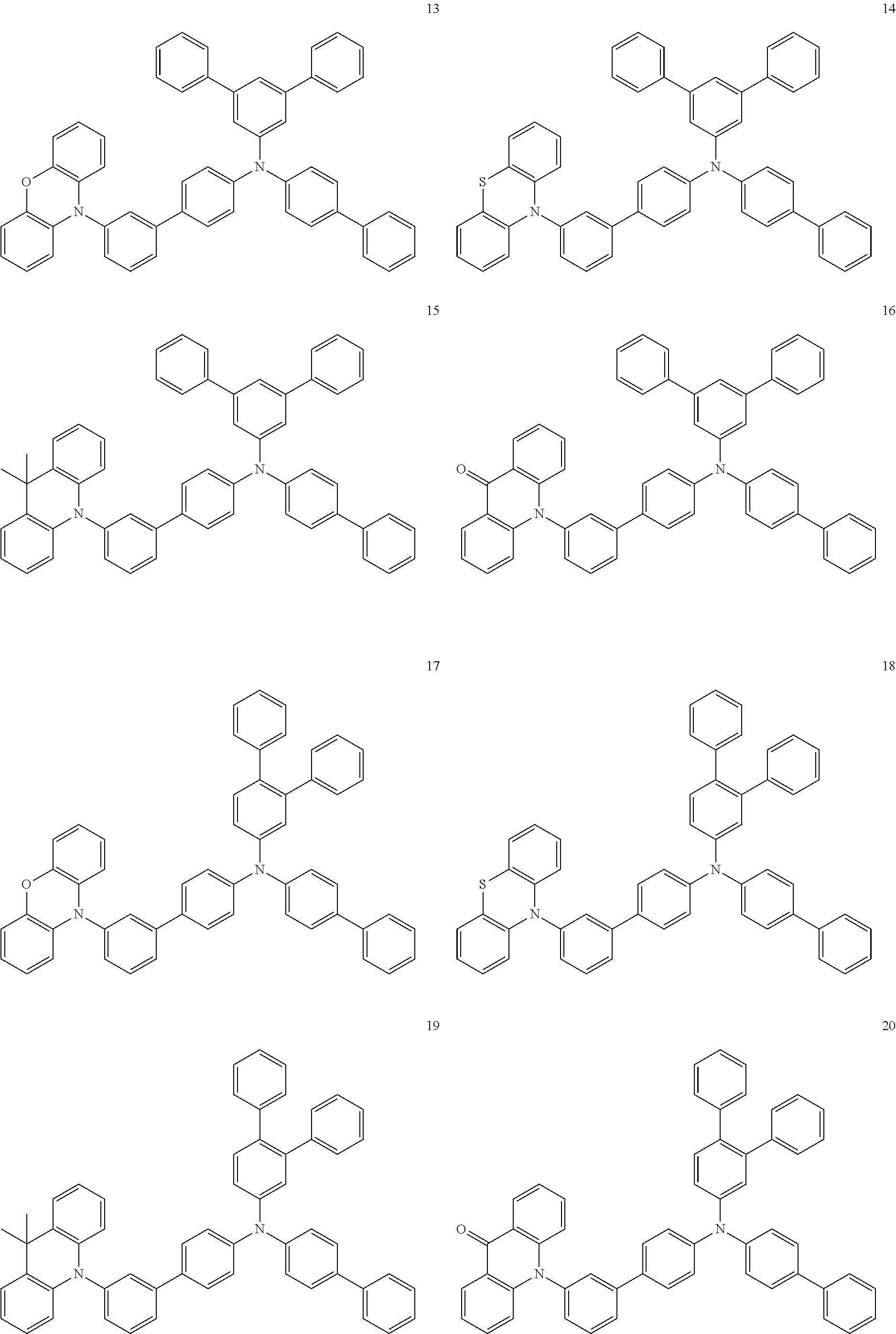

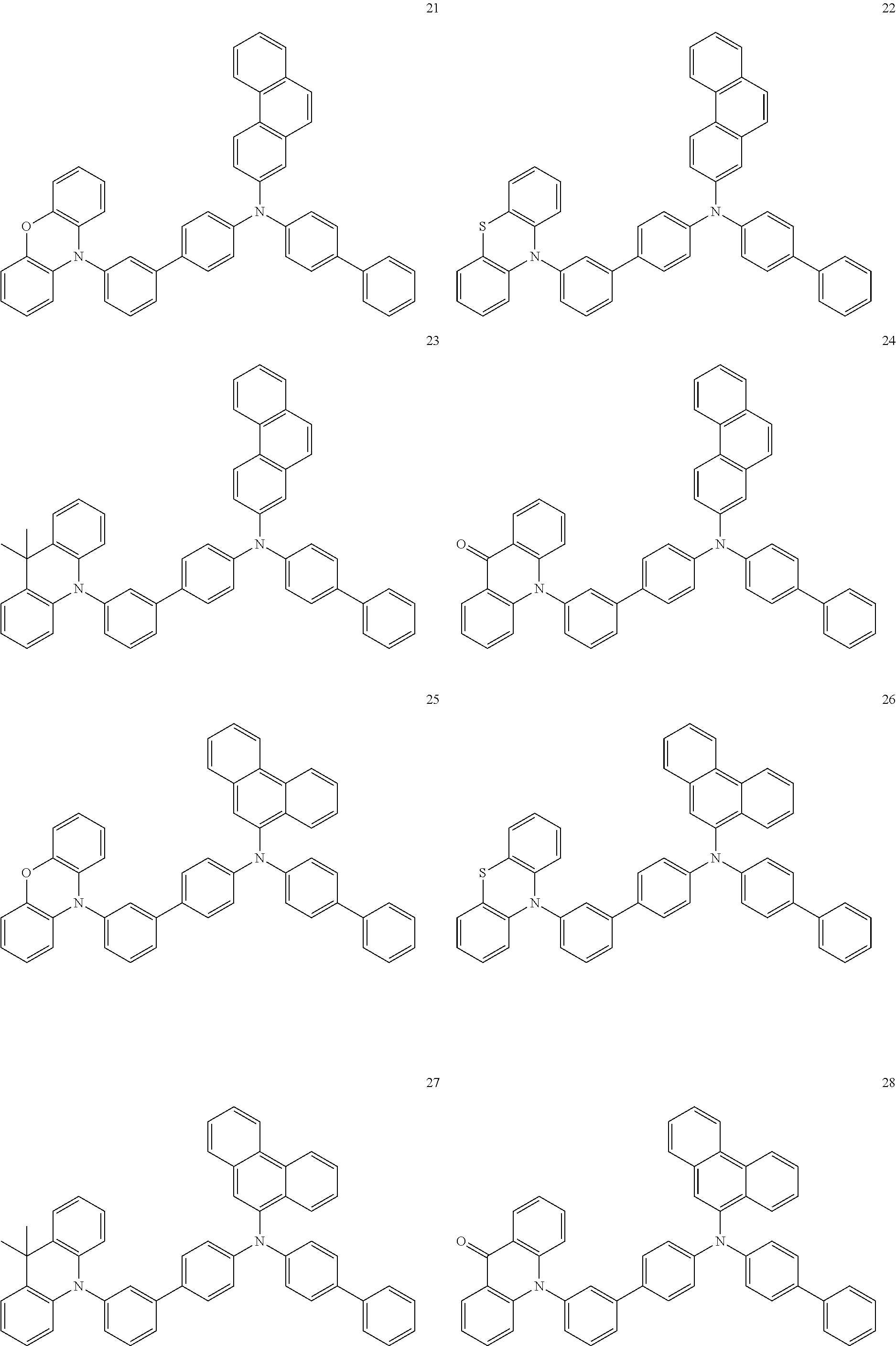

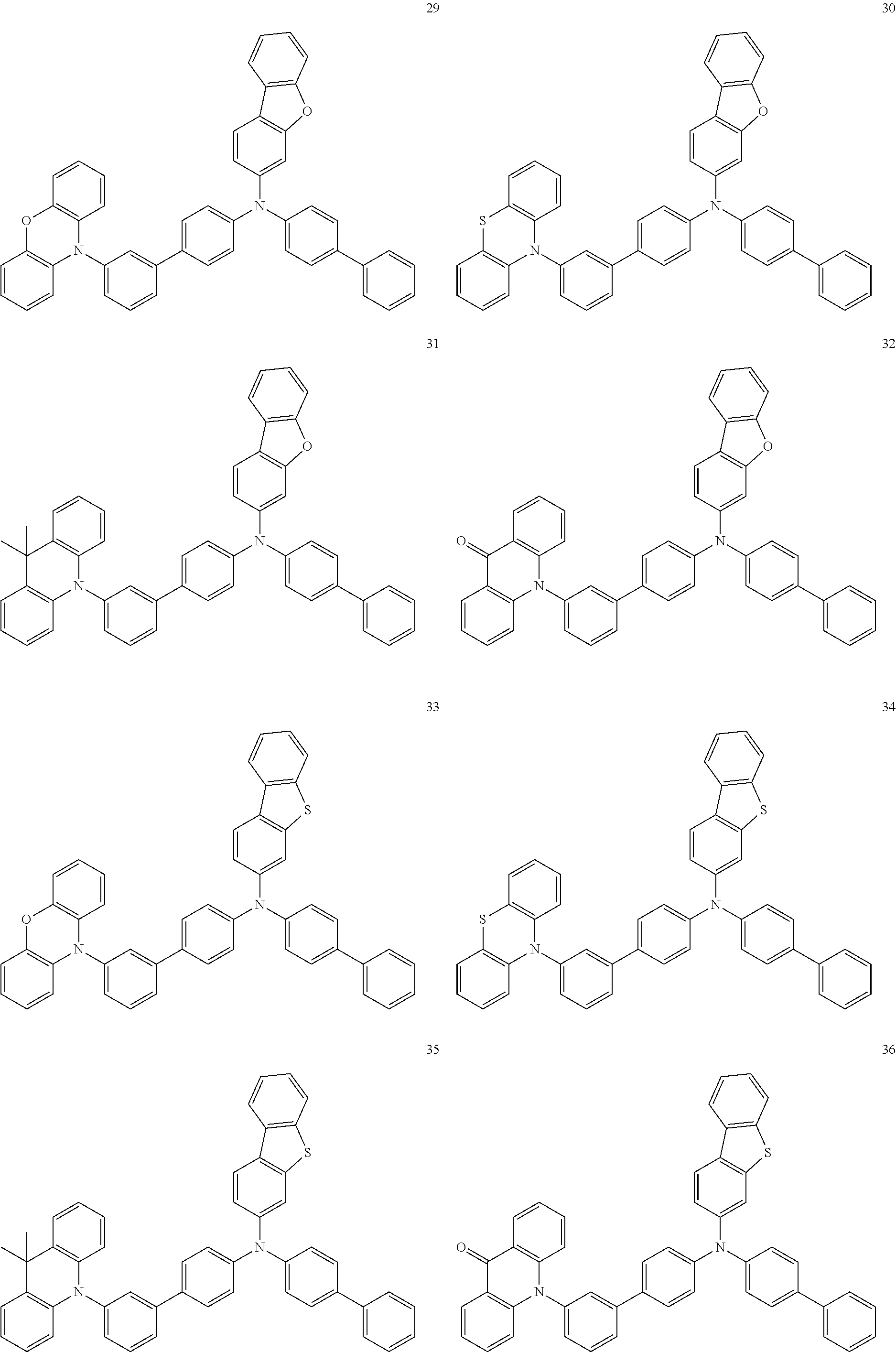

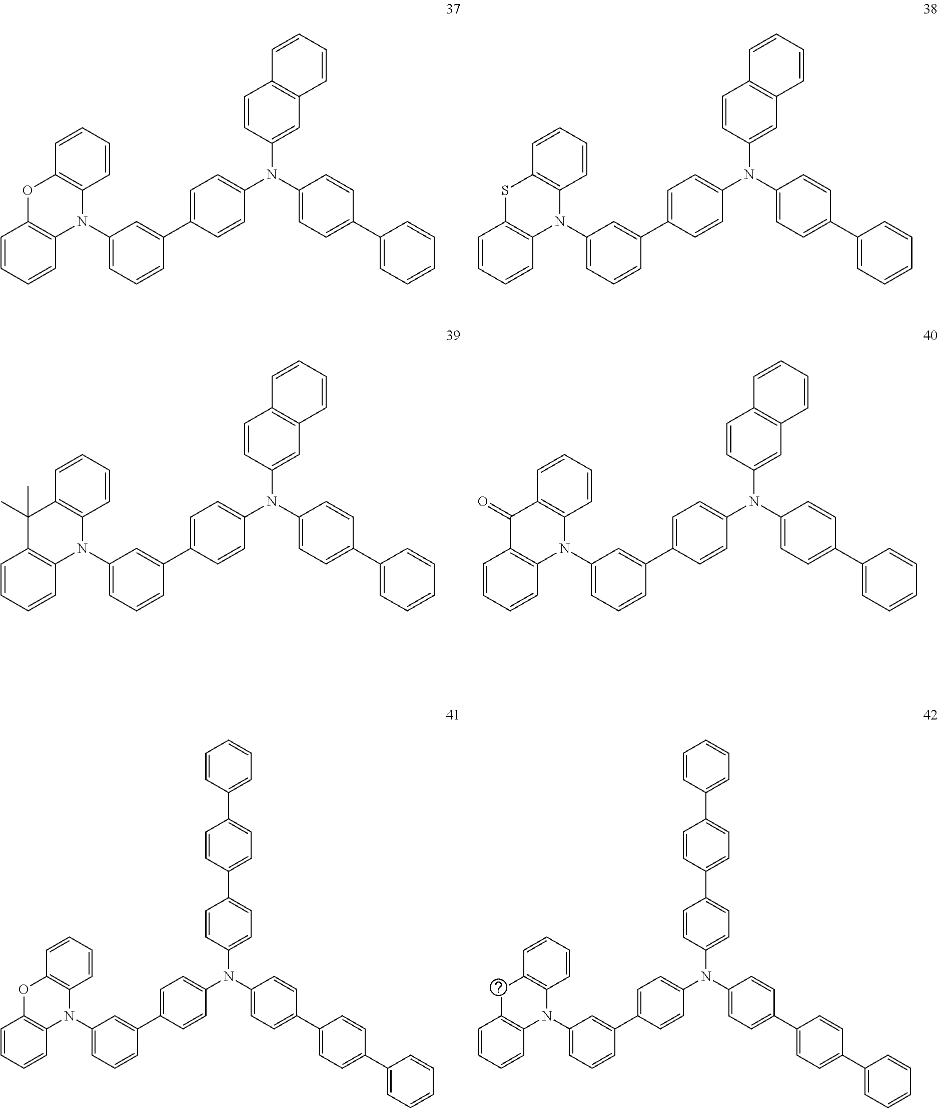

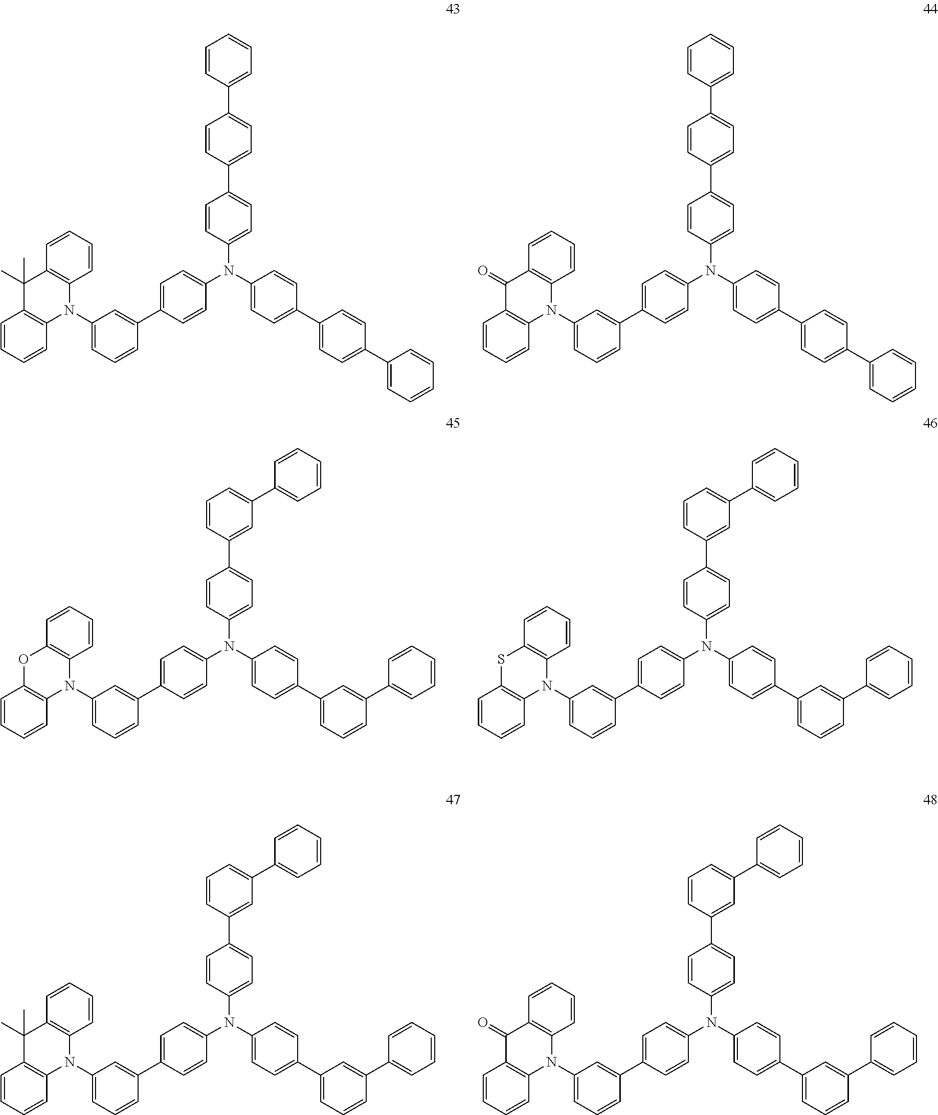

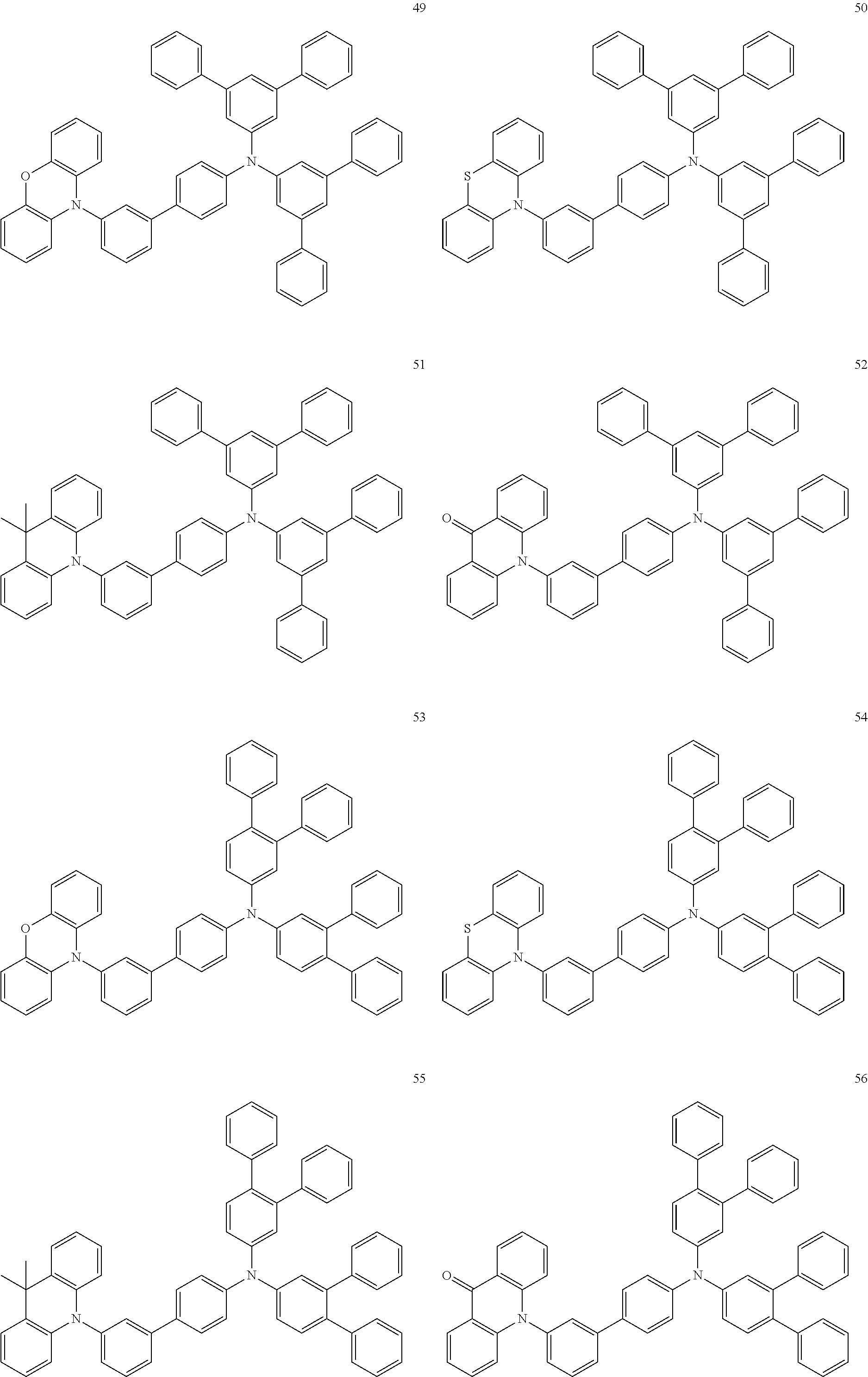

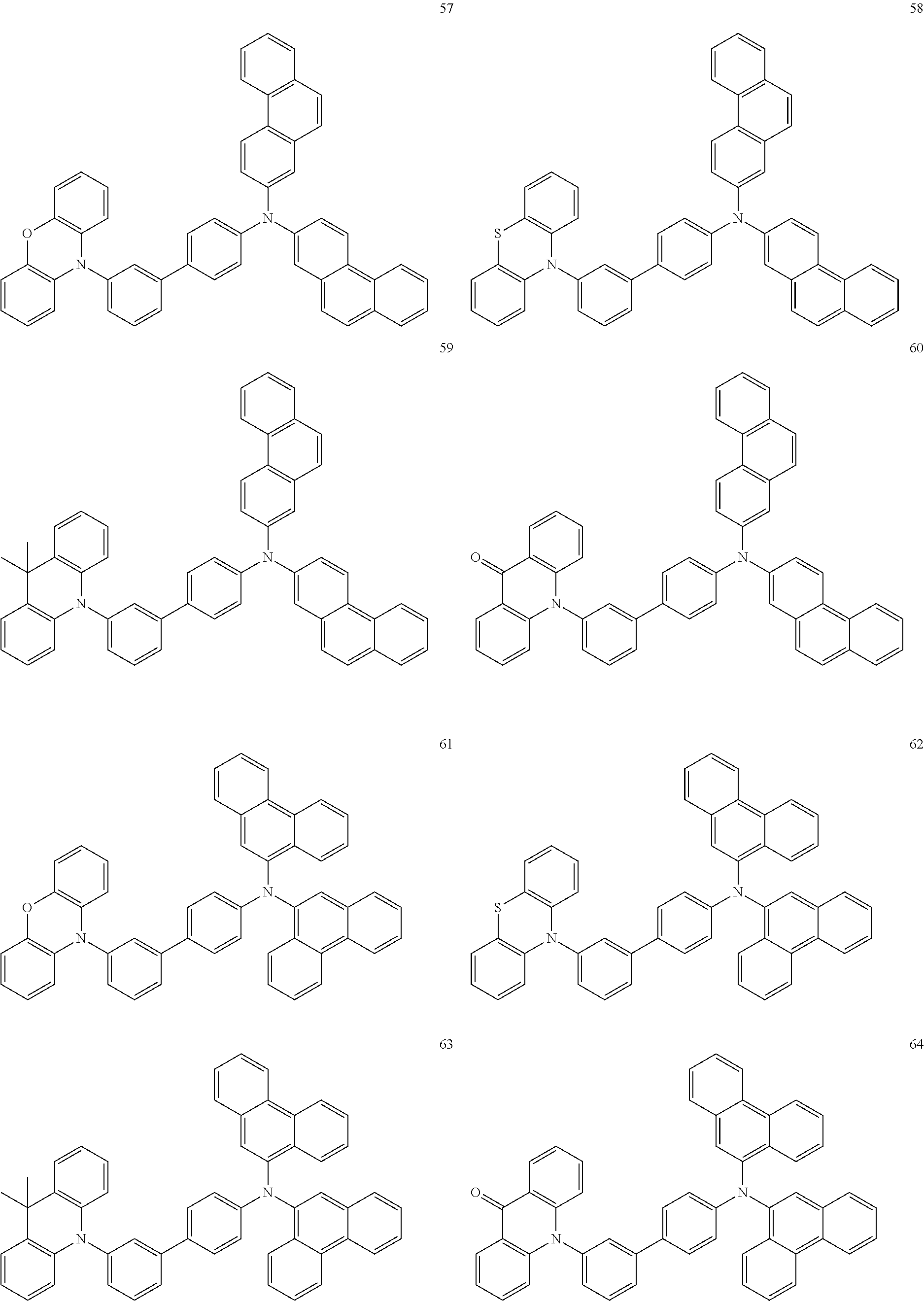

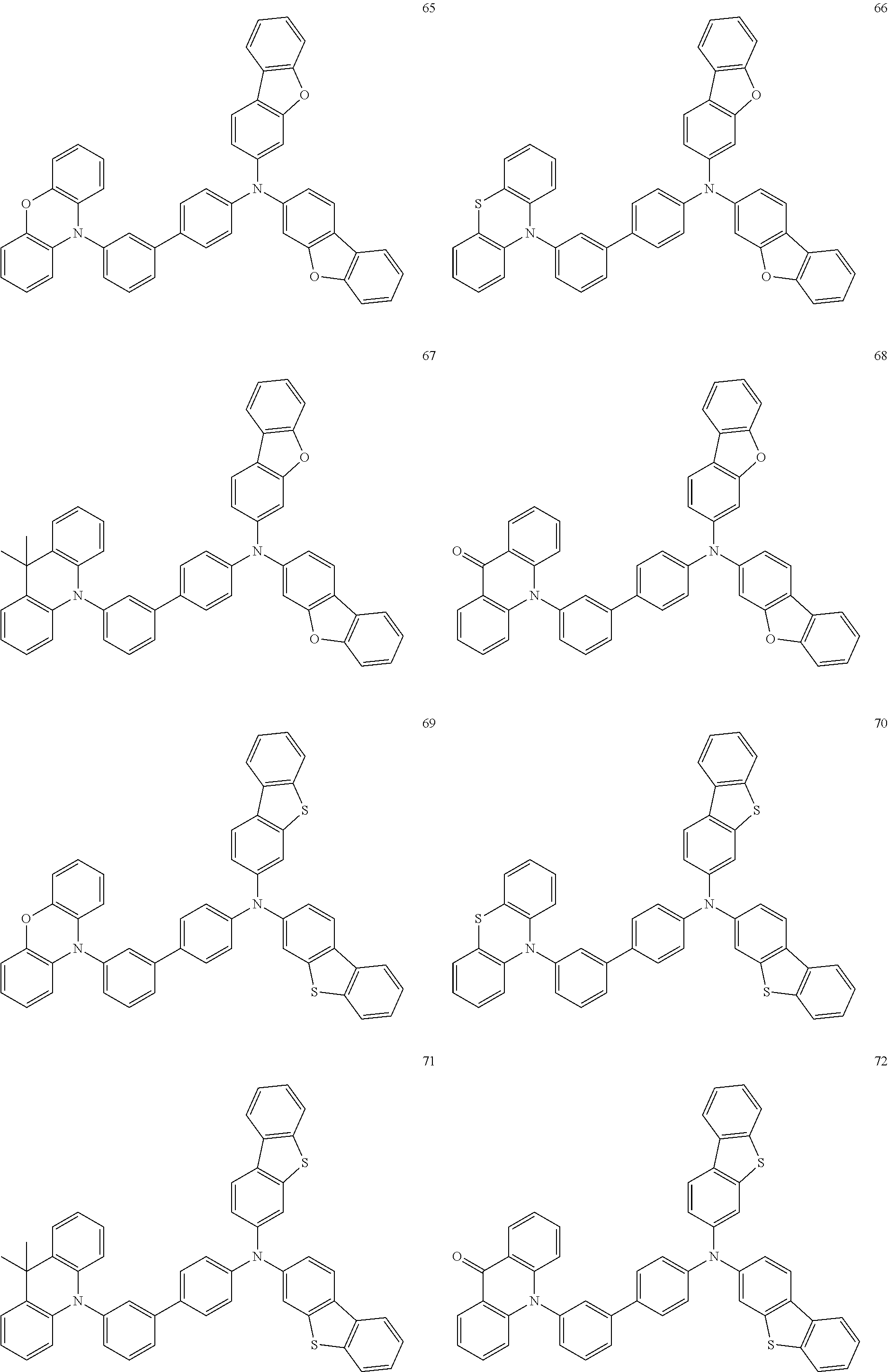

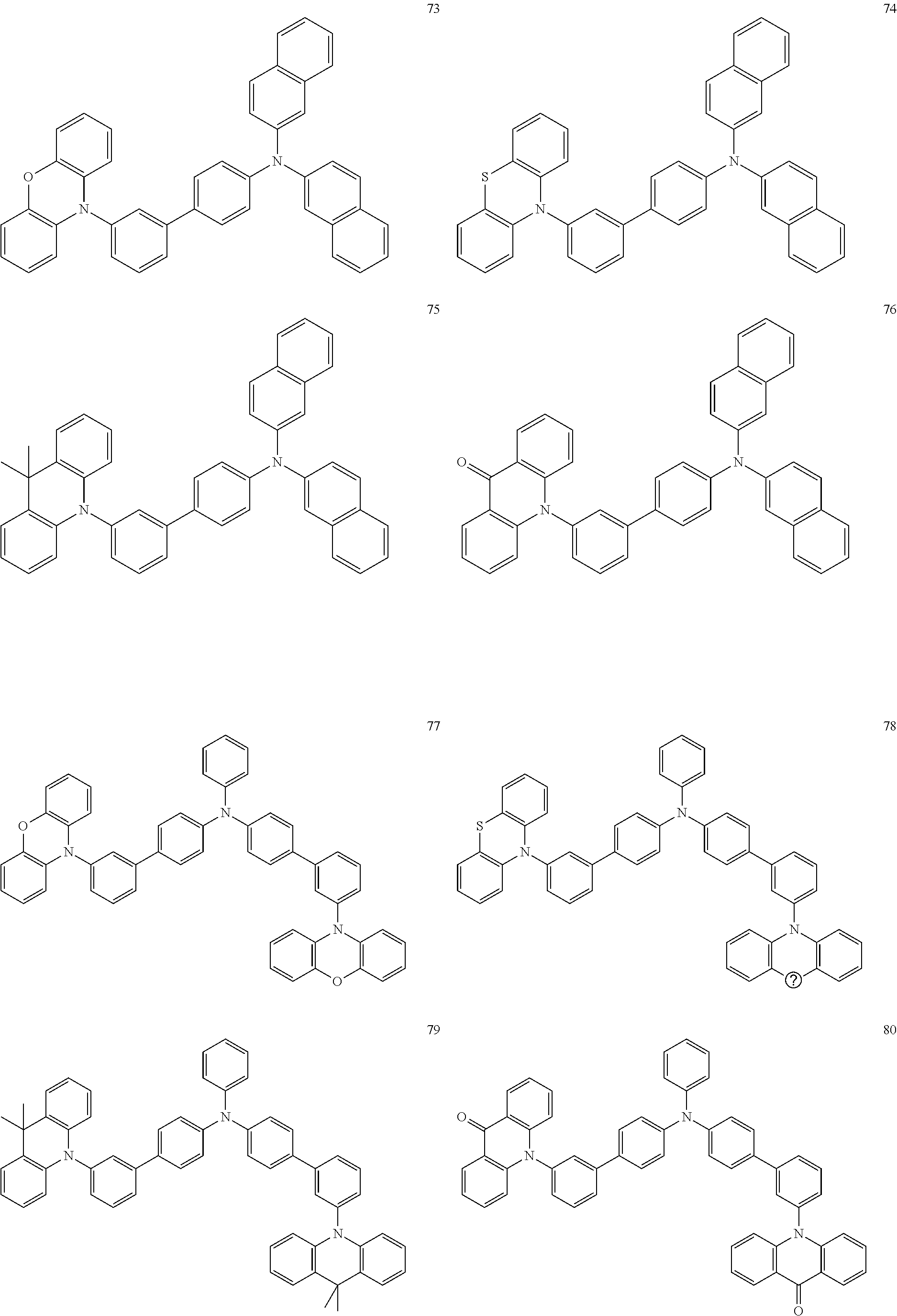

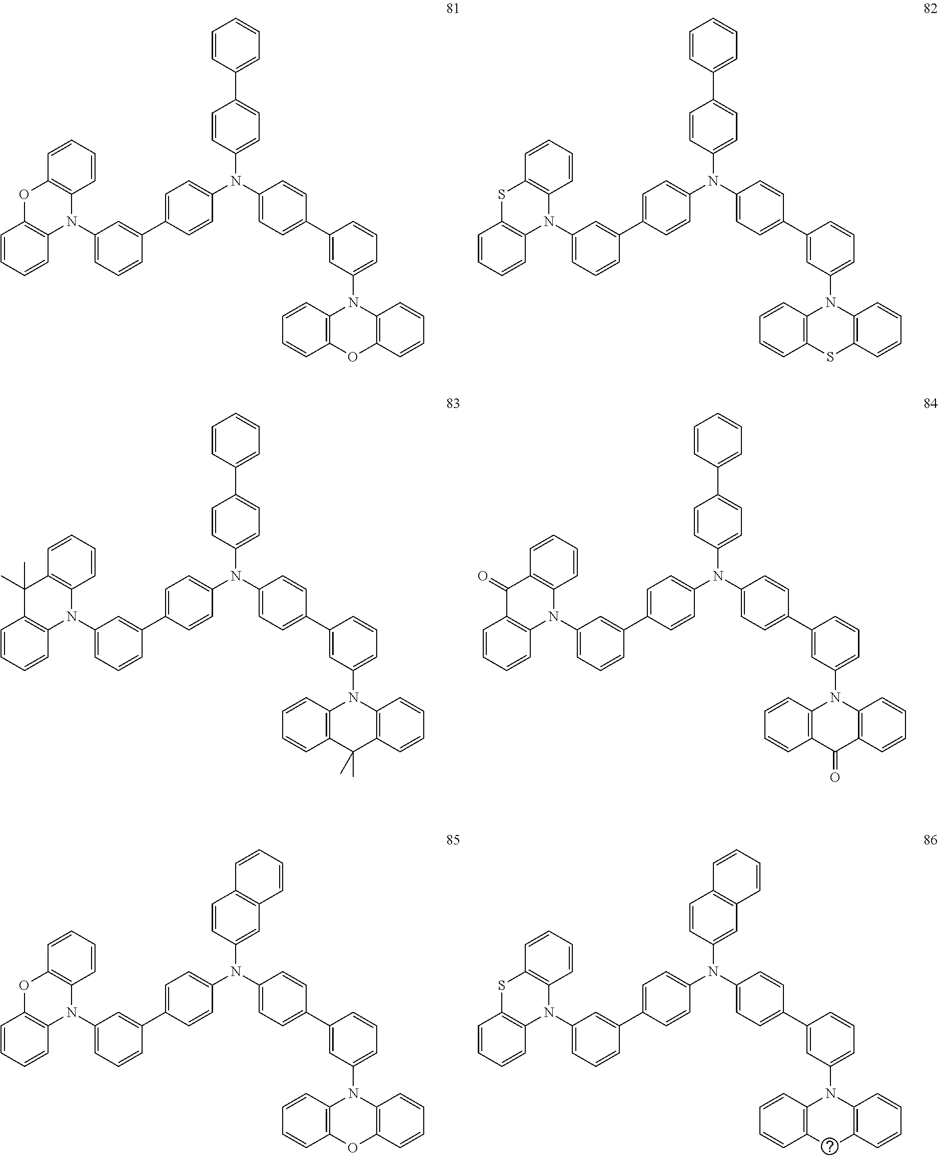

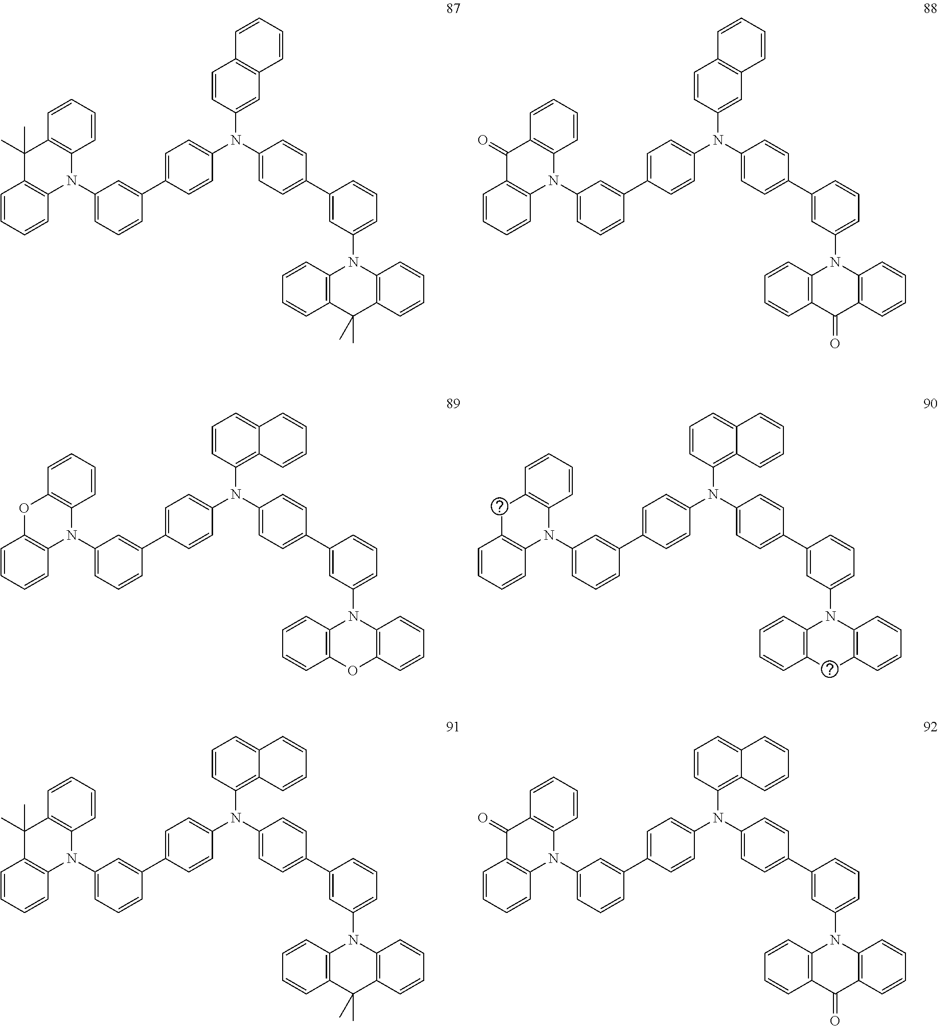

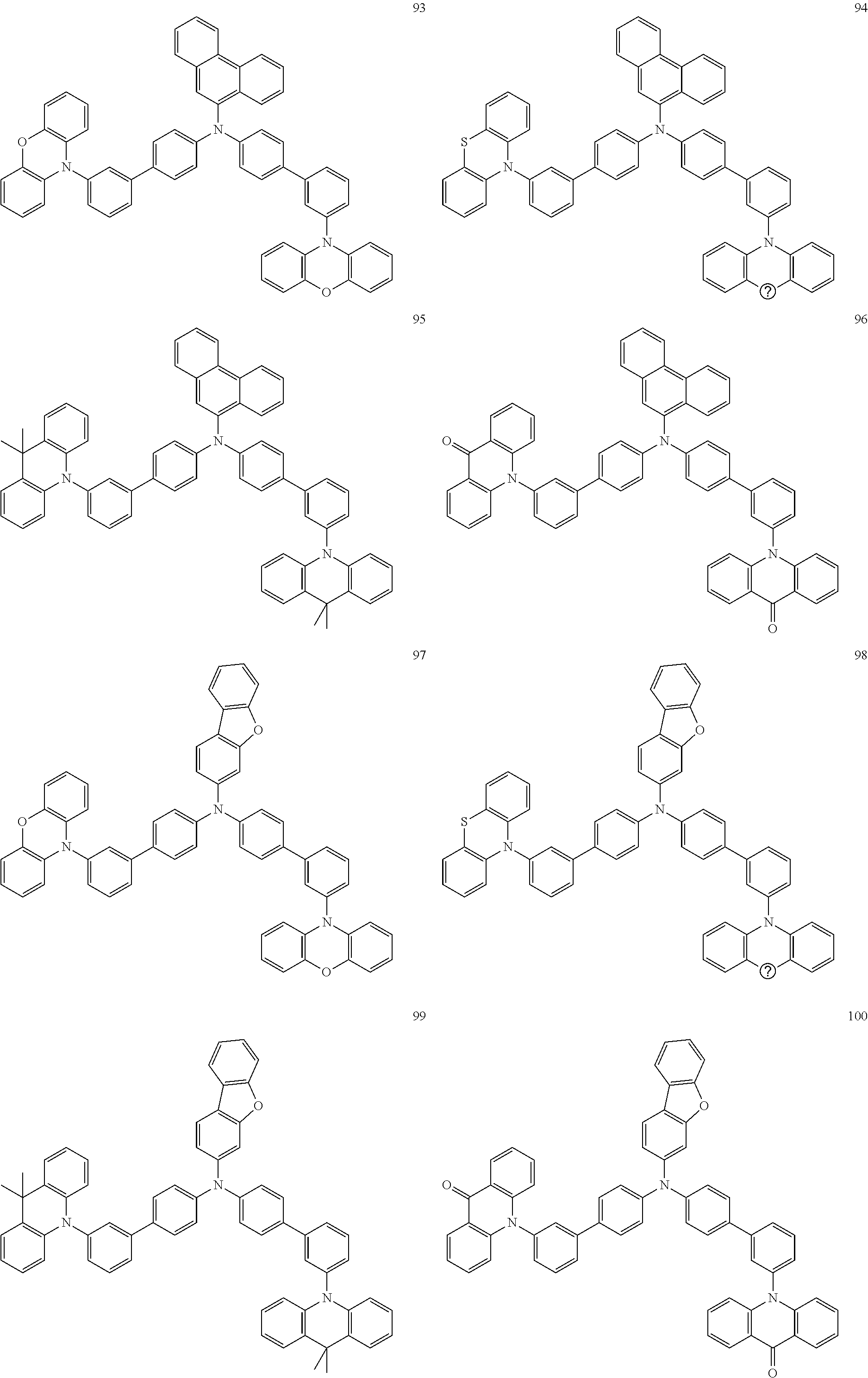

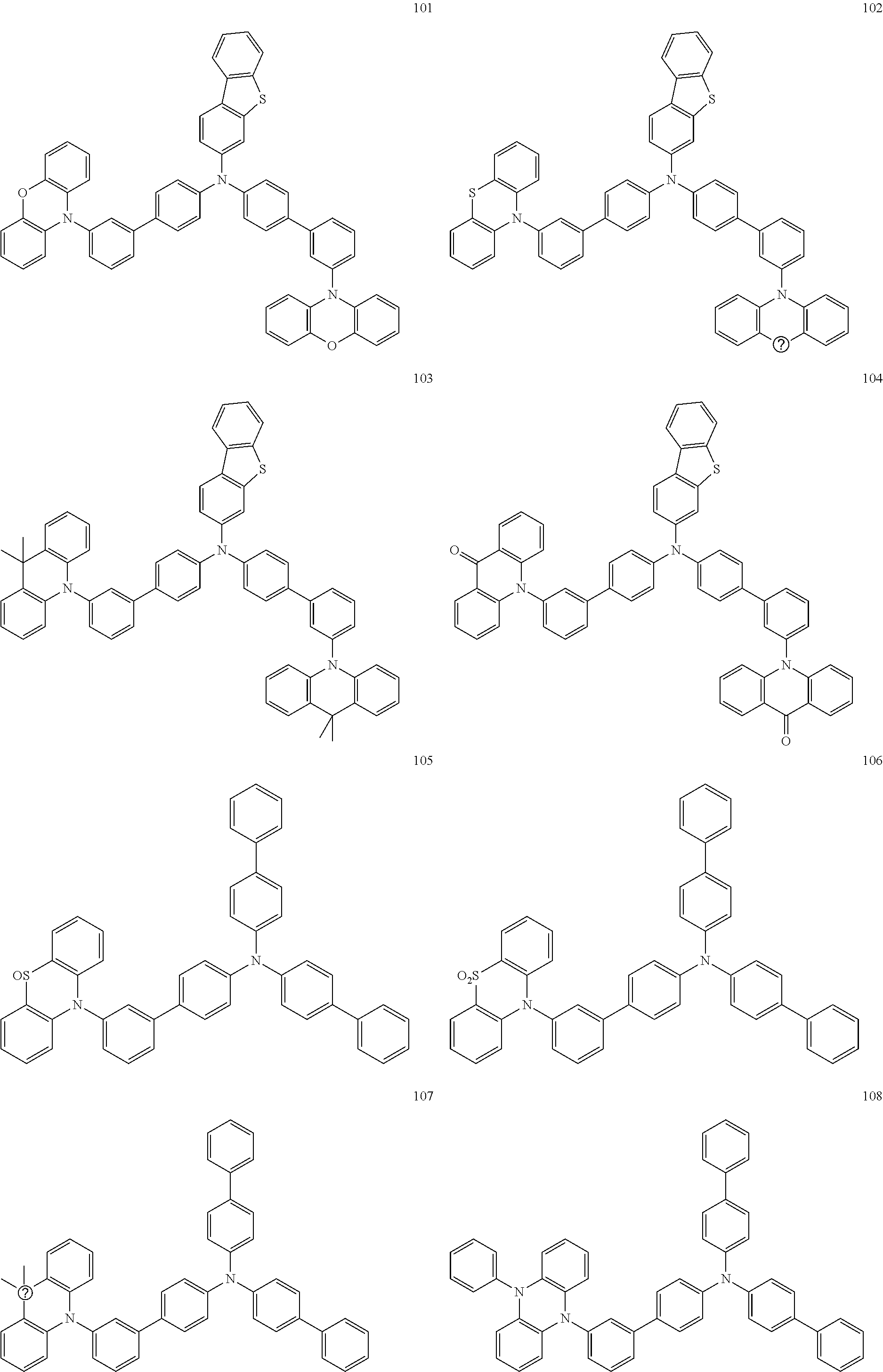

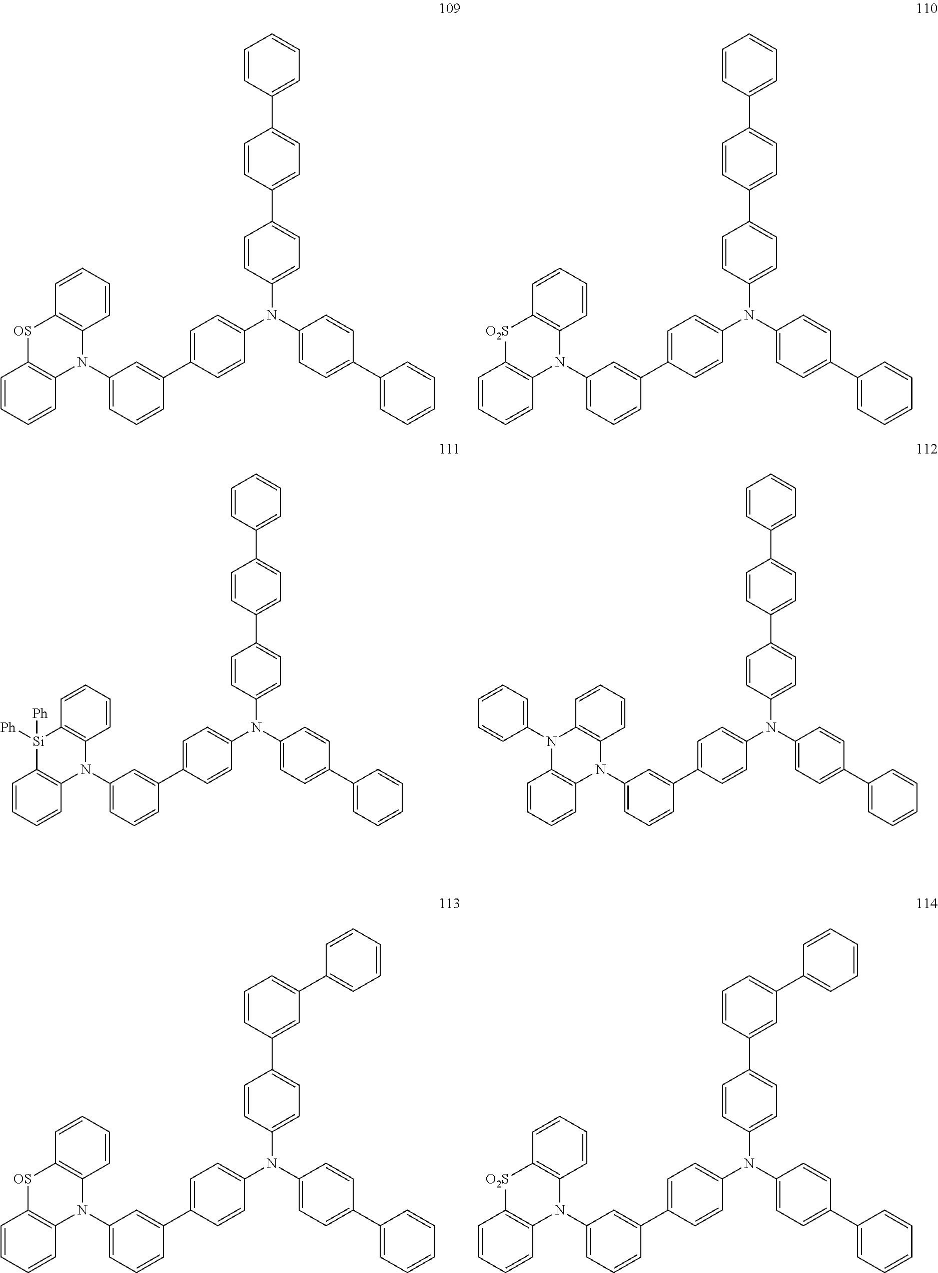

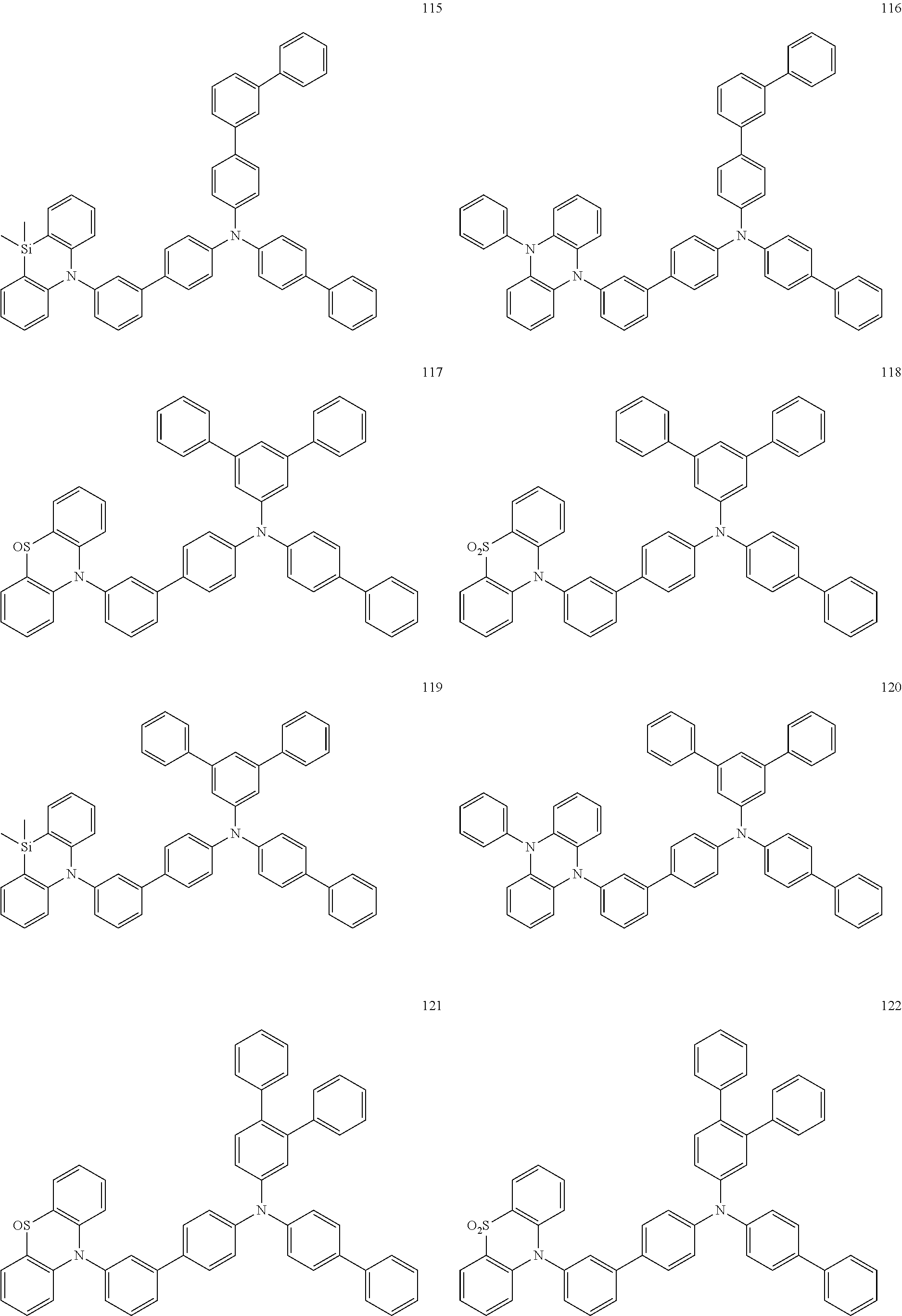

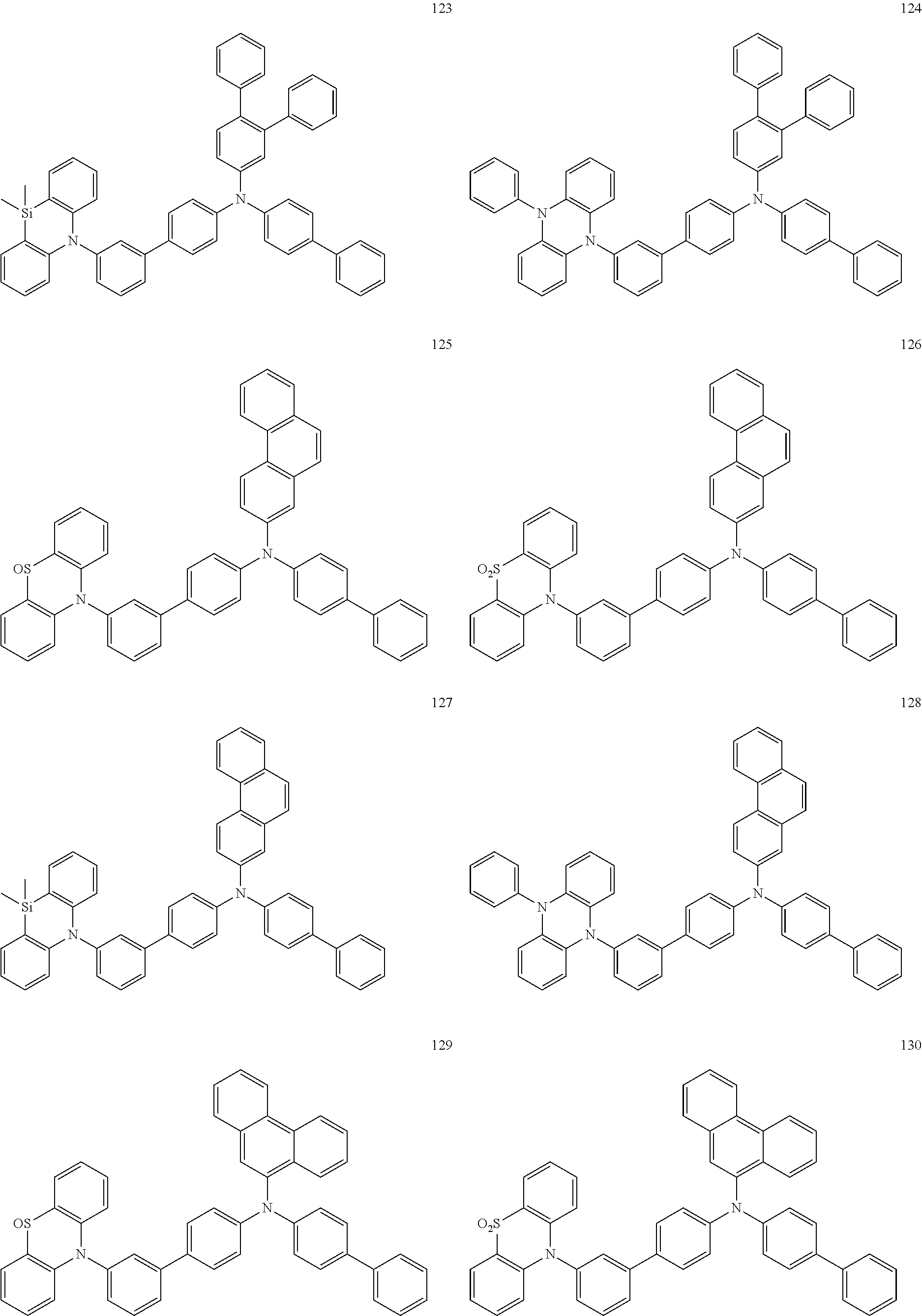

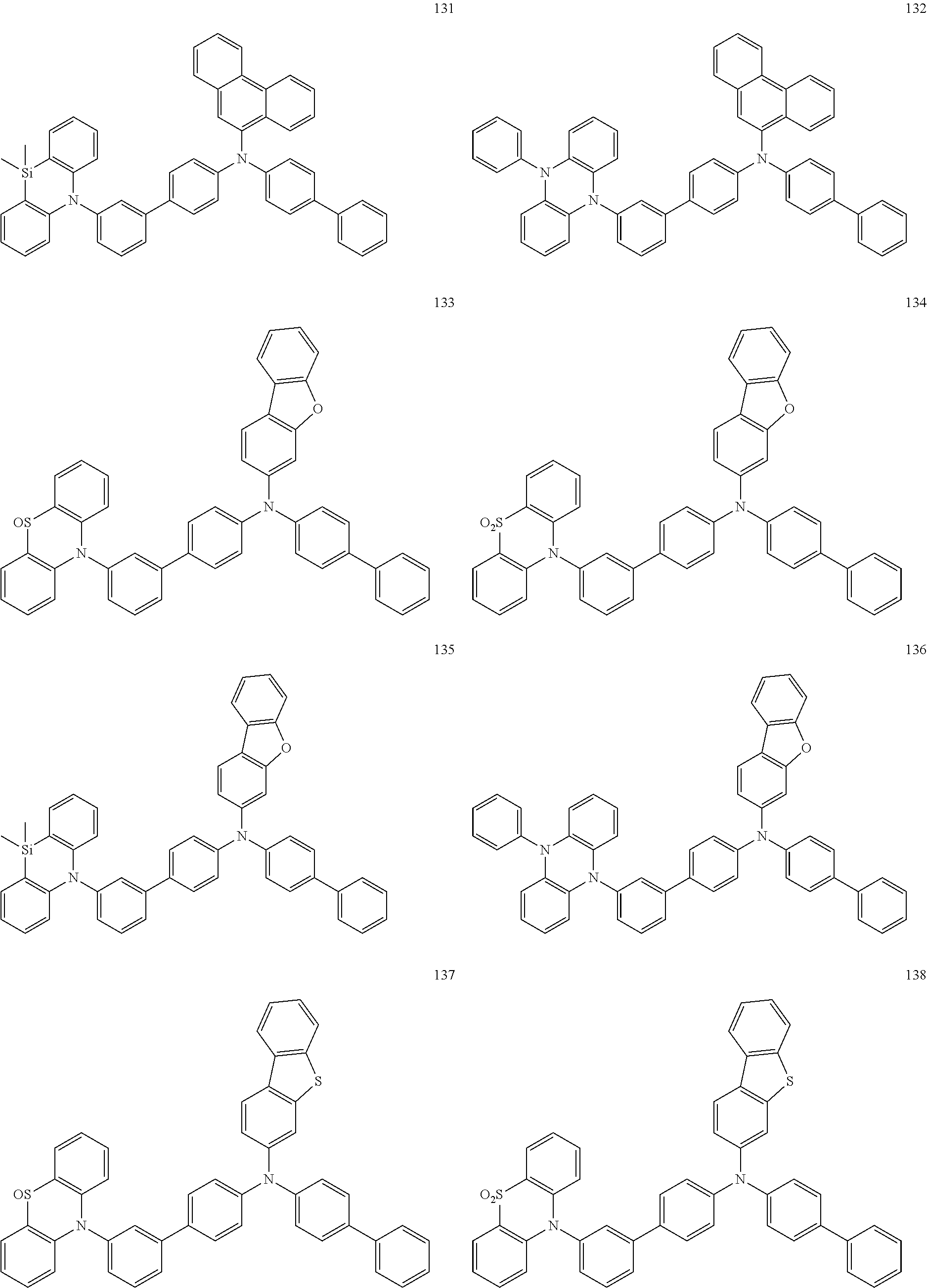

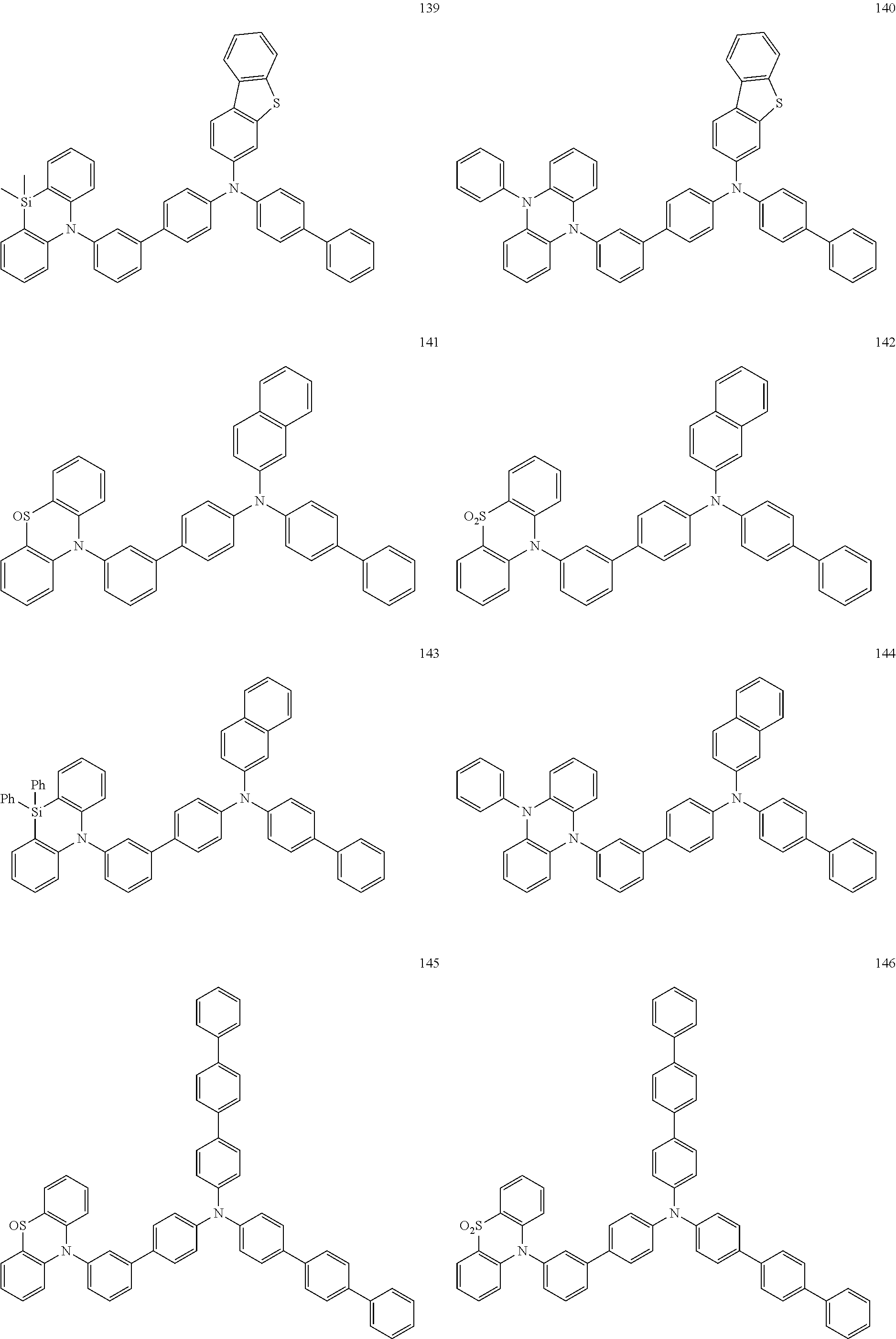

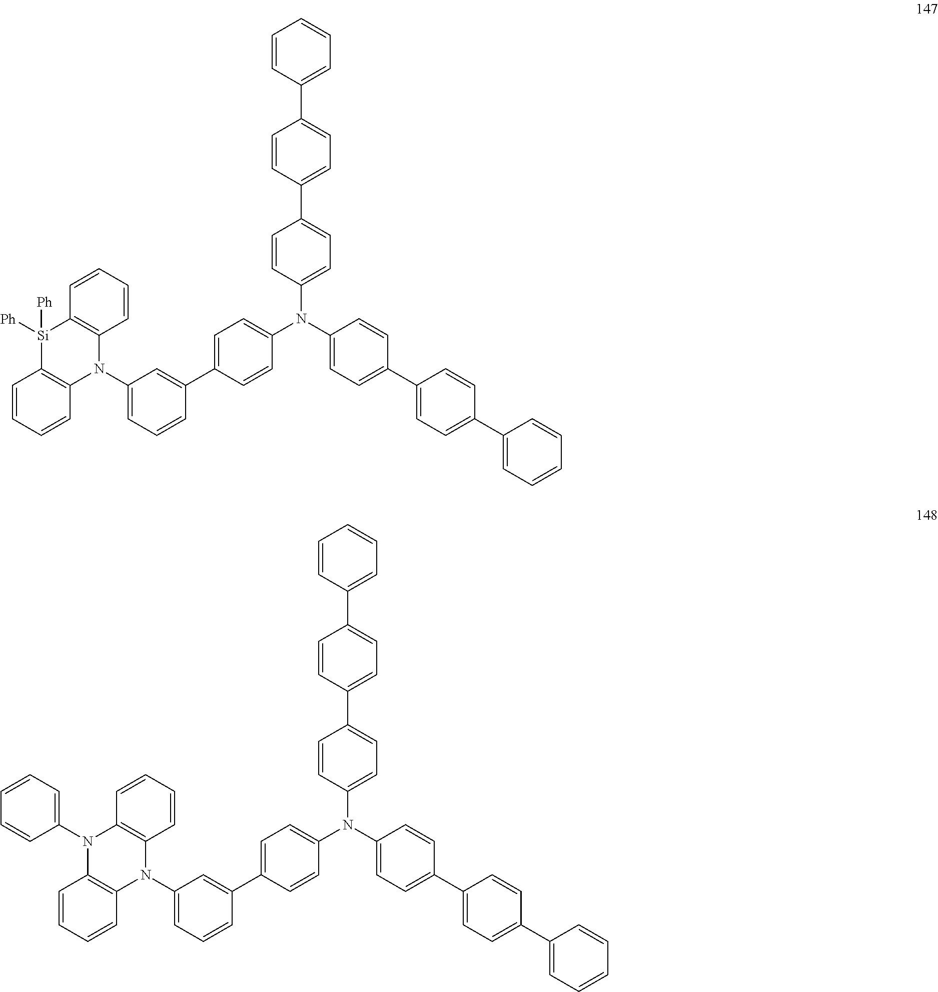 [SMILES: c1ccc(-c2ccc(-c3ccc(N(c4ccc(-c5ccc(-c6ccccc6)cc5)cc4)c4ccc(-c5cccc(N6c7ccccc7[Si](c7ccccc7)(c7ccccc7)c7ccccc76)c5)cc4)cc3)cc2)cc1 ; c1ccc(-c2ccc(-c3ccc(N(c4ccc(-c5ccc(-c6ccccc6)cc5)cc4)c4ccc(-c5cccc(N6c7ccccc7N(c7ccccc7)c7ccccc76)c5)cc4)cc3)cc2)cc1]

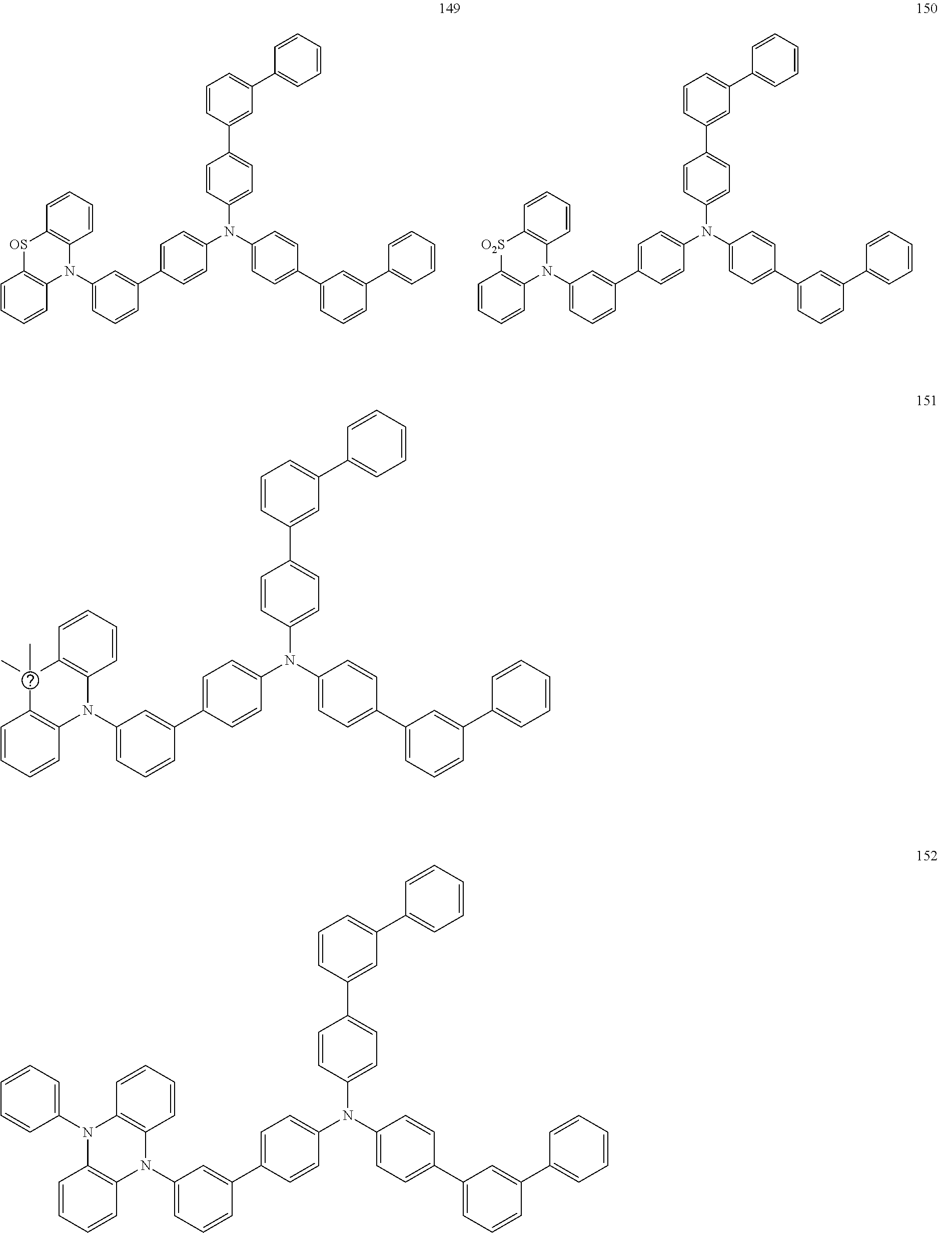

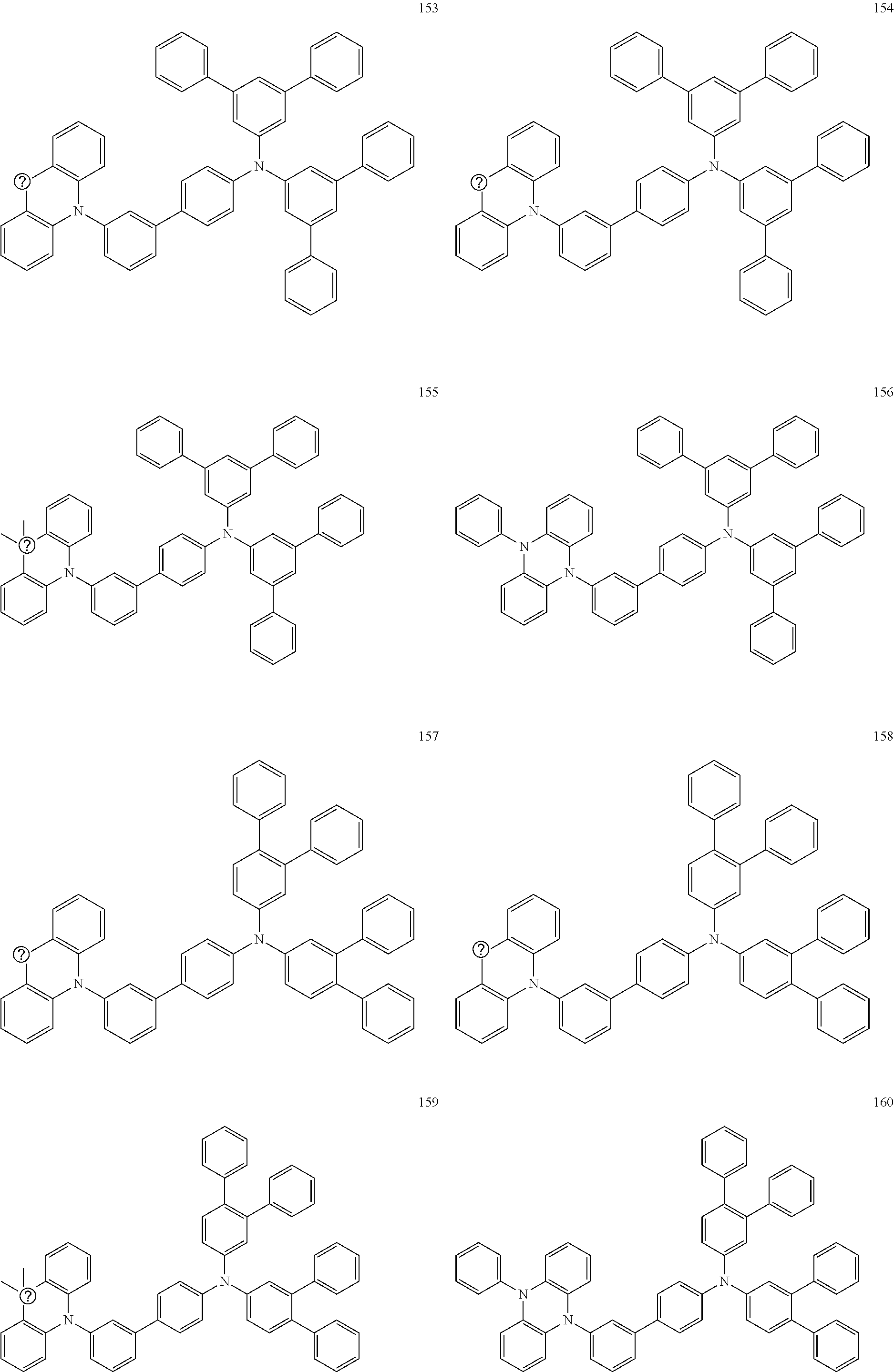

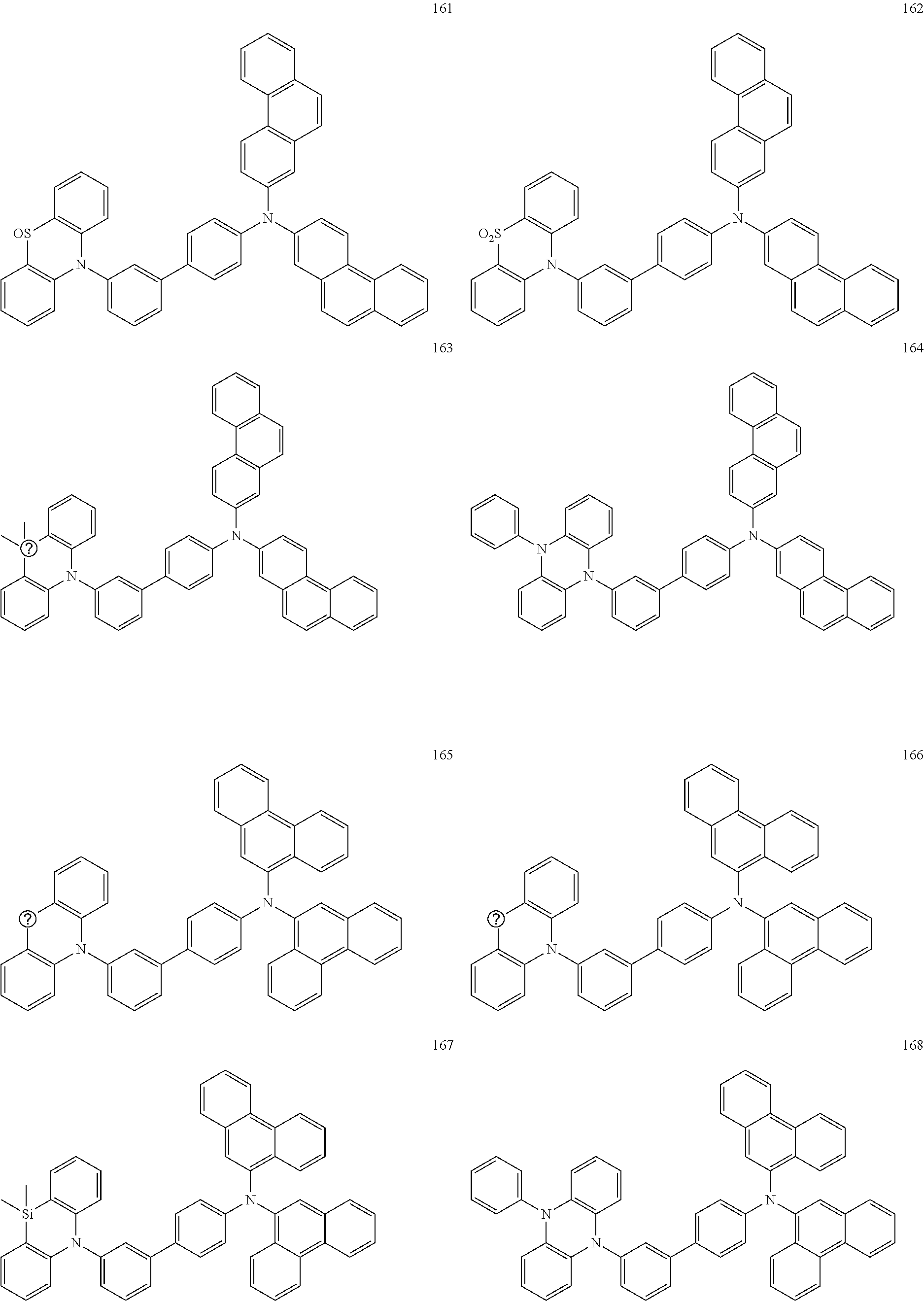

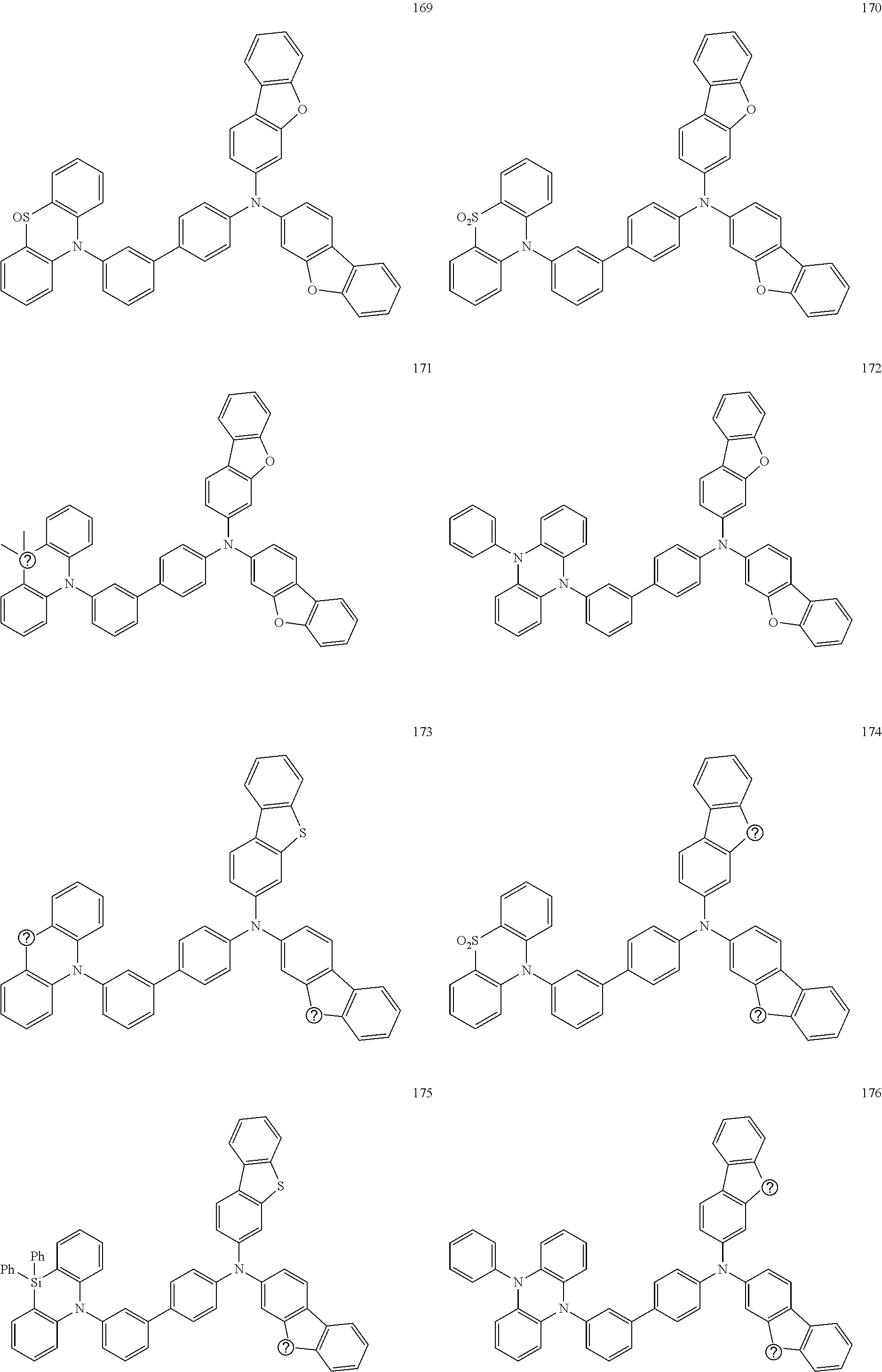

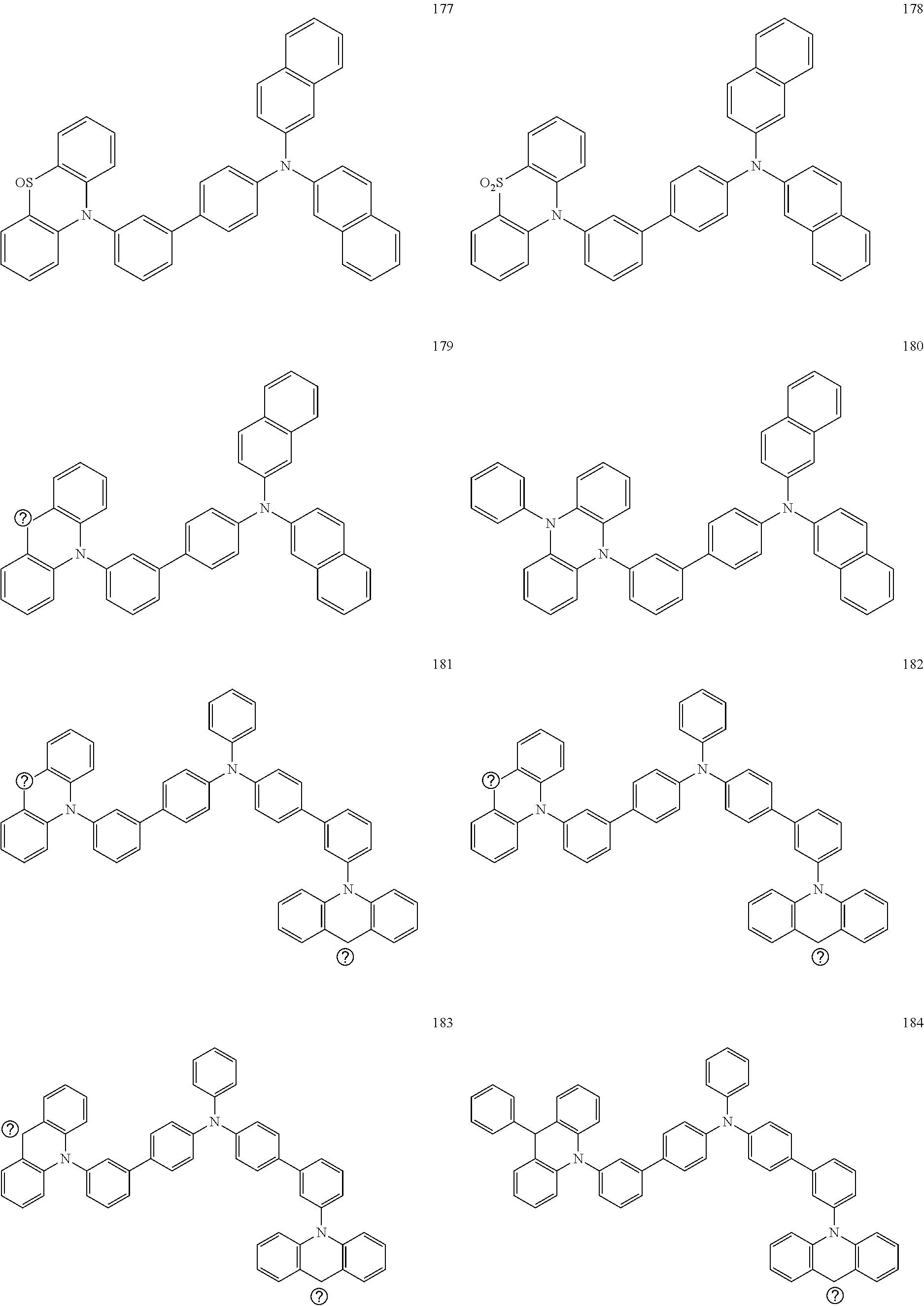

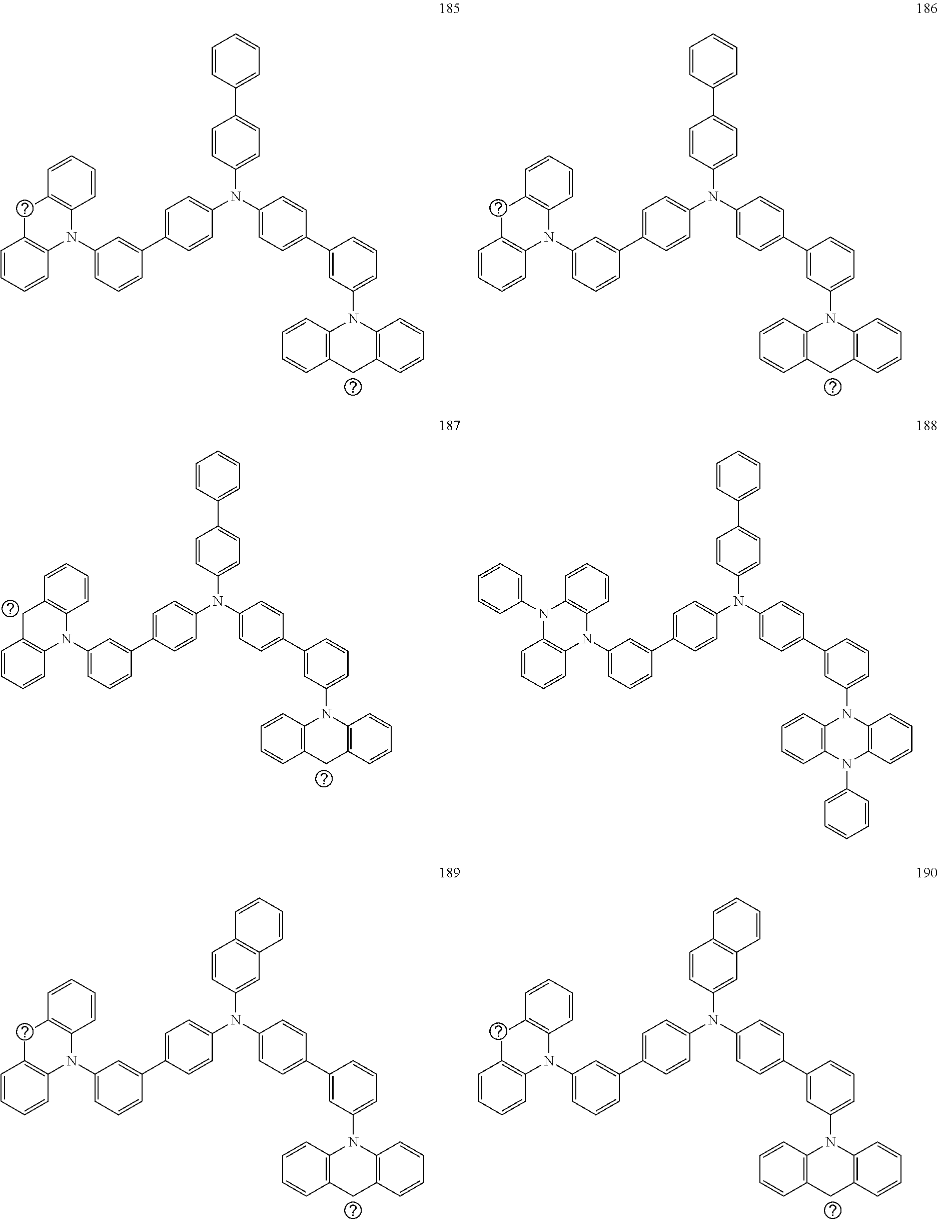

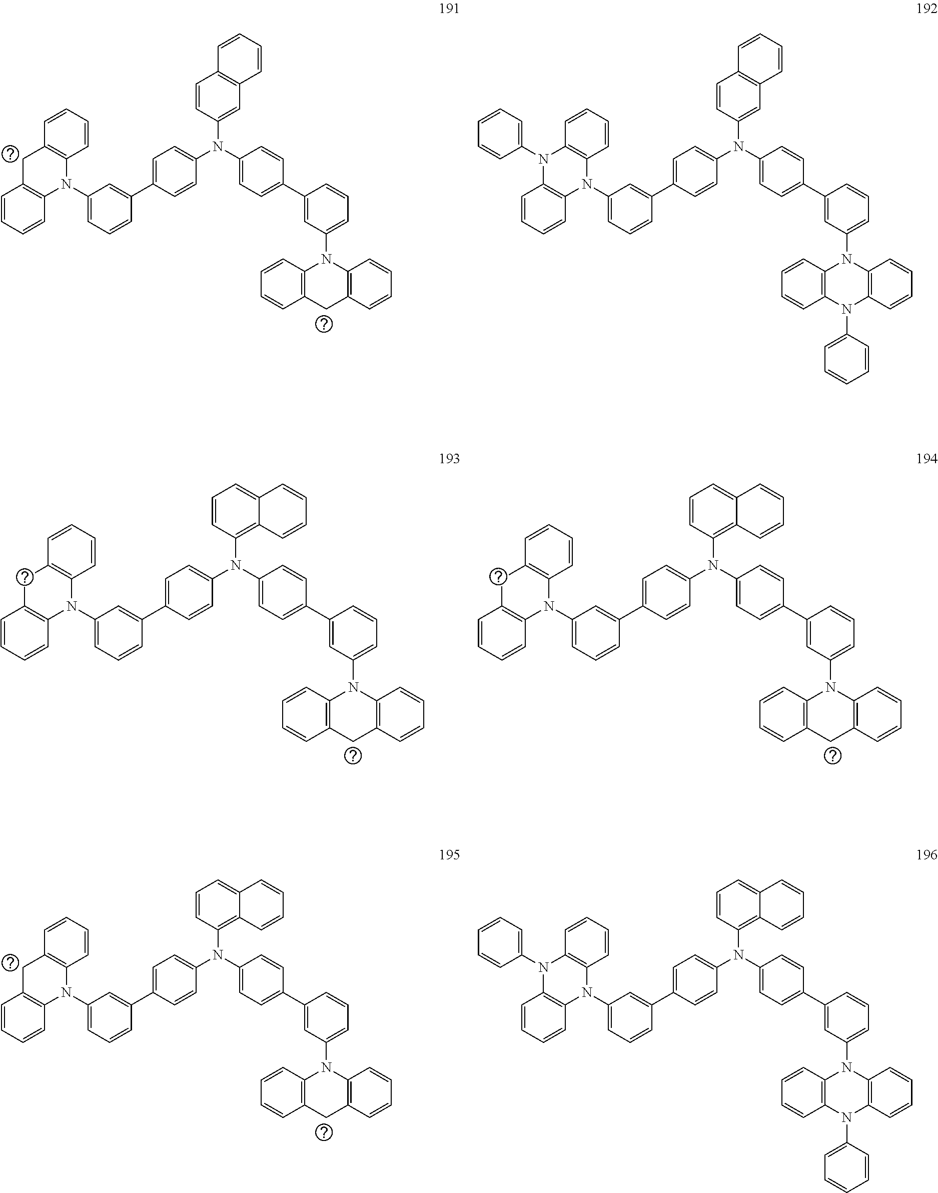

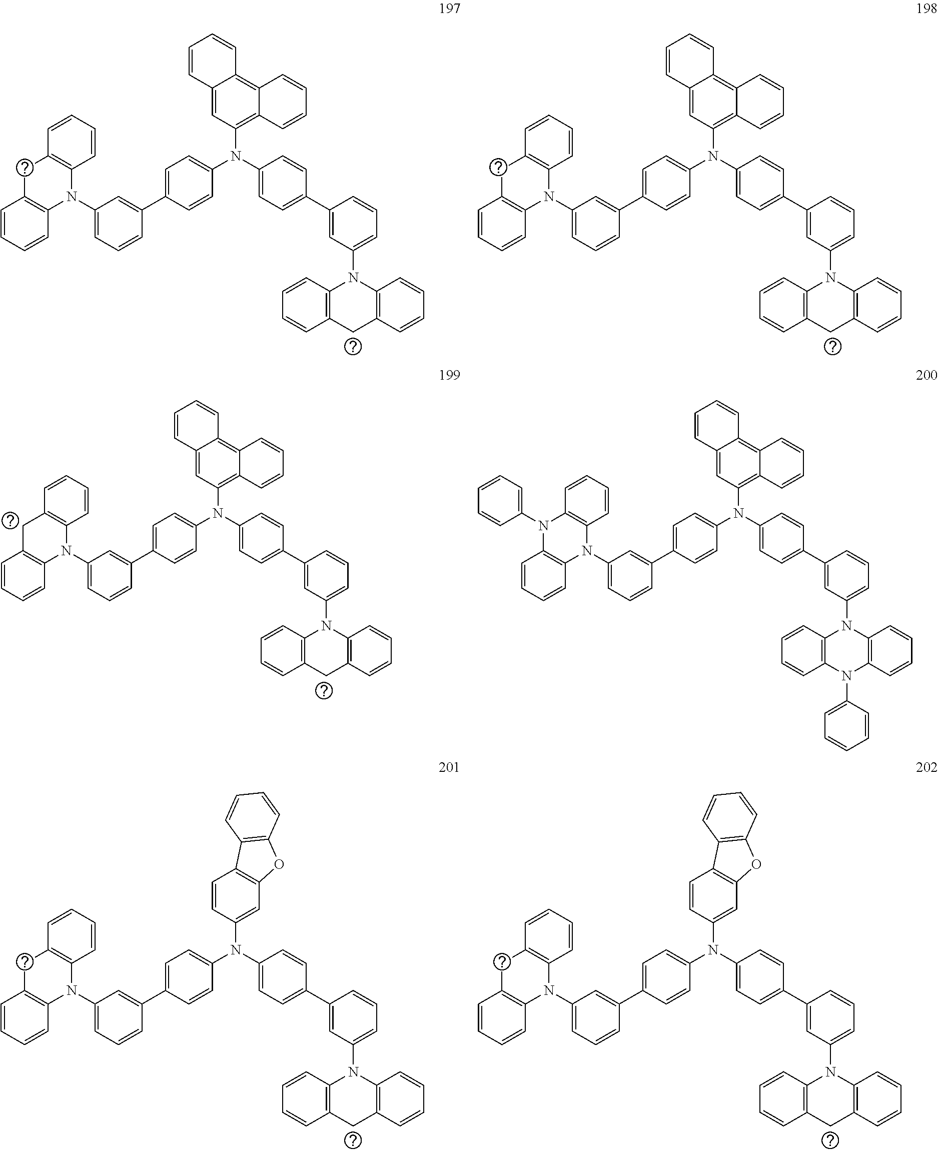

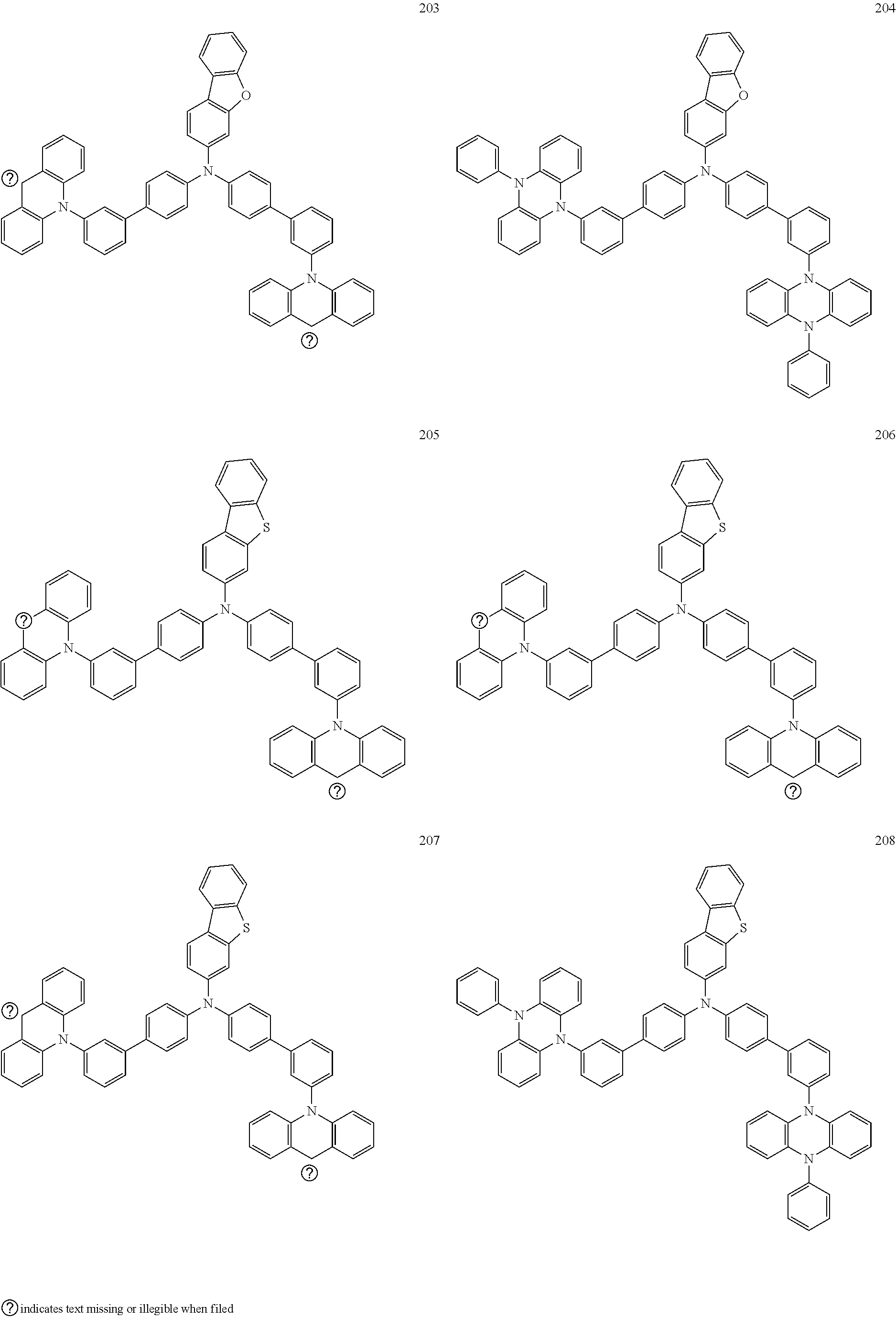

13. An organic electroluminescent (EL) device comprising:
an anode,
a cathode,
an emission layer between the anode and the cathode, and
a plurality of layers between the anode and the emission layer,
wherein a material for an organic EL device comprising an amine derivative represented by the following Formula 1 is in at least one layer selected from the plurality of layers between the anode and the emission layer: 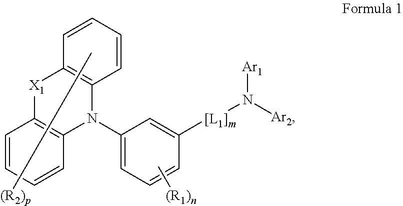
wherein Ar₁ and Ar₂ are each independently selected from a substituted or unsubstituted aryl group having 6 to 30 carbon atoms for forming a ring and a substituted or unsubstituted heteroaryl group having 2 to 30 carbon atoms for forming a ring,
L₁ is a divalent linker,
X₁ is selected from C(R₃)₂, C═O, Si(R₃)₂, N—R₃, O, S, SO and SO₂,
R₁, R₂ and R₃ are each independently selected from a substituted or unsubstituted linear, branched or cyclic alkyl group having 1 to 16 carbon atoms, a substituted or unsubstituted aryl group having 6 to 36 carbon atoms for forming a ring, a substituted or unsubstituted heteroaryl group having 2 to 32 carbon atoms for forming a ring, and an aryl group or a heteroaryl group formed via condensation of a plurality of adjacent substituents,
n is an integer selected from 0 to 4,
m is an integer selected from 1 to 4, and
p is an integer selected from 0 to 8.

14. The organic EL device of claim 13, wherein L₁ is selected from a substituted or unsubstituted arylene group having 6 to 30 carbon atoms for forming a ring and a substituted or unsubstituted heteroarylene group having 2 to 30 carbon atoms for forming a ring.

15. The organic EL device of claim 13, wherein Ar₁ and Ar₂ are each independently selected from a group substituted with a substituent not including a nitrogen atom and an unsubstituted aryl group or heteroaryl group, the aryl group or the heteroaryl group not including a nitrogen atom.

16. The organic EL device of claim 13, wherein Ar₁ is a group substituted with a substituent not including a nitrogen atom or an unsubstituted aryl group or heteroaryl group not including a nitrogen atom, and
Ar₂ is represented by the following Formula 2: 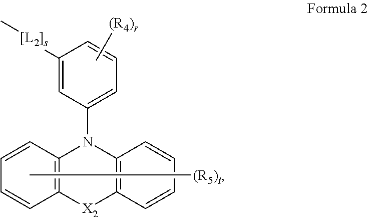
wherein L₂ is a divalent linker,
X₂ is selected from C(R₆)₂, C═O, Si(R₆)₂, N—R₆, O, S, SO and SO₂,
R₄, R₅ and R₆ are each independently selected from a substituted or unsubstituted linear, branched or cyclic alkyl group having 1 to 16 carbon atoms, a substituted or unsubstituted aryl group having 6 to 36 carbon atoms for forming a ring, a substituted or unsubstituted heteroaryl group having 2 to 32 carbon atoms for forming a ring, and an aryl group or a heteroaryl group formed via condensation of a plurality of adjacent substituents,
r is an integer selected from 0 to 4,
s is an integer selected from 1 to 4, and
t is an integer selected from 0 to 8.

17. The organic EL device of claim 16, wherein
s in Formula 2 is the same as m in Formula 1,
r in Formula 2 is the same as n in Formula 1, and
t in Formula 2 is the same as p in Formula 1.

18. The organic EL device of claim 13, wherein the amine derivative represented by Formula 1 is at least one selected from the following Compounds 1 to 208: 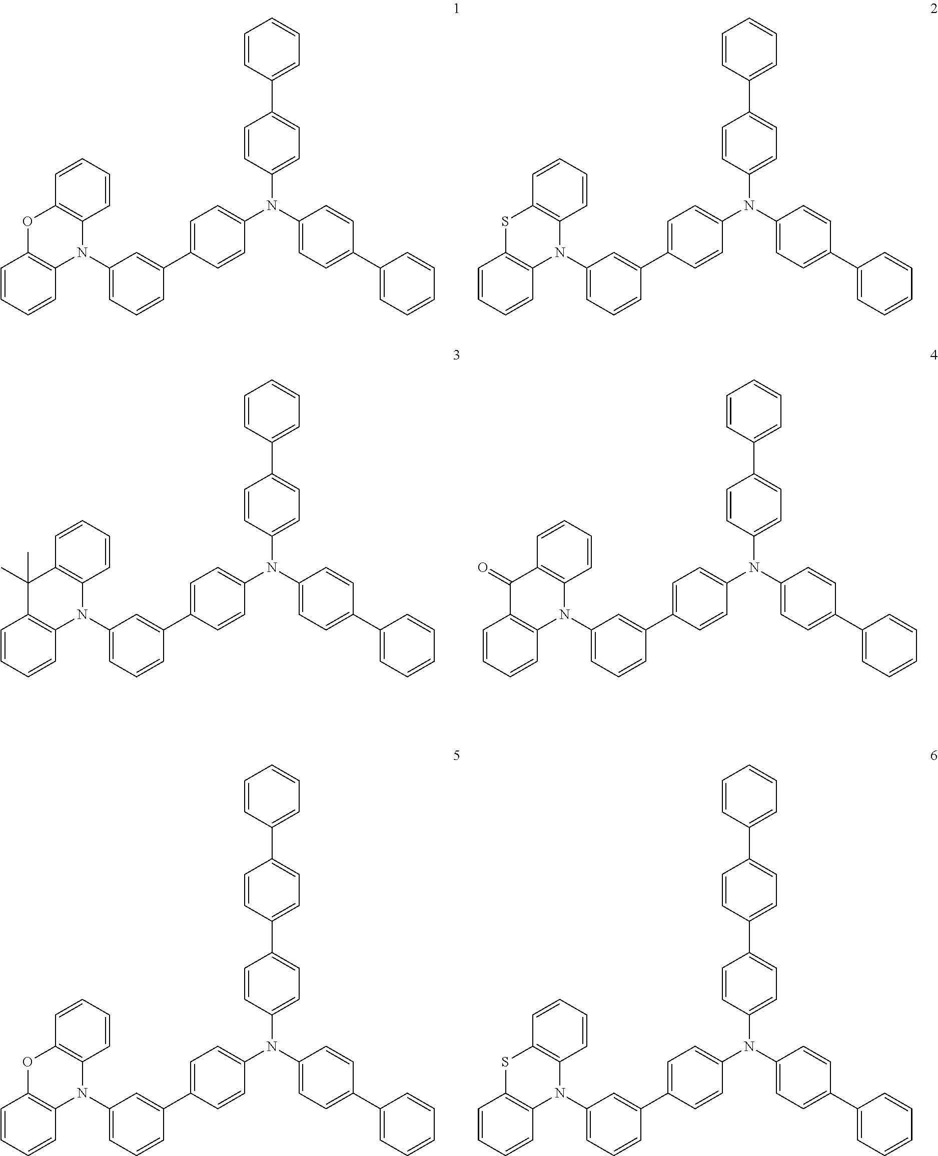 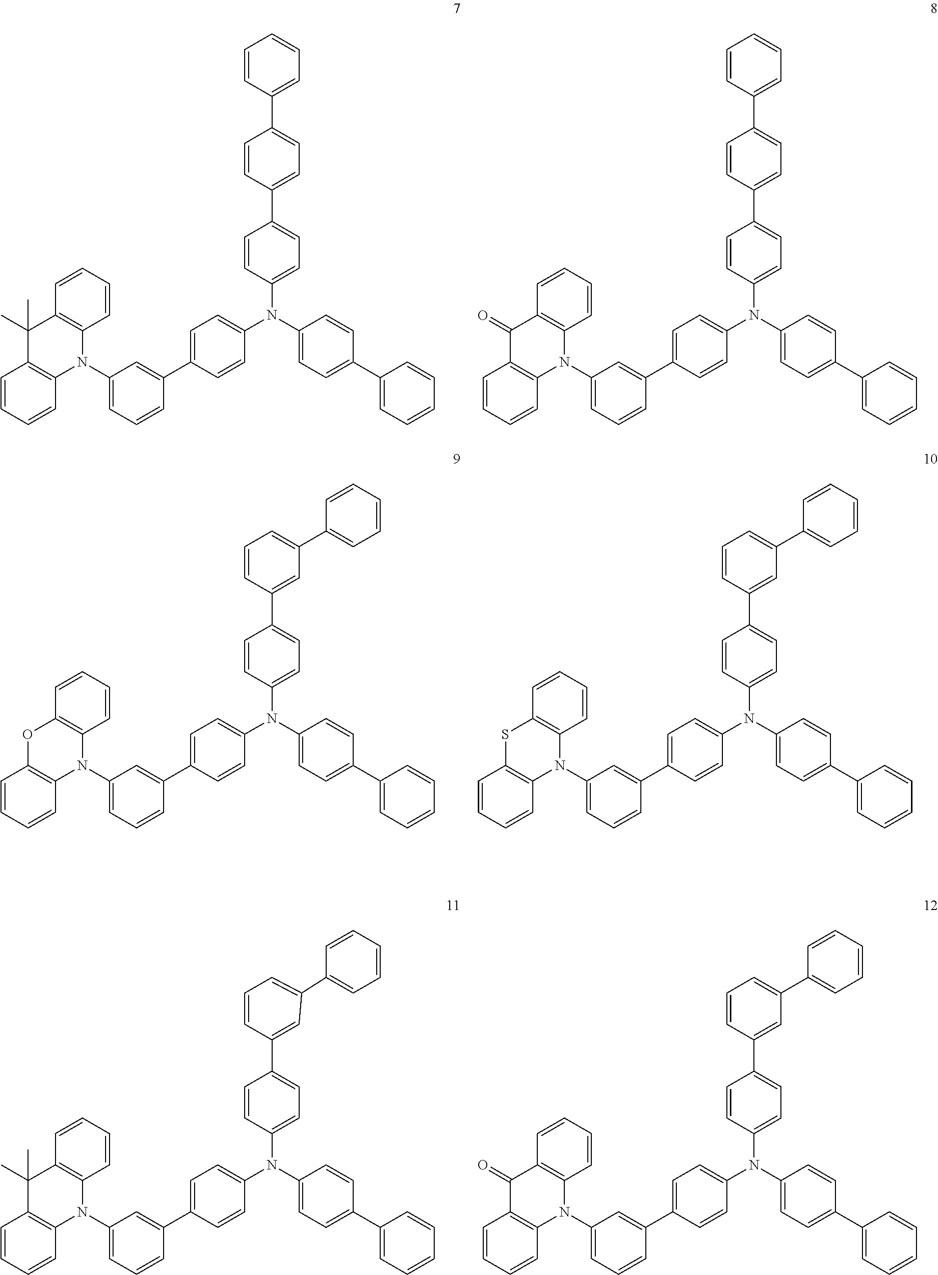 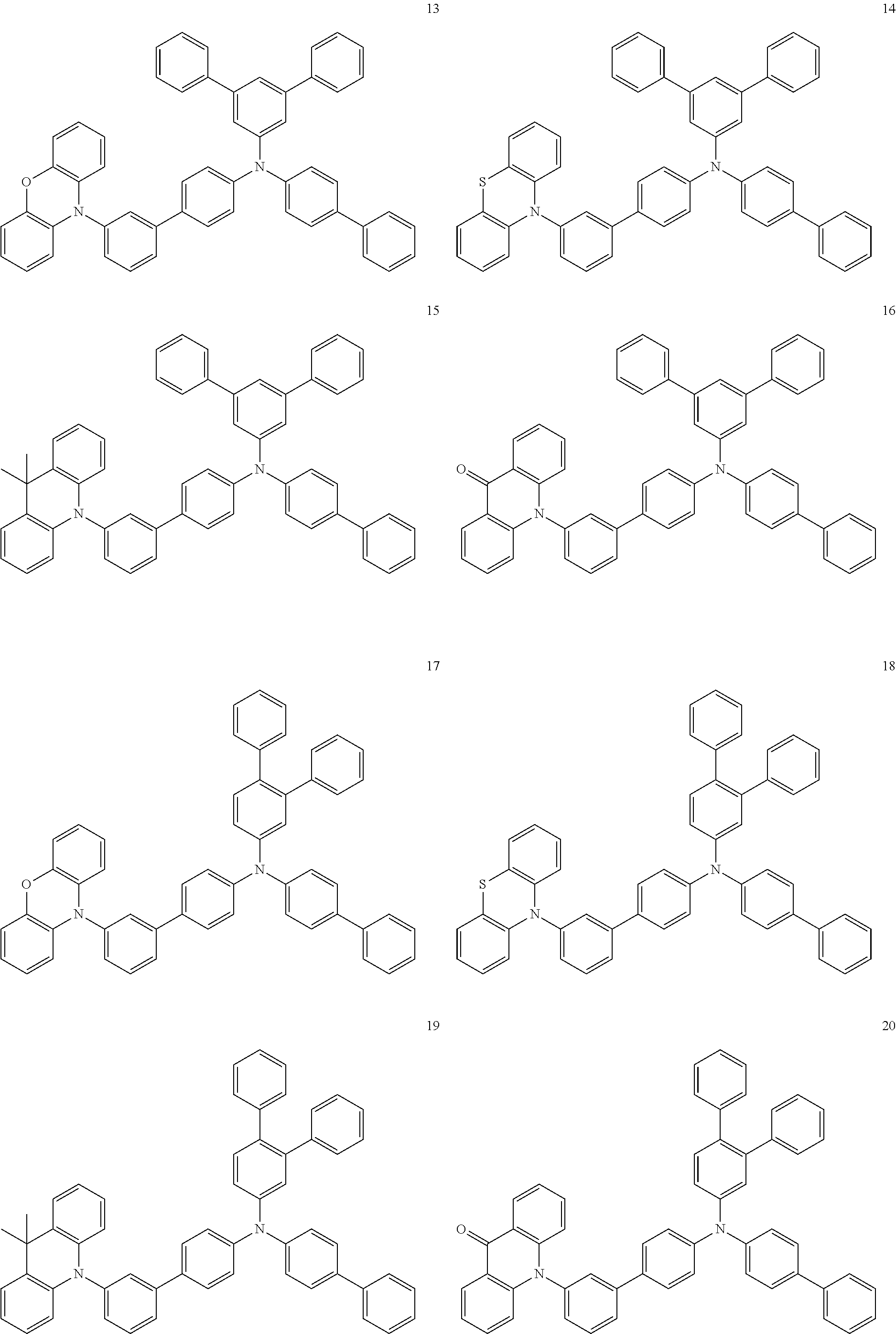 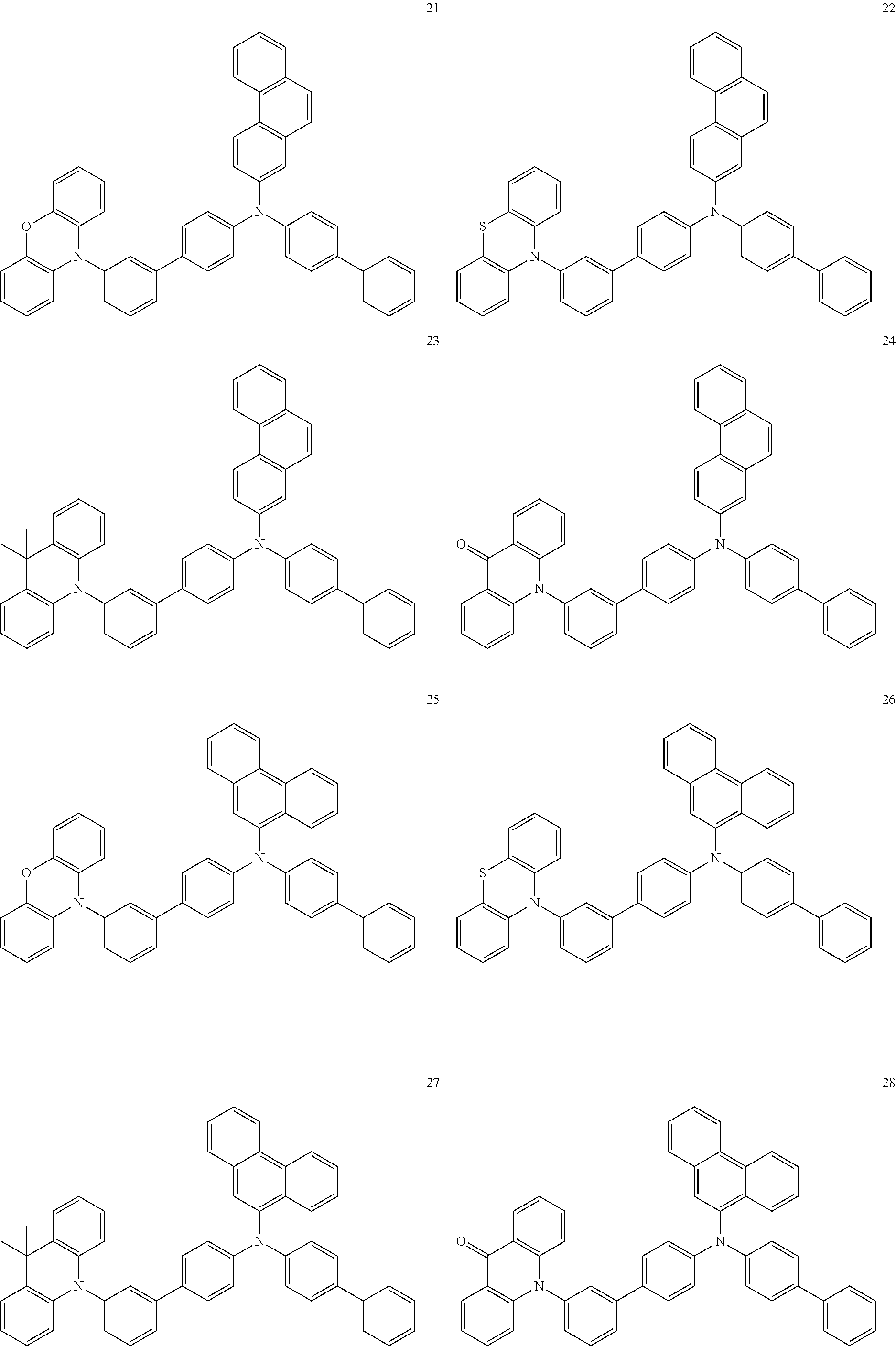 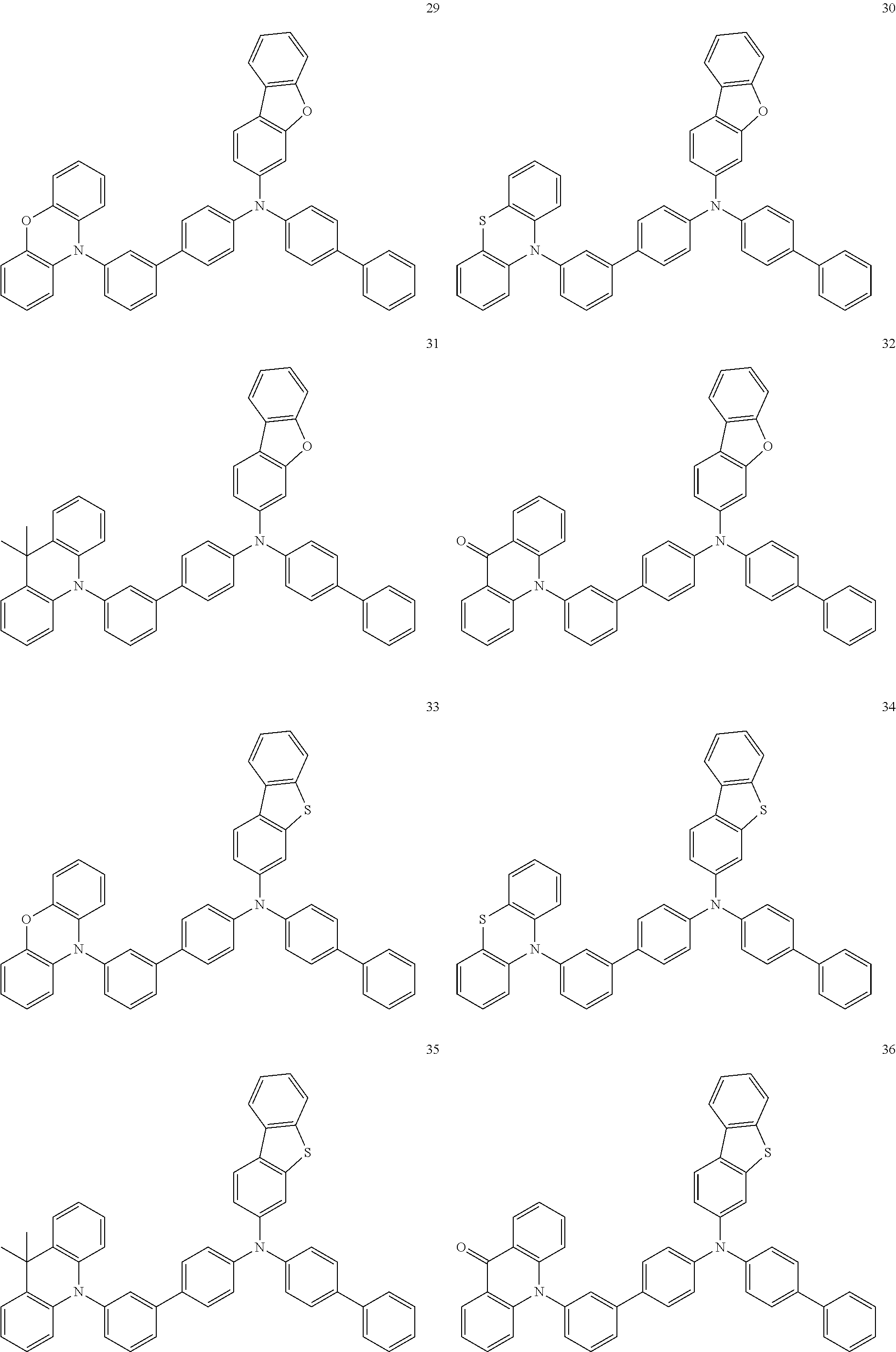 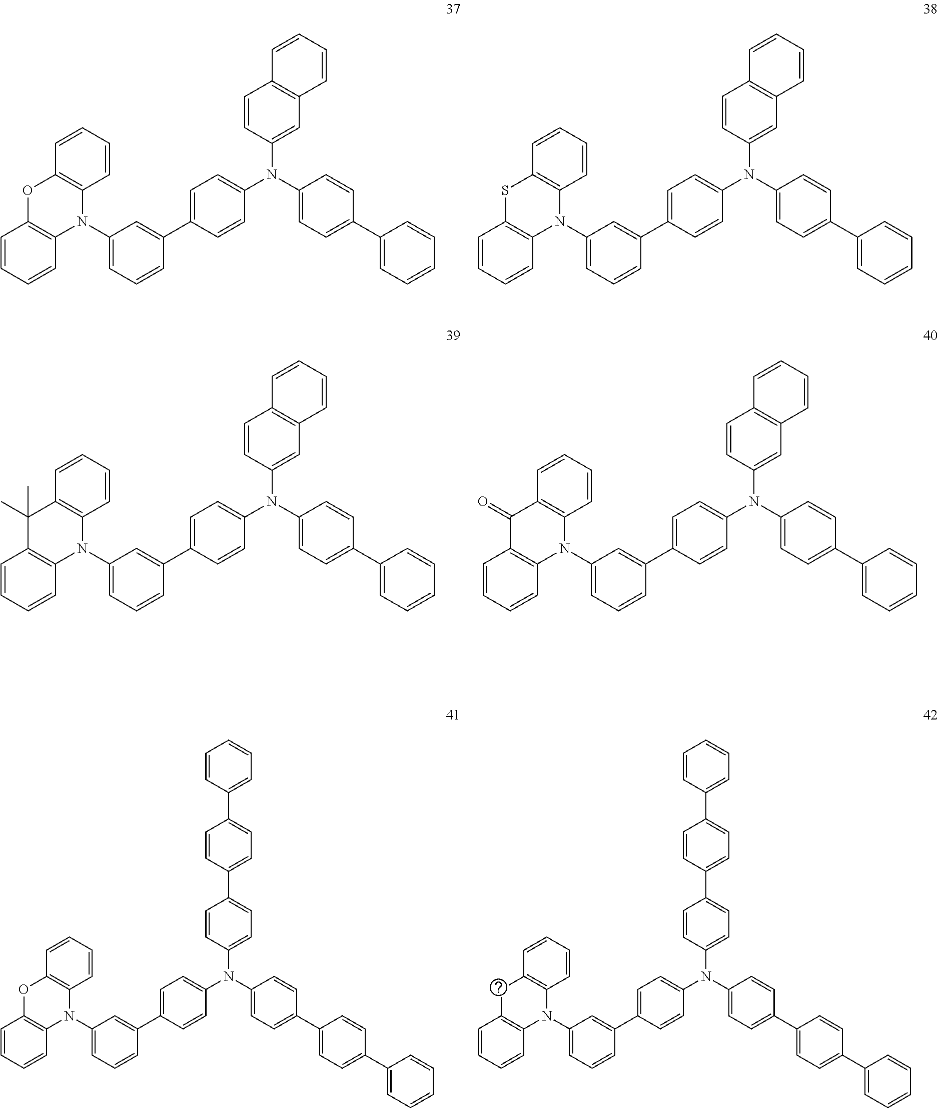 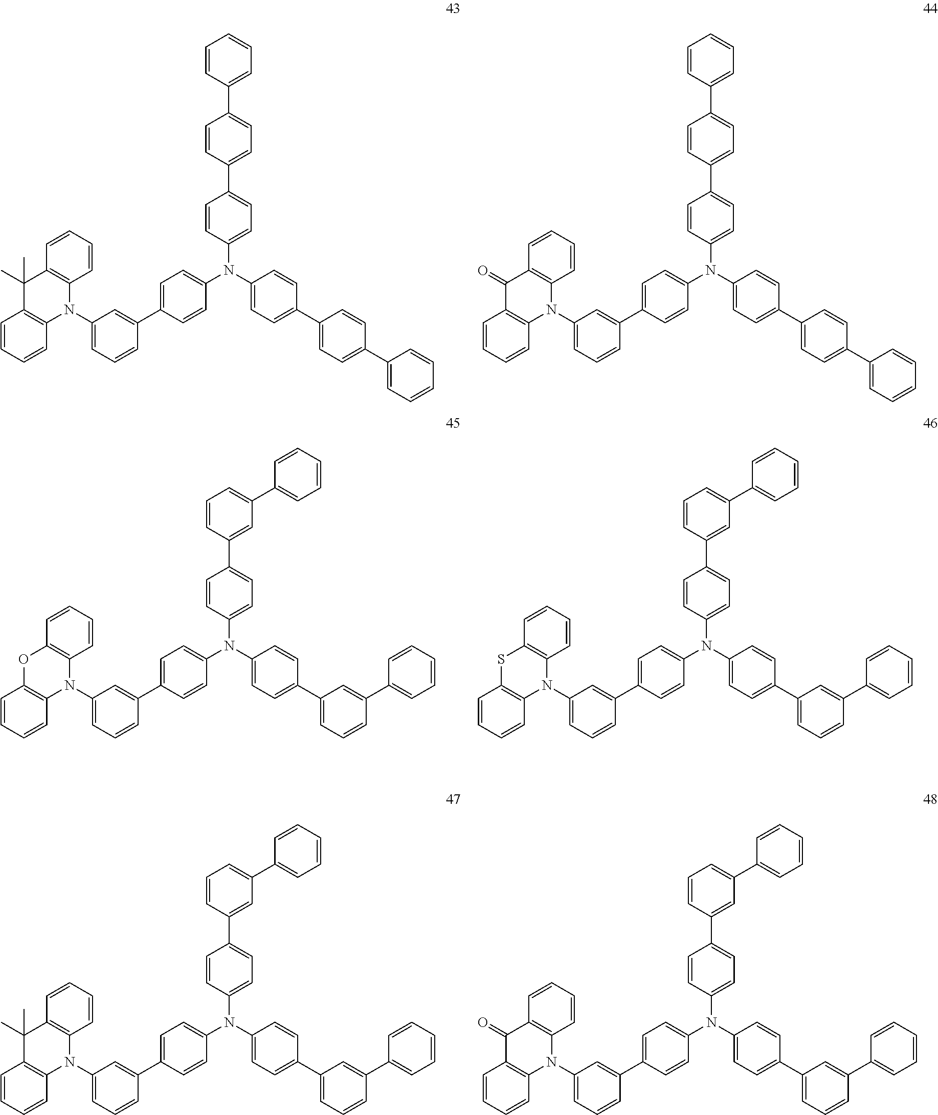 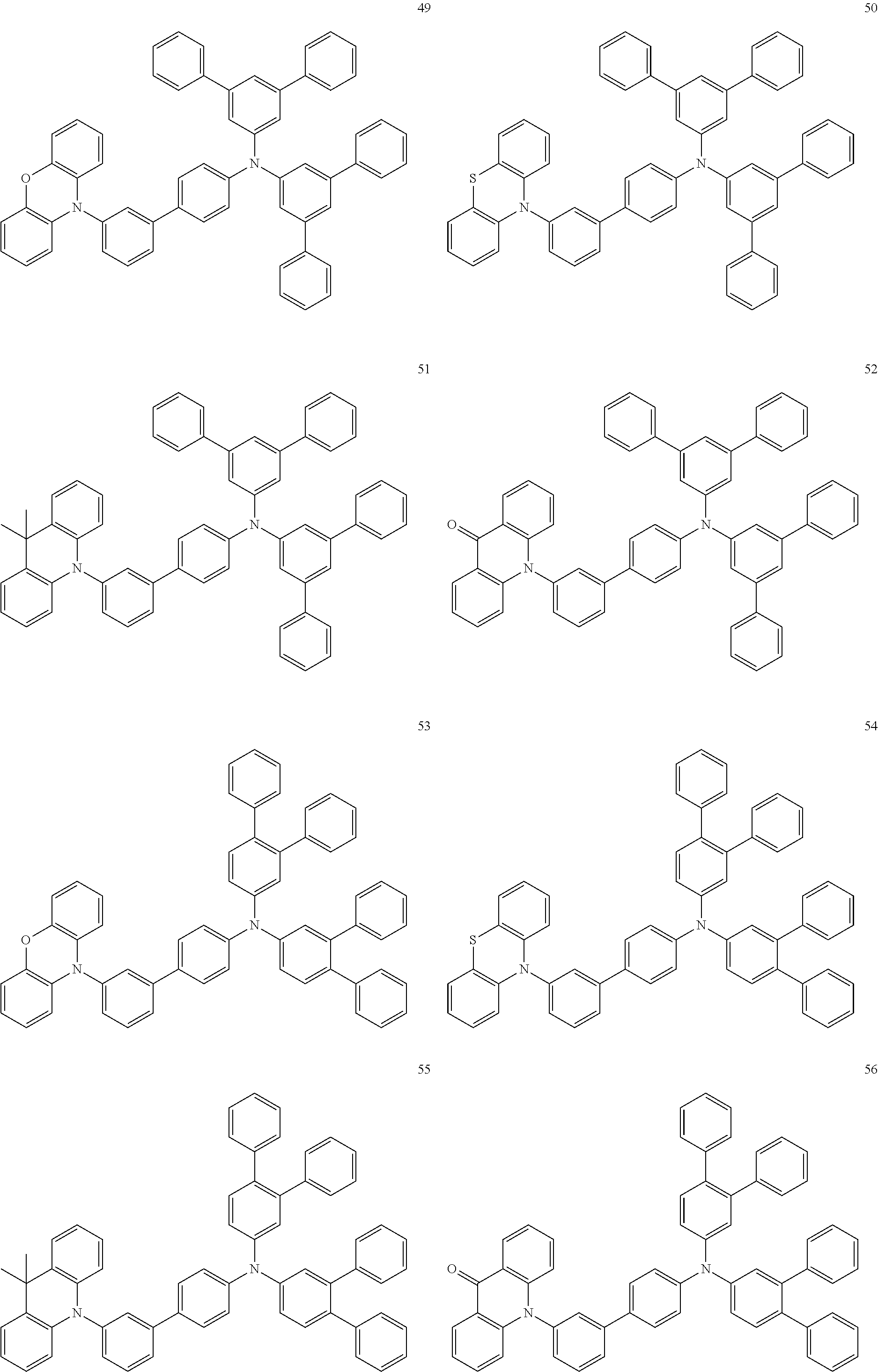 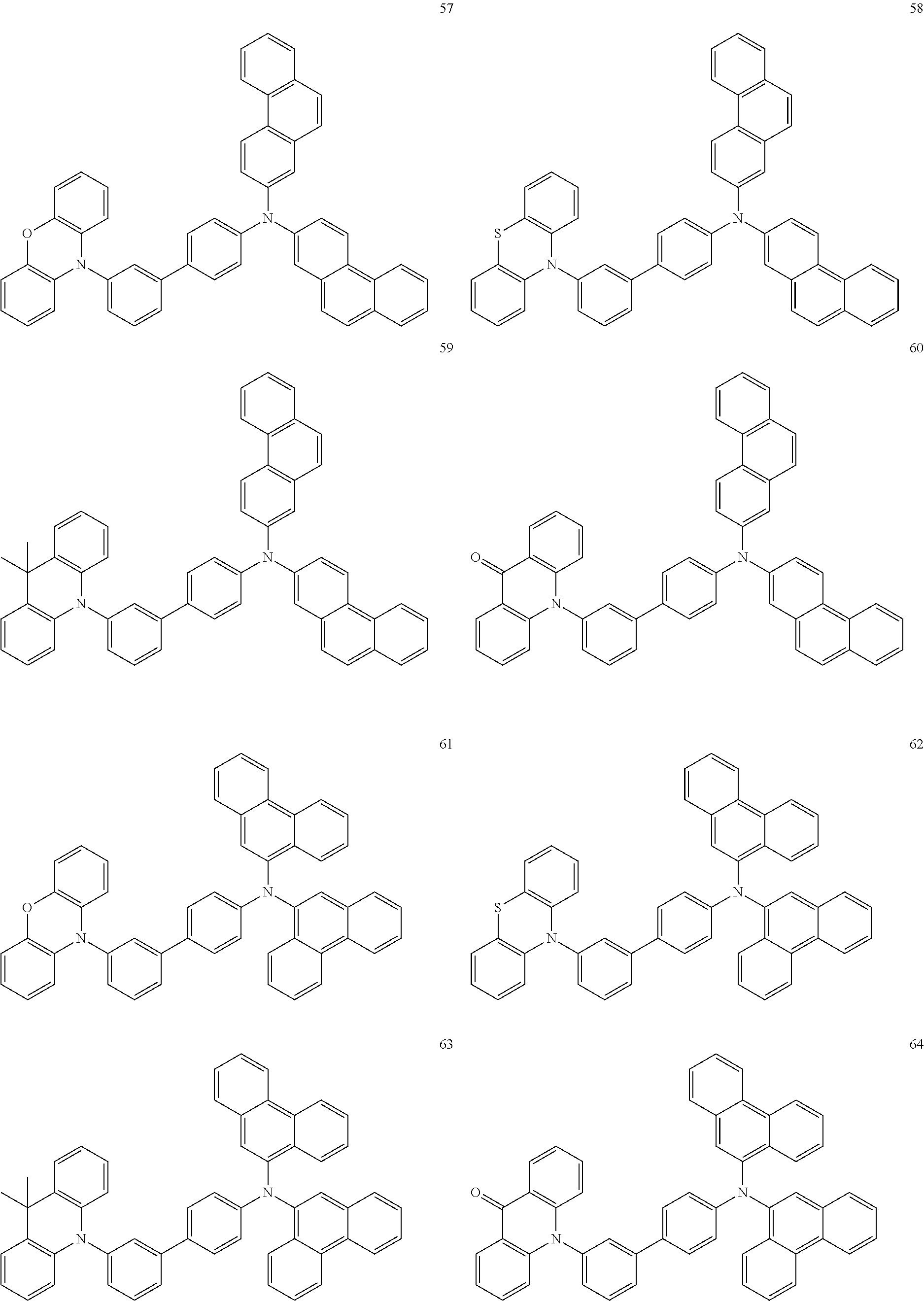 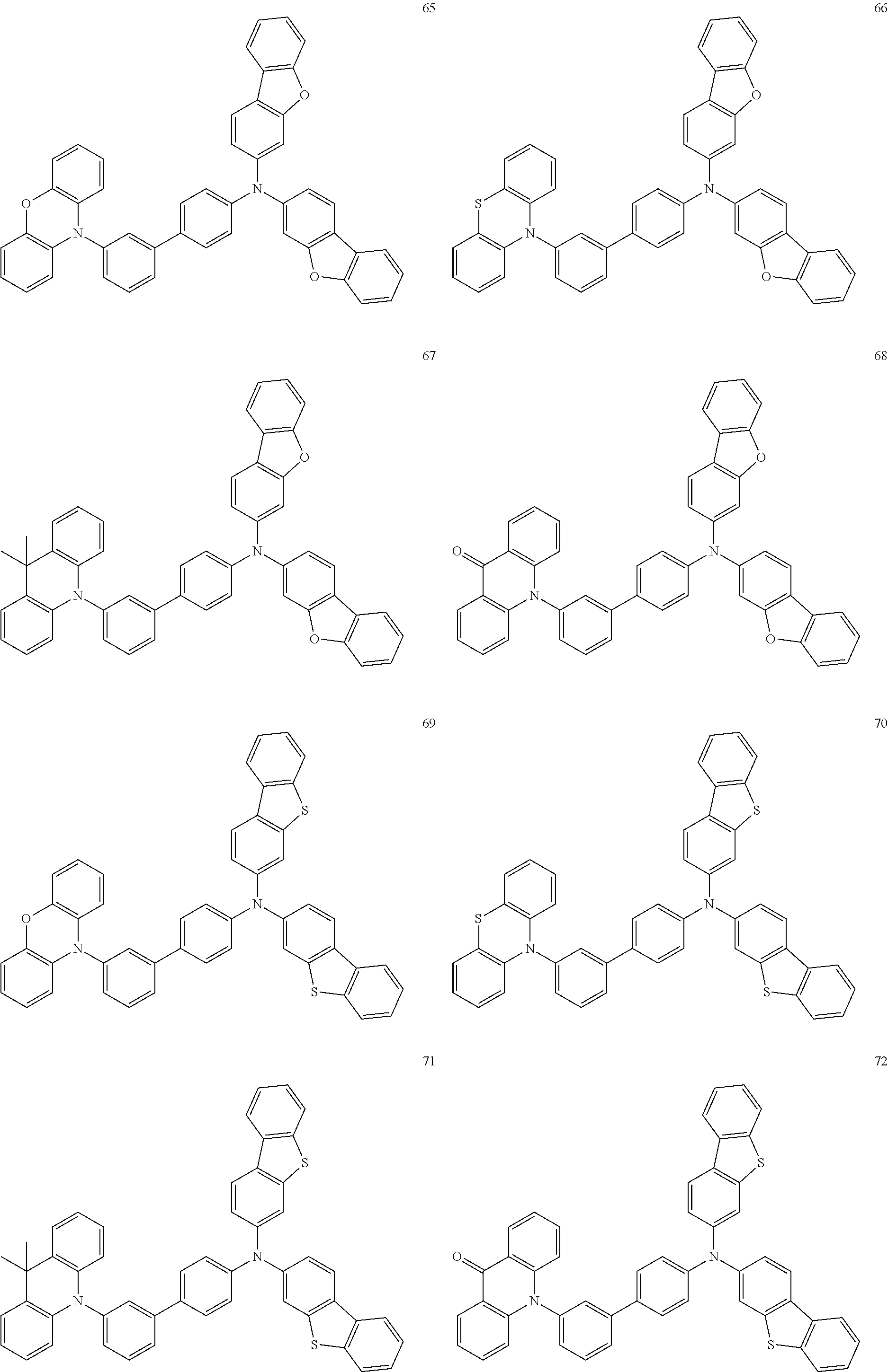 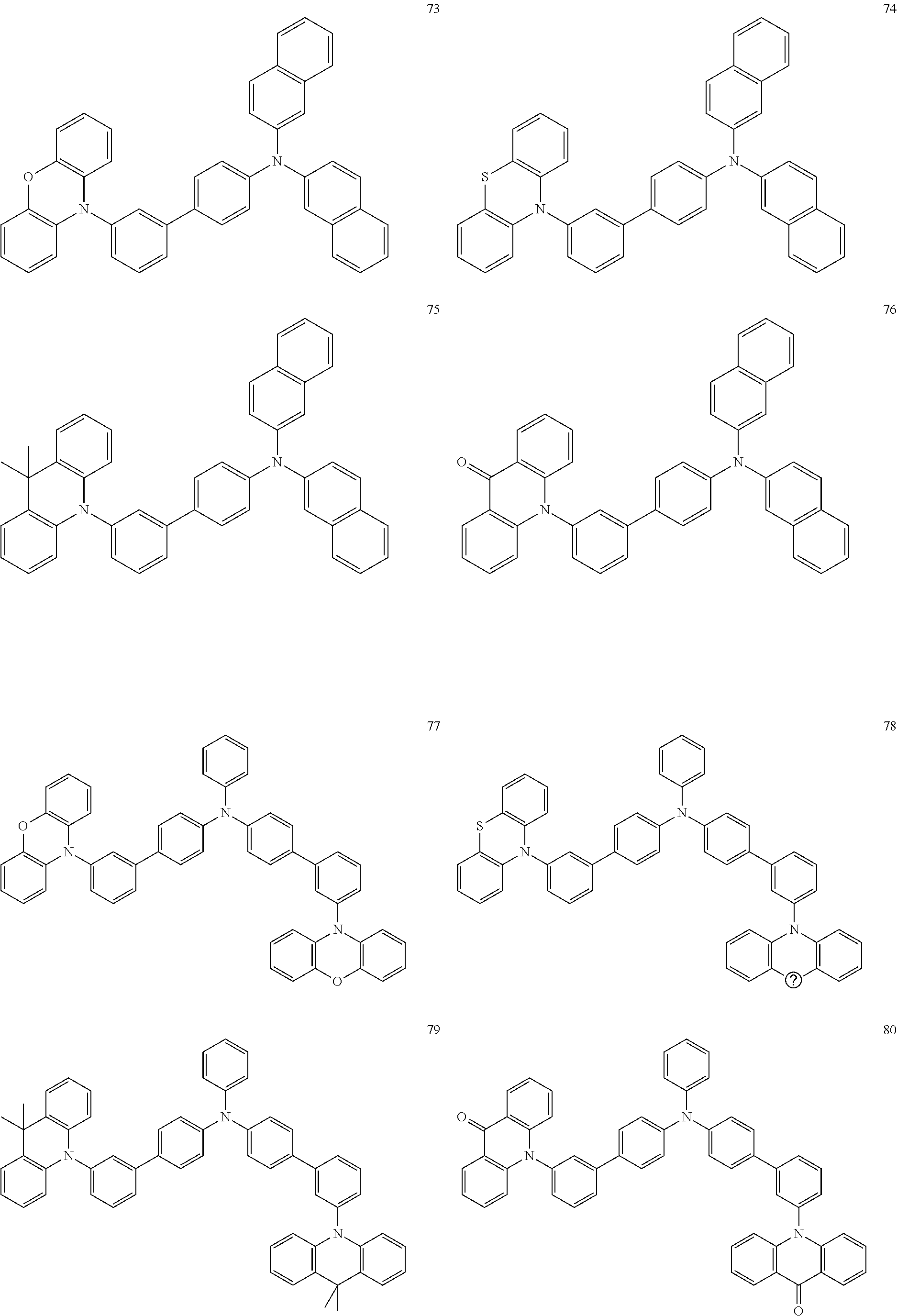 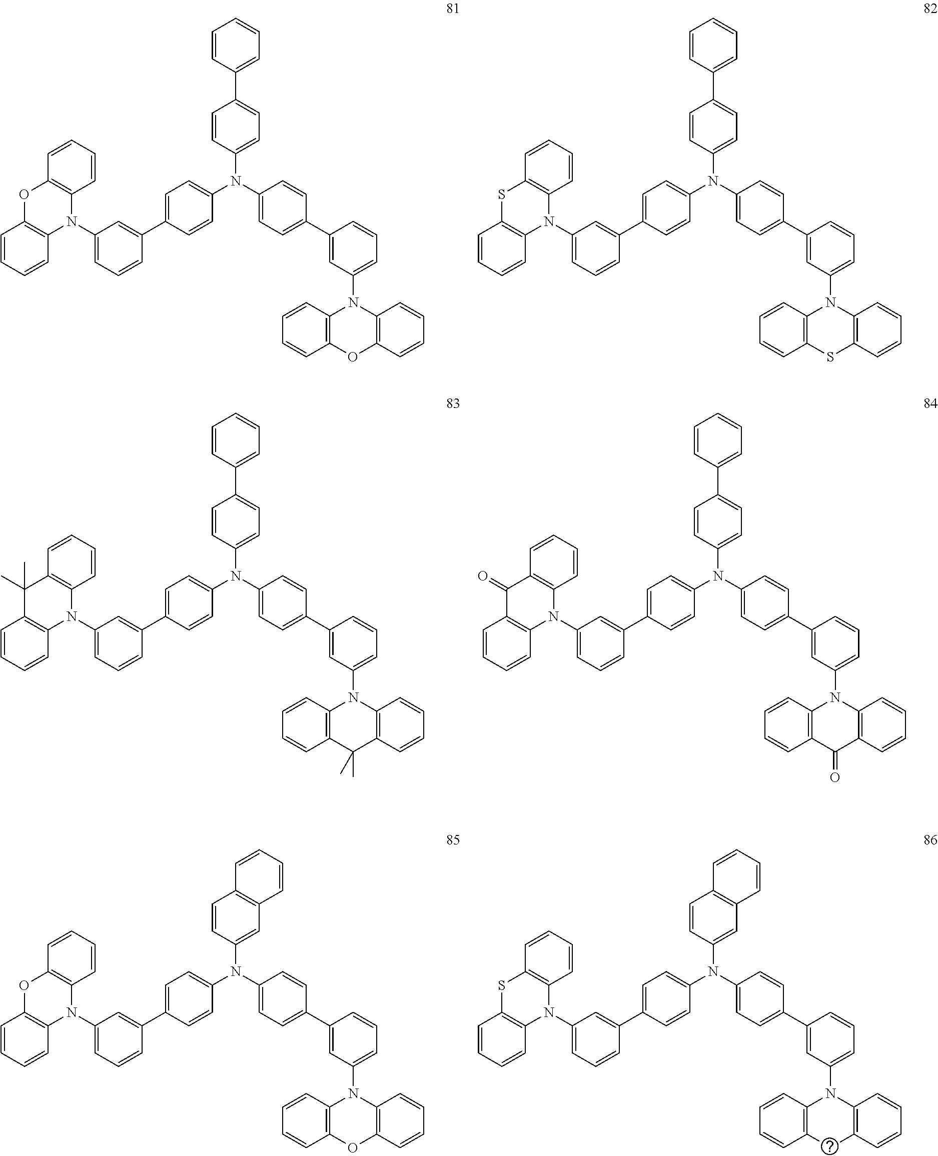 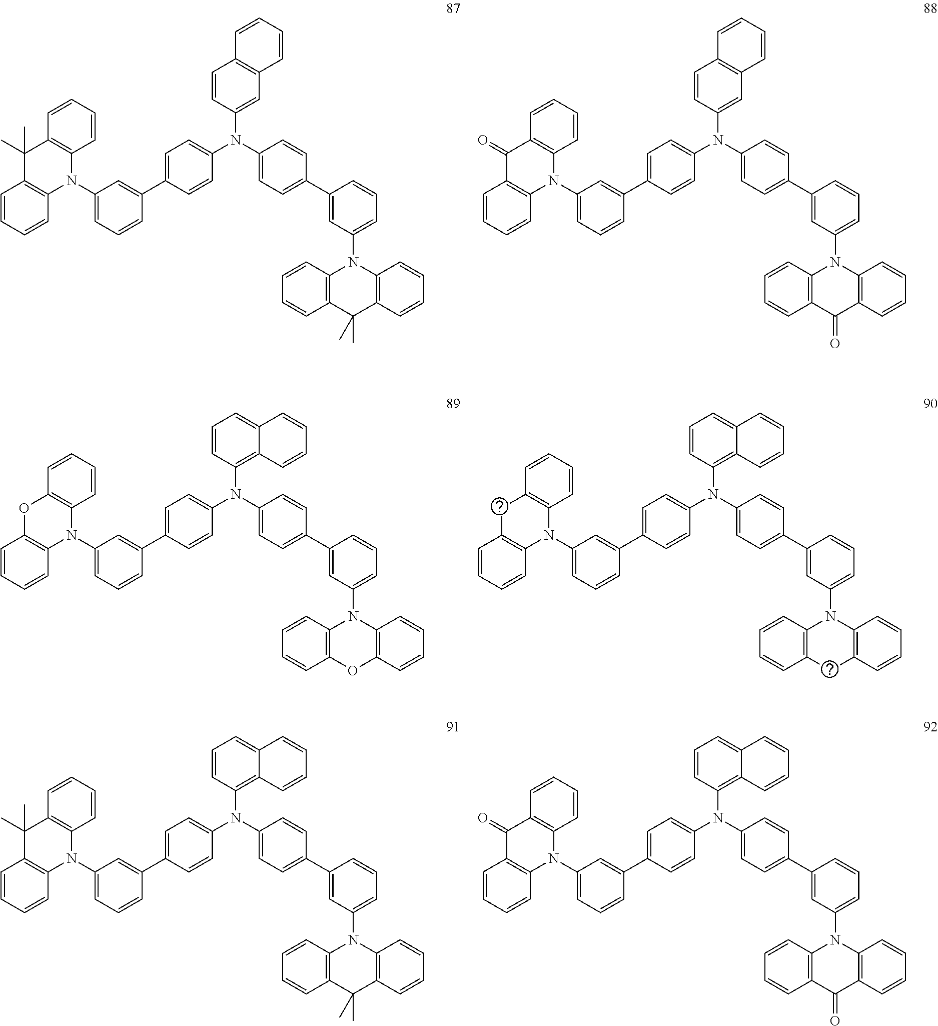 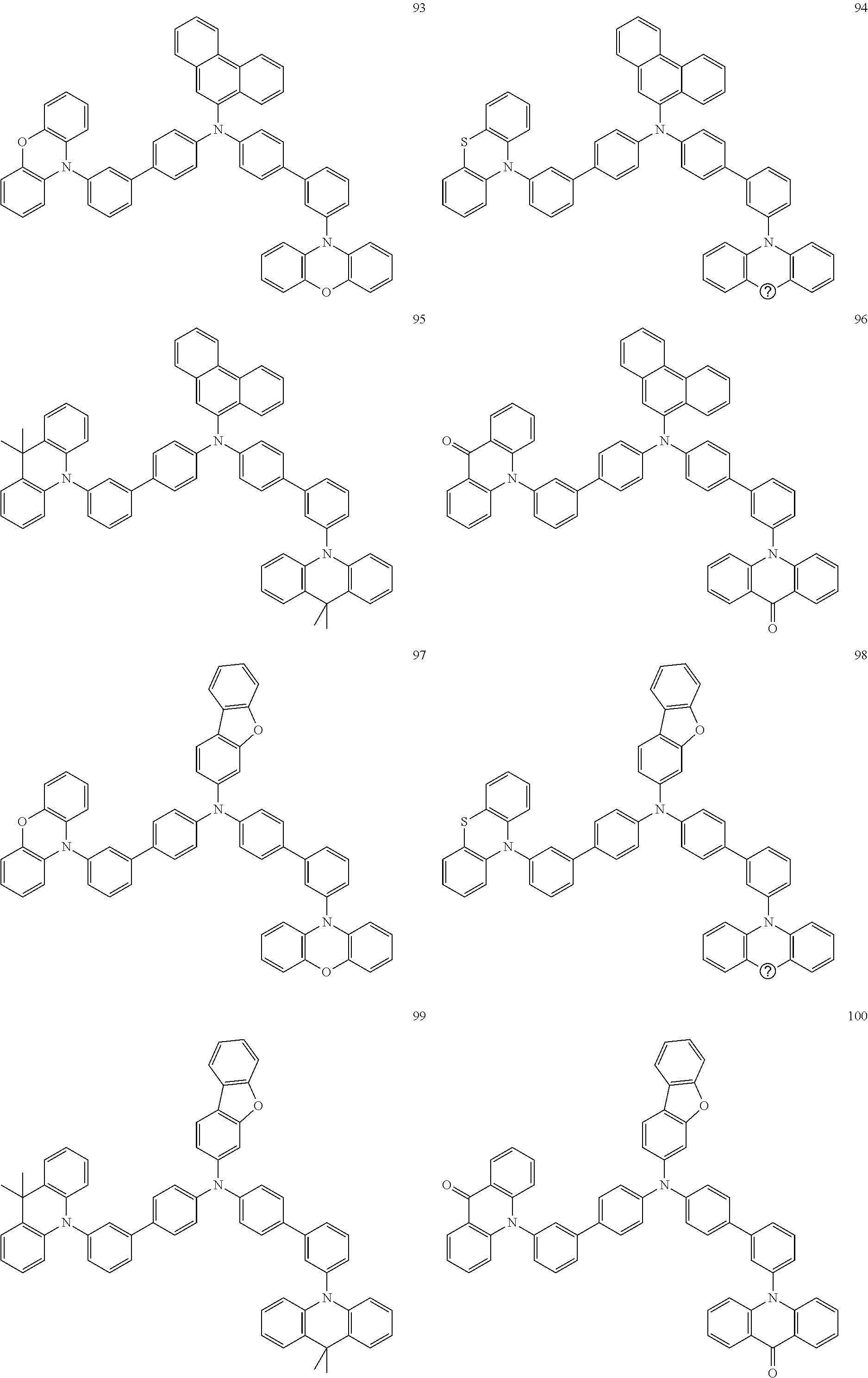 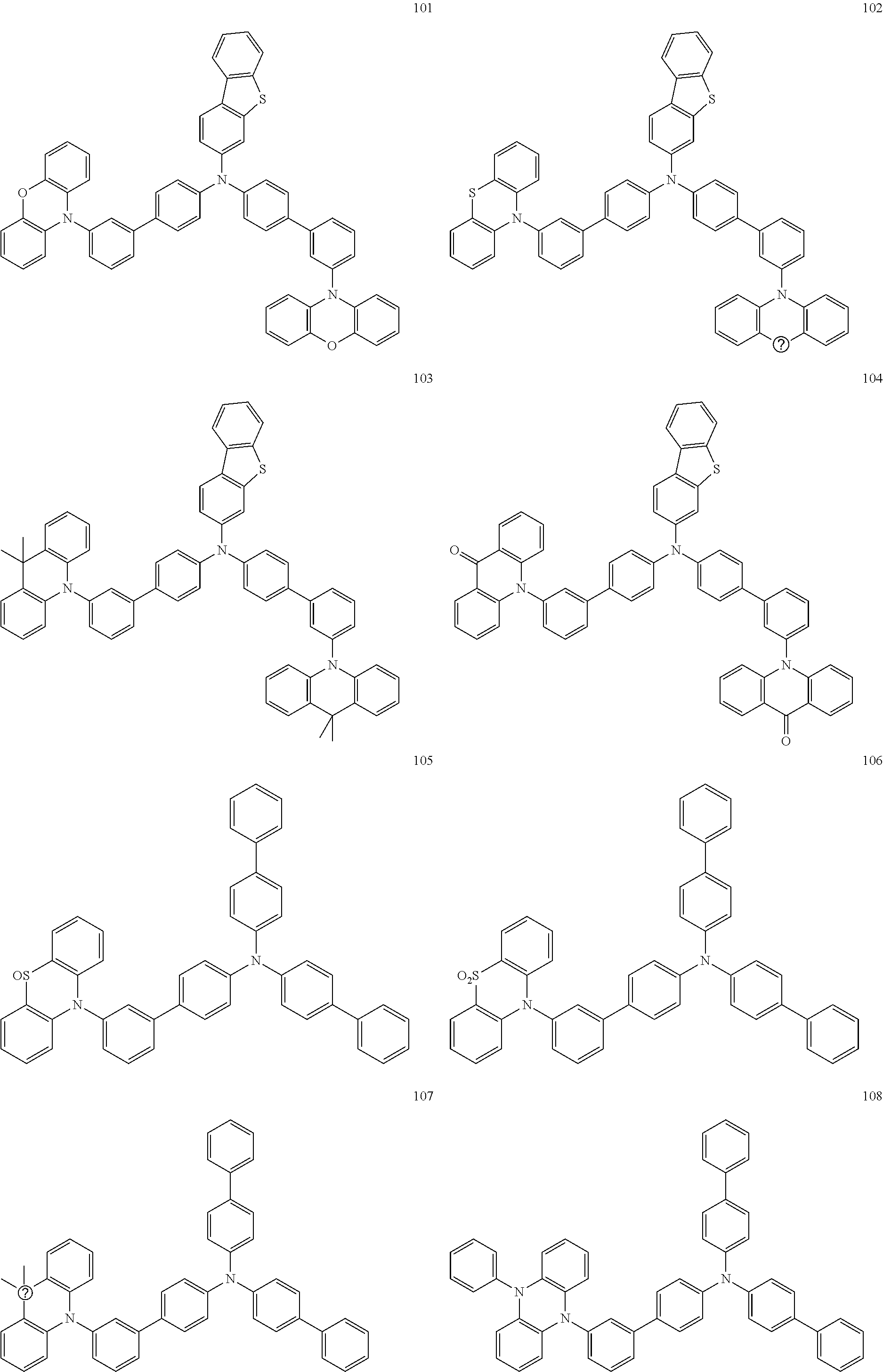 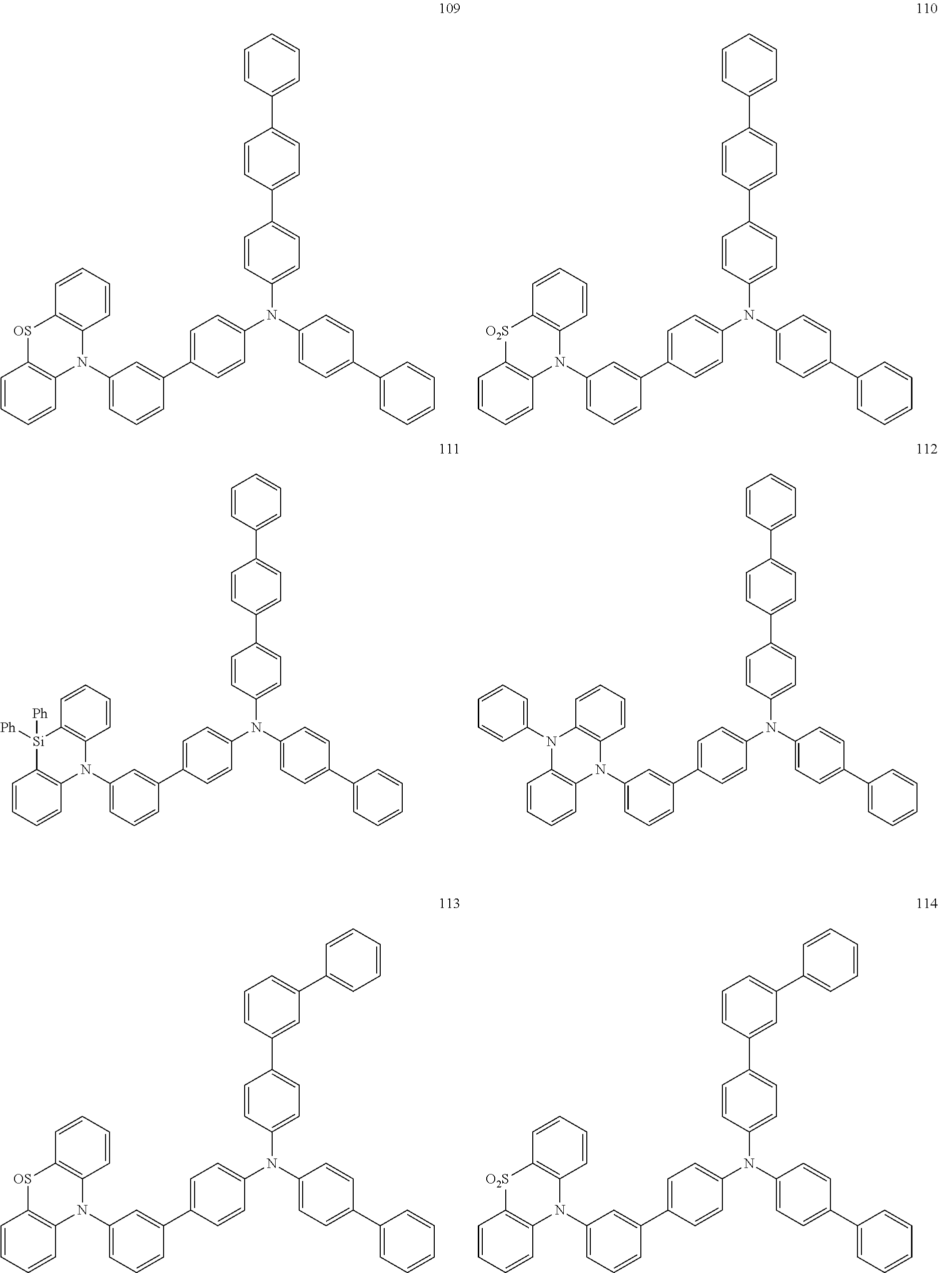 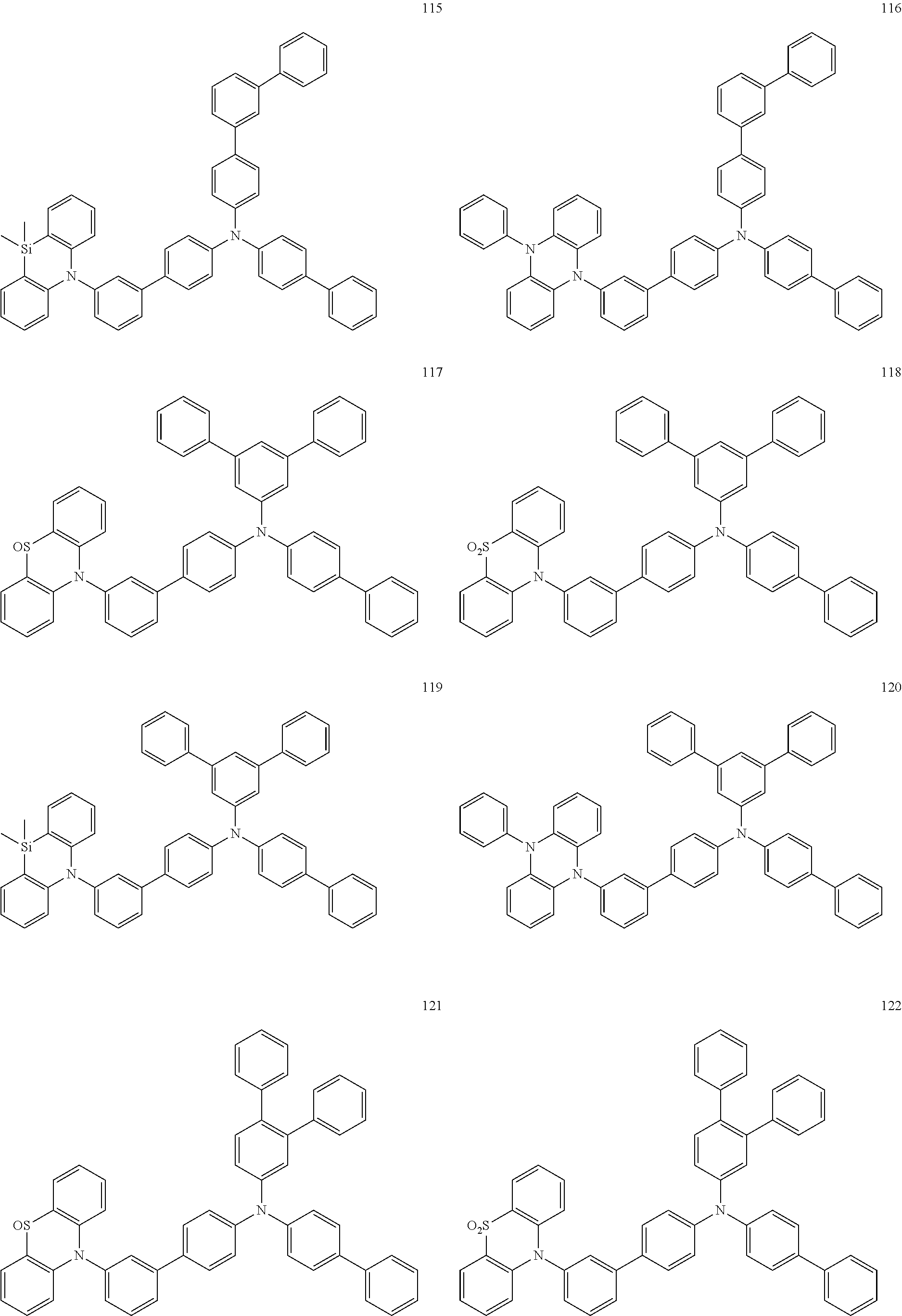 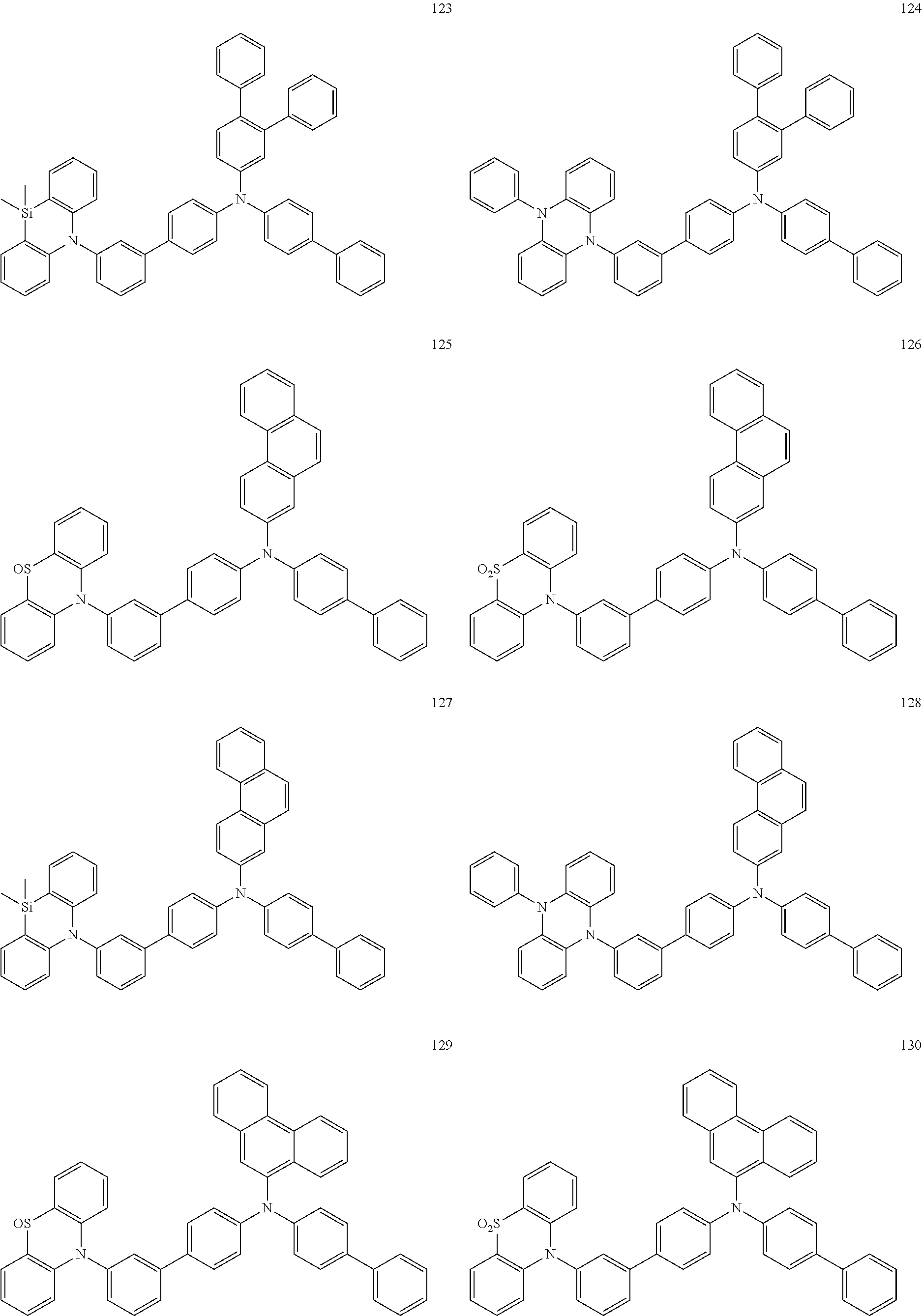 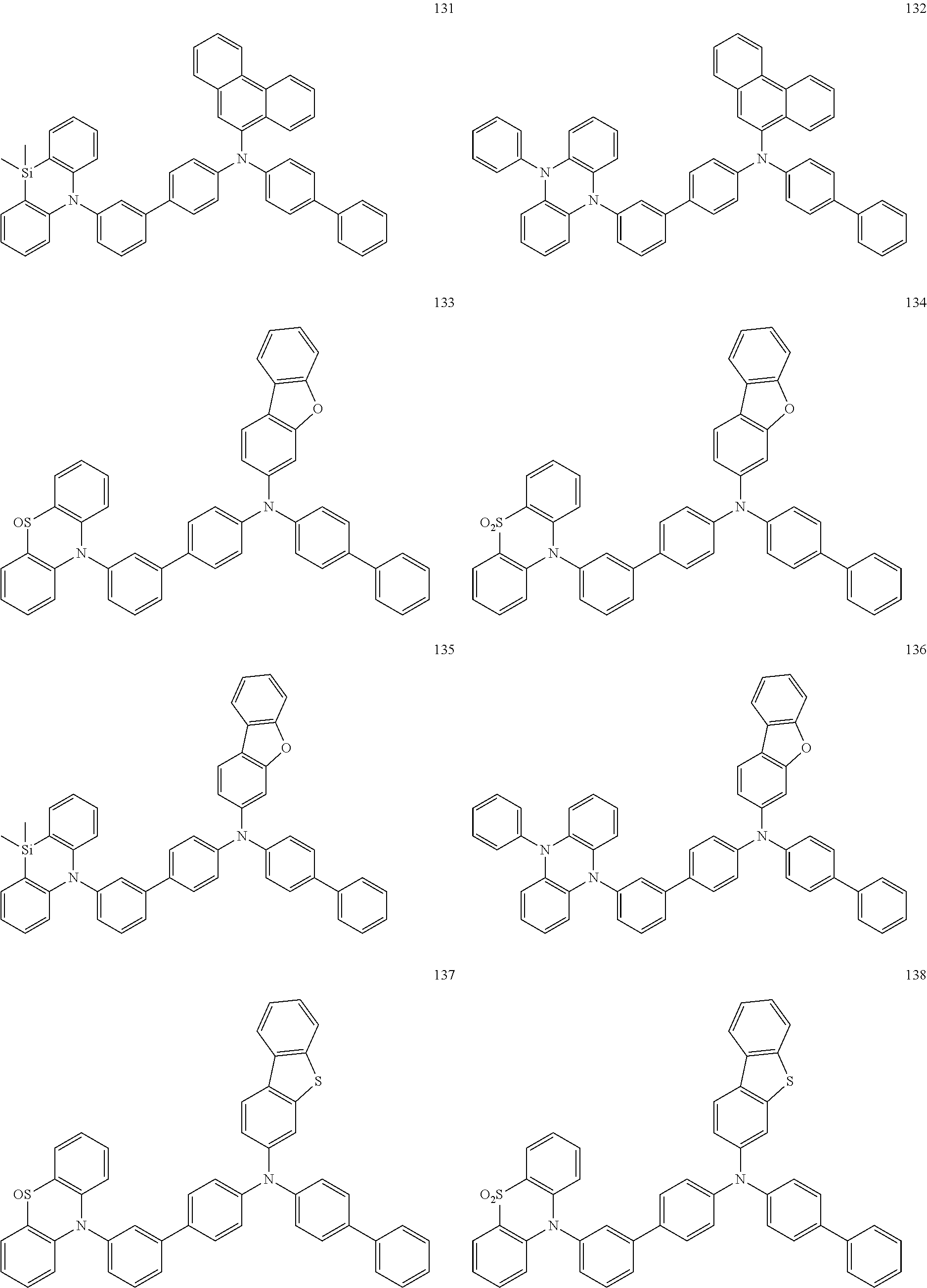 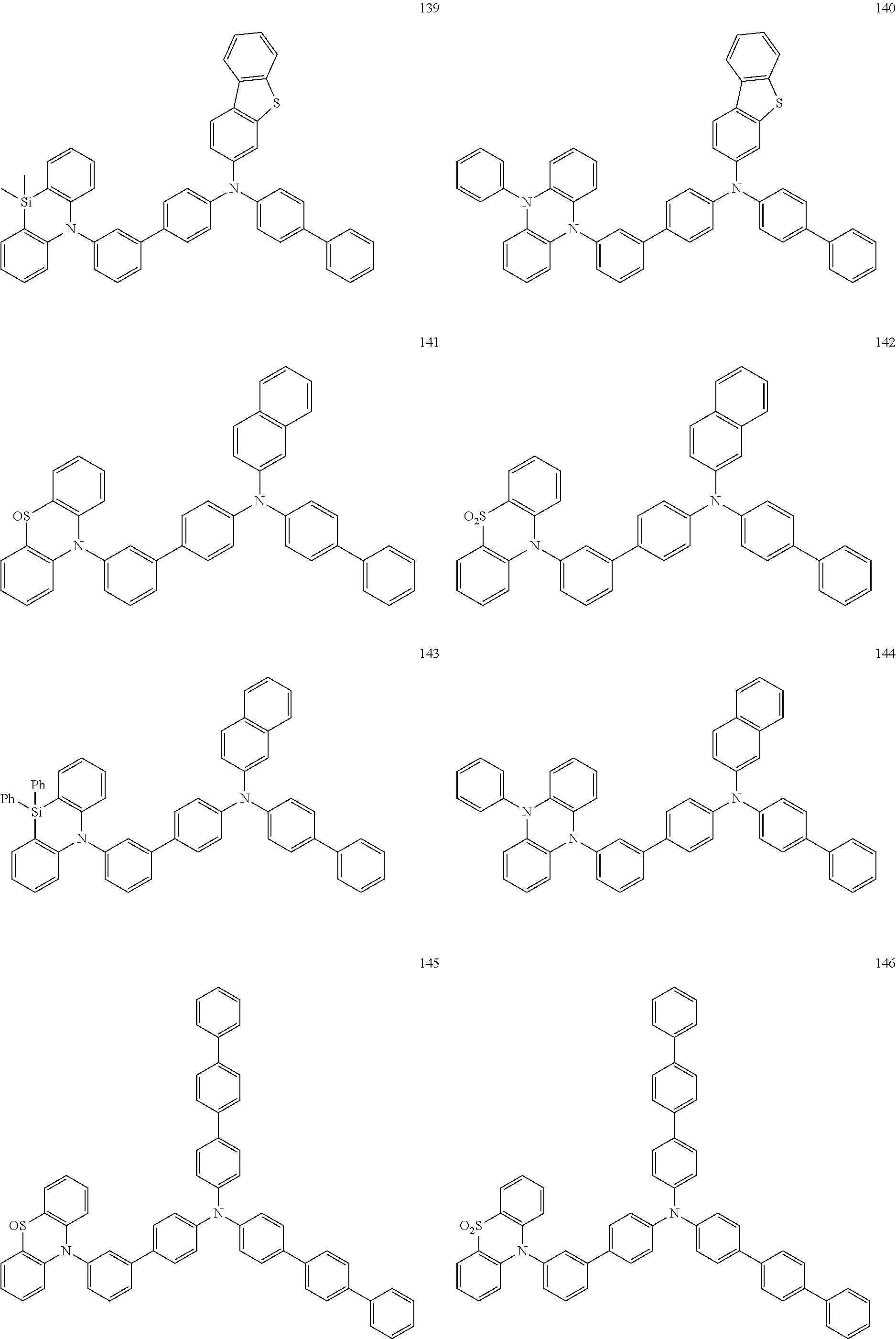 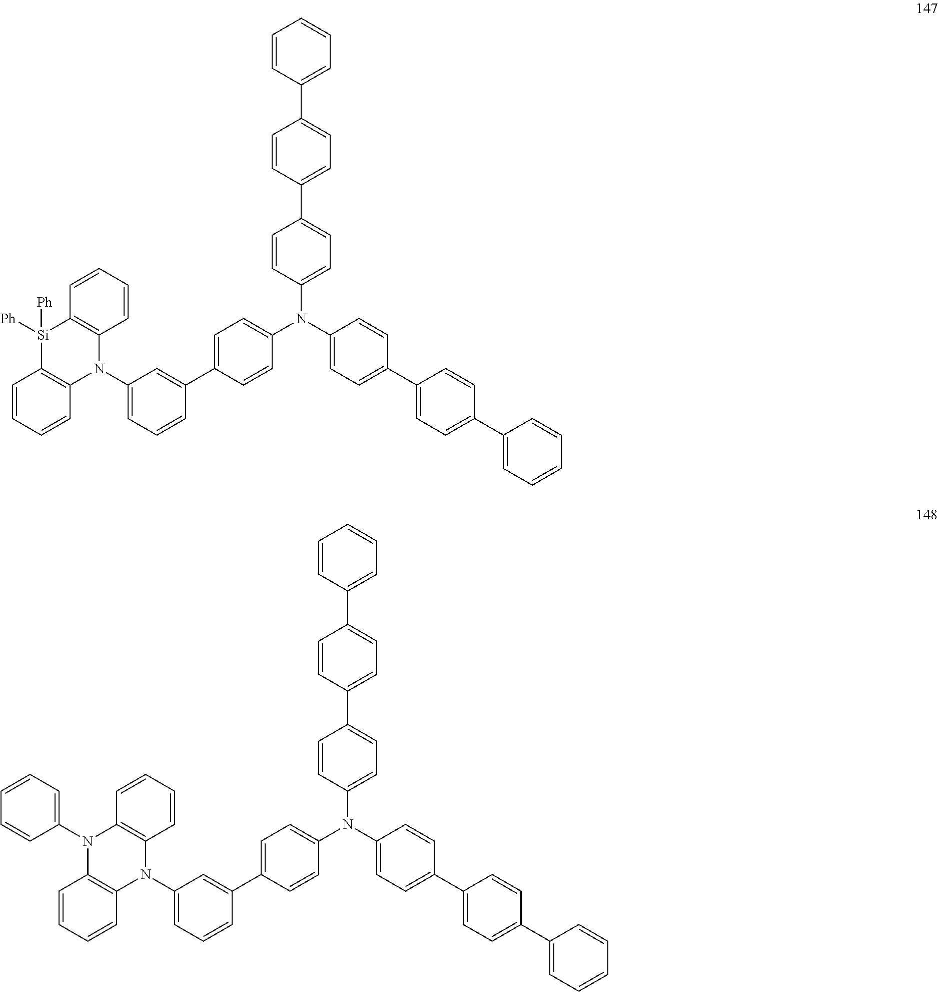 [SMILES: c1ccc(-c2ccc(-c3ccc(N(c4ccc(-c5ccc(-c6ccccc6)cc5)cc4)c4ccc(-c5cccc(N6c7ccccc7[Si](c7ccccc7)(c7ccccc7)c7ccccc76)c5)cc4)cc3)cc2)cc1 ; c1ccc(-c2ccc(-c3ccc(N(c4ccc(-c5ccc(-c6ccccc6)cc5)cc4)c4ccc(-c5cccc(N6c7ccccc7N(c7ccccc7)c7ccccc76)c5)cc4)cc3)cc2)cc1] 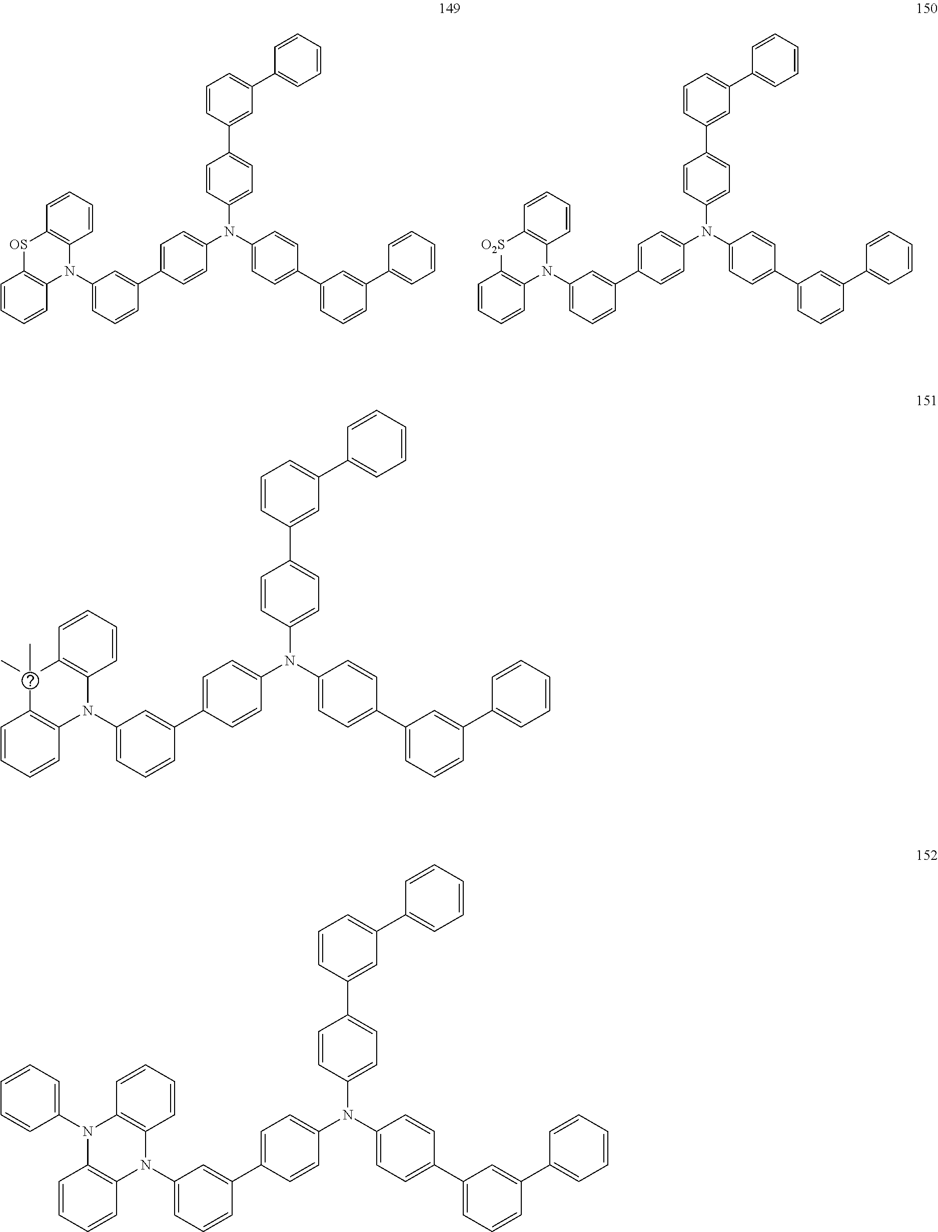 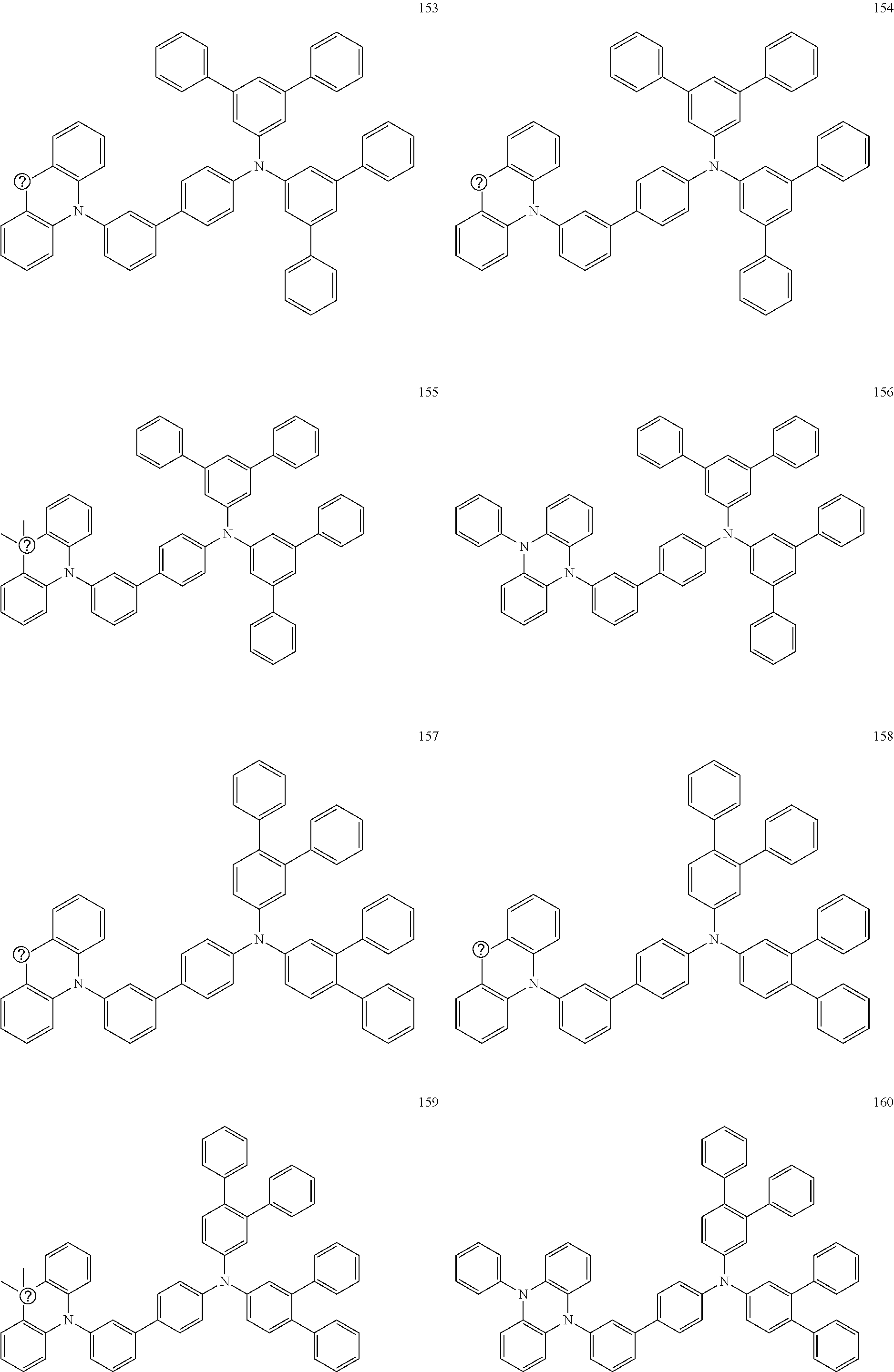 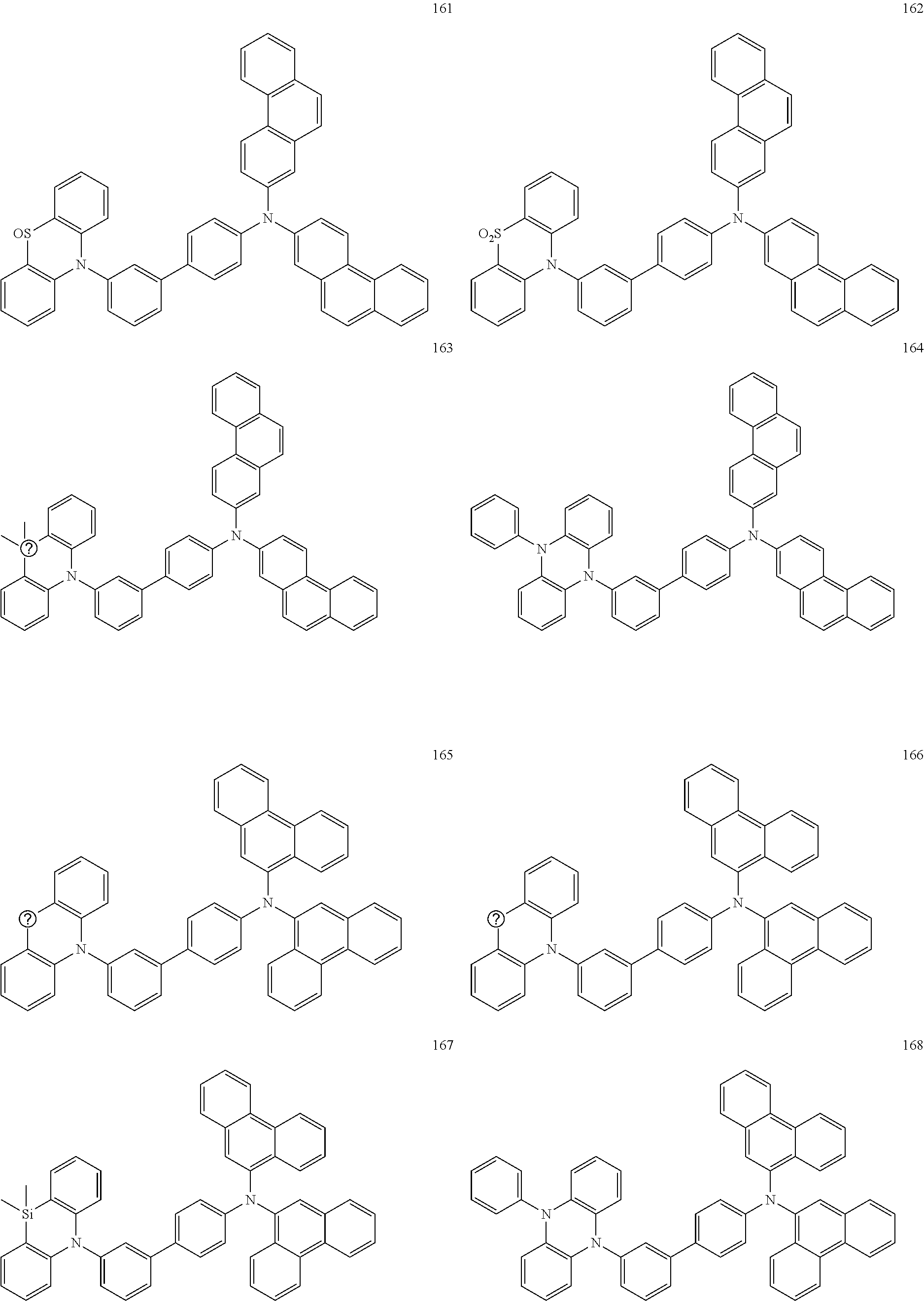 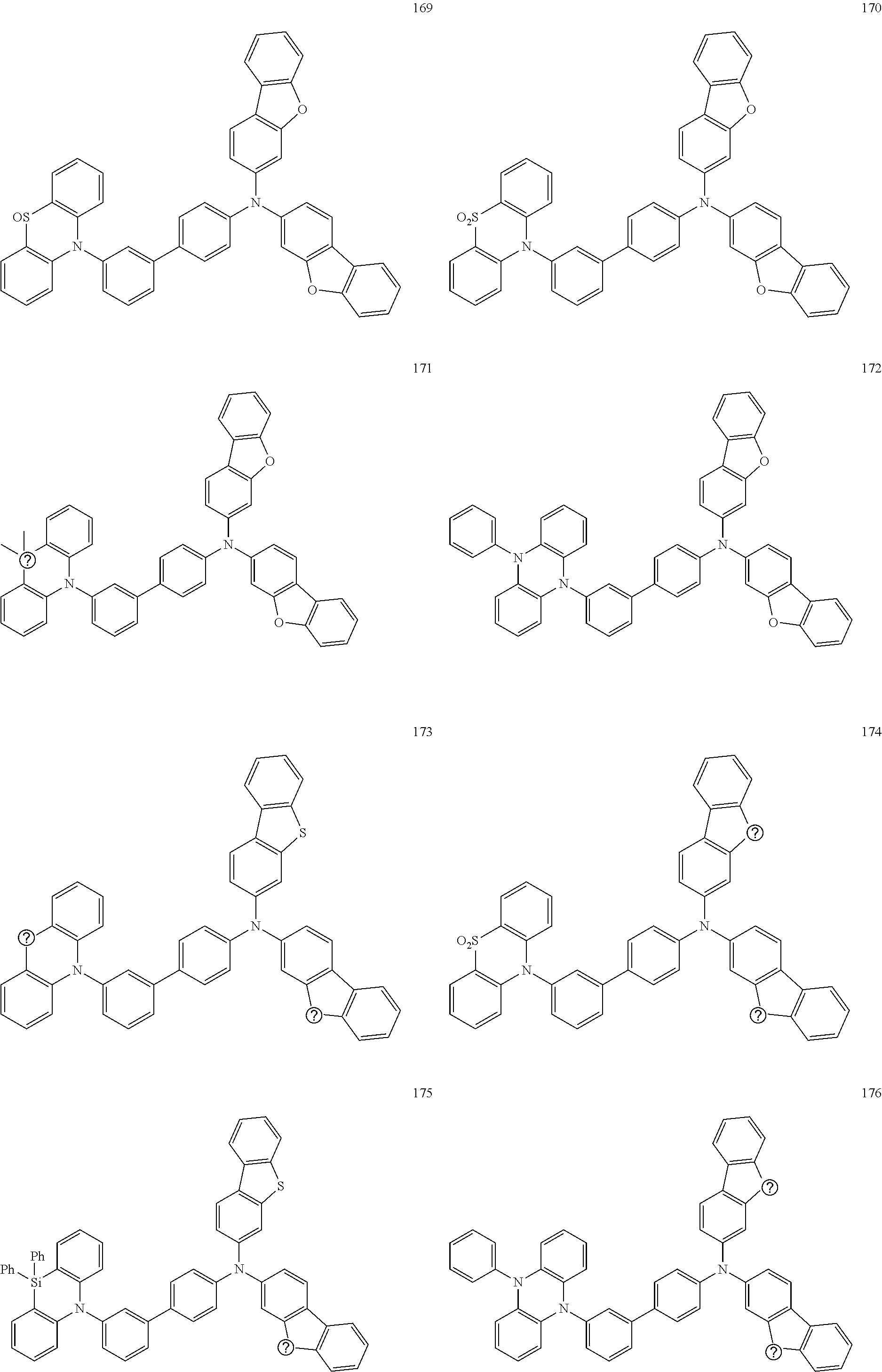 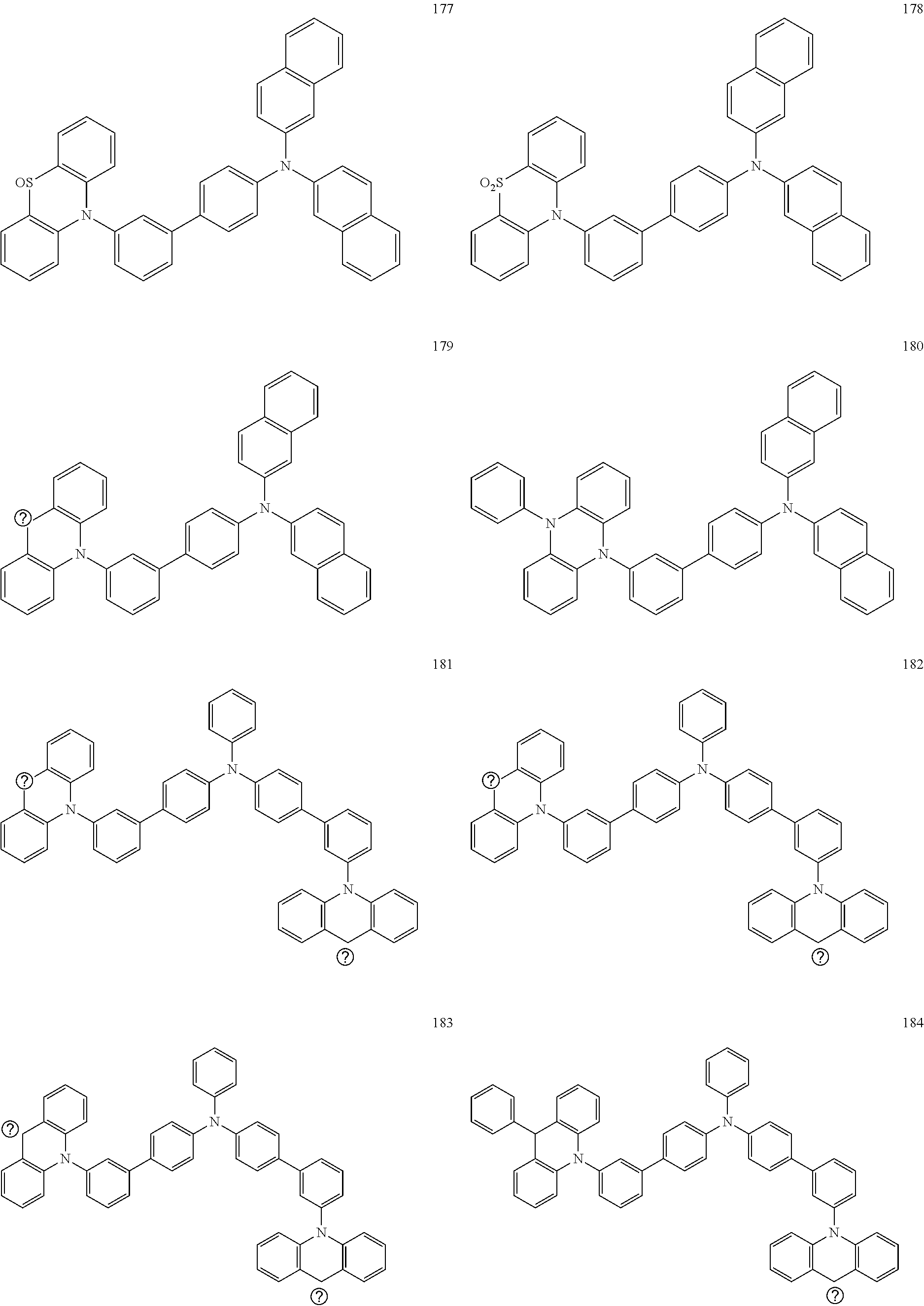 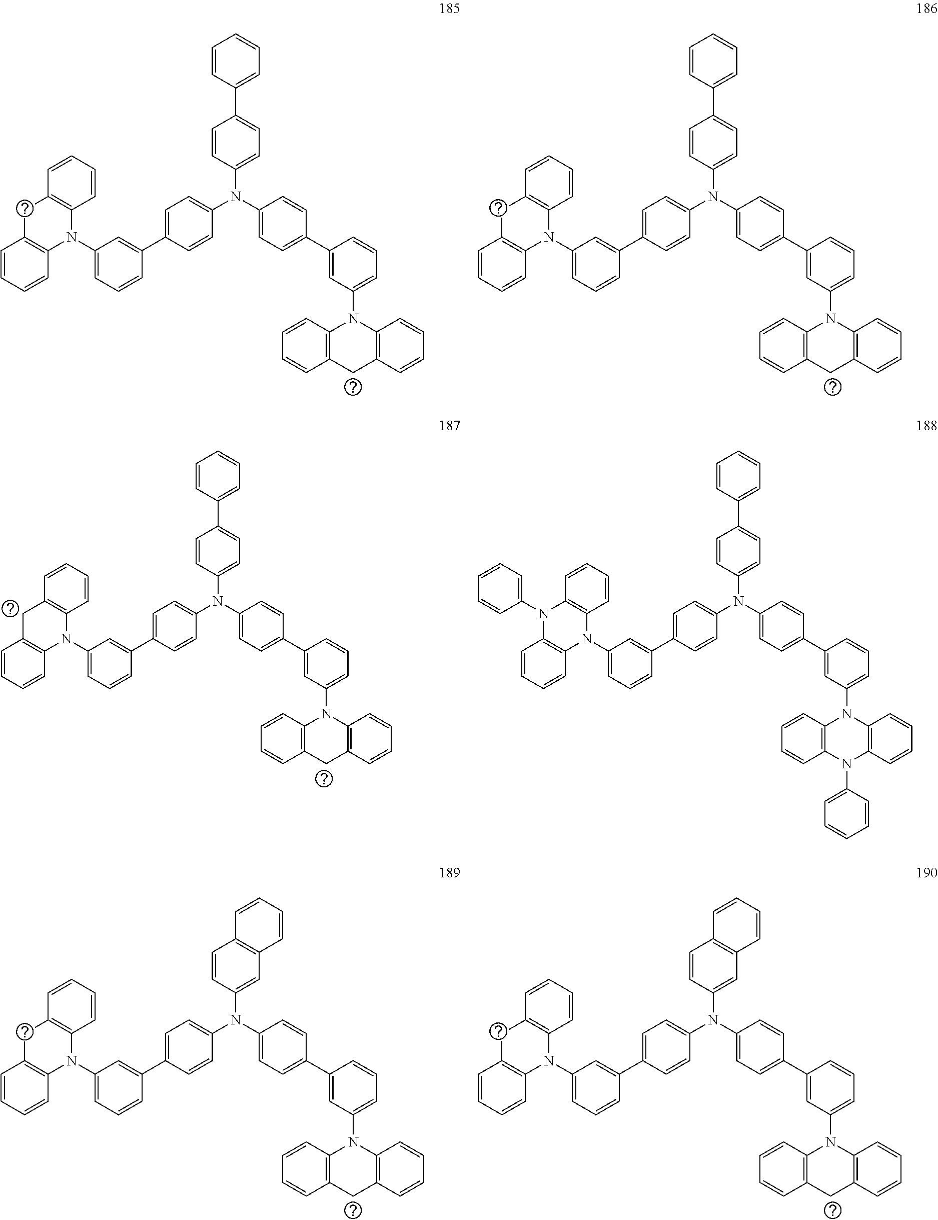 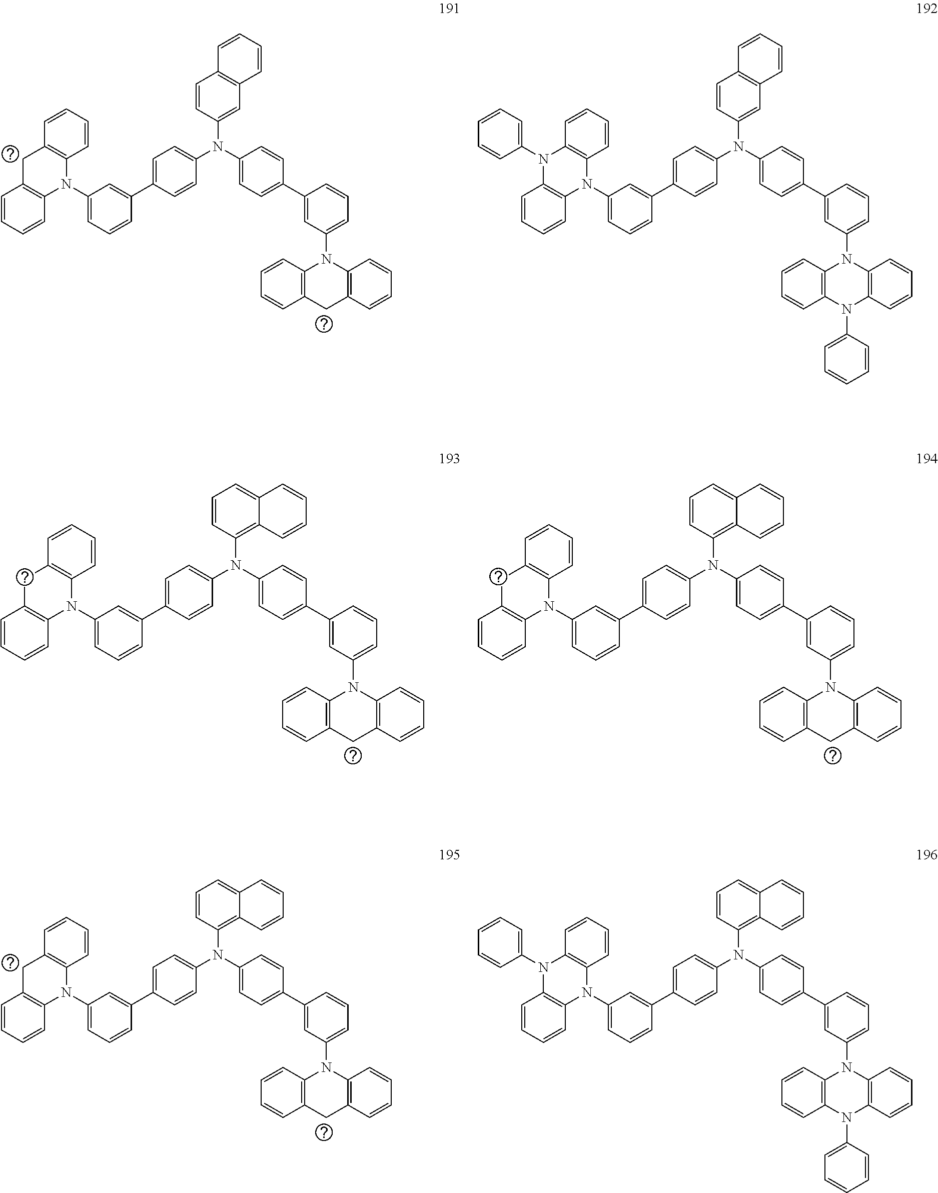 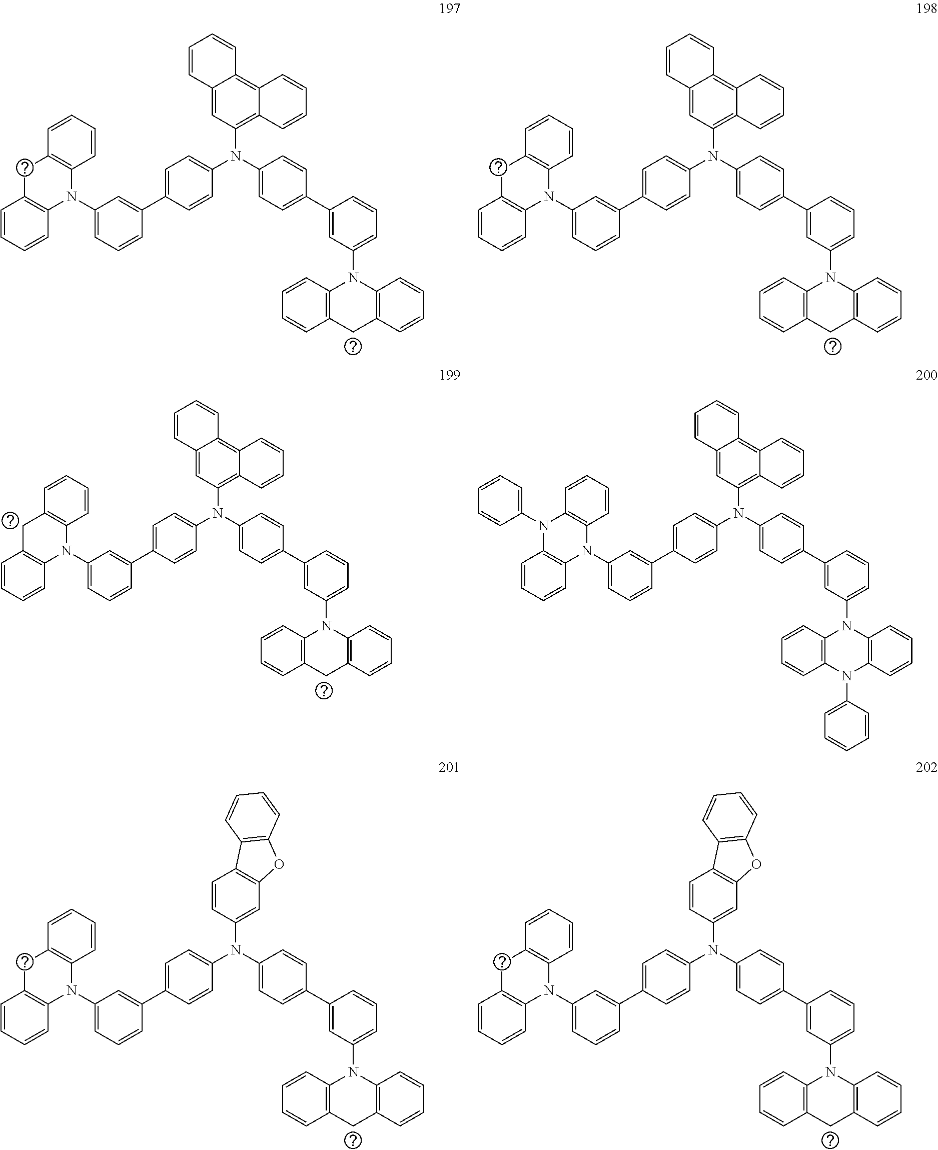 [chemical structure]
